(19) United States
(12) Patent Application Publication
(10) Pub. No.: US 2013/0184465 A1
(43) Pub. Date: Jul. 18, 2013

(54) Process for the synthesis of thio-triazolo-group containing compounds

(71) Applicants: Maximilian Dochnahl, Mannheim (DE); Michael Keil, Freinsheim (DE); Joachim Gebhardt, Wachenheim (DE); Uwe Josef Vogelbacher, Ludwigshafen (DE); Michael Rack, Eppelhiem (DE)

(72) Inventors: Maximilian Dochnahl, Mannheim (DE); Michael Keil, Freinsheim (DE); Joachim Gebhardt, Wachenheim (DE); Uwe Josef Vogelbacher, Ludwigshafen (DE); Michael Rack, Eppelhiem (DE)

(73) Assignee: BASF SE, Ludwigshafen (DE)

(21) Appl. No.: 13/876,326

(22) PCT Filed: Sep. 27, 2011
(86) PCT No.: PCT/EP2011/066777

§ 371 Date: Mar. 27, 2013

Related U.S. Application Data
(60) Provisional application No. 61/387,996, filed on Sep. 30, 2010.

(30) Foreign Application Priority Data
Sep. 30, 2010 (EP) 10183605.4

(51) Int. Cl. C07D 405/02

(52) U.S. Cl. CPC C07D 405/02
USPC 548/100

(57) ABSTRACT

The present invention relates to a process using specific magnesium reagents for providing thio-triazolo group-containing compounds and for the synthesis of precursors therefor. The invention furthermore relates to intermediates and to their preparation.

The present invention relates to a process using specific magnesium reagents for providing thio-triazolo group-containing compounds, in particular pesticidal compounds of the triazole class having phytopathogenic activity, and for the synthesis of precursors therefor. The invention furthermore relates to intermediates and to their preparation.

Magnesium amides and their use are, in principle, known from the literature: See for example WO 2007/082911 and the literature cited therein, for example M.-X. Zhang, P.-E. Eaton, Angew. Chem. Int. Ed. 2002, 41, 2169-2171. The use of lithium salts together with Grignard reagents is known from EP 1 582 523. WO 2007/082911 is particularly directed to mixed magnesium and lithium amides.

Important pesticidal compounds carry a thio-triazolo group. Specific thio-triazole compounds that are known as active ingredients having pesticidal, in particular fungicidal activity, are known, for example, from WO 96/38440. Also WO 2009/077471 (PCT/EP2008/067483), WO 2009/077443 (PCT/EP2008/067394) WO 2009/077500 (PCT/EP2008/067545), WO 2009/077497 (PCT/EP2008/067539), EP 09178224, EP 09178291, EP09178288 describe further specific thio-triazolo compounds. Therein, preparation routes for the disclosed compounds are explained.

In particular, it is known from the literature, for example, to introduce the thio-group into the respective triazole compounds using a strong base such as n-BuLi and sulfur powder. Alternatively, the triazole compounds are reacted with sulfur in the presence of an aprotic polar solvent, such as, for example, an amide (such as dimethylformamide (DMF)) or N-alkylpyrrolidone (such as N-octylpyrrolidone, N-dodecylpyrrolidone or N-methylpyrrolidone (NMP)). See also WO 99/19307, WO 97/06151, WO 97/05119 and WO 96/41804. The disadvantages of these methods are that the yield of the desired products is often not satisfying and that the reaction conditions often do not allow commercial scales. Regarding the reaction using n-BuLi, a further disadvantage is that the reagent is quite expensive and that the reaction has to be carried out at low temperatures, which necessitates special cooling equipment. Upscales are thus elaborate and expensive.

Consequently, the methods known from the literature are sometimes not suitable for the efficient synthesis of substituted thio-triazoles because the yield is not sufficient and/or the reaction conditions and parameters such as temperature and/or reactants are not suitable for an upscale to industrially relevant amounts. For example the reaction that involves strong bases often result in a high amount of side products and low yields of the desired products. Inter alia because some thio-triazolo compounds are promising fungicidally active compounds, there is an ongoing need for alternative processes, that are preferably improved at least in some regards and that make the thio-triazolo compounds as easily available as possible.

It has now surprisingly been found a highly efficient general synthesis for the introduction of sulfur into triazolo group-containing compounds involving the use of a magnesium amide reagent. The inventive process represents a new and general method for obtaining compounds containing a thio-triazolo group.

Thus, according to one aspect the present invention provides a process for the preparation of a thio-triazolo group-containing compound of the formula (I)

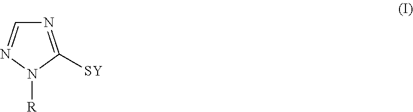

wherein the variables are defined as follows:
R is an organic group;
Y is hydrogen, halogen, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₆-C₁₀)-aryl, a five-, six-, seven-, eight-, nine- or ten-membered aromatic heterocycle that contains one, two, three or four heteroatoms from the group consisting of O, N and S, C(═S)R⁹, SO₂R¹⁰ or CN; wherein
R⁹ is NA⁴A⁵; wherein A⁴, A⁵ independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, C₃-C₈-cycloalkenyl, C₃-C₈-halocycloalkenyl, (C₆-C₁₀)-aryl, a five-, six-, seven-, eight-, nine- or ten-membered aromatic heterocycle that contains one, two, three or four heteroatoms from the group consisting of O, N and S;
R¹⁰ is (C₁-C₈)-alkyl, phenyl-(C₁-C₈)-alkyl or phenyl, where the phenyl groups are in each case unsubstituted or substituted by one, two or three groups independently selected from the group consisting of halogen and (C₁-C₄)-alkyl;
comprising either step (i) together with (ii) and (iii-1) or step (i) together with (ii) and (iii-2); or comprising step (i) together with (iv):
(i) reacting a compound of formula (IV)

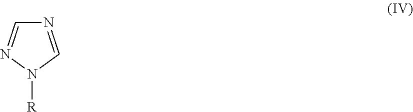

with a reagent (R¹R²N)MgQ (Va), wherein the variables are defined as follows:
Q is (C₁-C₁₀)-alkyl, (C₂-C₁₀)-alkenyl, (C₂-C₁₀)-alkynyl, (C₃-C₈)-cycloalkyl, (C₆-C₁₀)-aryl, wherein the aryl is unsubstituted or substituted by one, two or three groups independently selected from the group consisting of halogen and (C₁-C₄)-alkyl, NR¹R², X¹ or X³.zLiX²,
R¹ and R² are independently selected from (C₁-C₁₀)-alkyl, Si(A¹A²A³), (C₃-C₈)-cycloalkyl and (C₆-C₁₀)-aryl, wherein said groups may bear one, two or three identical or different substituents R^{a}; or
   R¹ and R², together with the nitrogen atom to which they are bonded form a five- or six-membered saturated or partially unsaturated heterocyclyl, which is bonded via N and, if it is a six-membered heterocyclyl, which may contain one or two additional heteroatoms selected from O, N and S, and wherein in each case, the heterocyclyl is un-substituted or carries one, two, three or four substituents selected from the group of halogen, C₁-C₄-alkyl, C₁-C₄-haloalkyl, C₂-C₆-alkenyl, C₂-C₆-haloalkenyl, C₁-C₄-alkoxy, C₁-C₄-alkoxycarbonyl, C₁-C₄-haloalkoxy, C₃-C₆-alkenyloxy, C₃-C₆-haloalkenyloxy and C₆-C₁₀-aryl; wherein
   A¹, A², A³ are independently selected from C₁-C₆-alkyl, trimethylsilyl and phenyl;
   R^{a} is in each case independently selected from halogen, C₁-C₄-alkyl, C₁-C₄-haloalkyl, C₁-C₄-alkoxy, C₁-C₄-haloalkoxy, C₁-C₄-alkylthio, C₁-C₄-alkylamino and di-C₁-C₄-alkylamino;
z is >0;
X¹ halogen; and
X³, X² are independently halogen;
(ii) reacting the reaction mixture resulting from step (i) with sulfur; and
(iii-1) reacting the product of step (ii) with a protonating agent, in order to obtain compounds of formula (I), wherein Y is hydrogen; or
(iii-2) reacting the product of step (ii) with an electrophilic compound Y¹-LG in order to obtain compounds of formula (I), wherein Y is Y¹, wherein
   Y¹ is (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, C(═S)R⁹, SO₂R¹⁰ or CN; wherein R⁹ and R¹⁰ are as defined above; and
   LG is a leaving group;
or
(iv) reacting the reaction mixture resulting from step (i) with an electrophile selected from
   (VI) a disulfide R³—S—S—R³, in order to obtain a compound of formula (I), wherein Y is R³;
   (VII) R⁴—S—SO₂—R⁴, in order to obtain a compound of formula (I), wherein Y is R⁴; or
   (VIII) R⁵—S-Hal, wherein Hal is halogen, in order to obtain a compound of formula (I), wherein Y is R⁵;
wherein
R³, R⁴ are independently from another (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₆-C₁₀)-aryl, a five-, six-, seven-, eight-, nine- or ten-membered aromatic heterocycle that contains one, two, three or four heteroatoms from the group consisting of O, N and S, C(═S)R⁹ or CN; and
R⁵ is halogen, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₆-C₁₀)-aryl or a five-, six-, seven-, eight-, nine- or ten-membered aromatic heterocycle that contains one, two, three or four heteroatoms from the group consisting of O, N and S, or CN.

A key step in the process according to the invention is the deprotonation of the respective triazole compounds (IV) using magnesium amide reagent, thereby resulting in the formation of a compound (IIIa) (see below).

Accordingly, another aspect of the present invention is a process for the preparation of a compound (IIIa)

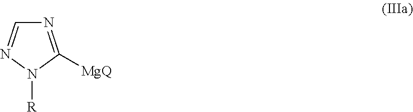

comprising the step
(i) reacting a triazolo compound of formula (IV)

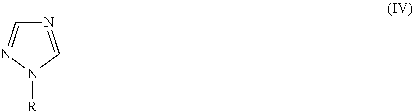

with a magnesium amide reagent (R¹R²N)MgQ (Va), wherein the variables are as defined above.

Compound (IIIa) is usually not isolated from the reaction mixture but directly further reacted to the desired end products (see below). Thus, it represents an intermediate of the overall reaction.

In particular, according to one aspect, compound (IIIa) can be further reacted with a suitable electrophile to result directly in a target thio-triazolo group containing compound of formula (I)

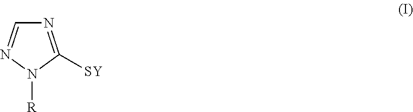

Alternatively, according to the invention, compound (IIIa) can be transformed into a magnesium thiolate (IIa)

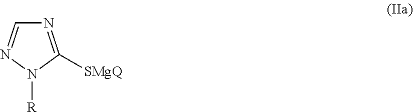

using sulfur. Intermediate (IIa) can be further reacted to a target compound (I) by protonating the magnesium thiolate (IIa) or by reacting the same with a suitable electrophilic compound.

Compound (IIa) is usually not isolated from the reaction mixture but directly further reacted according to the invention. Thus, it represents an intermediate of the overall reaction.

Another aspect of the present invention is a compound of formula (IIIa), wherein Q is NR¹R² or X³.zLiX² and the synthesis and use thereof. Still another aspect of the present invention is a compound of formula (IIa), wherein Q is NR¹R² or X³.zLiX² and the synthesis and use thereof.

The thio-triazolo groups of the general formula (I) can be present in two tautomeric forms (especially, in case “Y” is hydrogen)—the “thiol” form of the formula (Ia) or in the “thiono” form of the formula (Ib)

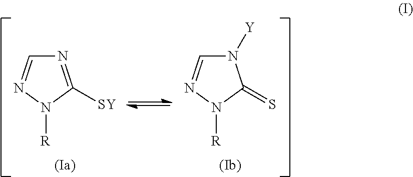

However, for the sake of simplicity, generally only one of the two forms, mostly the “thiol” form is shown here.

In some of the definitions of the symbols in the formulae given herein, collective terms are used which are generally representative of the following substituents:

halogen: fluorine, chlorine, bromine and iodine;
alkyl and the alkyl moieties of composite groups such as, for example, alkylamino: saturated straight-chain or branched hydrocarbon radicals having 1 to 4, 6, 8 or 12 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;
haloalkyl: alkyl as mentioned above, where some or all of the hydrogen atoms in these groups are replaced by halogen atoms as mentioned above; in particular C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl alkenyl and also the alkenyl moieties in composite groups, such as alkenyloxy: unsaturated straight-chain or branched hydrocarbon radicals having 2 to 4, 2 to 6 or 2 to 8 carbon atoms and one double bond in any position. According to the invention, it may be preferred to use small alkenyl groups, such as (C₂-C₄)-alkenyl; on the other hand, it may also be preferred to employ larger alkenyl groups, such as (C₅-C₈)-alkenyl. Examples of alkenyl groups are, for example, C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
haloalkenyl: alkenyl as defined above, where some or all of the hydrogen atoms in these groups are replaced by halogen atoms as described above under haloalkyl, in particular by fluorine, chlorine or bromine;
alkadienyl: unsaturated straight-chain or branched hydrocarbon radicals having 4 to 6 or 4 to 8 carbon atoms and two double bonds in any position;
alkynyl and the alkynyl moieties in composite groups: straight-chain or branched hydrocarbon groups having 2 to 4, 2 to 6 or 2 to 8 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and I-ethyl-1-methyl-2-propynyl;
haloalkynyl: alkynyl as defined above, where some or all of the hydrogen atoms in these groups are replaced by halogen atoms as described above under haloalkyl, in particular by fluorine, chlorine or bromine;
cycloalkyl and also the cycloalkyl moieties in composite groups: mono- or bicyclic saturated hydrocarbon groups having 3 to 8, in particular 3 to 6, carbon ring members, for example C₃-C₆-cycloalkyl, such as cyclopropyl, cyclobutyl, cyclopentyl, cyclohexyl;
halocycloalkyl: cycloalkyl as defined above, where some or all of the hydrogen atoms in these groups are replaced by halogen atoms as described above under haloalkyl, in particular by fluorine, chlorine or bromine;
cycloalkenyl: monocyclic monounsaturated hydrocarbon groups having preferably 3 to 8 or 4 to 6, in particular 5 to 6, carbon ring members, such as cyclopenten-1-yl, cyclopenten-3-yl, cyclohexen-1-yl, cyclohexen-3-yl, cyclohexen-4-yl and the like;
halocycloalkenyl: cycloalkenyl as defined above, where some or all of the hydrogen atoms in these groups are replaced by halogen atoms as described above under haloalkyl, in particular by fluorine, chlorine or bromine;
alkoxy: an alkyl group as defined above which is attached via an oxygen, preferably having 1 to 8, more preferably 2 to 6, carbon atoms. Examples are: methoxy, ethoxy, n-propoxy, 1-methylethoxy, butoxy, 1-methylpropoxy, 2-methylpropoxy or 1,1-dimethylethoxy, and also for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy;
haloalkoxy: alkoxy as defined above, where some or all of the hydrogen atoms in these groups are replaced by halogen atoms as described above under haloalkyl, in particular by fluorine, chlorine or bromine. Examples are OCH₂F, OCHF2, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy; and also 5-fluoropentoxy, 5-chloropentoxy, 5-bromopentoxy, 5-iodopentoxy, undecafluoropentoxy, 6-fluorohexoxy, 6-chlorohexoxy, 6-bromohexoxy, 6-iodohexoxy or dodecafluorohexoxy.

alkylene: divalent unbranched chains of CH₂ groups. Preference is given to (C₁-C₆)-alkylene, more preference to (C₂-C₄)-alkylene; furthermore, it may be preferred to use (C₁-C₃)-alkylene groups. Examples of preferred alkylene radicals are CH₂, CH₂CH₂, CH₂CH₂CH₂, CH₂(CH₂)₂CH₂, CH₂(CH₂)₃CH₂ and CH₂(CH₂)₄CH₂;

a 3-, 4-, 5-, 6-, 7-, 8-, 9- or 10-membered saturated or partially unsaturated heterocycle which contains 1, 2, 3 or 4 heteroatoms from the group consisting of O, N and S, where the heterocycle in question may be attached via a carbon atom or, if present, via a nitrogen atom. According to the invention, it may be preferred for the heterocycle in question to be attached via carbon, on the other hand, it may also be preferred for the heterocycle to be attached via nitrogen. In particular:
a three- or four-membered saturated heterocycle (hereinbelow also referred to as heterocyclyl) which contains one or two heteroatoms from the group consisting of O, N and S as ring members;
a five- or six-membered saturated or partially unsaturated heterocycle which contains one, two, three or four heteroatoms from the group consisting of O, N and S as ring members: for example monocyclic saturated or partially unsaturated heterocycles which, in addition to carbon ring members, contain one, two or three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl and also the corresponding -ylidene radicals;
a seven-membered saturated or partially unsaturated heterocycle which contains one, two, three or four heteroatoms from the group consisting of O, N and S as ring members: for example mono- and bicyclic heterocycles having 7 ring members which, in addition to carbon ring members, contain one, two or three nitrogen atoms and/or one oxygen or sulfur atom or one or two oxygen and/or sulfur atoms, for example tetra- and hexahydroazepinyl, such as 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, hexahydroazepin-1-, -2-, -3- or -4-yl, tetra- and hexahydrooxepinyl such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or -7-yl, hexahydroazepin-1-, -2-, -3- or -4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl and the corresponding ylidene radicals;
   a 5-, 6-, 7-, 8-, 9- or 10-membered aromatic heterocycle which contains 1, 2, 3 or 4 heteroatoms from the group consisting of O, N and S: in particular a five- or six-membered aromatic mono- or bicyclic heterocycle which contains one, two, three or four heteroatoms from the group consisting of O, N and S: the heterocycle in question may be attached via a carbon atom or, if present, via a nitrogen atom. According to the invention, it may be preferred for the heterocycle in question to be attached via carbon, on the other hand, it may also be preferred for the heterocycle to be attached via nitrogen. The heterocycle is in particular:
5-membered heteroaryl which contains one, two, three or four nitrogen atoms or one, two or three nitrogen atoms and/or one sulfur or oxygen atom, where the heteroaryl may be attached via carbon or nitrogen, if present: 5-membered heteroaryl groups which, in addition to carbon atoms, may contain one to four nitrogen atoms or one, two or three nitrogen atoms and/or one sulfur or oxygen atom as ring members, for example furyl, thienyl, pyrrolyl, pyrazolyl, imidazolyl, triazolyl (1,2,3-; 1,2,4-triazolyl), tetrazolyl, oxazolyl, isoxazolyl, 1,3,4-oxadiazolyl, thiazolyl, isothiazolyl and thiadiazolyl, in particular 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl;
-6-membered heteroaryl which contains one, two, three or four, preferably one, two or three, nitrogen atoms, where the heteroaryl may be attached via carbon or nitrogen, if present: 6-ring heteroaryl groups which, in addition to carbon atoms, may contain one to four or one, two or three nitrogen atoms as ring members, for example pyridinyl, pyrimidinyl, pyrazinyl, pyridazinyl, 1,2,3-triazinyl, 1,2,4-triazinyl, 1,3,5-triazinyl, in particular 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

The finding of the present invention is that, ultimately, it offers a very general method for introducing sulfur into triazole groups. Therefore, R in principle can be any organic group that allows carrying out the reaction steps according to the inventive process ultimately resulting in thio-group-containing triazole groups. If necessary, some reactive groups within the “organic group” can be protected via suitable protecting groups. It is within the skill of a person of the art to choose suitable groups and it is general knowledge of the skilled person how to insert and remove such groups.

Important pesticidal compounds carry a thio-triazolo group. In particular, there are compounds of formula (I) known that are effective against phytopathogenic fungi. According to one aspect of the present invention, compounds of formula (I) are active compounds for controlling phytopathogenic fungi. Thus, compounds that can advantageously be synthesized using the new inventive process are for example fungicidal compounds of the triazole compound class.

For example, the inventive process has shown to be very useful for the synthesis of fungicidal thio-triazole compounds of the triazole compound class that contain an epoxide group. Compounds that contain labile functional groups such as an epoxide group can often not be efficiently and/or economically be synthesized via prior art processes. Such compounds are for example described in WO 96/38440, WO 2009/077471 (PCT/EP2008/067483), WO 2009/077443 (PCT/EP2008/067394) WO 2009/077500 (PCT/EP2008/067545) and WO 2009/077497 (PCT/EP2008/067539), EP 09178224, EP 09178291 and EP09178288, wherein these documents also describe the fungicidal activity of said compounds. In said patent applications, also the respective triazole compounds (without sulfur group) and their synthesis are disclosed.

In the following, the meaning of the substituents of the compounds used according to the invention is further defined. Thereby, in each case the substituents are meant to have the given meanings and preferred meaning on their own or in any combination with the meanings or preferred meanings of any other substituent.

Accordingly, in one aspect of the inventive process, R in the compounds (I) and the precursors thereof, in particular in compounds (IV), has the following meaning (1):

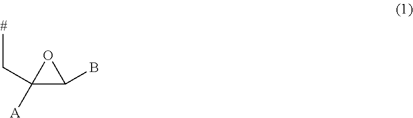

wherein # shall mean the point of attachment to the triazolo group and A and B are as defined as follows:
A or B is a three-, four-, five-, six-, seven-, eight-, nine- or ten-membered saturated or partially unsaturated heterocycle or five-, six-, seven-, eight-, nine- or ten-membered aromatic heterocycle, where the heterocycle contains in each case one, two, three or four heteroatoms from the group consisting of O, N and S; is naphthyl or phenyl;
   and the respective other variable B or A has
one of the meanings mentioned above for A or B or is C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, naphthyl or benzodioxolyl;
   where A and/or B independently of one another are unsubstituted or substituted by one, two, three or four independently selected substituents L; wherein
L is halogen, cyano, nitro, cyanato (OCN), C₁-C₈-alkyl, C₁-C₈-haloalkyl, phenyl-C₁-C₆-alkyloxy, C₂-C₈-alkenyl, C₂-C₈-haloalkenyl, C₂-C₈-alkynyl, C₂-C₈-haloalkynyl, C₄-C₁₀-alkadienyl, C₄-C₁₀-haloalkadienyl, C₁-C₈-alkoxy, C₁-C₈-haloalkoxy, C₁-C₈-alkylcarbonyloxy, C₁-C₈-alkylsulfonyloxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkenyl, C₃-C₈-halocycloalkenyl, C₃-C₈-cycloalkoxy, C₃-C₆-cycloalkenyloxy, hydroxyimino-C₁-C₈-alkyl, C₁-C₆-alkylene, oxy-C₂-C₄-alkylene, oxy-C₁-C₃-alkyleneoxy, C₁-C₈-alkoximino-C₁-C₈-alkyl, C₂-C₈-alkenyloximino-C₁-C₈-alkyl, C₂-C₈-alkynyloximino-C₁-C₈-alkyl, S(═O)ₙA⁶, C(═O)A⁷, C(═S)A⁷, NA⁸A⁹, phenyl-C₁-C₈-alkyl, phenyl, phenyloxy or a five- or six-membered saturated, partially unsaturated or aromatic heterocycle which contains one, two, three or four heteroatoms from the group consisting of O, N and S; where n, A⁶, A⁷, A⁸, A⁹ are as defined below:
n is 0, 1 or 2;
A⁶ is hydrogen, hydroxyl, C₁-C₈-alkyl, C₁-C₈-haloalkyl, amino, C₁-C₈-alkylamino or di-C₁-C₈-alkylamino,
A⁷ is one of the groups mentioned for A₁ or C₂-C₈-alkenyl, C₂-C₈-haloalkenyl, C₂-C₈-alkynyl, C₂-C₈-haloalkynyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkoxy or C₃-C₈-halocycloalkoxy;
A⁸, A⁹ independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, C₃-C₈-cycloalkenyl or C₃-C₈-halocycloalkenyl; where the aliphatic and/or alicyclic and/or aromatic groups of the radical definitions of L for their part may carry one, two, three or four identical or different groups R^{L}:
R^{L} is halogen, cyano, nitro, C₁-C₈-alkyl, C₁-C₈-haloalkyl, C₁-C₈-alkoxy, C₁-C₈-haloalkoxy, C₃-C₈-cycloalkyl, C₃-C₈-halocycloalkyl, C₃-C₈-cycloalkenyl, C₃-C₈-cycloalkoxy, C₃-C₈-halocycloalkoxy, C₁-C₈-alkylcarbonyl, C₁-C₈-alkylcarbonyloxy, C₁-C₈-alkoxycarbonyl, amino, C₁-C₈-alkylamino, di-C₁-C₈-alkylamino.

In group (1) particular preference is given to the following meanings of the substituents, in each case on their own or in combination.

According to one embodiment, A and B independently stand for unsubstituted phenyl or substituted phenyl containing one, two, three or four independently selected substituents L.

According to one specific embodiment, A is unsubstituted phenyl.

According to a further embodiment, A is phenyl, containing one, two, three or four, in particular one or two, independently selected substituents L, wherein L is as defined or as preferably defined herein. According to one aspect of this embodiment, one of the substituents is in 4-position (para) of the phenyl ring. According to a further aspect, L is in each case independently selected from F, Cl, Br, nitro, phenyl, phenoxy, methyl, ethyl, iso-propyl, tert-butyl, methoxy, ethoxy, trifluoromethyl, trichloromethyl, difluoromethyl, difluorochloromethyl, trifluoromethoxy, difluoromethoxy and trifluorochloromethyl. According to another specific aspect, L is in each case independently selected from F, Cl and Br, in particular F and Cl.

According to another embodiment, A is monosubstituted phenyl, containing one substituent L, wherein L is as defined or as preferably defined herein. According to one aspect, said substituent is in para-position.

According to a specific embodiment, A is 3-fluorophenyl.

According to another embodiment, A is phenyl, containing two or three independently selected substituents L.

According to another preferred embodiment of the invention, A is phenyl which is substituted by one F and contains a further substituent L, where the phenyl may additionally contain one or two substituents L selected independently of one another, wherein L is as defined or preferably defined herein. According to a preferred embodiment, A is a group A-1

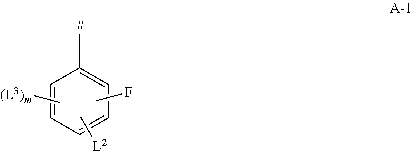

in which # is the point of attachment of the phenyl ring to the oxirane ring; and
L² is selected from the group consisting of F, Cl, NO₂, phenyl, halogenphenyl, phenoxy, halogenphenoxy, C₁-C₄-haloalkyl, C₁-C₄-alkoxy, haloalkoxy and C₁-C₄-haloalkylthio;
L³ is independently selected from the group consisting of F, Cl, Br, NO₂, phenyl, halogenphenyl, phenoxy, halogenphenoxy, C₁-C₄-alkoxy, C₁-C₄-haloalkoxy and C₁-C₄-haloalkylthio; and
m is 0, 1 or 2.

In one embodiment, L² is selected from the group consisting of F, Cl, methyl, methoxy, CF₃, CHF2, OCF₃, OCF₃ and OCHF2. According to a more specific embodiment, L² is F or Cl.

In one embodiment, L³ is independently selected from the group consisting of F, Cl, methyl, methoxy, CF₃, CHF2, OCF₃, OCF₃ or OCHF2. According to a more specific embodiment, L³ is independently F or Cl.

According to a preferred embodiment, m=0. According to a further preferred embodiment, m=1.

In the formula A-1, the fluorine substituent is, according to a preferred embodiment, in the 4-position.

According to still another embodiment, A is disubstituted phenyl, containing exactly two substituents L that are independently selected from each other, wherein L is as defined or as preferably defined herein. In particular, L is in each case independently selected from F, Cl, Br, C₁-C₄-alkyl, C₁-C₄-haloalkyl and C₁-C₄-alkoxy, in particular selected from F, Cl, C₁-C₄-alkyl, C₁-C₄-haloalkyl and C₁-C₄-alkoxy, in particular selected from F, Cl, methyl, trifluoromethyl and methoxy. According to a further aspect of this embodiment, the second substituent L is selected from methyl, methoxy and chloro. According to another aspect, one of the substituents is in the 4-position of the phenyl ring. According to another specific aspect, A is phenyl containing one F and exactly one further substituent L as defined or preferably defined herein.

According to yet a further preferred embodiment, A is disubstituted phenyl which contains one F and a further substituent L selected from the group consisting of Cl, C₁-C₄-alkyl, C₁-C₄-haloalkyl and C₁-C₄-alkoxy, in particular selected from the group consisting of Cl, methyl, trifluoromethyl and methoxy. The second substituent L is specifically selected from the group consisting of methyl, methoxy and chlorine. According to one aspect thereof, one of the substituents is located in the 4-position of the phenyl ring.

According to another specific embodiment, A is 2,4-disubstituted phenyl. According to still another specific embodiment, A is 2,3-disubstituted phenyl. According to still another specific embodiment, A is 2,5-disubstituted phenyl. According to still another specific embodiment, A is 2,6-disubstituted phenyl. According to still another specific embodiment, A is 3,4-disubstituted phenyl. According to still another specific embodiment, A is 3,5-disubstituted phenyl.

According to a further preferred embodiment of the invention, A is phenyl which is substituted by exactly two F. According to one aspect, A is 2,3-difluoro-substituted. According to a further aspect, A is 2,4-difluoro-substituted. According to yet a further aspect, A is 2,5-difluoro-substituted. According to yet a further aspect, A is 2,6-difluoro-substituted. According to yet a further aspect, A is 3,4-difluoro-substituted. According to yet a further aspect, A is 3,5-difluoro-substituted.

According to a further embodiment, A is trisubstituted phenyl containing exactly three independently selected substitutents L, wherein L is as defined or preferably defined herein. According to yet a further embodiment, A is phenyl which is substituted by exactly three F. According to one aspect, A is 2,3,4-trisubstituted, in particular 2,3,4-trifluoro-substituted. According to another aspect, A is 2,3,5-trisubstituted, in particular 2,3,5-trifluoro-substituted. According to still another aspect, A is 2,3,6-trisubstituted, in particular 2,3,6-trifluoro-substituted. According to still another aspect, A is 2,4,6-trisubstituted, in particular 2,4,6-trifluoro-substituted. According to still another aspect, A is 3,4,5-trisubstituted, in particular 3,4,5-trifluoro-substituted. According to still another aspect, A is 2,4,5-trisubstituted, in particular 2,4,5-trifluoro-substituted.

According to a preferred embodiment, B is phenyl, that is unsubstituted or phenyl which contains one, two, three or four independently selected substituents L, wherein L is as defined or preferably defined herein.

According to one embodiment of the invention, B is unsubstituted phenyl.

According to a further embodiment, B is phenyl which contains one, two, three or four independently selected substituents L, wherein L is as defined or preferably defined herein.

According to a further embodiment, B is phenyl which contains one, two or three, preferably one or two, independently selected substituents L, wherein L is as defined or preferably defined herein. According to a specific aspect, L is in each case independently selected from F, Cl, Br, methyl, methoxy and trifluoromethyl. According to still another embodiment, B is phenyl, which contains one, two or three, preferably, one or two, halogen substituents.

According to a further embodiment, B is phenyl which contains one, two, three or four substituents L, wherein L is independently selected from F, Cl, Br, methyl, ethyl, iso-propyl, tert-butyl, methoxy, ethoxy, trifluoromethyl, trichloromethyl, difluoromethyl, difluorochloromethyl, trifluoromethoxy, difluoromethoxy and difluorochloromethyl. According to a specific aspect, L is in each case independently selected from F, Cl and Br.

According to still a further embodiment, B is unsubstituted phenyl or phenyl which contains one, two or three substituents independently selected from halogen, NO₂, amino, C₁-C₄-alkyl, C₁-C₄-alkoxy, C₁-C₄-haloalkoxy, C₁-C₄-alkylamino, C₁-C₄-dialkylamino, thio and C₁-C₄-alkylthio.

According to a further embodiment, B is a phenyl ring that is monosubstituted by one substituent L, where according to a special aspect of this embodiment, L is located in the ortho-position to the point of attachment of the phenyl ring to the oxirane ring. L is as defined or preferably defined herein. According to a further specific embodiment, B is monochloro-substituted phenyl, in particular 2-chlorophenyl.

According to a further embodiment, B is phenyl, which contains two or three, in particular two, independently selected substitutents L, wherein L is as defined or preferably defined herein.

According to a further embodiment of the invention, B is a phenyl ring which contains a substituent L in the ortho-position and furthermore has one further independently selected substituent L. According to one aspect, the phenyl ring is 2,3-disubstituted. According to a further aspect, the phenyl ring is 2,4-disubstituted. According to yet a further aspect, the phenyl ring is 2,5-disubstituted. According to yet a further aspect, the phenyl ring is 2,6-disubstituted.

According to a further embodiment of the invention, B is a phenyl ring which contains a substituent L in the ortho-position and furthermore contains two further independently selected substituents L. According to one aspect, the phenyl ring is 2,3,5-trisubstituted. According to a further aspect, the phenyl ring is 2,3,4-trisubstituted. According to yet a further aspect, the phenyl ring is 2,4,5-trisubstituted.

In a further embodiment, B is phenyl which contains one substituent L in the 2-position and one, two or three further independently selected substituents L. According to a preferred embodiment, B is a group B-1

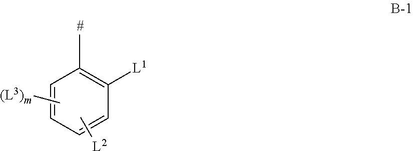

in which # denotes the point of attachment of the phenyl ring to the oxirane ring; and
L¹ is selected from the group consisting of halogen, C₁-C₄-alkyl, C₁-C₄-haloalkyl, C₁-C₄-alkoxy, C₁-C₄-haloalkoxy and C₁-C₄-haloalkylthio, preferably selected from the group consisting of F, Cl, methyl, ethyl, methoxy, ethoxy, CF₃, CHF2, OCF₃, OCHF2 and SCF₃;
L² is selected from the group consisting of halogen, C₁-C₄-alkyl, C₁-C₄-haloalkyl, C₁-C₄-alkoxy, C₁-C₄-haloalkoxy and C₁-C₄-haloalkylthio, preferably selected from the group consisting of F, Cl, methyl, ethyl, methoxy, ethoxy, CF₃, CHF2, OCF₃, OCHF2 and SCF₃;
L³ is independently selected from the group consisting of halogen, C₁-C₄-alkyl, C₁-C₄-haloalkyl, C₁-C₄-alkoxy, C₁-C₄-haloalkoxy and C₁-C₄-haloalkylthio, preferably selected from the group consisting of F, Cl, methyl, ethyl, methoxy, ethoxy, CF₃, CHF2, OCF₃, OCHF2 and SCF₃; and
m is 0, 1 or 2.

According to a preferred embodiment, L¹ is F. According to another preferred embodiment, L¹ is Cl. According to a further preferred embodiment, L¹ is methyl. According to yet a further preferred embodiment, L¹ is methoxy. According to yet a further preferred embodiment, L¹ is CF₃. According to yet a further preferred embodiment, L¹ is OCF₃ or OCHF2. According to a preferred embodiment, in the compounds of the formula I according to the invention, B is thus phenyl which contains a substituent selected from the group consisting of F, Cl, CH₃, OCH₃, CF₃, CHF2, OCF₃ and OCHF2 in the 2-position and one or two further independently selected substituents L.

According to a further preferred embodiment, L² is F. According to another preferred embodiment, L² is Cl. According to a further preferred embodiment, L² is methyl. According to yet a further preferred embodiment, L² is methoxy. According to yet a further preferred embodiment, L² is CF₃. According to yet a further preferred embodiment, L² is OCF₃ or OCHF2.

According to a preferred embodiment, L³ is F. According to another preferred embodiment, L³ is Cl. According to a further preferred embodiment, L³ is methyl. According to yet a further preferred embodiment, L³ is methoxy. According to yet a further preferred embodiment, L³ is CF₃. According to yet a further preferred embodiment, L³ is OCF₃ or OCHF2.

According to a preferred embodiment, m=0; i.e. B is a disubstituted phenyl ring. According to a preferred aspect, B is a 2,3-disubstituted phenyl ring. According to a further preferred aspect, the phenyl ring B is 2,4-disubstituted. According to yet a further preferred aspect, the phenyl ring B is 2,5-disubstituted. According to yet a further preferred aspect, the phenyl ring is 2,6-disubstituted.

According to a further preferred embodiment, m=1; i.e. B is a trisubstituted phenyl ring. According to a preferred aspect, the phenyl ring B is 2,3,5-trisubstituted. According to another preferred further aspect, the phenyl ring B is 2,3,4-trisubstituted. According to yet a further preferred embodiment, the phenyl ring B is 2,4,5-trisubstituted.

Unless indicated otherwise, in group (1) L independently has the following preferred meanings:

According to one embodiment, L is independently selected from the group consisting of halogen, cyano, nitro, cyanato (OCN), C₁-C₄-haloalkyl, C₁-C₄-alkoxy, C₁-C₄-haloalkoxy, C₃-C₆-cycloalkyl, C₃-C₆-halocycloalkyl, S-A⁶, C(═O)A⁷, C(═S)A⁷, NA⁸A⁹; where A⁶, A⁷, A⁸, A⁹ are as defined below:
A⁶ is hydrogen, hydroxy, C₁-C₄-haloalkyl;
A⁷ is one of the groups mentioned under A₁ or C₁-C₄-alkoxy, C₁-C₄-haloalkoxy, C₃-C₆-cycloalkyl, C₃-C₆-halocycloalkyl, C₃-C₆-cycloalkoxy or C₃-C₆-halocycloalkoxy;
A⁸, A⁹ independently of one another are hydrogen, C₁-C₄-alkyl, C₁-C₄-haloalkyl;
where the aliphatic and/or alicyclic groups of the radical definitions of L for their part may carry one, two, three or four identical or different groups R^{L}:
R^{L} is halogen, cyano, nitro, C₁-C₄-alkyl, C₁-C₄-haloalkyl, C₁-C₄-alkoxy, C₁-C₄-haloalkoxy, C₃-C₆-cycloalkyl, C₃-C₆-halocycloalkyl, amino, C₁-C₈-alkylamino, di-C₁-C₈-alkylamino.

Furthermore preferably, L is independently selected from the group consisting of halogen, NO₂, amino, C₁-C₄-alkyl, C₁-C₄-alkoxy, C₁-C₄-haloalkoxy, C₁-C₄-alkylamino, thio and C₁-C₄-alkylthio.

Furthermore preferably, L is independently selected from the group consisting of halogen, C₁-C₄-alkyl, C₁-C₄-alkoxy, C₁-C₄-haloalkoxy and C₁-C₄-haloalkylthio, in particular halogen, C₁-C₄-alkyl and C₁-C₄-haloalkyl.

According to a further preferred embodiment, L is independently selected from the group consisting of F, Cl, Br, CH₃, C₂H₅, i-C₃H₇, t-C₄H₉, OCH₃, OC₂H₅, CF₃, CCl₃, CHF2, CClF2, OCF₃, OCHF2 and SCF₃, in particular selected from the group consisting of F, Cl, CH₃, C₂H₅, OCH₃, OC₂H₅, CF₃, CHF2, OCF₃, OCHF2 and SCF₃. According to one aspect, L is independently selected from the group consisting of F, Cl, CH₃, OCH₃, CF₃, OCF₃ and OCHF2. It may be preferred for L to be independently F or Cl.

According to one preferred embodiment, A and B are as defined as follows:

A phenyl, which is unsubstituted or substituted by one, two or three substituents L that may be the same or different, independently selected from F, Cl, Br, nitro, phenyl, phenoxy, methyl, ethyl, tert-butyl, methoxy, ethoxy, trifluoromethyl, trichloromethyl, difluoromethyl, difluorochloromethyl, trifluoromethoxy, difluoromethoxy and trifluoromethylthio; and

B phenyl, that is substituted by one, two or three substituents L that may be the same or different, independently selected from F, Cl, Br, methyl, ethyl, iso-propyl, tert-butyl, methoxy, ethoxy, trifluoromethyl, trichloromethyl, difluoromethyl, difluorochloromethyl, trifluoromethoxy, difluoromethoxy and trifluoromethylthio.

In specific groups (1) A and B are defined as follows:

A is phenyl, 4-chlorophenyl, 2,4-chlorophenyl, 2-chlorophenyl, 2-fluorophenyl, 4-fluorophenyl, 4-methylphenyl, 3-bromo-4-fluorophenyl, 4-bromophenyl, 3,4-dichlorophenyl, 4-tert-butyl-phenyl, 3-chlorophenyl, 3,5-dichlorophenyl or 4-trifluoromethoxyphenyl and B is 2-chlorophenyl. In one specific group (1) A is 4-flourphenyl and B is 2-chlorophenyl.

A is 4-fluorophenyl and B is 2-difluoromethoxyphenyl.

A is phenyl, 4-chlorophenyl, 2,4-chlorophenyl, 2-chlorophenyl, 2-fluorophenyl, 4-methylphenyl, 4-fluorophenyl, 3-bromo-4-fluorophenyl, 4-bromophenyl, 3,4-dichlorophenyl, 4-tert-butyl-phenyl, 3-chlorophenyl, 3,5-dichlorophenyl or 4-trifluoromethoxyphenyl, and B is 2-fluorophenyl.

A is phenyl, 4-chlorophenyl, 2,4-chlorophenyl, 2-chlorophenyl, 2-fluorophenyl, 4-methylphenyl, 4-fluorophenyl, 3-bromo-4-fluorophenyl, 4-bromophenyl, 3,4-dichlorophenyl, 4-tert-butyl-phenyl, 3-chlorophenyl, 3,5-dichlorophenyl or 4-trifluoromethoxyphenyl, and B is 2-bromophenyl.

In further specific groups (1) A and B are defined as follows:

A is 2,4-difluorophenyl and B is 2-chlorophenyl.

A is 3,4-difluorophenyl and B is 2-chlorophenyl.

A is 2,4-difluorophenyl and B is 2-fluorophenyl.

A is 3,4-difluorophenyl and B is 2-fluorophenyl.

A is 2,4-difluorophenyl and B is 2-trifluoromethylphenyl.

A is 3,4-difluorophenyl and B is 2-trifluoromethylphenyl.

A is 3,4-difluorophenyl and B is 2-methylphenyl

In further specific groups (1) A and B are defined as follows:

A is phenyl and B is 2,4-dichlorophenyl.

A is phenyl and B is 2-fluoro-3-chlorophenyl.

A is phenyl and B is 2,3,4-trichlorophenyl.

A is 4-fluorophenyl and B is 2,4-dichlorophenyl.

A is 4-fluorophenyl and B is 2-fluoro-3-chlorophenyl.

A is 4-fluorophenyl and B is 2,3,4-trichlorophenyl.

A is 2-chlorophenyl and B is 2,4-dichlorophenyl.

A is 2-chlorophenyl and B is 2-fluoro-3-chlorophenyl.

A is 2-chlorophenyl and B is 2,3,4-trichlorophenyl.

The meanings described above of the variables A, B and L for groups (1) apply for R=group (1) in compounds (I) and, unless indicated otherwise, correspondingly to the precursors of the compounds (I) and side products.

The precursors for compounds (I)-(1), such as the respective compounds (IV)-(1) can be synthesized as described in the above mentioned patent applications.

The compounds (IV)-(1) can be prepared in an advantageous manner from compounds of the formula (XI)

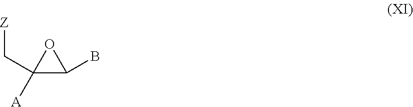

in which Z is a leaving group, such as, for example, halogen (for example Cl or Br) or OSO₂R^{xx}, where R^{xx} is C₁-C₆-alkyl, C₁-C₆-haloalkyl, aryl or substituted aryl; OSO₂R^{xx} is in particular a mesylate, triflate, phenyl or toluenesulfonate group. To obtain compounds of the formula (IV)-(1), compounds of the formula (XI), are reacted with 1,2,4-triazole and a base such as, for example, sodium hydride, for example in DMF. See also, for example, EP 0 421 125 A2.

Compounds of the formula (XI) can be obtained from compounds (XI), wherein Z is a hydroxy group by introducing the leaving group Z by methods known to the person skilled in the art. Thus, the respective hydroxy compound is reacted, for example, with R^{yy}—SO₂Y, where R^{yy} is as defined for formula (XI) and Y is halogen, where R^{yy}—SO₂Y is, for example, mesyl chloride, in the presence of a base (for example NEt₃) (see also EP386557). To obtain compounds (XI), in which Z is halogen, the corresponding hydroxy compound can be reacted with C(Hal)₄ (Hal=Br or Cl) with PPh₃, for example in CH₂Cl₂. Alternatively, SOCl₂/pyridine can be used (see also WO 2005/056548). The hydroxy compounds of the formula (XI) (Z═OH) can be obtained from α,β-disubstituted acroleins of the type of the formula

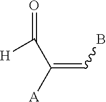

by initial epoxidation, for example with H₂O₂ in the presence of a base such as, for example, NaOH or by reaction with a peracid (for example MCPBA=m-chloroperoxybenzoic acid) or tert-butyl hydroperoxide). The resulting aldehyde can then be reduced to the hydroxy compound, for example with NaBH₄ (see also EP 0 386 557A1). Processes for epoxidation and reduction of the aldehyde group are well known to the person skilled in the art. The double bond can be present either in the (E) or in the (Z) configuration. This is indicated by the zig-zag bond between B and the double bond. The acrolein compounds can be synthesized, for example, analogously to the procedure described in DE3601927. According to one alternative, they can be prepared via an aldol synthesis according to the scheme below:

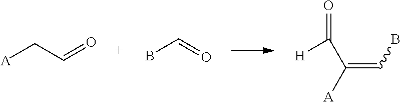

Another way to prepare the compounds (XI) consists in converting the double bond in compounds of the formula

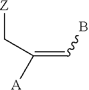

to the epoxide. Suitable epoxidation methods are known to the person skilled in the art. It is possible, for example, to use hydrogen peroxide/maleic anhydride for this purpose. The double bond may be present either in (E) or in (Z) configuration. This is indicated by the zigzag bond between B and the double bond. These compounds can be obtained from compounds

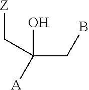

by reacting, for example, with acetic acid/H₂SO₄ in a suitable organic solvent such as, for example, an ether, such as Et₂O or 1,4-dioxane, to form the double bond. Suitable methods are known to the person skilled in the art. These compounds can be obtained, for example, by a Grignard reaction according to the following scheme:

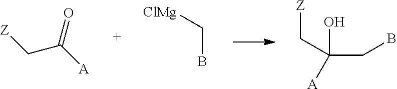

See also EP 409049.

According to the inventive process, the pure enantiomers or a mixture of enantiomers (racemic or enantiomerically enriched) of the reactants, in particular of compounds of formula (IV), can be used. According to a preferred embodiment, the racemic mixture is used. Depending on the use of the respective reactant, in particular of compound of formula (IV), it is possible to obtain compounds of formula (I) having a certain stereochemistry. For example, the following different stereoisomers of compounds (I)-(1) can be obtained using the inventive process:

compound (I)-(1)-a1):
Formula (I)-(1), wherein A is 4-fluoro-phenyl and B is 2-chlorophenyl; Y is SH:
2-[(2S,3S)-3-(2-Chloro-phenyl)-2-(4-fluoro-phenyl)-oxiranylmethyl]-2H-[1,2,4]triazole-3-thiol (“cis”)
   compound (I)-(1)-a2):
   Formula (I)-(1), wherein A is 4-fluoro-phenyl and B is 2-chlorophenyl; Y is SH:
2-[(2R,3R)-3-(2-Chloro-phenyl)-2-(4-fluoro-phenyl)-oxiranylmethyl]-2H-[1,2,4]triazole-3-thiol (“cis”)
   compound (I)-(1)-a3):
   Formula (I)-(1), wherein A is 4-fluoro-phenyl and B is 2-chlorophenyl; Y is SH:
2-[(2S,3R)-3-(2-Chloro-phenyl)-2-(4-fluoro-phenyl)-oxiranylmethyl]-2H-[1,2,4]triazole-3-thiol (“trans”)
   compound (I)-(1)-a4):
   Formula (I)-(1), wherein A is 4-fluoro-phenyl and B is 2-chlorophenyl; Y is SH:
2-[(2R,3S)-3-(2-Chloro-phenyl)-2-(4-fluoro-phenyl)-oxiranylmethyl]-2H-[1,2,4]triazole-3-thiol (“trans”)
   compound (I)-(1)-b1):
   Formula (I)-(1), wherein A is 2,4-difluoro-phenyl and B is 2-chlorophenyl; Y is SH
2-[(2S,3S)-3-(2-Chloro-phenyl)-2-(2,4-difluoro-phenyl)-oxiranylmethyl]-2H-[1,2,4]triazole-3-thiol (“cis”)
   compound (I)-(1)-b2):
   Formula (I)-(1), wherein A is 2,4-difluoro-phenyl and B is 2-chlorophenyl; Y is SH:
2-[(2R,3R)-3-(2-Chloro-phenyl)-2-(2,4-difluoro-phenyl)-oxiranylmethyl]-2H-[1,2,4]triazole-3-thiol (“cis”)
   compound (I)-(1)-b3):
   Formula (I)-(1), wherein A is 2,4-difluoro-phenyl and B is 2-chlorophenyl; Y is SH:
2-[(2S,3R)-3-(2-Chloro-phenyl)-2-(2,4-difluoro-phenyl)-oxiranylmethyl]-2H-[1,2,4]triazole-3-thiol (“trans”)
   compound (I)-(1)-b4):
   Formula (I)-(1), wherein A is 2,4-difluoro-phenyl and B is 2-chlorophenyl; Y is SH:
2-[(2R,3S)-3-(2-Chloro-phenyl)-2-(2,4-difluoro-phenyl)-oxiranylmethyl]-2H-[1,2,4]triazole-3-thiol (“trans”)

With respect to the fungicidal activity of the end products, it may be preferred, if the “trans” diastereomers are synthesized in the inventive process.

According to one specific embodiment of the present invention, R in compounds (IIa) and (IIIa) is a group (1) as defined above, including the specific embodiments thereof. In particular, compounds (IIa)-(1) and compounds (IIIa)-(1),

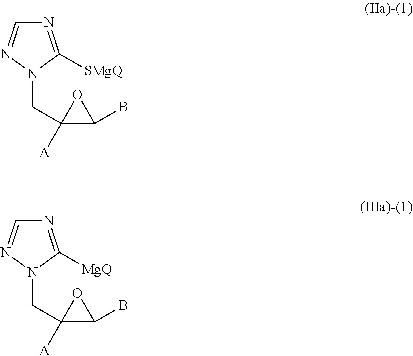

according to the invention compiled in tables 1a to 257a in combination with rows 1 to 2313 of table A below are suitable for the synthesis of the respective fungicides of formula (I) and are obtained by the inventive process. The groups mentioned for a substituent in the tables are furthermore per se, independently of the combination in which they are mentioned, a particularly preferred aspect of the substituent in question.

### Table 1a

Compounds (IIa)-(1) and (IIIa)-(1), wherein A is 2,3-difluorophenyl and the combination of B and Q corresponds in each case to one row of table A (compounds (IIa)-(1).1aA-1 to (IIa)-(1).1aA-2313; (IIIa)-(1).1aA-1 to (IIIa)-(1).1aA-2313)

### Table 2a

Compounds (IIa)-(1) and (IIIa)-(1), wherein A is 2,4-difluorophenyl and the combination of B and Q corresponds in each case to one row of table A (compounds (IIa)-(1).2aA-1 to (IIa)-(1).2aA-2313; (IIIa)-(1).2aA-1 to (IIIa)-(1).2aA-2313)

### Table 3a

Compounds (IIa)-(1) and (IIIa)-(1), wherein A is 2,5-difluorophenyl and the combination of B and Q corresponds in each case to one row of table A (compounds (IIa)-(1).3aA-1 to (IIa)-(1).3aA-2313; (IIIa)-(1).3aA-1 to (IIIa)-(1).3aA-2313)

### Table 4a

Compounds (IIa)-(1) and (IIIa)-(1), wherein A is 2,6-difluorophenyl and the combination of B and Q corresponds in each case to one row of table A (compounds (IIa)-(1).4aA-1 to (IIa)-(1).4aA-2313; (IIIa)-(1).4aA-1 to (IIIa)-(1).4aA-2313)

### Table 5a

Compounds (IIa)-(1) and (IIIa)-(1), wherein A is 3,4-difluorophenyl and the combination of B and Q corresponds in each case to one row of table A (compounds (IIa)-(1).5aA-1 to (IIa)-(1).5aA-2313; (IIIa)-(1).5aA-1 to (IIIa)-(1).5aA-2313)

### Table 6a

Compounds (IIa)-(1) and (IIIa)-(1), wherein A is 3,5-difluorophenyl and the combination of B and Q corresponds in each case to one row of table A (compounds (IIa)-(1).6aA-1 to (IIa)-(1).6aA-2313; (IIIa)-(1).6aA-1 to (IIIa)-(1).6aA-2313)

### Table 7a

Compounds (IIa)-(1) and (IIIa)-(1), wherein A is 2-fluoro-3-chlorophenyl and B corresponds to one row of table A (compounds (IIa)-(1).7aA-1 to (IIa)-(1).7aA-2313; (IIIa)-(1).7aA-1 to (IIIa)-(1).7aA-2313)

### Table 8a

Compounds (IIa)-(1) and (IIIa)-(1), wherein A is 2-fluoro-4-chlorophenyl and the combination of B and Q corresponds in each case to one row of table A (compounds (IIa)-(1).8aA-1 to (IIa)-(1).8aA-2313; (IIIa)-(1).8aA-1 to (IIIa)-(1).8aA-2313)

### Table 9a

Compounds (IIa)-(1) and (IIIa)-(1), wherein A is 2-fluoro-5-chlorophenyl and B corresponds to one row of table A (compounds (IIa)-(1).9aA-1 to (IIa)-(1).9aA-2313; (IIIa)-(1).9aA-1 to (IIIa)-(1).9aA-2313)

### Table 10a

Compounds (IIa)-(1) and (IIIa)-(1), wherein A is 2-fluoro-6-chlorophenyl I and the combination of B and Q corresponds in each case to one row of table A (compounds (IIa)-(1).10aA-1 to (IIa)-(1).10aA-2313; (IIIa)-(1).10aA-1 to (IIIa)-(1).10aA-2313)

### Table 11a

Compounds (IIa)-(1) and (IIIa)-(1), wherein A is 3-fluoro-4-chlorophenyl and the combination of B and Q corresponds in each case to one row of table A (compounds (IIa)-(1).11aA-1 to (IIa)-(1).11aA-2313; (IIIa)-(1).11aA-1 to (IIIa)-(1).11aA-2313)

### Table 12a

Compounds (IIa)-(1) and (IIIa)-(1), wherein A is 3-fluoro-5-chlorophenyl and B corresponds to one row of table A (compounds (IIa)-(1).12aA-1 to (IIa)-(1).12aA-2313; (IIIa)-(1).12aA-1 to (IIIa)-(1).12aA-2313)

### Table 13a

Compounds (IIa)-(1) and (IIIa)-(1), wherein A is 2-chloro-3-fluorophenyl and the combination of B and Q corresponds in each case to one row of table A (compounds (IIa)-(1).13aA-1 to (IIa)-(1).13aA-2313; (IIIa)-(1).13aA-1 to (IIIa)-(1).13aA-2313)

### Table 14a

Compounds (IIa)-(1) and (IIIa)-(1), wherein A is 2-chloro-4-fluorophenyl and B corresponds to one row of table A (compounds (IIa)-(1).14aA-1 to (IIa)-(1).14aA-2313; (IIIa)-(1).14aA-1 to (IIIa)-(1).14aA-2313)

### Table 15a

Compounds (IIa)-(1) and (IIIa)-(1), wherein A is 2-chloro-5-fluorophenyl and the combination of B and Q corresponds in each case to one row of table A (compounds (IIa)-(1).15aA-1 to (IIa)-(1).15aA-2313; (IIIa)-(1).15aA-1 to (IIIa)-(1).15aA-2313)

### Table 16a

Compounds (IIa)-(1) and (IIIa)-(1), wherein A is 3-chloro-4-fluorophenyl and the combination of B and Q corresponds in each case to one row of table A (compounds (IIa)-(1).16aA-1 to (IIa)-(1).16aA-2313; (IIIa)-(1).16aA-1 to (IIIa)-(1).16aA-2313)

### Table 17a

Compounds (IIa)-(1) and (IIIa)-(1), wherein A is 2-methyl-3-fluorophenyl and the combination of B and Q corresponds in each case to one row of table A (compounds (IIa)-(1).17aA-1 to (IIa)-(1).17aA-2313; (IIIa)-(1).17aA-1 to (IIIa)-(1).17aA-2313)

### Table 18a

Compounds (IIa)-(1) and (IIIa)-(1), wherein A is 2-methyl-4-fluorophenyl and the combination of B and Q corresponds in each case to one row of table A (compounds (IIa)-(1).18aA-1 to (IIa)-(1).18aA-2313; (IIIa)-(1).18aA-1 to (IIIa)-(1).18aA-2313)

### Table 19a

Compounds (IIa)-(1) and (IIIa)-(1), wherein A is 2-methyl-5-fluorophenyl and the combination of B and Q corresponds in each case to one row of table A (compounds (IIa)-(1).19aA-1 to (IIa)-(1).19aA-2313; (IIIa)-(1).19aA-1 to (IIIa)-(1).19aA-2313)

### Table 20a

Compounds (IIa)-(1) and (IIIa)-(1), wherein A is 2-methyl-6-fluorophenyl and the combination of B and Q corresponds in each case to one row of table A (compounds (IIa)-(1).20aA-1 to (IIa)-(1).20aA-2313; (IIIa)-(1).20aA-1 to (IIIa)-(1).20aA-2313)
   Table 21a Compounds (IIa)-(1) and (IIIa)-(1), wherein A is 3-methyl-4-fluorophenyl and the combination of B and Q corresponds in each case to one row of table A (compounds (IIa)-(1).21aA-1 to (IIa)-(1).21aA-2313; (IIIa)-(1).21aA-1 to (IIIa)-(1).21aA-2313)

### Table 22a

Compounds (IIa)-(1) and (IIIa)-(1), wherein A is 3-methyl-5-fluorophenyl and the combination of B and Q corresponds in each case to one row of table A (compounds (IIa)-(1).22aA-1 to (IIa)-(1).22aA-2313; (IIIa)-(1).22aA-1 to (IIIa)-(1).22aA-2313)

### Table 23a

Compounds (IIa)-(1) and (IIIa)-(1), wherein A is 2-fluoro-3-methylphenyl and the combination of B and Q corresponds in each case to one row of table A (compounds (IIa)-(1).23aA-1 to (IIa)-(1).23aA-2313; (IIIa)-(1).23aA-1 to (IIIa)-(1).23aA-2313)

### Table 24a

Compounds (IIa)-(1) and (IIIa)-(1), wherein A is 2-fluoro-4-methylphenyl and the combination of B and Q corresponds in each case to one row of table A (compounds (IIa)-(1).24aA-1 to (IIa)-(1).24aA-2313; (IIIa)-(1).24aA-1 to (IIIa)-(1).24aA-2313)

### Table 25a

Compounds (IIa)-(1) and (IIIa)-(1), wherein A is 2-fluoro-5-methylphenyl and the combination of B and Q corresponds in each case to one row of table A (compounds (IIa)-(1).25aA-1 to (IIa)-(1).25aA-2313; (IIIa)-(1).25aA-1 to (IIIa)-(1).25aA-2313)

### Table 26a

Compounds (IIa)-(1) and (IIIa)-(1), wherein A is 3-fluoro-4-methylphenyl and the combination of B and Q corresponds in each case to one row of table A (compounds (IIa)-(1).26aA-1 to (IIa)-(1).26aA-2313; (IIIa)-(1).26aA-1 to (IIIa)-(1).26aA-2313)

### Table 27a

Compounds (IIa)-(1) and (IIIa)-(1), wherein A is 2-ethyl-3-fluorophenyl and the combination of B and Q corresponds in each case to one row of table A (compounds (IIa)-(1).27aA-1 to (IIa)-(1).27aA-2313; (IIIa)-(1).27aA-1 to (IIIa)-(1).27aA-2313)

### Table 28a

Compounds (IIa)-(1) and (IIIa)-(1), wherein A is 2-ethyl-4-fluorophenyl and the combination of B and Q corresponds in each case to one row of table A (compounds (IIa)-(1).28aA-1 to (IIa)-(1).28aA-2313; (IIIa)-(1).28aA-1 to (IIIa)-(1).28aA-2313)

### Table 29a

Compounds (IIa)-(1) and (IIIa)-(1), wherein A is 2-ethyl-5-fluorophenyl and the combination of B and Q corresponds in each case to one row of table A (compounds (IIa)-(1).29aA-1 to (IIa)-(1).29aA-2313; (IIIa)-(1).29aA-1 to (IIIa)-(1).29aA-2313)

### Table 30a

Compounds (IIa)-(1) and (IIIa)-(1), wherein A is 2-ethyl-6-fluorophenyl and the combination of B and Q corresponds in each case to one row of table A (compounds (IIa)-(1).30aA-1 to (IIa)-(1).30aA-2313; (IIIa)-(1).30aA-1 to (IIIa)-(1).30aA-2313)

### Table 31a

Compounds (IIa)-(1) and (IIIa)-(1), wherein A is 3-ethyl-4-fluorophenyl and the combination of B and Q corresponds in each case to one row of table A (compounds (IIa)-(1).31aA-1 to (IIa)-(1).31aA-2313; (IIIa)-(1).31aA-1 to (IIIa)-(1).31aA-2313)

### Table 32a

Compounds (IIa)-(1) and (IIIa)-(1), wherein A is 3-ethyl-5-fluorophenyl and the combination of B and Q corresponds in each case to one row of table A (compounds (IIa)-(1).32aA-1 to (IIa)-(1).32aA-2313; (IIIa)-(1).32aA-1 to (IIIa)-(1).32aA-2313)

### Table 33a

Compounds (IIa)-(1) and (IIIa)-(1), wherein A is 2-fluoro-3-ethylphenyl and the combination of B and Q corresponds in each case to one row of table A (compounds (IIa)-(1).33aA-1 to (IIa)-(1).33aA-2313; (IIIa)-(1).33aA-1 to (IIIa)-(1).33aA-2313)

### Table 34a

Compounds (IIa)-(1) and (IIIa)-(1), wherein A is 2-fluoro-4-ethylphenyl and the combination of B and Q corresponds in each case to one row of table A (compounds (IIa)-(1).34aA-1 to (IIa)-(1).34aA-2313; (IIIa)-(1).34aA-1 to (IIIa)-(1).34aA-2313)

### Table 35a

Compounds (IIa)-(1) and (IIIa)-(1), wherein A is 2-fluoro-5-ethylphenyl and the combination of B and Q corresponds in each case to one row of table A (compounds (IIa)-(1).35aA-1 to (IIa)-(1).35aA-2313; (IIIa)-(1).35aA-1 to (IIIa)-(1).35aA-2313)

### Table 36a

Compounds (IIa)-(1) and (IIIa)-(1), wherein A is 3-fluoro-4-ethylphenyl and the combination of B and Q corresponds in each case to one row of table A (compounds (IIa)-(1).36aA-1 to (IIa)-(1).36aA-2313; (IIIa)-(1).36aA-1 to (IIIa)-(1).36aA-2313)

### Table 37a

Compounds (IIa)-(1) and (IIIa)-(1), wherein A is 2-methoxy-3-fluorophenyl and the combination of B and Q corresponds in each case to one row of table A (compounds (IIa)-(1).37aA-1 to (IIa)-(1).37aA-2313; (IIIa)-(1).37aA-1 to (IIIa)-(1).37aA-2313)

### Table 38a

Compounds (IIa)-(1) and (IIIa)-(1), wherein A is 2-methoxy-4-fluorophenyl and the combination of B and Q corresponds in each case to one row of table A (compounds (IIa)-(1).38aA-1 to (IIa)-(1).38aA-2313; (IIIa)-(1).38aA-1 to (IIIa)-(1).38aA-2313)

### Table 39a

Compounds (IIa)-(1) and (IIIa)-(1), wherein A is 2-methoxy-5-fluorophenyl and the combination of B and Q corresponds in each case to one row of table A (compounds (IIa)-(1).39aA-1 to (IIa)-(1).39aA-2313; (IIIa)-(1).39aA-1 to (IIIa)-(1).39aA-2313)

### Table 40a

Compounds (IIa)-(1) and (IIIa)-(1), wherein A is 2-methoxy-6-fluorophenyl and the combination of B and Q corresponds in each case to one row of table A (compounds (IIa)-(1).40aA-1 to (IIa)-(1).40aA-2313; (IIIa)-(1).40aA-1 to (IIIa)-(1).40aA-2313)

### Table 41a

Compounds (IIa)-(1) and (IIIa)-(1), wherein A is 3-methoxy-4-fluorophenyl and the combination of B and Q corresponds in each case to one row of table A (compounds (IIa)-(1).41aA-1 to (IIa)-(1).41aA-2313; (IIIa)-(1).41aA-1 to (IIIa)-(1).41aA-2313)

### Table 42a

Compounds (IIa)-(1) and (IIIa)-(1), wherein A is 3-methoxy-5-fluorophenyl and the combination of B and Q corresponds in each case to one row of table A (compounds (IIa)-(1).42aA-1 to (IIa)-(1).42aA-2313; (IIIa)-(1).42aA-1 to (IIIa)-(1).42aA-2313)

### Table 43a

Compounds (IIa)-(1) and (IIIa)-(1), wherein A is 2-fluoro-3-methoxyphenyl and the combination of B and Q corresponds in each case to one row of table A (compounds (IIa)-(1).43aA-1 to (IIa)-(1).43aA-2313; (IIIa)-(1).43aA-1 to (IIIa)-(1).43aA-2313)

### Table 44a

Compounds (IIa)-(1) and (IIIa)-(1), wherein A is 2-fluoro-4-methoxyphenyl and the combination of B and Q corresponds in each case to one row of table A (compounds (IIa)-(1).44aA-1 to (IIa)-(1).44aA-2313; (IIIa)-(1).44aA-1 to (IIIa)-(1).44aA-2313)

### Table 45a

Compounds (IIa)-(1) and (IIIa)-(1), wherein A is 2-fluoro-5-methoxyphenyl and the combination of B and Q corresponds in each case to one row of table A (compounds (IIa)-(1).45aA-1 to (IIa)-(1).45aA-2313; (IIIa)-(1).45aA-1 to (IIIa)-(1).45aA-2313)

### Table 46a

Compounds (IIa)-(1) and (IIIa)-(1), wherein A is 3-fluoro-4-methoxyphenyl and the combination of B and Q corresponds in each case to one row of table A (compounds (IIa)-(1).46aA-1 to (IIa)-(1).46aA-2313; (IIIa)-(1).46aA-1 to (IIIa)-(1).46aA-2313)

### Table 47a

Compounds (IIa)-(1) and (IIIa)-(1), wherein A is 3-fluoro-5-methoxyphenyl and the combination of B and Q corresponds in each case to one row of table A (compounds (IIa)-(1).47aA-1 to (IIa)-(1).47aA-2313; (IIIa)-(1).47aA-1 to (IIIa)-(1).47aA-2313)

### Table 48a

Compounds (IIa)-(1) and (IIIa)-(1), wherein A is 2-(trifluoromethyl)-3-fluorophenyl and the combination of B and Q corresponds in each case to one row of table A (compounds (IIa)-(1).48aA-1 to (IIa)-(1).48aA-2313; (IIIa)-(1).48aA-1 to (IIIa)-(1).48aA-2313)

### Table 49a

Compounds (IIa)-(1) and (IIIa)-(1), wherein A is 2-(trifluoromethyl)-4-fluorophenyl and the combination of B and Q corresponds in each case to one row of table A (compounds (IIa)-(1).49aA-1 to (IIa)-(1).49aA-2313; (IIIa)-(1).49aA-1 to (IIIa)-(1).49aA-2313))

### Table 50a

Compounds (IIa)-(1) and (IIIa)-(1), wherein A is 2-(trifluoromethyl)-5-fluorophenyl and the combination of B and Q corresponds in each case to one row of table A (compounds (IIa)-(1).50aA-1 to (IIa)-(1).50aA-2313; (IIIa)-(1).50aA-1 to (IIIa)-(1).50aA-2313)

### Table 51a

Compounds (IIa)-(1) and (IIIa)-(1), wherein A is 2-(trifluoromethyl)-6-fluorophenyl and the combination of B and Q corresponds in each case to one row of table A (compounds (IIa)-(1).51aA-1 to (IIa)-(1).51aA-2313; (IIIa)-(1).51aA-1 to (IIIa)-(1).51aA-2313)

### Table 52a

Compounds (IIa)-(1) and (IIIa)-(1), wherein A is 3-(trifluoromethyl)-4-fluorophenyl and the combination of B and Q corresponds in each case to one row of table A (compounds (IIa)-(1).52aA-1 to (IIa)-(1).52aA-2313; (IIIa)-(1).52aA-1 to (IIIa)-(1).52aA-2313)

### Table 53a

Compounds (IIa)-(1) and (IIIa)-(1), wherein A is 3-(trifluoromethyl)-5-fluorophenyl and the combination of B and Q corresponds in each case to one row of table A (compounds (IIa)-(1).53aA-1 to (IIa)-(1).53aA-2313; (IIIa)-(1).53aA-1 to (IIIa)-(1).53aA-2313)

### Table 54a

Compounds (IIa)-(1) and (IIIa)-(1), wherein A is 2-fluoro-3-(trifluoromethyl)phenyl and the combination of B and Q corresponds in each case to one row of table A (compounds (IIa)-(1).54aA-1 to (IIa)-(1).54aA-2313; (IIIa)-(1).54aA-1 to (IIIa)-(1).54aA-2313))

### Table 55a

Compounds (IIa)-(1) and (IIIa)-(1), wherein A is 2-fluoro-4-(trifluoromethyl)phenyl and the combination of B and Q corresponds in each case to one row of table A (compounds (IIa)-(1).55aA-1 to (IIa)-(1).55aA-2313; (IIIa)-(1).55aA-1 to (IIIa)-(1).55aA-2313)

### Table 56a

Compounds (IIa)-(1) and (IIIa)-(1), wherein A is 2-fluoro-5-(trifluoromethyl)phenyl and the combination of B and Q corresponds in each case to one row of table A (compounds (IIa)-(1).56aA-1 to (IIa)-(1).56aA-2313; (IIIa)-(1).56aA-1 to (IIIa)-(1).56aA-2313))

### Table 57a

Compounds (IIa)-(1) and (IIIa)-(1), wherein A is 3-fluoro-4-(trifluoromethyl)phenyl and the combination of B and Q corresponds in each case to one row of table A (compounds (IIa)-(1).57aA-1 to (IIa)-(1).57aA-2313; (IIIa)-(1).57aA-1 to (IIIa)-(1).57aA-2313))

### Table 58a

Compounds (IIa)-(1) and (IIIa)-(1), wherein A is 2-(trifluoromethoxy)-3-fluorophenyl and the combination of B and Q corresponds in each case to one row of table A (compounds (IIa)-(1).58aA-1 to (IIa)-(1).58aA-2313; (IIIa)-(1).58aA-1 to (IIIa)-(1).58aA-2313)

### Table 59a

Compounds (IIa)-(1) and (IIIa)-(1), wherein A is 2-(trifluoromethoxy)-4-fluorophenyl and the combination of B and Q corresponds in each case to one row of table A (compounds (IIa)-(1).59aA-1 to (IIa)-(1).59aA-2313; (IIIa)-(1).59aA-1 to (IIIa)-(1).59aA-2313)

### Table 60a

Compounds (IIa)-(1) and (IIIa)-(1), wherein A is 2-(trifluoromethoxy)-5-fluorophenyl and the combination of B and Q corresponds in each case to one row of table A (compounds (IIa)-(1).60aA-1 to (IIa)-(1).60aA-2313; (IIIa)-(1).60aA-1 to (IIIa)-(1).60aA-2313)

### Table 61a

Compounds (IIa)-(1) and (IIIa)-(1), wherein A is 2-(trifluoromethoxy)-6-fluorophenyl and the combination of B and Q corresponds in each case to one row of table A (compounds (IIa)-(1).61aA-1 to (IIa)-(1).61aA-2313; (IIIa)-(1).61aA-1 to (IIIa)-(1).61aA-2313))

### Table 62a

Compounds (IIa)-(1) and (IIIa)-(1), wherein A is 3-(trifluoromethoxy)-4-fluorophenyl and the combination of B and Q corresponds in each case to one row of table A (compounds (IIa)-(1).62aA-1 to (IIa)-(1).62aA-2313; (IIIa)-(1).62aA-1 to (IIIa)-(1).62aA-2313))

### Table 63a

Compounds (IIa)-(1) and (IIIa)-(1), wherein A is 3-(trifluoromethoxy)-5-fluorophenyl and the combination of B and Q corresponds in each case to one row of table A (compounds (IIa)-(1).63aA-1 to (IIa)-(1).63aA-2313; (IIIa)-(1).63aA-1 to (IIIa)-(1).63aA-2313)

### Table 64a

Compounds (IIa)-(1) and (IIIa)-(1), wherein A is 2-fluoro-3-(trifluoromethoxy)phenyl and the combination of B and Q corresponds in each case to one row of table A (compounds (IIa)-(1).64aA-1 to (IIa)-(1).64aA-2313; (IIIa)-(1).64aA-1 to (IIIa)-(1).64aA-2313)

### Table 65a

Compounds (IIa)-(1) and (IIIa)-(1), wherein A is 2-fluoro-4-(trifluoromethoxy)phenyl and the combination of B and Q corresponds in each case to one row of table A (compounds (IIa)-(1).65aA-1 to (IIa)-(1).65aA-2313; (IIIa)-(1).65aA-1 to (IIIa)-(1).65aA-2313)

### Table 66a

Compounds (IIa)-(1) and (IIIa)-(1), wherein A is 2-fluoro-5-(trifluoromethoxy)phenyl and the combination of B and Q corresponds in each case to one row of table A (compounds (IIa)-(1).66aA-1 to (IIa)-(1).66aA-2313; (IIIa)-(1).66aA-1 to (IIIa)-(1).66aA-2313)

### Table 67a

Compounds (IIa)-(1) and (IIIa)-(1), wherein A is 3-fluoro-4-(trifluoromethoxy)phenyl and the combination of B and Q corresponds in each case to one row of table A (compounds (IIa)-(1).67aA-1 to (IIa)-(1).67aA-2313; (IIIa)-(1).67aA-1 to (IIIa)-(1).67aA-2313)

### Table 68a

Compounds (IIa)-(1) and (IIIa)-(1), wherein A is 2-(difluoromethoxy)-3-fluorophenyl and the combination of B and Q corresponds in each case to one row of table A (compounds (IIa)-(1).68aA-1 to (IIa)-(1).68aA-2313; (IIIa)-(1).68aA-1 to (IIIa)-(1).68aA-2313)

### Table 69a

Compounds (IIa)-(1) and (IIIa)-(1), wherein A is 2-(difluoromethoxy)-4-fluorophenyl and the combination of B and Q corresponds in each case to one row of table A (compounds (IIa)-(1).69aA-1 to (IIa)-(1).69aA-2313; (IIIa)-(1).69aA-1 to (IIIa)-(1).69aA-2313)

### Table 70a

Compounds (IIa)-(1) and (IIIa)-(1), wherein A is 2-(difluoromethoxy)-5-fluorophenyl and the combination of B and Q corresponds in each case to one row of table A (compounds (IIa)-(1).70aA-1 to (IIa)-(1).70aA-2313; (IIIa)-(1).70aA-1 to (IIIa)-(1).70aA-2313)

### Table 71a

Compounds (IIa)-(1) and (IIIa)-(1), wherein A is 2-(difluoromethoxy)-6-fluorophenyl and the combination of B and Q corresponds in each case to one row of table A (compounds (IIa)-(1).71aA-1 to (IIa)-(1).71aA-2313; (IIIa)-(1).71aA-1 to (IIIa)-(1).71aA-2313)

### Table 72a

Compounds (IIa)-(1) and (IIIa)-(1), wherein A is 3-(difluoromethoxy)-4-fluorophenyl and the combination of B and Q corresponds in each case to one row of table A (compounds (IIa)-(1).72aA-1 to (IIa)-(1).72aA-2313; (IIIa)-(1).72aA-1 to (IIIa)-(1).72aA-2313)

### Table 73a

Compounds (IIa)-(1) and (IIIa)-(1), wherein A is 3-(difluoromethoxy)-5-fluorophenyl and the combination of B and Q corresponds in each case to one row of table A (compounds (IIa)-(1).73aA-1 to (IIa)-(1).73aA-2313; (IIIa)-(1).73aA-1 to (IIIa)-(1).73aA-2313)

### Table 74a

Compounds (IIa)-(1) and (IIIa)-(1), wherein A is 2-fluoro-3-(difluoromethoxy)phenyl and the combination of B and Q corresponds in each case to one row of table A (compounds (IIa)-(1).74aA-1 to (IIa)-(1).74aA-2313; (IIIa)-(1).74aA-1 to (IIIa)-(1).74aA-2313)

### Table 75a

Compounds (IIa)-(1) and (IIIa)-(1), wherein A is 2-fluoro-4-(difluoromethoxy)phenyl and the combination of B and Q corresponds in each case to one row of table A (compounds (IIa)-(1).75aA-1 to (IIa)-(1).75aA-2313; (IIIa)-(1).75aA-1 to (IIIa)-(1).75aA-2313)

### Table 76a

Compounds (IIa)-(1) and (IIIa)-(1), wherein A is 2-fluoro-5-(difluoromethoxy)phenyl and the combination of B and Q corresponds in each case to one row of table A (compounds (IIa)-(1).76aA-1 to (IIa)-(1).76aA-2313; (IIIa)-(1).76aA-1 to (IIIa)-(1).76aA-2313)

### Table 77a

Compounds (IIa)-(1) and (IIIa)-(1), wherein A is 3-fluoro-4-(difluoromethoxy)phenyl and the combination of B and Q corresponds in each case to one row of table A (compounds (IIa)-(1).77aA-1 to (IIa)-(1).77aA-2313; (IIIa)-(1).77aA-1 to (IIIa)-(1).77aA-2313)

### Table 78a

Compounds (IIa)-(1) and (IIIa)-(1), wherein A is 2-(trifluoromethylthio)-3-fluorophenyl and the combination of B and Q corresponds in each case to one row of table A (compounds (IIa)-(1).78aA-1 to (IIa)-(1).78aA-2313; (IIIa)-(1).78aA-1 to (IIIa)-(1).78aA-2313)

### Table 79a

Compounds (IIa)-(1) and (IIIa)-(1), wherein A is 2-(trifluoromethylthio)-4-fluorophenyl and the combination of B and Q corresponds in each case to one row of table A (compounds (IIa)-(1).79aA-1 to (IIa)-(1).79aA-2313; (IIIa)-(1).79aA-1 to (IIIa)-(1).79aA-2313)

### Table 80a

Compounds (IIa)-(1) and (IIIa)-(1), wherein A is 2-(trifluoromethylthio)-5-fluorophenyl and the combination of B and Q corresponds in each case to one row of table A (compounds (IIa)-(1).80aA-1 to (IIa)-(1).80aA-2313; (IIIa)-(1).80aA-1 to (IIIa)-(1).80aA-2313)

### Table 81a

Compounds (IIa)-(1) and (IIIa)-(1), wherein A is 2-(trifluoromethylthio)-6-fluorophenyl and the combination of B and Q corresponds in each case to one row of table A (compounds (IIa)-(1).81aA-1 to (IIa)-(1).81aA-2313; (IIIa)-(1).81aA-1 to (IIIa)-(1).81aA-2313)

### Table 82a

Compounds (IIa)-(1) and (IIIa)-(1), wherein A is 3-(trifluoromethylthio)-4-fluorophenyl and the combination of B and Q corresponds in each case to one row of table A (compounds (IIa)-(1).82aA-1 to (IIa)-(1).82aA-2313; (IIIa)-(1).82aA-1 to (IIIa)-(1).82aA-2313)

### Table 83a

Compounds (IIa)-(1) and (IIIa)-(1), wherein A is 3-(trifluoromethylthio)-5-fluorophenyl and the combination of B and Q corresponds in each case to one row of table A (compounds (IIa)-(1).83aA-1 to (IIa)-(1).83aA-2313; (IIIa)-(1).83aA-1 to (IIIa)-(1).83aA-2313)

### Table 84a

Compounds (IIa)-(1) and (IIIa)-(1), wherein A is 2-fluoro-3-(trifluoromethylthio)phenyl and the combination of B and Q corresponds in each case to one row of table A (compounds (IIa)-(1).84aA-1 to (IIa)-(1).84aA-2313; (IIIa)-(1).84aA-1 to (IIIa)-(1).84aA-2313)

### Table 85a

Compounds (IIa)-(1) and (IIIa)-(1), wherein A is 2-fluoro-4-(trifluoromethylthio)phenyl and the combination of B and Q corresponds in each case to one row of table A (compounds (IIa)-(1).85aA-1 to (IIa)-(1).85aA-2313; (IIIa)-(1).85aA-1 to (IIIa)-(1).85aA-2313)

### Table 86a

Compounds (IIa)-(1) and (IIIa)-(1), wherein A is 2-fluoro-5-(trifluoromethylthio)phenyl and the combination of B and Q corresponds in each case to one row of table A (compounds (IIa)-(1).86aA-1 to (IIa)-(1).86aA-2313; (IIIa)-(1).86aA-1 to (IIIa)-(1).86aA-2313)

### Table 87a

Compounds (IIa)-(1) and (IIIa)-(1), wherein A is 3-fluoro-4-(trifluoromethylthio)phenyl and the combination of B and Q corresponds in each case to one row of table A (compounds (IIa)-(1).87aA-1 to (IIa)-(1).87aA-2313; (IIIa)-(1).87aA-1 to (IIIa)-(1).87aA-2313)

### Table 88a

Compounds (IIa)-(1) and (IIIa)-(1), wherein A is 2,3,4-trifluorophenyl and the combination of B and Q corresponds in each case to one row of table A (compounds (IIa)-(1).88aA-1 to (IIa)-(1).88aA-2313; (IIIa)-(1).88aA-1 to (IIIa)-(1).88aA-2313)

### Table 89a

Compounds (IIa)-(1) and (IIIa)-(1), wherein A is 2,3,5-trifluorophenyl and the combination of B and Q corresponds in each case to one row of table A (compounds (IIa)-(1).89aA-1 to (IIa)-(1).89aA-2313; (IIIa)-(1).89aA-1 to (IIIa)-(1).89aA-2313)

### Table 90a

Compounds (IIa)-(1) and (IIIa)-(1), wherein A is 2,3,6-trifluorophenyl and the combination of B and Q corresponds in each case to one row of table A (compounds (IIa)-(1).90aA-1 to (IIa)-(1).90aA-2313; (IIIa)-(1).90aA-1 to (IIIa)-(1).90aA-2313)

### Table 91a

Compounds (IIa)-(1) and (IIIa)-(1), wherein A is 2,4,5-trifluorophenyl and the combination of B and Q corresponds in each case to one row of table A (compounds (IIa)-(1).91aA-1 to (IIa)-(1).91aA-2313; (IIIa)-(1).91aA-1 to (IIIa)-(1).91aA-2313)

### Table 92a

Compounds (IIa)-(1) and (IIIa)-(1), wherein A is 2,4,6-trifluorophenyl and the combination of B and Q corresponds in each case to one row of table A (compounds (IIa)-(1).92aA-1 to (IIa)-(1).92aA-2313; (IIIa)-(1).92aA-1 to (IIIa)-(1).92aA-2313))

### Table 93a

Compounds (IIa)-(1) and (IIIa)-(1), wherein A is 3,4,5-trifluorophenyl and the combination of B and Q corresponds in each case to one row of table A (compounds (IIa)-(1).93aA-1 to (IIa)-(1).93aA-2313; (IIIa)-(1).93aA-1 to (IIIa)-(1).93aA-2313)

### Table 94a

Compounds (IIa)-(1) and (IIIa)-(1), wherein A is phenyl and the combination of B and Q corresponds in each case to one row of table A (compounds (IIa)-(1).94aA-1 to (IIa)-(1).94aA-2313; (IIIa)-(1).94aA-1 to (IIIa)-(1).94aA-2313)

### Table 95a

Compounds (IIa)-(1) and (IIIa)-(1), wherein A is 2-chlorophenyl and the combination of B and Q corresponds in each case to one row of table A (compounds (IIa)-(1).95aA-1 to (IIa)-(1).95aA-2313; (IIIa)-(1).95aA-1 to (IIIa)-(1).95aA-2313)

### Table 96a

Compounds (IIa)-(1) and (IIIa)-(1), wherein A is 3-chlorophenyl and the combination of B and Q corresponds in each case to one row of table A (compounds (IIa)-(1).96aA-1 to (IIa)-(1).96aA-2313; (IIIa)-(1).96aA-1 to (IIIa)-(1).96aA-2313)

### Table 97a

Compounds (IIa)-(1) and (IIIa)-(1), wherein A is 4-chlorophenyl and the combination of B and Q corresponds in each case to one row of table A (compounds (IIa)-(1).97aA-1 to (IIa)-(1).97aA-2313; (IIIa)-(1).97aA-1 to (IIIa)-(1).97aA-2313)

### Table 98a

Compounds (IIa)-(1) and (IIIa)-(1), wherein A is 2-fluorophenyl and the combination of B and Q corresponds in each case to one row of table A (compounds (IIa)-(1).98aA-1 to (IIa)-(1).98aA-2313; (IIIa)-(1).98aA-1 to (IIIa)-(1).98aA-2313)

### Table 99a

Compounds (IIa)-(1) and (IIIa)-(1), wherein A is 3-fluorophenyl and the combination of B and Q corresponds in each case to one row of table A (compounds (IIa)-(1).99aA-1 to (IIa)-(1).99aA-2313; (IIIa)-(1).99aA-1 to (IIIa)-(1).99aA-2313)

### Table 100a

Compounds (IIa)-(1) and (IIIa)-(1), wherein A is 4-fluorophenyl and the combination of B and Q corresponds in each case to one row of table A (compounds (IIa)-(1).100aA-1 to (IIa)-(1).100aA-2313; (IIIa)-(1).100aA-1 to (IIIa)-(1).100aA-2313)

### Table 101a

Compounds (IIa)-(1) and (IIIa)-(1), wherein A is 2-methylphenyl and the combination of B and Q corresponds in each case to one row of table A (compounds (IIa)-(1).101aA-1 to (IIa)-(1).101aA-2313; (IIIa)-(1).101aA-1 to (IIIa)-(1).101aA-2313))

### Table 102a v

Compounds (IIa)-(1) and (IIIa)-(1), wherein A is 3-methylphenyl and the combination of B and Q corresponds in each case to one row of table A (compounds (IIa)-(1).102aA-1 to (IIa)-(1).102aA-2313; (IIIa)-(1).102aA-1 to (IIIa)-(1).102aA-2313)

### Table 103a

Compounds (IIa)-(1) and (IIIa)-(1), wherein A is 4-methylphenyl and the combination of B and Q corresponds in each case to one row of table A (compounds (IIa)-(1).103aA-1 to (IIa)-(1).103aA-2313; (IIIa)-(1).103aA-1 to (IIIa)-(1).103aA-2313)

### Table 104a

Compounds (IIa)-(1) and (IIIa)-(1), wherein A is 2-ethylphenyl and the combination of B and Q corresponds in each case to one row of table A (compounds (IIa)-(1).104aA-1 to (IIa)-(1).104aA-2313; (IIIa)-(1).104aA-1 to (IIIa)-(1).104aA-2313)

### Table 105a

Compounds (IIa)-(1) and (IIIa)-(1), wherein A is 3-ethylphenyl and the combination of B and Q corresponds in each case to one row of table A (compounds (IIa)-(1).105aA-1 to (IIa)-(1).105aA-2313; (IIIa)-(1).105aA-1 to (IIIa)-(1).105aA-2313)

### Table 106a

Compounds (IIa)-(1) and (IIIa)-(1), wherein A is 4-ethylphenyl and the combination of B and Q corresponds in each case to one row of table A (compounds (IIa)-(1).106aA-1 to (IIa)-(1).106aA-2313; (IIIa)-(1).106aA-1 to (IIIa)-(1).106aA-2313)

### Table 107a

Compounds (IIa)-(1) and (IIIa)-(1), wherein A is 2-methoxyphenyl and the combination of B and Q corresponds in each case to one row of table A (compounds (IIa)-(1).107aA-1 to (IIa)-(1).107aA-2313; (IIIa)-(1).107aA-1 to (IIIa)-(1).107aA-2313)

### Table 108a

Compounds (IIa)-(1) and (IIIa)-(1), wherein A is 3-methoxyphenyl and the combination of B and Q corresponds in each case to one row of table A (compounds (IIa)-(1).108aA-1 to (IIa)-(1).108aA-2313; (IIIa)-(1).108aA-1 to (IIIa)-(1).108aA-2313)

### Table 109a

Compounds (IIa)-(1) and (IIIa)-(1), wherein A is 4-methoxyphenyl and the combination of B and Q corresponds in each case to one row of table A (compounds (IIa)-(1).109aA-1 to (IIa)-(1).109aA-2313; (IIIa)-(1).109aA-1 to (IIIa)-(1).109aA-2313)

### Table 110a

Compounds (IIa)-(1) and (IIIa)-(1), wherein A is 2-trifluoromethylphenyl and the combination of B and Q corresponds in each case to one row of table A (compounds (IIa)-(1).110aA-1 to (IIa)-(1).110aA-2313; (IIIa)-(1).110aA-1 to (IIIa)-(1).110aA-2313))

### Table 111a

Compounds (IIa)-(1) and (IIIa)-(1), wherein A is 3-trifluoromethylphenyl and the combination of B and Q corresponds in each case to one row of table A (compounds (IIa)-(1).111aA-1 to (IIa)-(1).111aA-2313; (IIIa)-(1).111aA-1 to (IIIa)-(1).111aA-2313)

### Table 112a

Compounds (IIa)-(1) and (IIIa)-(1), wherein A is 4-trifluoromethylphenyl and the combination of B and Q corresponds in each case to one row of table A (compounds (IIa)-(1).112aA-1 to (IIa)-(1).112aA-2313; (IIIa)-(1).112aA-1 to (IIIa)-(1).112aA-2313)

### Table 113a

Compounds (IIa)-(1) and (IIIa)-(1), wherein A is 2-trifluoromethoxyphenyl and the combination of B and Q corresponds in each case to one row of table A (compounds (IIa)-(1).113aA-1 to (IIa)-(1).113aA-2313; (IIIa)-(1).113aA-1 to (IIIa)-(1).113aA-2313)

### Table 114a

Compounds (IIa)-(1) and (IIIa)-(1), wherein A is 3-trifluoromethoxyphenyl and the combination of B and Q corresponds in each case to one row of table A (compounds (IIa)-(1).114aA-1 to (IIa)-(1).114aA-2313; (IIIa)-(1).114aA-1 to (IIIa)-(1).114aA-2313)

### Table 115a

Compounds (IIa)-(1) and (IIIa)-(1), wherein A is 4-trifluoromethoxyphenyl and the combination of B and Q corresponds in each case to one row of table A (compounds (IIa)-(1).115aA-1 to (IIa)-(1).115aA-2313; (IIIa)-(1).115aA-1 to (IIIa)-(1).115aA-2313))

### Table 116a

Compounds (IIa)-(1) and (IIIa)-(1), wherein A is 2-difluoromethoxyphenyl and the combination of B and Q corresponds in each case to one row of table A (compounds (IIa)-(1).116aA-1 to (IIa)-(1).116aA-2313; (IIIa)-(1).116aA-1 to (IIIa)-(1).116aA-2313)

### Table 117a

Compounds (IIa)-(1) and (IIIa)-(1), wherein A is 3-difluoromethoxyphenyl and the combination of B and Q corresponds in each case to one row of table A (compounds (IIa)-(1).117aA-1 to (IIa)-(1).117aA-2313; (IIIa)-(1).117aA-1 to (IIIa)-(1).117aA-2313)

### Table 118a

Compounds (IIa)-(1) and (IIIa)-(1), wherein A is 4-difluoromethoxyphenyl and the combination of B and Q corresponds in each case to one row of table A (compounds (IIa)-(1).118aA-1 to (IIa)-(1).118aA-2313; (IIIa)-(1).118aA-1 to (IIIa)-(1).118aA-2313)

### Table 119a

Compounds (IIa)-(1) and (IIIa)-(1), wherein A is 2-trifluoromethylthiophenyl and the combination of B and Q corresponds in each case to one row of table A (compounds (IIa)-(1).119aA-1 to (IIa)-(1).119aA-2313; (IIIa)-(1).119aA-1 to (IIIa)-(1).119aA-2313)

### Table 120a

Compounds (IIa)-(1) and (IIIa)-(1), wherein A is 3-trifluoromethylthiophenyl and the combination of B and Q corresponds in each case to one row of table A (compounds (IIa)-(1).120aA-1 to (IIa)-(1).120aA-2313; (IIIa)-(1).120aA-1 to (IIIa)-(1).120aA-2313)

### Table 121a

Compounds (IIa)-(1) and (IIIa)-(1), wherein A is 4-trifluoromethylthiophenyl and the combination of B and Q corresponds in each case to one row of table A (compounds (IIa)-(1).121aA-1 to (IIa)-(1).121aA-2313; (IIIa)-(1).121aA-1 to (IIIa)-(1).121aA-2313)

### Table 122a

Compounds (IIa)-(1) and (IIIa)-(1), wherein A is 2,3-dichlorophenyl and the combination of B and Q corresponds in each case to one row of table A (compounds (IIa)-(1).122aA-1 to (IIa)-(1).122aA-2313; (IIIa)-(1).122aA-1 to (IIIa)-(1).122aA-2313)

### Table 123a

Compounds (IIa)-(1) and (IIIa)-(1), wherein A is 2,4-dichlorophenyl and the combination of B and Q corresponds in each case to one row of table A (compounds (IIa)-(1).123aA-1 to (IIa)-(1).123aA-2313; (IIIa)-(1).123aA-1 to (IIIa)-(1).123aA-2313)

### Table 124a

Compounds (IIa)-(1) and (IIIa)-(1), wherein A is 2,5-dichlorophenyl and the combination of B and Q corresponds in each case to one row of table A (compounds (IIa)-(1).124aA-1 to (IIa)-(1).124aA-2313; (IIIa)-(1).124aA-1 to (IIIa)-(1).124aA-2313)

### Table 125a

Compounds (IIa)-(1) and (IIIa)-(1), wherein A is 2,6-dichlorophenyl and the combination of B and Q corresponds in each case to one row of table A (compounds (IIa)-(1).125aA-1 to (IIa)-(1).125aA-2313; (IIIa)-(1).125aA-1 to (IIIa)-(1).125aA-2313)

### Table 126a

Compounds (IIa)-(1) and (IIIa)-(1), wherein A is 3,4-dichlorophenyl and the combination of B and Q corresponds in each case to one row of table A (compounds (IIa)-(1).126aA-1 to (IIa)-(1).126aA-2313; (IIIa)-(1).126aA-1 to (IIIa)-(1).126aA-2313)

### Table 127a

Compounds (IIa)-(1) and (IIIa)-(1), wherein A is 3,5-dichlorophenyl and the combination of B and Q corresponds in each case to one row of table A (compounds (IIa)-(1).127aA-1 to (IIa)-(1).127aA-2313; (IIIa)-(1).127aA-1 to (IIIa)-(1).127aA-2313)

### Table 128a

Compounds (IIa)-(1) and (IIIa)-(1), wherein A is 2,3-dimethylphenyl and the combination of B and Q corresponds in each case to one row of table A (compounds (IIa)-(1).128aA-1 to (IIa)-(1).128aA-2313; (IIIa)-(1).128aA-1 to (IIIa)-(1).128aA-2313)

### Table 129a

Compounds (IIa)-(1) and (IIIa)-(1), wherein A is 2,4-dimethylphenyl and the combination of B and Q corresponds in each case to one row of table A (compounds (IIa)-(1).129aA-1 to (IIa)-(1).129aA-2313; (IIIa)-(1).129aA-1 to (IIIa)-(1).129aA-2313)

### Table 130a

Compounds (IIa)-(1) and (IIIa)-(1), wherein A is 2,5-dimethylphenyl and the combination of B and Q corresponds in each case to one row of table A (compounds (IIa)-(1).130aA-1 to (IIa)-(1).130aA-2313; (IIIa)-(1).130aA-1 to (IIIa)-(1).130aA-2313)

### Table 131a

Compounds (IIa)-(1) and (IIIa)-(1), wherein A is 2,6-dimethylphenyl and the combination of B and Q corresponds in each case to one row of table A (compounds (IIa)-(1).131aA-1 to (IIa)-(1).131aA-2313; (IIIa)-(1).131aA-1 to (IIIa)-(1).131aA-2313)

### Table 132a

Compounds (IIa)-(1) and (IIIa)-(1), wherein A is 3,4-dimethylphenyl and the combination of B and Q corresponds in each case to one row of table A (compounds (IIa)-(1).132aA-1 to (IIa)-(1).132aA-2313; (IIIa)-(1).132aA-1 to (IIIa)-(1).132aA-2313)

### Table 133a

Compounds (IIa)-(1) and (IIIa)-(1), wherein A is 3,5-dimethylphenyl and the combination of B and Q corresponds in each case to one row of table A (compounds (IIa)-(1).133aA-1 to (IIa)-(1).133aA-2313; (IIIa)-(1).133aA-1 to (IIIa)-(1).133aA-2313)

### Table 134a

Compounds (IIa)-(1) and (IIIa)-(1), wherein A is 2,3-diethylphenyl and the combination of B and Q corresponds in each case to one row of table A (compounds (IIa)-(1).134aA-1 to (IIa)-(1).134aA-2313; (IIIa)-(1).134aA-1 to (IIIa)-(1).134aA-2313)

### Table 135a

Compounds (IIa)-(1) and (IIIa)-(1), wherein A is 2,4-diethylphenyl and the combination of B and Q corresponds in each case to one row of table A (compounds (IIa)-(1).135aA-1 to (IIa)-(1).135aA-2313; (IIIa)-(1).135aA-1 to (IIIa)-(1).135aA-2313)

### Table 136a

Compounds (IIa)-(1) and (IIIa)-(1), wherein A is 2,5-diethylphenyl and the combination of B and Q corresponds in each case to one row of table A (compounds (IIa)-(1).136aA-1 to (IIa)-(1).136aA-2313; (IIIa)-(1).136aA-1 to (IIIa)-(1).136aA-2313)

### Table 137a

Compounds (IIa)-(1) and (IIIa)-(1), wherein A is 2,6-diethylphenyl and the combination of B and Q corresponds in each case to one row of table A (compounds (IIa)-(1).137aA-1 to (IIa)-(1).137aA-2313; (IIIa)-(1).137aA-1 to (IIIa)-(1).137aA-2313)

### Table 138a

Compounds (IIa)-(1) and (IIIa)-(1), wherein A is 3,4-diethylphenyl and the combination of B and Q corresponds in each case to one row of table A (compounds (IIa)-(1).138aA-1 to (IIa)-(1).138aA-2313; (IIIa)-(1).138aA-1 to (IIIa)-(1).138aA-2313)

### Table 139a

Compounds (IIa)-(1) and (IIIa)-(1), wherein A is 3,5-diethylphenyl and the combination of B and Q corresponds in each case to one row of table A (compounds (IIa)-(1).139aA-1 to (IIa)-(1).139aA-2313; (IIIa)-(1).139aA-1 to (IIIa)-(1).139aA-2313)

### Table 140a

Compounds (IIa)-(1) and (IIIa)-(1), wherein A is 2,3-dimethoxyphenyl and the combination of B and Q corresponds in each case to one row of table A (compounds (IIa)-(1).140aA-1 to (IIa)-(1).140aA-2313; (IIIa)-(1).140aA-1 to (IIIa)-(1).140aA-2313)

### Table 141a

Compounds (IIa)-(1) and (IIIa)-(1), wherein A is 2,4-dimethoxyphenyl and the combination of B and Q corresponds in each case to one row of table A (compounds (IIa)-(1).141aA-1 to (IIa)-(1).141aA-2313; (IIIa)-(1).141aA-1 to (IIIa)-(1).141aA-2313)

### Table 142a

Compounds (IIa)-(1) and (IIIa)-(1), wherein A is 2,5-dimethoxyphenyl and the combination of B and Q corresponds in each case to one row of table A (compounds (IIa)-(1).142aA-1 to (IIa)-(1).142aA-2313; (IIIa)-(1).142aA-1 to (IIIa)-(1).142aA-2313)

### Table 143a

Compounds (IIa)-(1) and (IIIa)-(1), wherein A is 2,6-dimethoxyphenyl and the combination of B and Q corresponds in each case to one row of table A (compounds (IIa)-(1).143aA-1 to (IIa)-(1).143aA-2313; (IIIa)-(1).143aA-1 to (IIIa)-(1).143aA-2313)

### Table 144a

Compounds (IIa)-(1) and (IIIa)-(1), wherein A is 3,4-dimethoxyphenyl and the combination of B and Q corresponds in each case to one row of table A (compounds (IIa)-(1).144aA-1 to (IIa)-(1).144aA-2313; (IIIa)-(1).144aA-1 to (IIIa)-(1).144aA-2313)

### Table 145a

Compounds (IIa)-(1) and (IIIa)-(1), wherein A is 3,5-dimethoxyphenyl and the combination of B and Q corresponds in each case to one row of table A (compounds (IIa)-(1).145aA-1 to (IIa)-(1).145aA-2313; (IIIa)-(1).145aA-1 to (IIIa)-(1).145aA-2313)

### Table 146a

Compounds (IIa)-(1) and (IIIa)-(1), wherein A is 2,3-di(trifluoromethyl)phenyl and the combination of B and Q corresponds in each case to one row of table A (compounds (IIa)-(1).146aA-1 to (IIa)-(1).146aA-2313; (IIIa)-(1).146aA-1 to (IIIa)-(1).146aA-2313)

### Table 147a

Compounds (IIa)-(1) and (IIIa)-(1), wherein A is 2,4-di(trifluoromethyl)phenyl and the combination of B and Q corresponds in each case to one row of table A (compounds (IIa)-(1).147aA-1 to (IIa)-(1).147aA-2313; (IIIa)-(1).147aA-1 to (IIIa)-(1).147aA-2313)

### Table 148a

Compounds (IIa)-(1) and (IIIa)-(1), wherein A is 2,5-di(trifluoromethyl)phenyl and the combination of B and Q corresponds in each case to one row of table A (compounds (IIa)-(1).148aA-1 to (IIa)-(1).148aA-2313; (IIIa)-(1).148aA-1 to (IIIa)-(1).148aA-2313)

### Table 149a

Compounds (IIa)-(1) and (IIIa)-(1), wherein A is 2,6-di(trifluoromethyl)phenyl and the combination of B and Q corresponds in each case to one row of table A (compounds (IIa)-(1).149aA-1 to (IIa)-(1).149aA-2313; (IIIa)-(1).149aA-1 to (IIIa)-(1).149aA-2313)

### Table 150a

Compounds (IIa)-(1) and (IIIa)-(1), wherein A is 3,4-di(trifluoromethyl)phenyl and the combination of B and Q corresponds in each case to one row of table A (compounds (IIa)-(1).150aA-1 to (IIa)-(1).150aA-2313; (IIIa)-(1).150aA-1 to (IIIa)-(1).150aA-2313)

### Table 151a

Compounds (IIa)-(1) and (IIIa)-(1), wherein A is 3,5-di(trifluoromethyl)phenyl and the combination of B and Q corresponds in each case to one row of table A (compounds (IIa)-(1).151aA-1 to (IIa)-(1).151aA-2313; (IIIa)-(1).151aA-1 to (IIIa)-(1).151aA-2313)

### Table 152a

Compounds (IIa)-(1) and (IIIa)-(1), wherein A is 2,3-di(trifluoromethoxy)phenyl and the combination of B and Q corresponds in each case to one row of table A (compounds (IIa)-(1).152aA-1 to (IIa)-(1).152aA-2313; (IIIa)-(1).152aA-1 to (IIIa)-(1).152aA-2313)

### Table 153a

Compounds (IIa)-(1) and (IIIa)-(1), wherein A is 2,4-di(trifluoromethoxy)phenyl and the combination of B and Q corresponds in each case to one row of table A (compounds (IIa)-(1).153aA-1 to (IIa)-(1).153aA-2313; (IIIa)-(1).153aA-1 to (IIIa)-(1).153aA-2313)

### Table 154a

Compounds (IIa)-(1) and (IIIa)-(1), wherein A is 2,5-di(trifluoromethoxy)phenyl and the combination of B and Q corresponds in each case to one row of table A (compounds (IIa)-(1).154aA-1 to (IIa)-(1).154aA-2313; (IIIa)-(1).154aA-1 to (IIIa)-(1).154aA-2313)

### Table 155a

Compounds (IIa)-(1) and (IIIa)-(1), wherein A is 2,6-di(trifluoromethoxy)phenyl and the combination of B and Q corresponds in each case to one row of table A (compounds (IIa)-(1).155aA-1 to (IIa)-(1).155aA-2313; (IIIa)-(1).155aA-1 to (IIIa)-(1).155aA-2313)

### Table 156a

Compounds (IIa)-(1) and (IIIa)-(1), wherein A is 3,4-di(trifluoromethoxy)phenyl and the combination of B and Q corresponds in each case to one row of table A (compounds (IIa)-(1).156aA-1 to (IIa)-(1).156aA-2313; (IIIa)-(1).156aA-1 to (IIIa)-(1).156aA-2313)

### Table 157a

Compounds (IIa)-(1) and (IIIa)-(1), wherein A is 3,5-di(trifluoromethoxy)phenyl and the combination of B and Q corresponds in each case to one row of table A (compounds (IIa)-(1).157aA-1 to (IIa)-(1).157aA-2313; (IIIa)-(1).157aA-1 to (IIIa)-(1).157aA-2313)

### Table 158a

Compounds (IIa)-(1) and (IIIa)-(1), wherein A is 2,3-di(difluoromethoxy)phenyl and the combination of B and Q corresponds in each case to one row of table A (compounds (IIa)-(1).158aA-1 to (IIa)-(1).158aA-2313; (IIIa)-(1).158aA-1 to (IIIa)-(1).158aA-2313)

### Table 159a

Compounds (IIa)-(1) and (IIIa)-(1), wherein A is 2,4-di(difluoromethoxy)phenyl and the combination of B and Q corresponds in each case to one row of table A (compounds (IIa)-(1).159aA-1 to (IIa)-(1).159aA-2313; (IIIa)-(1).159aA-1 to (IIIa)-(1).159aA-2313)

### Table 160a

Compounds (IIa)-(1) and (IIIa)-(1), wherein A is 2,5-di(difluoromethoxy)phenyl and the combination of B and Q corresponds in each case to one row of table A (compounds (IIa)-(1).160aA-1 to (IIa)-(1).160aA-2313; (IIIa)-(1).160aA-1 to (IIIa)-(1).160aA-2313)

### Table 161a

Compounds (IIa)-(1) and (IIIa)-(1), wherein A is 2,6-di(difluoromethoxy)phenyl and the combination of B and Q corresponds in each case to one row of table A (compounds (IIa)-(1).161aA-1 to (IIa)-(1).161aA-2313; (IIIa)-(1).161aA-1 to (IIIa)-(1).161aA-2313)

### Table 162a

Compounds (IIa)-(1) and (IIIa)-(1), wherein A is 3,4-di(difluoromethoxy)phenyl and the combination of B and Q corresponds in each case to one row of table A (compounds (IIa)-(1).162aA-1 to (IIa)-(1).162aA-2313; (IIIa)-(1).162aA-1 to (IIIa)-(1).162aA-2313)

### Table 163a

Compounds (IIa)-(1) and (IIIa)-(1), wherein A is 3,5-di(difluoromethoxy)phenyl and the combination of B and Q corresponds in each case to one row of table A (compounds (IIa)-(1).163aA-1 to (IIa)-(1).163aA-2313; (IIIa)-(1).163aA-1 to (IIIa)-(1).163aA-2313)

### Table 164a

Compounds (IIa)-(1) and (IIIa)-(1), wherein A is 2,3-di(trifluoromethylthio)phenyl and the combination of B and Q corresponds in each case to one row of table A (compounds (IIa)-(1).164aA-1 to (IIa)-(1).164aA-2313; (IIIa)-(1).164aA-1 to (IIIa)-(1).164aA-2313)

### Table 165a

Compounds (IIa)-(1) and (IIIa)-(1), wherein A is 2,4-di(trifluoromethylthio)phenyl and the combination of B and Q corresponds in each case to one row of table A (compounds (IIa)-(1).165aA-1 to (IIa)-(1).165aA-2313; (IIIa)-(1).165aA-1 to (IIIa)-(1).165aA-2313)

### Table 166a

Compounds (IIa)-(1) and (IIIa)-(1), wherein A is 2,5-di(trifluoromethylthio)phenyl and the combination of B and Q corresponds in each case to one row of table A (compounds (IIa)-(1).166aA-1 to (IIa)-(1).166aA-2313; (IIIa)-(1).166aA-1 to (IIIa)-(1).166aA-2313)

### Table 167a

Compounds (IIa)-(1) and (IIIa)-(1), wherein A is 2,6-di(trifluoromethylthio)phenyl and the combination of B and Q corresponds in each case to one row of table A (compounds (IIa)-(1).167aA-1 to (IIa)-(1).167aA-2313; (IIIa)-(1).167aA-1 to (IIIa)-(1).167aA-2313)

### Table 168a

Compounds (IIa)-(1) and (IIIa)-(1), wherein A is 3,4-di(trifluoromethylthio)phenyl and the combination of B and Q corresponds in each case to one row of table A (compounds (IIa)-(1).168aA-1 to (IIa)-(1).168aA-2313; (IIIa)-(1).168aA-1 to (IIIa)-(1).168aA-2313)

### Table 169a

Compounds (IIa)-(1) and (IIIa)-(1), wherein A is 3,5-di(trifluoromethylthio)phenyl and the combination of B and Q corresponds in each case to one row of table A (compounds (IIa)-(1).169aA-1 to (IIa)-(1).169aA-2313; (IIIa)-(1).169aA-1 to (IIIa)-(1).169aA-2313)

### Table 170a

Compounds (IIa)-(1) and (IIIa)-(1), wherein A is 2-methyl-3-chlorophenyl and the combination of B and Q corresponds in each case to one row of table A (compounds (IIa)-(1).170aA-1 to (IIa)-(1).170aA-2313; (IIIa)-(1).170aA-1 to (IIIa)-(1).170aA-2313)

### Table 171a

Compounds (IIa)-(1) and (IIIa)-(1), wherein A is 2-methyl-4-chlorophenyl and the combination of B and Q corresponds in each case to one row of table A (compounds (IIa)-(1).171aA-1 to (IIa)-(1).171aA-2313; (IIIa)-(1).171aA-1 to (IIIa)-(1).171aA-2313)

### Table 172a

Compounds (IIa)-(1) and (IIIa)-(1), wherein A is 2-methyl-5-chlorophenyl and the combination of B and Q corresponds in each case to one row of table A (compounds (IIa)-(1).172aA-1 to (IIa)-(1).172aA-2313; (IIIa)-(1).172aA-1 to (IIIa)-(1).172aA-2313)

### Table 173a

Compounds (IIa)-(1) and (IIIa)-(1), wherein A is 2-methyl-6-chlorophenyl and the combination of B and Q corresponds in each case to one row of table A (compounds (IIa)-(1).173aA-1 to (IIa)-(1).173aA-2313; (IIIa)-(1).173aA-1 to (IIIa)-(1).173aA-2313)

### Table 174a

Compounds (IIa)-(1) and (IIIa)-(1), wherein A is 3-methyl-4-chlorophenyl and the combination of B and Q corresponds in each case to one row of table A (compounds (IIa)-(1).174aA-1 to (IIa)-(1).174aA-2313; (IIIa)-(1).174aA-1 to (IIIa)-(1).174aA-2313)

### Table 175a

Compounds (IIa)-(1) and (IIIa)-(1), wherein A is 3-methyl-5-chlorophenyl and the combination of B and Q corresponds in each case to one row of table A (compounds (IIa)-(1).175aA-1 to (IIa)-(1).175aA-2313; (IIIa)-(1).175aA-1 to (IIIa)-(1).175aA-2313)

### Table 176a

Compounds (IIa)-(1) and (IIIa)-(1), wherein A is 2-chloro-3-methylphenyl and the combination of B and Q corresponds in each case to one row of table A (compounds (IIa)-(1).176aA-1 to (IIa)-(1).176aA-2313; (IIIa)-(1).176aA-1 to (IIIa)-(1).176aA-2313)

### Table 177a

Compounds (IIa)-(1) and (IIIa)-(1), wherein A is 2-chloro-4-methylphenyl and the combination of B and Q corresponds in each case to one row of table A (compounds (IIa)-(1).177aA-1 to (IIa)-(1).177aA-2313; (IIIa)-(1).177aA-1 to (IIIa)-(1).177aA-2313)

### Table 178a

Compounds (IIa)-(1) and (IIIa)-(1), wherein A is 2-chloro-5-methylphenyl and the combination of B and Q corresponds in each case to one row of table A (compounds (IIa)-(1).178aA-1 to (IIa)-(1).178aA-2313; (IIIa)-(1).178aA-1 to (IIIa)-(1).178aA-2313)

### Table 179a

Compounds (IIa)-(1) and (IIIa)-(1), wherein A is 3-chloro-4-methylphenyl and the combination of B and Q corresponds in each case to one row of table A (compounds (IIa)-(1).179aA-1 to (IIa)-(1).179aA-2313; (IIIa)-(1).179aA-1 to (IIIa)-(1).179aA-2313)

### Table 180a

Compounds (IIa)-(1) and (IIIa)-(1), wherein A is 2-ethyl-3-chlorophenyl and the combination of B and Q corresponds in each case to one row of table A (compounds (IIa)-(1).180aA-1 to (IIa)-(1).180aA-2313; (IIIa)-(1).180aA-1 to (IIIa)-(1).180aA-2313)

### Table 181a

Compounds (IIa)-(1) and (IIIa)-(1), wherein A is 2-ethyl-4-chlorophenyl and the combination of B and Q corresponds in each case to one row of table A (compounds (IIa)-(1).181aA-1 to (IIa)-(1).181aA-2313; (IIIa)-(1).181aA-1 to (IIIa)-(1).181aA-2313)

### Table 182a

Compounds (IIa)-(1) and (IIIa)-(1), wherein A is 2-ethyl-5-chlorophenyl and the combination of B and Q corresponds in each case to one row of table A (compounds (IIa)-(1).182aA-1 to (IIa)-(1).182aA-2313; (IIIa)-(1).182aA-1 to (IIIa)-(1).182aA-2313)

### Table 183a

Compounds (IIa)-(1) and (IIIa)-(1), wherein A is 2-ethyl-6-chlorophenyl and the combination of B and Q corresponds in each case to one row of table A (compounds (IIa)-(1).183aA-1 to (IIa)-(1).183aA-2313; (IIIa)-(1).183aA-1 to (IIIa)-(1).183aA-2313)

### Table 184a

Compounds (IIa)-(1) and (IIIa)-(1), wherein A is 3-ethyl-4-chlorophenyl and the combination of B and Q corresponds in each case to one row of table A (compounds (IIa)-(1).184aA-1 to (IIa)-(1).184aA-2313; (IIIa)-(1).184aA-1 to (IIIa)-(1).184aA-2313)

### Table 185a

Compounds (IIa)-(1) and (IIIa)-(1), wherein A is 3-ethyl-5-chlorophenyl and the combination of B and Q corresponds in each case to one row of table A (compounds (IIa)-(1).185aA-1 to (IIa)-(1).185aA-2313; (IIIa)-(1).185aA-1 to (IIIa)-(1).185aA-2313)

### Table 186a

Compounds (IIa)-(1) and (IIIa)-(1), wherein A is 2-chloro-3-ethylphenyl and the combination of B and Q corresponds in each case to one row of table A (compounds (IIa)-(1).186aA-1 to (IIa)-(1).186aA-2313; (IIIa)-(1).186aA-1 to (IIIa)-(1).186aA-2313)

### Table 187a

Compounds (IIa)-(1) and (IIIa)-(1), wherein A is 2-chloro-4-ethylphenyl and the combination of B and Q corresponds in each case to one row of table A (compounds (IIa)-(1).187aA-1 to (IIa)-(1).187aA-2313; (IIIa)-(1).187aA-1 to (IIIa)-(1).187aA-2313)

### Table 188a

Compounds (IIa)-(1) and (IIIa)-(1), wherein A is 2-chloro-5-ethylphenyl and the combination of B and Q corresponds in each case to one row of table A (compounds (IIa)-(1).188aA-1 to (IIa)-(1).188aA-2313; (IIIa)-(1).188aA-1 to (IIIa)-(1).188aA-2313)

### Table 189a

Compounds (IIa)-(1) and (IIIa)-(1), wherein A is 3-chloro-4-ethylphenyl and the combination of B and Q corresponds in each case to one row of table A (compounds (IIa)-(1).189aA-1 to (IIa)-(1).189aA-2313; (IIIa)-(1).189aA-1 to (IIIa)-(1).189aA-2313)

### Table 190a

Compounds (IIa)-(1) and (IIIa)-(1), wherein A is 2-methoxy-3-chlorophenyl and the combination of B and Q corresponds in each case to one row of table A (compounds (IIa)-(1).190aA-1 to (IIa)-(1).190aA-2313; (IIIa)-(1).190aA-1 to (IIIa)-(1).190aA-2313)

### Table 191a

Compounds (IIa)-(1) and (IIIa)-(1), wherein A is 2-methoxy-4-chlorophenyl and the combination of B and Q corresponds in each case to one row of table A (compounds (IIa)-(1).191aA-1 to (IIa)-(1).191aA-2313; (IIIa)-(1).191aA-1 to (IIIa)-(1).191aA-2313)

### Table 192a

Compounds (IIa)-(1) and (IIIa)-(1), wherein A is 2-methoxy-5-chlorophenyl and the combination of B and Q corresponds in each case to one row of table A (compounds (IIa)-(1).192aA-1 to (IIa)-(1).192aA-2313; (IIIa)-(1).192aA-1 to (IIIa)-(1).192aA-2313)

### Table 193a

Compounds (IIa)-(1) and (IIIa)-(1), wherein A is 2-methoxy-6-chlorophenyl and the combination of B and Q corresponds in each case to one row of table A (compounds (IIa)-(1).193aA-1 to (IIa)-(1).193aA-2313; (IIIa)-(1).193aA-1 to (IIIa)-(1).193aA-2313)

### Table 194a

Compounds (IIa)-(1) and (IIIa)-(1), wherein A is 3-methoxy-4-chlorophenyl and the combination of B and Q corresponds in each case to one row of table A (compounds (IIa)-(1).194aA-1 to (IIa)-(1).194aA-2313; (IIIa)-(1).194aA-1 to (IIIa)-(1).194aA-2313)

### Table 195a

Compounds (IIa)-(1) and (IIIa)-(1), wherein A is 3-methoxy-5-chlorophenyl and the combination of B and Q corresponds in each case to one row of table A (compounds (IIa)-(1).195aA-1 to (IIa)-(1).195aA-2313; (IIIa)-(1).195aA-1 to (IIIa)-(1).195aA-2313)

### Table 196a

Compounds (IIa)-(1) and (IIIa)-(1), wherein A is 2-chloro-3-methoxyphenyl and the combination of B and Q corresponds in each case to one row of table A (compounds (IIa)-(1).196aA-1 to (IIa)-(1).196aA-2313; (IIIa)-(1).196aA-1 to (IIIa)-(1).196aA-2313)

### Table 197a

Compounds (IIa)-(1) and (IIIa)-(1), wherein A is 2-chloro-4-methoxyphenyl and the combination of B and Q corresponds in each case to one row of table A (compounds (IIa)-(1).197aA-1 to (IIa)-(1).197aA-2313; (IIIa)-(1).197aA-1 to (IIIa)-(1).197aA-2313)

### Table 198a

Compounds (IIa)-(1) and (IIIa)-(1), wherein A is 2-chloro-5-methoxyphenyl and the combination of B and Q corresponds in each case to one row of table A (compounds (IIa)-(1).198aA-1 to (IIa)-(1).198aA-2313; (IIIa)-(1).198aA-1 to (IIIa)-(1).198aA-2313)

### Table 199a

Compounds (IIa)-(1) and (IIIa)-(1), wherein A is 3-chloro-4-methoxyphenyl and the combination of B and Q corresponds in each case to one row of table A (compounds (IIa)-(1).199aA-1 to (IIa)-(1).199aA-2313; (IIIa)-(1).199aA-1 to (IIIa)-(1).199aA-2313)

### Table 200a

Compounds (IIa)-(1) and (IIIa)-(1), wherein A is 2-(trifluoromethyl)-3-chlorophenyl and the combination of B and Q corresponds in each case to one row of table A (compounds (IIa)-(1).200aA-1 to (IIa)-(1).200aA-2313; (IIIa)-(1).200aA-1 to (IIIa)-(1).200aA-2313)

### Table 201a

Compounds (IIa)-(1) and (IIIa)-(1), wherein A is 2-(trifluoromethyl)-4-chlorophenyl and the combination of B and Q corresponds in each case to one row of table A (compounds (IIa)-(1).201aA-1 to (IIa)-(1).201aA-2313; (IIIa)-(1).201aA-1 to (IIIa)-(1).201aA-2313)

### Table 202a

Compounds (IIa)-(1) and (IIIa)-(1), wherein A is 2-(trifluoromethyl)-5-chlorophenyl and the combination of B and Q corresponds in each case to one row of table A (compounds (IIa)-(1).202aA-1 to (IIa)-(1).202aA-2313; (IIIa)-(1).202aA-1 to (IIIa)-(1).202aA-2313)

### Table 203a

Compounds (IIa)-(1) and (IIIa)-(1), wherein A is 2-(trifluoromethyl)-6-chlorophenyl and the combination of B and Q corresponds in each case to one row of table A (compounds (IIa)-(1).203aA-1 to (IIa)-(1).203aA-2313; (IIIa)-(1).203aA-1 to (IIIa)-(1).203aA-2313)

### Table 204a

Compounds (IIa)-(1) and (IIIa)-(1), wherein A is 3-(trifluoromethyl)-4-chlorophenyl and the combination of B and Q corresponds in each case to one row of table A (compounds (IIa)-(1).204aA-1 to (IIa)-(1).204aA-2313; (IIIa)-(1).204aA-1 to (IIIa)-(1).204aA-2313)

### Table 205a

Compounds (IIa)-(1) and (IIIa)-(1), wherein A is 3-(trifluoromethyl)-5-chlorophenyl and the combination of B and Q corresponds in each case to one row of table A (compounds (IIa)-(1).205aA-1 to (IIa)-(1).205aA-2313; (IIIa)-(1).205aA-1 to (IIIa)-(1).205aA-2313)

### Table 206a

Compounds (IIa)-(1) and (IIIa)-(1), wherein A is 2-chloro-3-(trifluoromethyl)phenyl and the combination of B and Q corresponds in each case to one row of table A (compounds (IIa)-(1).206aA-1 to (IIa)-(1).206aA-2313; (IIIa)-(1).206aA-1 to (IIIa)-(1).206aA-2313)

### Table 207a

Compounds (IIa)-(1) and (IIIa)-(1), wherein A is 2-chloro-4-(trifluoromethyl)phenyl and the combination of B and Q corresponds in each case to one row of table A (compounds (IIa)-(1).207aA-1 to (IIa)-(1).207aA-2313; (IIIa)-(1).207aA-1 to (IIIa)-(1).207aA-2313)

### Table 208a

Compounds (IIa)-(1) and (IIIa)-(1), wherein A is 2-chloro-5-(trifluoromethyl)phenyl and the combination of B and Q corresponds in each case to one row of table A (compounds (IIa)-(1).208aA-1 to (IIa)-(1).208aA-2313; (IIIa)-(1).208aA-1 to (IIIa)-(1).208aA-2313)

### Table 209a

Compounds (IIa)-(1) and (IIIa)-(1), wherein A is 3-chloro-4-(trifluoromethyl)phenyl and the combination of B and Q corresponds in each case to one row of table A (compounds (IIa)-(1).209aA-1 to (IIa)-(1).209aA-2313; (IIIa)-(1).209aA-1 to (IIIa)-(1).209aA-2313)

### Table 210a

Compounds (IIa)-(1) and (IIIa)-(1), wherein A is 2-(trifluoromethoxy)-3-chlorophenyl and the combination of B and Q corresponds in each case to one row of table A (compounds (IIa)-(1).210aA-1 to (IIa)-(1).210aA-2313; (IIIa)-(1).210aA-1 to (IIIa)-(1).210aA-2313)

### Table 211a

Compounds (IIa)-(1) and (IIIa)-(1), wherein A is 2-(trifluoromethoxy)-4-chlorophenyl and the combination of B and Q corresponds in each case to one row of table A (compounds (IIa)-(1).211aA-1 to (IIa)-(1).211aA-2313; (IIIa)-(1).211aA-1 to (IIIa)-(1).211aA-2313)

### Table 212a

Compounds (IIa)-(1) and (IIIa)-(1), wherein A is 2-(trifluoromethoxy)-5-chlorophenyl and the combination of B and Q corresponds in each case to one row of table A (compounds (IIa)-(1).212aA-1 to (IIa)-(1).212aA-2313; (IIIa)-(1).212aA-1 to (IIIa)-(1).212aA-2313)

### Table 213a

Compounds (IIa)-(1) and (IIIa)-(1), wherein A is 2-(trifluoromethoxy)-6-chlorophenyl and the combination of B and Q corresponds in each case to one row of table A (compounds (IIa)-(1).213aA-1 to (IIa)-(1).213aA-2313; (IIIa)-(1).213aA-1 to (IIIa)-(1).213aA-2313)

### Table 214a

Compounds (IIa)-(1) and (IIIa)-(1), wherein A is 3-(trifluoromethoxy)-4-chlorophenyl and the combination of B and Q corresponds in each case to one row of table A (compounds (IIa)-(1).214aA-1 to (IIa)-(1).214aA-2313; (IIIa)-(1).214aA-1 to (IIIa)-(1).214aA-2313)

### Table 215a

Compounds (IIa)-(1) and (IIIa)-(1), wherein A is 3-(trifluoromethoxy)-5-chlorophenyl and the combination of B and Q corresponds in each case to one row of table A (compounds (IIa)-(1).215aA-1 to (IIa)-(1).215aA-2313; (IIIa)-(1).215aA-1 to (IIIa)-(1).215aA-2313)

### Table 216a

Compounds (IIa)-(1) and (IIIa)-(1), wherein A is 2-chloro-3-(trifluoromethoxy)phenyl and the combination of B and Q corresponds in each case to one row of table A (compounds (IIa)-(1).216aA-1 to (IIa)-(1).216aA-2313; (IIIa)-(1).216aA-1 to (IIIa)-(1).216aA-2313)

### Table 217a

Compounds (IIa)-(1) and (IIIa)-(1), wherein A is 2-chloro-4-(trifluoromethoxy)phenyl and the combination of B and Q corresponds in each case to one row of table A (compounds (IIa)-(1).217aA-1 to (IIa)-(1).217aA-2313; (IIIa)-(1).217aA-1 to (IIIa)-(1).217aA-2313)
   Table 218a Compounds (IIa)-(1) and (IIIa)-(1), wherein A is 2-chloro-5-(trifluoromethoxy)phenyl and the combination of B and Q corresponds in each case to one row of table A (compounds (IIa)-(1).218aA-1 to (IIa)-(1).218aA-2313; (IIIa)-(1).218aA-1 to (IIIa)-(1).218aA-2313)

### Table 219a

Compounds (IIa)-(1) and (IIIa)-(1), wherein A is 3-chloro-4-(trifluoromethoxy)phenyl and the combination of B and Q corresponds in each case to one row of table A (compounds (IIa)-(1).219aA-1 to (IIa)-(1).219aA-2313; (IIIa)-(1).219aA-1 to (IIIa)-(1).219aA-2313)

### Table 220a

Compounds (IIa)-(1) and (IIIa)-(1), wherein A is 2-(difluoromethoxy)-3-chlorophenyl and the combination of B and Q corresponds in each case to one row of table A (compounds (IIa)-(1).220aA-1 to (IIa)-(1).220aA-2313; (IIIa)-(1).220aA-1 to (IIIa)-(1).220aA-2313)

### Table 221a

Compounds (IIa)-(1) and (IIIa)-(1), wherein A is 2-(difluoromethoxy)-4-chlorophenyl and the combination of B and Q corresponds in each case to one row of table A (compounds (IIa)-(1).221aA-1 to (IIa)-(1).221aA-2313; (IIIa)-(1).221aA-1 to (IIIa)-(1).221aA-2313)

### Table 222a

Compounds (IIa)-(1) and (IIIa)-(1), wherein A is 2-(difluoromethoxy)-5-chlorophenyl and the combination of B and Q corresponds in each case to one row of table A (compounds (IIa)-(1).222aA-1 to (IIa)-(1).222aA-2313; (IIIa)-(1).222aA-1 to (IIIa)-(1).222aA-2313)

### Table 223a

Compounds (IIa)-(1) and (IIIa)-(1), wherein A is 2-(difluoromethoxy)-6-chlorophenyl and the combination of B and Q corresponds in each case to one row of table A (compounds (IIa)-(1).223aA-1 to (IIa)-(1).223aA-2313; (IIIa)-(1).223aA-1 to (IIIa)-(1).223aA-2313)

### Table 224a

Compounds (IIa)-(1) and (IIIa)-(1), wherein A is 3-(difluoromethoxy)-4-chlorophenyl and the combination of B and Q corresponds in each case to one row of table A (compounds (IIa)-(1).224aA-1 to (IIa)-(1).224aA-2313; (IIIa)-(1).224aA-1 to (IIIa)-(1).224aA-2313)

### Table 225a

Compounds (IIa)-(1) and (IIIa)-(1), wherein A is 3-(difluoromethoxy)-5-chlorophenyl and the combination of B and Q corresponds in each case to one row of table A (compounds (IIa)-(1).225aA-1 to (IIa)-(1).225aA-2313; (IIIa)-(1).225aA-1 to (IIIa)-(1).225aA-2313)

### Table 226a

Compounds (IIa)-(1) and (IIIa)-(1), wherein A is 2-chloro-3-(difluoromethoxy)phenyl and the combination of B and Q corresponds in each case to one row of table A (compounds (IIa)-(1).226aA-1 to (IIa)-(1).226aA-2313; (IIIa)-(1).226aA-1 to (IIIa)-(1).226aA-2313)

### Table 227a

Compounds (IIa)-(1) and (IIIa)-(1), wherein A is 2-chloro-4-(difluoromethoxy)phenyl and the combination of B and Q corresponds in each case to one row of table A (compounds (IIa)-(1).227aA-1 to (IIa)-(1).227aA-2313; (IIIa)-(1).227aA-1 to (IIIa)-(1).227aA-2313)

### Table 228a

Compounds (IIa)-(1) and (IIIa)-(1), wherein A is 2-chloro-5-(difluoromethoxy)phenyl and the combination of B and Q corresponds in each case to one row of table A (compounds (IIa)-(1).228aA-1 to (IIa)-(1).228aA-2313; (IIIa)-(1).228aA-1 to (IIIa)-(1).228aA-2313)

### Table 229a

Compounds (IIa)-(1) and (IIIa)-(1), wherein A is 3-chloro-4-(difluoromethoxy)phenyl and the combination of B and Q corresponds in each case to one row of table A (compounds (IIa)-(1).229aA-1 to (IIa)-(1). 229aA-2313; (IIIa)-(1).229aA-1 to (IIIa)-(1).229aA-2313)

### Table 230a

Compounds (IIa)-(1) and (IIIa)-(1), wherein A is 2-(trifluoromethylthio)-3-chlorophenyl and the combination of B and Q corresponds in each case to one row of table A (compounds (IIa)-(1).230aA-1 to (IIa)-(1).230aA-2313; (IIIa)-(1).230aA-1 to (IIIa)-(1).230aA-2313)

### Table 231a

Compounds (IIa)-(1) and (IIIa)-(1), wherein A is 2-(trifluoromethylthio)-4-chlorophenyl and the combination of B and Q corresponds in each case to one row of table A (compounds (IIa)-(1).231aA-1 to (IIa)-(1).231aA-2313; (IIIa)-(1).231aA-1 to (IIIa)-(1).231aA-2313)

### Table 232a

Compounds (IIa)-(1) and (IIIa)-(1), wherein A is 2-(trifluoromethylthio)-5-chlorophenyl and the combination of B and Q corresponds in each case to one row of table A (compounds (IIa)-(1).232aA-1 to (IIa)-(1).232aA-2313; (IIIa)-(1).232aA-1 to (IIIa)-(1).232aA-2313)

### Table 233a

Compounds (IIa)-(1) and (IIIa)-(1), wherein A is 2-(trifluoromethylthio)-6-chlorophenyl and the combination of B and Q corresponds in each case to one row of table A (compounds (IIa)-(1).233aA-1 to (IIa)-(1).233aA-2313; (IIIa)-(1).233aA-1 to (IIIa)-(1).233aA-2313)

### Table 234a

Compounds (IIa)-(1) and (IIIa)-(1), wherein A is 3-(trifluoromethylthio)-4-chlorophenyl and the combination of B and Q corresponds in each case to one row of table A (compounds (IIa)-(1).234aA-1 to (IIa)-(1).234aA-2313; (IIIa)-(1).234aA-1 to (IIIa)-(1).234aA-2313)

### Table 235a

Compounds (IIa)-(1) and (IIIa)-(1), wherein A is 3-(trifluoromethylthio)-5-chlorophenyl and the combination of B and Q corresponds in each case to one row of table A (compounds (IIa)-(1).235aA-1 to (IIa)-(1).235aA-2313; (IIIa)-(1).235aA-1 to (IIIa)-(1).235aA-2313)

### Table 236a

Compounds (IIa)-(1) and (IIIa)-(1), wherein A is 2-chloro-3-(trifluoromethylthio)phenyl and the combination of B and Q corresponds in each case to one row of table A (compounds (IIa)-(1).236aA-1 to (IIa)-(1).236aA-2313; (IIIa)-(1).236aA-1 to (IIIa)-(1).236aA-2313)

### Table 237a

Compounds (IIa)-(1) and (IIIa)-(1), wherein A is 2-chloro-4-(trifluoromethylthio)phenyl and the combination of B and Q corresponds in each case to one row of table A (compounds (IIa)-(1).237aA-1 to (IIa)-(1).237aA-2313; (IIIa)-(1).237aA-1 to (IIIa)-(1).237aA-2313)

### Table 238a

Compounds (IIa)-(1) and (IIIa)-(1), wherein A is 2-chloro-5-(trifluoromethylthio)phenyl and the combination of B and Q corresponds in each case to one row of table A (compounds (IIa)-(1).238aA-1 to (IIa)-(1).238aA-2313; (IIIa)-(1).238aA-1 to (IIIa)-(1).238aA-2313)

### Table 239a

Compounds (IIa)-(1) and (IIIa)-(1), wherein A is 3-chloro-4-(trifluoromethylthio)phenyl and the combination of B and Q corresponds in each case to one row of table A (compounds (IIa)-(1).239aA-1 to (IIa)-(1).239aA-2313; (IIIa)-(1).239aA-1 to (IIIa)-(1).239aA-2313)

### Table 240a

Compounds (IIa)-(1) and (IIIa)-(1), wherein A is 2,3,4-trichlorophenyl and the combination of B and Q corresponds in each case to one row of table A (compounds (IIa)-(1).240aA-1 to (IIa)-(1).240aA-2313; (IIIa)-(1).240aA-1 to (IIIa)-(1).240aA-2313)

### Table 241a

Compounds (IIa)-(1) and (IIIa)-(1), wherein A is 2,3,5-trichlorophenyl and the combination of B and Q corresponds in each case to one row of table A (compounds (IIa)-(1).241aA-1 to (IIa)-(1).241aA-2313; (IIIa)-(1).241aA-1 to (IIIa)-(1).241aA-2313)

### Table 242a

Compounds (IIa)-(1) and (IIIa)-(1), wherein A is 2,3,6-trichlorophenyl and the combination of B and Q corresponds in each case to one row of table A (compounds (IIa)-(1).242aA-1 to (IIa)-(1).242aA-2313; (IIIa)-(1).242aA-1 to (IIIa)-(1).242aA-2313)

### Table 243a

Compounds (IIa)-(1) and (IIIa)-(1), wherein A is 2,4,5-trichlorophenyl and the combination of B and Q corresponds in each case to one row of table A (compounds (IIa)-(1).243aA-1 to (IIa)-(1).243aA-2313; (IIIa)-(1).243aA-1 to (IIIa)-(1).243aA-2313)

### Table 244a

Compounds (IIa)-(1) and (IIIa)-(1), wherein A is 2,4,6-trichlorophenyl and the combination of B and Q corresponds in each case to one row of table A (compounds (IIa)-(1).244aA-1 to (IIa)-(1).244aA-2313; (IIIa)-(1).244aA-1 to (IIIa)-(1).244aA-2313)

### Table 245a

Compounds (IIa)-(1) and (IIIa)-(1), wherein A is 3,4,5-trichlorophenyl and the combination of B and Q corresponds in each case to one row of table A (compounds (IIa)-(1).245aA-1 to (IIa)-(1).245aA-2313; (IIIa)-(1).245aA-1 to (IIIa)-(1).245aA-2313)

### Table 246a

Compounds (IIa)-(1) and (IIIa)-(1), wherein A is 2,3,4-trimethylphenyl and the combination of B and Q corresponds in each case to one row of table A (compounds (IIa)-(1).246aA-1 to (IIa)-(1).246aA-2313; (IIIa)-(1).246aA-1 to (IIIa)-(1).246aA-2313)

### Table 247a

Compounds (IIa)-(1) and (IIIa)-(1), wherein A is 2,3,5-trimethylphenyl and the combination of B and Q corresponds in each case to one row of table A (compounds (IIa)-(1).247aA-1 to (IIa)-(1).247aA-2313; (IIIa)-(1).247aA-1 to (IIIa)-(1).247aA-2313)

### Table 248a

Compounds (IIa)-(1) and (IIIa)-(1), wherein A is 2,3,6-trimethylphenyl and the combination of B and Q corresponds in each case to one row of table A (compounds (IIa)-(1).248aA-1 to (IIa)-(1).248aA-2313; (IIIa)-(1).248aA-1 to (IIIa)-(1).248aA-2313)

### Table 249a

Compounds (IIa)-(1) and (IIIa)-(1), wherein A is 2,4,5-trimethylphenyl and the combination of B and Q corresponds in each case to one row of table A (compounds (IIa)-(1).249aA-1 to (IIa)-(1).249aA-2313; (IIIa)-(1).249aA-1 to (IIIa)-(1).249aA-2313)

### Table 250a

Compounds (IIa)-(1) and (IIIa)-(1), wherein A is 2,4,6-trimethylphenyl and the combination of B and Q corresponds in each case to one row of table A (compounds (IIa)-(1).250aA-1 to (IIa)-(1).250aA-2313; (IIIa)-(1).250aA-1 to (IIIa)-(1).250aA-2313)

### Table 251a

Compounds (IIa)-(1) and (IIIa)-(1), wherein A is 3,4,5-trimethylphenyl and the combination of B and Q corresponds in each case to one row of table A (compounds (IIa)-(1).251aA-1 to (IIa)-(1).251aA-2313; (IIIa)-(1).251aA-1 to (IIIa)-(1).251aA-2313)

### Table 252a

Compounds (IIa)-(1) and (IIIa)-(1), wherein A is 2,3,4-trimethoxyphenyl and the combination of B and Q corresponds in each case to one row of table A (compounds (IIa)-(1).252aA-1 to (IIa)-(1).252aA-2313; (IIIa)-(1).252aA-1 to (IIIa)-(1).252aA-2313)

### Table 253a

Compounds (IIa)-(1) and (IIIa)-(1), wherein A is 2,3,5-trimethoxyphenyl and the combination of B and Q corresponds in each case to one row of table A (compounds (IIa)-(1).253aA-1 to (IIa)-(1).253aA-2313; (IIIa)-(1).253aA-1 to (IIIa)-(1).253aA-2313)

### Table 254a

Compounds (IIa)-(1) and (IIIa)-(1), wherein A is 2,3,6-trimethoxyphenyl and the combination of B and Q corresponds in each case to one row of table A (compounds (IIa)-(1).254aA-1 to (IIa)-(1).254aA-2313; (IIIa)-(1).254aA-1 to (IIIa)-(1).254aA-2313)

### Table 255a

Compounds (IIa)-(1) and (IIIa)-(1), wherein A is 2,4,5-trimethoxyphenyl and the combination of B and Q corresponds in each case to one row of table A (compounds (IIa)-(1).255aA-1 to (IIa)-(1).255aA-2313; (IIIa)-(1).255aA-1 to (IIIa)-(1).255aA-2313)

### Table 256a

Compounds (IIa)-(1) and (IIIa)-(1), wherein A is 2,4,6-trimethoxyphenyl and the combination of B and Q corresponds in each case to one row of table A (compounds (IIa)-(1).256aA-1 to (IIa)-(1).256aA-2313; (IIIa)-(1).256aA-1 to (IIIa)-(1).256aA-2313)

### Table 257a

Compounds (IIa)-(1) and (IIIa)-(1), wherein A is 3,4,5-trimethoxyphenyl and the combination of B and Q corresponds in each case to one row of table A (compounds (IIa)-(1).257aA-1 to (IIa)-(1).257aA-2313; (IIIa)-(1).257aA-1 to (IIIa)-(1).257aA-2313)

TABLE A

| row | B | Q |
|---|---|---|
| A-1 | phenyl | Cl × LiCl |
| A-2 | 2-chlorophenyl | Cl × LiCl |
| A-3 | 3-chlorophenyl | Cl × LiCl |
| A-4 | 4-chlorophenyl | Cl × LiCl |
| A-5 | 2-fluorophenyl | Cl × LiCl |
| A-6 | 3-fluorophenyl | Cl × LiCl |
| A-7 | 4-fluorophenyl | Cl × LiCl |
| A-8 | 2-methylphenyl | Cl × LiCl |
| A-9 | 3-methylphenyl | Cl × LiCl |
| A-10 | 4-methylphenyl | Cl × LiCl |
| A-11 | 2-ethylphenyl | Cl × LiCl |
| A-12 | 3-ethylphenyl | Cl × LiCl |
| A-13 | 4-ethylphenyl | Cl × LiCl |
| A-14 | 2-methoxyphenyl | Cl × LiCl |
| A-15 | 3-methoxyphenyl | Cl × LiCl |
| A-16 | 4-methoxyphenyl | Cl × LiCl |
| A-17 | 2-trifluoromethylphenyl | Cl × LiCl |
| A-18 | 3-trifluoromethylphenyl | Cl × LiCl |
| A-19 | 4-trifluoromethylphenyl | Cl × LiCl |
| A-20 | 2-trifluoromethoxyphenyl | Cl × LiCl |
| A-21 | 3-trifluoromethoxyphenyl | Cl × LiCl |
| A-22 | 4-trifluoromethoxyphenyl | Cl × LiCl |
| A-23 | 2-difluoromethoxyphenyl | Cl × LiCl |
| A-24 | 3-difluoromethoxyphenyl | Cl × LiCl |
| A-25 | 4-difluoromethoxyphenyl | Cl × LiCl |
| A-26 | 2-trifluoromethylthiophenyl | Cl × LiCl |
| A-27 | 3-trifluoromethylthiophenyl | Cl × LiCl |
| A-28 | 4-trifluoromethylthiophenyl | Cl × LiCl |
| A-29 | 2,3-dichlorophenyl | Cl × LiCl |
| A-30 | 2,4-dichlorophenyl | Cl × LiCl |
| A-31 | 2,5-dichlorophenyl | Cl × LiCl |
| A-32 | 2,6-dichlorophenyl | Cl × LiCl |
| A-33 | 3,4-dichlorophenyl | Cl × LiCl |
| A-34 | 3,5-dichlorophenyl | Cl × LiCl |
| A-35 | 2,3-difluorophenyl | Cl × LiCl |
| A-36 | 2,4-difluorophenyl | Cl × LiCl |
| A-37 | 2,5-difluorophenyl | Cl × LiCl |
| A-38 | 2,6-difluorophenyl | Cl × LiCl |
| A-39 | 3,4-difluorophenyl | Cl × LiCl |
| A-40 | 3,5-difluorophenyl | Cl × LiCl |
| A-41 | 2,3-dimethylphenyl | Cl × LiCl |
| A-42 | 2,4-dimethylphenyl | Cl × LiCl |
| A-43 | 2,5-dimethylphenyl | Cl × LiCl |
| A-44 | 2,6-dimethylphenyl | Cl × LiCl |
| A-45 | 3,4-dimethylphenyl | Cl × LiCl |
| A-46 | 3,5-dimethylphenyl | Cl × LiCl |
| A-47 | 2,3-diethylphenyl | Cl × LiCl |
| A-48 | 2,4-diethylphenyl | Cl × LiCl |
| A-49 | 2,5-diethylphenyl | Cl × LiCl |
| A-50 | 2,6-diethylphenyl | Cl × LiCl |
| A-51 | 3,5-diethylphenyl | Cl × LiCl |
| A-52 | 3,4-diethylphenyl | Cl × LiCl |
| A-53 | 2,3-dimethoxyphenyl | Cl × LiCl |
| A-54 | 2,4-dimethoxyphenyl | Cl × LiCl |
| A-55 | 2,5-dimethoxyphenyl | Cl × LiCl |
| A-56 | 2,6-dimethoxyphenyl | Cl × LiCl |
| A-57 | 3,4-dimethoxyphenyl | Cl × LiCl |
| A-58 | 3,5-dimethoxyphenyl | Cl × LiCl |
| A-59 | 2,3-di(trifluoromethyl)phenyl | Cl × LiCl |
| A-60 | 2,4-di(trifluoromethyl)phenyl | Cl × LiCl |
| A-61 | 2,5-di(trifluoromethyl)phenyl | Cl × LiCl |
| A-62 | 2,6-di(trifluoromethyl)phenyl | Cl × LiCl |
| A-63 | 3,4-di(trifluoromethyl)phenyl | Cl × LiCl |
| A-64 | 3,5-di(trifluoromethyl)phenyl | Cl × LiCl |
| A-65 | 2,3-di(trifluoromethoxy)phenyl | Cl × LiCl |
| A-66 | 2,4-di(trifluoromethoxy)phenyl | Cl × LiCl |
| A-67 | 2,5-di(trifluoromethoxy)phenyl | Cl × LiCl |
| A-68 | 2,6-di(trifluoromethoxy)phenyl | Cl × LiCl |
| A-69 | 3,4-di(trifluoromethoxy)phenyl | Cl × LiCl |
| A-70 | 3,5-di(trifluoromethoxy)phenyl | Cl × LiCl |
| A-71 | 2,3-di(difluoromethoxy)phenyl | Cl × LiCl |
| A-72 | 2,4-di(difluoromethoxy)phenyl | Cl × LiCl |
| A-73 | 2,5-di(difluoromethoxy)phenyl | Cl × LiCl |
| A-74 | 2,6-di(difluoromethoxy)phenyl | Cl × LiCl |
| A-75 | 3,4-di(difluoromethoxy)phenyl | Cl × LiCl |
| A-76 | 3,5-di(difluoromethoxy)phenyl | Cl × LiCl |
| A-77 | 2,3-di(trifluoromethylthio)phenyl | Cl × LiCl |
| A-78 | 2,4-di(trifluoromethylthio)phenyl | Cl × LiCl |
| A-79 | 2,5-di(trifluoromethylthio)phenyl | Cl × LiCl |
| A-80 | 2,6-di(trifluoromethylthio)phenyl | Cl × LiCl |
| A-81 | 3,4-di(trifluoromethylthio)phenyl | Cl × LiCl |
| A-82 | 3,5-di(trifluoromethylthio)phenyl | Cl × LiCl |
| A-83 | 2-fluoro-3-chlorophenyl | Cl × LiCl |
| A-84 | 2-fluoro-4-chlorophenyl | Cl × LiCl |
| A-85 | 2-fluoro-5-chlorophenyl | Cl × LiCl |
| A-86 | 2-fluoro-6-chlorophenyl | Cl × LiCl |
| A-87 | 3-fluoro-4-chlorophenyl | Cl × LiCl |
| A-88 | 3-fluoro-5-chlorophenyl | Cl × LiCl |
| A-89 | 2-chloro-3-fluorophenyl | Cl × LiCl |
| A-90 | 2-chloro-4-fluorophenyl | Cl × LiCl |
| A-91 | 2-chloro-5-fluorophenyl | Cl × LiCl |
| A-92 | 3-chloro-4-fluorophenyl | Cl × LiCl |
| A-93 | 2-methyl-3-chlorophenyl | Cl × LiCl |
| A-94 | 2-methyl-4-chlorophenyl | Cl × LiCl |
| A-95 | 2-methyl-5-chlorophenyl | Cl × LiCl |
| A-96 | 2-methyl-6-chlorophenyl | Cl × LiCl |
| A-97 | 3-methyl-4-chlorophenyl | Cl × LiCl |
| A-98 | 3-methyl-5-chlorophenyl | Cl × LiCl |
| A-99 | 2-chloro-3-methylphenyl | Cl × LiCl |
| A-100 | 2-chloro-4-methylphenyl | Cl × LiCl |
| A-101 | 2-chloro-5-methylphenyl | Cl × LiCl |
| A-102 | 3-chloro-4-methylphenyl | Cl × LiCl |
| A-103 | 2-methyl-3-fluorophenyl | Cl × LiCl |
| A-104 | 2-methyl-4-fluorophenyl | Cl × LiCl |
| A-105 | 2-methyl-5-fluorophenyl | Cl × LiCl |
| A-106 | 2-methyl-6-fluorophenyl | Cl × LiCl |
| A-107 | 3-methyl-4-fluorophenyl | Cl × LiCl |
| A-108 | 3-methyl-5-fluorophenyl | Cl × LiCl |
| A-109 | 2-fluoro-3-methylphenyl | Cl × LiCl |
| A-110 | 2-fluoro-4-methylphenyl | Cl × LiCl |
| A-111 | 2-fluoro-5-methylphenyl | Cl × LiCl |
| A-112 | 3-fluoro-4-methylphenyl | Cl × LiCl |
| A-113 | 2-chloro-3-ethylphenyl | Cl × LiCl |
| A-114 | 2-chloro-4-ethylphenyl | Cl × LiCl |
| A-115 | 2-chloro-5-ethylphenyl | Cl × LiCl |
| A-116 | 3-chloro-4-ethylphenyl | Cl × LiCl |
| A-117 | 2-ethyl-3-chlorophenyl | Cl × LiCl |
| A-118 | 2-ethyl-4-chlorophenyl | Cl × LiCl |
| A-119 | 2-ethyl-5-chlorophenyl | Cl × LiCl |
| A-120 | 2-ethyl-6-chlorophenyl | Cl × LiCl |
| A-121 | 3-ethyl-4-chlorophenyl | Cl × LiCl |
| A-122 | 3-ethyl-5-chlorophenyl | Cl × LiCl |
| A-123 | 2-ethyl-3-fluorophenyl | Cl × LiCl |
| A-124 | 2-ethyl-4-fluorophenyl | Cl × LiCl |
| A-125 | 2-ethyl-5-fluorophenyl | Cl × LiCl |
| A-126 | 2-ethyl-6-fluorophenyl | Cl × LiCl |
| A-127 | 3-ethyl-4-fluorophenyl | Cl × LiCl |
| A-128 | 3-ethyl-5-fluorophenyl | Cl × LiCl |
| A-129 | 2-fluoro-3-ethylphenyl | Cl × LiCl |
| A-130 | 2-fluoro-4-ethylphenyl | Cl × LiCl |
| A-131 | 2-fluoro-5-ethylphenyl | Cl × LiCl |
| A-132 | 3-fluoro-4-ethylphenyl | Cl × LiCl |
| A-133 | 2-methoxy-3-chlorophenyl | Cl × LiCl |
| A-134 | 2-methoxy-4-chlorophenyl | Cl × LiCl |
| A-135 | 2-methoxy-5-chlorophenyl | Cl × LiCl |
| A-136 | 2-methoxy-6-chlorophenyl | Cl × LiCl |
| A-137 | 3-methoxy-4-chlorophenyl | Cl × LiCl |
| A-138 | 3-methoxy-5-chlorophenyl | Cl × LiCl |
| A-139 | 2-chloro-3-methoxyphenyl | Cl × LiCl |
| A-140 | 2-chloro-4-methoxyphenyl | Cl × LiCl |
| A-141 | 2-chloro-5-methoxyphenyl | Cl × LiCl |
| A-142 | 3-chloro-4-methoxyphenyl | Cl × LiCl |
| A-143 | 2-methoxy-3-fluorophenyl | Cl × LiCl |
| A-144 | 2-methoxy-4-fluorophenyl | Cl × LiCl |
| A-145 | 2-methoxy-5-fluorophenyl | Cl × LiCl |
| A-146 | 2-methoxy-6-fluorophenyl | Cl × LiCl |
| A-147 | 3-methoxy-4-fluorophenyl | Cl × LiCl |
| A-148 | 3-methoxy-5-fluorophenyl | Cl × LiCl |
| A-149 | 2-fluoro-3-methoxyphenyl | Cl × LiCl |
| A-150 | 2-fluoro-4-methoxyphenyl | Cl × LiCl |
| A-151 | 2-fluoro-5-methoxyphenyl | Cl × LiCl |
| A-152 | 3-fluoro-4-methoxyphenyl | Cl × LiCl |
| A-153 | 3-fluoro-5-methoxyphenyl | Cl × LiCl |
| A-154 | 2-(trifluoromethyl)-3-chlorophenyl | Cl × LiCl |
| A-155 | 2-(trifluoromethyl)-4-chlorophenyl | Cl × LiCl |
| A-156 | 2-(trifluoromethyl)-5-chlorophenyl | Cl × LiCl |
| A-157 | 2-(trifluoromethyl)-6-chlorophenyl | Cl × LiCl |
| A-158 | 3-(trifluoromethyl)-4-chlorophenyl | Cl × LiCl |
| A-159 | 3-(trifluoromethyl)-5-chlorophenyl | Cl × LiCl |
| A-160 | 2-chloro-3-(trifluoromethyl)phenyl | Cl × LiCl |
| A-161 | 2-chloro-4-(trifluoromethyl)phenyl | Cl × LiCl |
| A-162 | 2-chloro-5-(trifluoromethyl)phenyl | Cl × LiCl |
| A-163 | 3-chloro-4-(trifluoromethyl)phenyl | Cl × LiCl |
| A-164 | 2-(trifluoromethyl)-3-fluorophenyl | Cl × LiCl |
| A-165 | 2-(trifluoromethyl)-4-fluorophenyl | Cl × LiCl |
| A-166 | 2-(trifluoromethyl)-5-fluorophenyl | Cl × LiCl |
| A-167 | 2-(trifluoromethyl)-6-fluorophenyl | Cl × LiCl |
| A-168 | 3-(trifluoromethyl)-4-fluorophenyl | Cl × LiCl |
| A-169 | 3-(trifluoromethyl)-5-fluorophenyl | Cl × LiCl |
| A-170 | 2-fluoro-3-(trifluoromethyl)phenyl | Cl × LiCl |
| A-171 | 2-fluoro-4-(trifluoromethyl)phenyl | Cl × LiCl |
| A-172 | 2-fluoro-5-(trifluoromethyl)phenyl | Cl × LiCl |
| A-173 | 3-fluoro-4-(trifluoromethyl)phenyl | Cl × LiCl |
| A-174 | 2-(trifluoromethoxy)-3-chlorophenyl | Cl × LiCl |
| A-175 | 2-(trifluoromethoxy)-4-chlorophenyl | Cl × LiCl |
| A-176 | 2-(trifluoromethoxy)-5-chlorophenyl | Cl × LiCl |
| A-177 | 2-(trifluoromethoxy)-6-chlorophenyl | Cl × LiCl |
| A-178 | 3-(trifluoromethoxy)-4-chlorophenyl | Cl × LiCl |
| A-179 | 3-(trifluoromethoxy)-5-chlorophenyl | Cl × LiCl |
| A-180 | 2-chloro-3-(trifluoromethoxy)phenyl | Cl × LiCl |
| A-181 | 2-chloro-4-(trifluoromethoxy)phenyl | Cl × LiCl |
| A-182 | 2-chloro-5-(trifluoromethoxy)phenyl | Cl × LiCl |
| A-183 | 3-chloro-4-(trifluoromethoxy)phenyl | Cl × LiCl |
| A-184 | 2-(trifluoromethoxy)-3-fluorophenyl | Cl × LiCl |
| A-185 | 2-(trifluoromethoxy)-4-fluorophenyl | Cl × LiCl |
| A-186 | 2-(trifluoromethoxy)-5-fluorophenyl | Cl × LiCl |
| A-187 | 2-(trifluoromethoxy)-6-fluorophenyl | Cl × LiCl |
| A-188 | 3-(trifluoromethoxy)-4-fluorophenyl | Cl × LiCl |
| A-189 | 3-(trifluoromethoxy)-5-fluorophenyl | Cl × LiCl |
| A-190 | 2-fluoro-3-(trifluoromethoxy)phenyl | Cl × LiCl |
| A-191 | 2-fluoro-4-(trifluoromethoxy)phenyl | Cl × LiCl |
| A-192 | 2-fluoro-5-(trifluoromethoxy)phenyl | Cl × LiCl |
| A-193 | 3-fluoro-4-(trifluoromethoxy)phenyl | Cl × LiCl |
| A-194 | 2-(difluoromethoxy)-3-chlorophenyl | Cl × LiCl |
| A-195 | 2-(difluoromethoxy)-4-chlorophenyl | Cl × LiCl |
| A-196 | 2-(difluoromethoxy)-5-chlorophenyl | Cl × LiCl |
| A-197 | 2-(difluoromethoxy)-6-chlorophenyl | Cl × LiCl |
| A-198 | 3-(difluoromethoxy)-4-chlorophenyl | Cl × LiCl |
| A-199 | 3-(difluoromethoxy)-5-chlorophenyl | Cl × LiCl |
| A-200 | 2-chloro-3-(difluoromethoxy)phenyl | Cl × LiCl |
| A-201 | 2-chloro-4-(difluoromethoxy)phenyl | Cl × LiCl |
| A-202 | 2-chloro-5-(difluoromethoxy)phenyl | Cl × LiCl |
| A-203 | 3-chloro-4-(difluoromethoxy)phenyl | Cl × LiCl |
| A-204 | 2-(difluoromethoxy)-3-fluorophenyl | Cl × LiCl |
| A-205 | 2-(difluoromethoxy)-4-fluorophenyl | Cl × LiCl |
| A-206 | 2-(difluoromethoxy)-5-fluorophenyl | Cl × LiCl |
| A-207 | 2-(difluoromethoxy)-6-fluorophenyl | Cl × LiCl |
| A-208 | 3-(difluoromethoxy)-4-fluorophenyl | Cl × LiCl |
| A-209 | 3-(difluoromethoxy)-5-fluorophenyl | Cl × LiCl |
| A-210 | 2-fluoro-3-(difluoromethoxy)phenyl | Cl × LiCl |
| A-211 | 2-fluoro-4-(difluoromethoxy)phenyl | Cl × LiCl |
| A-212 | 2-fluoro-5-(difluoromethoxy)phenyl | Cl × LiCl |
| A-213 | 3-fluoro-4-(difluoromethoxy)phenyl | Cl × LiCl |
| A-214 | 2-(trifluoromethylthio)-3- | Cl × LiCl |
| | chlorophenyl | |
| A-215 | 2-(trifluoromethylthio)-4- | Cl × LiCl |
| | chlorophenyl | |
| A-216 | 2-(trifluoromethylthio)-5- | Cl × LiCl |
| | chlorophenyl | |
| A-217 | 2-(trifluoromethylthio)-6- | Cl × LiCl |
| | chlorophenyl | |
| A-218 | 3-(trifluoromethylthio)-4- | Cl × LiCl |
| | chlorophenyl | |
| A-219 | 3-(trifluoromethylthio)-5- | Cl × LiCl |
| | chlorophenyl | |
| A-220 | 2-chloro-3- | Cl × LiCl |
| | (trifluoromethylthio)phenyl | |
| A-221 | 2-chloro-4- | Cl × LiCl |
| | (trifluoromethylthio)phenyl | |
| A-222 | 2-chloro-5- | Cl × LiCl |
| | (trifluoromethylthio)phenyl | |
| A-223 | 3-chloro-4- | Cl × LiCl |
| | (trifluoromethylthio)phenyl | |
| A-224 | 2-(trifluoromethylthio)-3-fluorophenyl | Cl × LiCl |
| A-225 | 2-(trifluoromethylthio)-4-fluorophenyl | Cl × LiCl |
| A-226 | 2-(trifluoromethylthio)-5-fluorophenyl | Cl × LiCl |
| A-227 | 2-(trifluoromethylthio)-6-fluorophenyl | Cl × LiCl |
| A-228 | 3-(trifluoromethylthio)-4-fluorophenyl | Cl × LiCl |
| A-229 | 3-(trifluoromethylthio)-5-fluorophenyl | Cl × LiCl |
| A-230 | 2-fluoro-3-(trifluoromethylthio)phenyl | Cl × LiCl |
| A-231 | 2-fluoro-4-(trifluoromethylthio)phenyl | Cl × LiCl |
| A-232 | 2-fluoro-5-(trifluoromethylthio)phenyl | Cl × LiCl |
| A-233 | 3-fluoro-4-(trifluoromethylthio)phenyl | Cl × LiCl |
| A-234 | 2,3,4-trichlorophenyl | Cl × LiCl |
| A-235 | 2,3,5-trichlorophenyl | Cl × LiCl |
| A-236 | 2,3,6-trichlorophenyl | Cl × LiCl |
| A-237 | 2,4,5-trichlorophenyl | Cl × LiCl |
| A-238 | 2,4,6-trichlorophenyl | Cl × LiCl |
| A-239 | 3,4,5-trichlorophenyl | Cl × LiCl |
| A-240 | 2,3,4-trifluorophenyl | Cl × LiCl |
| A-241 | 2,3,5-trifluorophenyl | Cl × LiCl |
| A-242 | 2,3,6-trifluorophenyl | Cl × LiCl |
| A-243 | 2,4,5-trifluorophenyl | Cl × LiCl |
| A-244 | 2,4,6-trifluorophenyl | Cl × LiCl |
| A-245 | 3,4,5-trifluorophenyl | Cl × LiCl |
| A-246 | 2,3,4-trimethylphenyl | Cl × LiCl |
| A-247 | 2,3,5-trimethylphenyl | Cl × LiCl |
| A-248 | 2,3,6-trimethylphenyl | Cl × LiCl |
| A-249 | 2,4,5-trimethylphenyl | Cl × LiCl |
| A-250 | 2,4,6-trimethylphenyl | Cl × LiCl |
| A-251 | 3,4,5-trimethylphenyl | Cl × LiCl |
| A-252 | 2,3,4-trimethoxyphenyl | Cl × LiCl |
| A-253 | 2,3,5-trimethoxyphenyl | Cl × LiCl |
| A-254 | 2,3,6-trimethoxyphenyl | Cl × LiCl |
| A-255 | 2,4,5-trimethoxyphenyl | Cl × LiCl |
| A-256 | 2,4,6-trimethoxyphenyl | Cl × LiCl |
| A-257 | 3,4,5-trimethoxyphenyl | Cl × LiCl |
| A-258 | phenyl | Br × LiCl |
| A-259 | 2-chlorophenyl | Br × LiCl |
| A-260 | 3-chlorophenyl | Br × LiCl |
| A-261 | 4-chlorophenyl | Br × LiCl |
| A-262 | 2-fluorophenyl | Br × LiCl |
| A-263 | 3-fluorophenyl | Br × LiCl |
| A-264 | 4-fluorophenyl | Br × LiCl |
| A-265 | 2-methylphenyl | Br × LiCl |
| A-266 | 3-methylphenyl | Br × LiCl |
| A-267 | 4-methylphenyl | Br × LiCl |
| A-268 | 2-ethylphenyl | Br × LiCl |
| A-269 | 3-ethylphenyl | Br × LiCl |
| A-270 | 4-ethylphenyl | Br × LiCl |
| A-271 | 2-methoxyphenyl | Br × LiCl |
| A-272 | 3-methoxyphenyl | Br × LiCl |
| A-273 | 4-methoxyphenyl | Br × LiCl |
| A-274 | 2-trifluoromethylphenyl | Br × LiCl |
| A-275 | 3-trifluoromethylphenyl | Br × LiCl |
| A-276 | 4-trifluoromethylphenyl | Br × LiCl |
| A-277 | 2-trifluoromethoxyphenyl | Br × LiCl |
| A-278 | 3-trifluoromethoxyphenyl | Br × LiCl |
| A-279 | 4-trifluoromethoxyphenyl | Br × LiCl |
| A-280 | 2-difluoromethoxyphenyl | Br × LiCl |
| A-281 | 3-difluoromethoxyphenyl | Br × LiCl |
| A-282 | 4-difluoromethoxyphenyl | Br × LiCl |
| A-283 | 2-trifluoromethylthiophenyl | Br × LiCl |
| A-284 | 3-trifluoromethylthiophenyl | Br × LiCl |
| A-285 | 4-trifluoromethylthiophenyl | Br × LiCl |
| A-286 | 2,3-dichlorophenyl | Br × LiCl |
| A-287 | 2,4-dichlorophenyl | Br × LiCl |
| A-288 | 2,5-dichlorophenyl | Br × LiCl |
| A-289 | 2,6-dichlorophenyl | Br × LiCl |
| A-290 | 3,4-dichlorophenyl | Br × LiCl |
| A-291 | 3,5-dichlorophenyl | Br × LiCl |
| A-292 | 2,3-difluorophenyl | Br × LiCl |
| A-293 | 2,4-difluorophenyl | Br × LiCl |
| A-294 | 2,5-difluorophenyl | Br × LiCl |
| A-295 | 2,6-difluorophenyl | Br × LiCl |
| A-296 | 3,4-difluorophenyl | Br × LiCl |
| A-297 | 3,5-difluorophenyl | Br × LiCl |
| A-298 | 2,3-dimethylphenyl | Br × LiCl |
| A-299 | 2,4-dimethylphenyl | Br × LiCl |
| A-300 | 2,5-dimethylphenyl | Br × LiCl |
| A-301 | 2,6-dimethylphenyl | Br × LiCl |
| A-302 | 3,4-dimethylphenyl | Br × LiCl |
| A-303 | 3,5-dimethylphenyl | Br × LiCl |
| A-304 | 2,3-diethylphenyl | Br × LiCl |
| A-305 | 2,4-diethylphenyl | Br × LiCl |
| A-306 | 2,5-diethylphenyl | Br × LiCl |
| A-307 | 2,6-diethylphenyl | Br × LiCl |
| A-308 | 3,5-diethylphenyl | Br × LiCl |
| A-309 | 3,4-diethylphenyl | Br × LiCl |
| A-310 | 2,3-dimethoxyphenyl | Br × LiCl |
| A-311 | 2,4-dimethoxyphenyl | Br × LiCl |
| A-312 | 2,5-dimethoxyphenyl | Br × LiCl |
| A-313 | 2,6-dimethoxyphenyl | Br × LiCl |
| A-314 | 3,4-dimethoxyphenyl | Br × LiCl |
| A-315 | 3,5-dimethoxyphenyl | Br × LiCl |
| A-316 | 2,3-di(trifluoromethyl)phenyl | Br × LiCl |
| A-317 | 2,4-di(trifluoromethyl)phenyl | Br × LiCl |
| A-318 | 2,5-di(trifluoromethyl)phenyl | Br × LiCl |
| A-319 | 2,6-di(trifluoromethyl)phenyl | Br × LiCl |
| A-320 | 3,4-di(trifluoromethyl)phenyl | Br × LiCl |
| A-321 | 3,5-di(trifluoromethyl)phenyl | Br × LiCl |
| A-322 | 2,3-di(trifluoromethoxy)phenyl | Br × LiCl |
| A-323 | 2,4-di(trifluoromethoxy)phenyl | Br × LiCl |
| A-324 | 2,5-di(trifluoromethoxy)phenyl | Br × LiCl |
| A-325 | 2,6-di(trifluoromethoxy)phenyl | Br × LiCl |
| A-326 | 3,4-di(trifluoromethoxy)phenyl | Br × LiCl |
| A-327 | 3,5-di(trifluoromethoxy)phenyl | Br × LiCl |
| A-328 | 2,3-di(difluoromethoxy)phenyl | Br × LiCl |
| A-329 | 2,4-di(difluoromethoxy)phenyl | Br × LiCl |
| A-330 | 2,5-di(difluoromethoxy)phenyl | Br × LiCl |
| A-331 | 2,6-di(difluoromethoxy)phenyl | Br × LiCl |
| A-332 | 3,4-di(difluoromethoxy)phenyl | Br × LiCl |
| A-333 | 3,5-di(difluoromethoxy)phenyl | Br × LiCl |
| A-334 | 2,3-di(trifluoromethylthio)phenyl | Br × LiCl |
| A-335 | 2,4-di(trifluoromethylthio)phenyl | Br × LiCl |
| A-336 | 2,5-di(trifluoromethylthio)phenyl | Br × LiCl |
| A-337 | 2,6-di(trifluoromethylthio)phenyl | Br × LiCl |
| A-338 | 3,4-di(trifluoromethylthio)phenyl | Br × LiCl |
| A-339 | 3,5-di(trifluoromethylthio)phenyl | Br × LiCl |
| A-340 | 2-fluoro-3-chlorophenyl | Br × LiCl |
| A-341 | 2-fluoro-4-chlorophenyl | Br × LiCl |
| A-342 | 2-fluoro-5-chlorophenyl | Br × LiCl |
| A-343 | 2-fluoro-6-chlorophenyl | Br × LiCl |
| A-344 | 3-fluoro-4-chlorophenyl | Br × LiCl |
| A-345 | 3-fluoro-5-chlorophenyl | Br × LiCl |
| A-346 | 2-chloro-3-fluorophenyl | Br × LiCl |
| A-347 | 2-chloro-4-fluorophenyl | Br × LiCl |
| A-348 | 2-chloro-5-fluorophenyl | Br × LiCl |
| A-349 | 3-chloro-4-fluorophenyl | Br × LiCl |
| A-350 | 2-methyl-3-chlorophenyl | Br × LiCl |
| A-351 | 2-methyl-4-chlorophenyl | Br × LiCl |
| A-352 | 2-methyl-5-chlorophenyl | Br × LiCl |
| A-353 | 2-methyl-6-chlorophenyl | Br × LiCl |
| A-354 | 3-methyl-4-chlorophenyl | Br × LiCl |
| A-355 | 3-methyl-5-chlorophenyl | Br × LiCl |
| A-356 | 2-chloro-3-methylphenyl | Br × LiCl |
| A-357 | 2-chloro-4-methylphenyl | Br × LiCl |
| A-358 | 2-chloro-5-methylphenyl | Br × LiCl |
| A-359 | 3-chloro-4-methylphenyl | Br × LiCl |
| A-360 | 2-methyl-3-fluorophenyl | Br × LiCl |
| A-361 | 2-methyl-4-fluorophenyl | Br × LiCl |
| A-362 | 2-methyl-5-fluorophenyl | Br × LiCl |
| A-363 | 2-methyl-6-fluorophenyl | Br × LiCl |
| A-364 | 3-methyl-4-fluorophenyl | Br × LiCl |
| A-365 | 3-methyl-5-fluorophenyl | Br × LiCl |
| A-366 | 2-fluoro-3-methylphenyl | Br × LiCl |
| A-367 | 2-fluoro-4-methylphenyl | Br × LiCl |
| A-368 | 2-fluoro-5-methylphenyl | Br × LiCl |
| A-369 | 3-fluoro-4-methylphenyl | Br × LiCl |
| A-370 | 2-chloro-3-ethylphenyl | Br × LiCl |
| A-371 | 2-chloro-4-ethylphenyl | Br × LiCl |
| A-372 | 2-chloro-5-ethylphenyl | Br × LiCl |
| A-373 | 3-chloro-4-ethylphenyl | Br × LiCl |
| A-374 | 2-ethyl-3-chlorophenyl | Br × LiCl |
| A-375 | 2-ethyl-4-chlorophenyl | Br × LiCl |
| A-376 | 2-ethyl-5-chlorophenyl | Br × LiCl |
| A-377 | 2-ethyl-6-chlorophenyl | Br × LiCl |
| A-378 | 3-ethyl-4-chlorophenyl | Br × LiCl |
| A-379 | 3-ethyl-5-chlorophenyl | Br × LiCl |
| A-380 | 2-ethyl-3-fluorophenyl | Br × LiCl |
| A-381 | 2-ethyl-4-fluorophenyl | Br × LiCl |
| A-382 | 2-ethyl-5-fluorophenyl | Br × LiCl |
| A-383 | 2-ethyl-6-fluorophenyl | Br × LiCl |
| A-384 | 3-ethyl-4-fluorophenyl | Br × LiCl |
| A-385 | 3-ethyl-5-fluorophenyl | Br × LiCl |
| A-386 | 2-fluoro-3-ethylphenyl | Br × LiCl |
| A-387 | 2-fluoro-4-ethylphenyl | Br × LiCl |
| A-388 | 2-fluoro-5-ethylphenyl | Br × LiCl |
| A-389 | 3-fluoro-4-ethylphenyl | Br × LiCl |
| A-390 | 2-methoxy-3-chlorophenyl | Br × LiCl |
| A-391 | 2-methoxy-4-chlorophenyl | Br × LiCl |
| A-392 | 2-methoxy-5-chlorophenyl | Br × LiCl |
| A-393 | 2-methoxy-6-chlorophenyl | Br × LiCl |
| A-394 | 3-methoxy-4-chlorophenyl | Br × LiCl |
| A-395 | 3-methoxy-5-chlorophenyl | Br × LiCl |
| A-396 | 2-chloro-3-methoxyphenyl | Br × LiCl |
| A-397 | 2-chloro-4-methoxyphenyl | Br × LiCl |
| A-398 | 2-chloro-5-methoxyphenyl | Br × LiCl |
| A-399 | 3-chloro-4-methoxyphenyl | Br × LiCl |
| A-400 | 2-methoxy-3-fluorophenyl | Br × LiCl |
| A-401 | 2-methoxy-4-fluorophenyl | Br × LiCl |
| A-402 | 2-methoxy-5-fluorophenyl | Br × LiCl |
| A-403 | 2-methoxy-6-fluorophenyl | Br × LiCl |
| A-404 | 3-methoxy-4-fluorophenyl | Br × LiCl |
| A-405 | 3-methoxy-5-fluorophenyl | Br × LiCl |
| A-406 | 2-fluoro-3-methoxyphenyl | Br × LiCl |
| A-407 | 2-fluoro-4-methoxyphenyl | Br × LiCl |
| A-408 | 2-fluoro-5-methoxyphenyl | Br × LiCl |
| A-409 | 3-fluoro-4-methoxyphenyl | Br × LiCl |
| A-410 | 3-fluoro-5-methoxyphenyl | Br × LiCl |
| A-411 | 2-(trifluoromethyl)-3-chlorophenyl | Br × LiCl |
| A-412 | 2-(trifluoromethyl)-4-chlorophenyl | Br × LiCl |
| A-413 | 2-(trifluoromethyl)-5-chlorophenyl | Br × LiCl |
| A-414 | 2-(trifluoromethyl)-6-chlorophenyl | Br × LiCl |
| A-415 | 3-(trifluoromethyl)-4-chlorophenyl | Br × LiCl |
| A-416 | 3-(trifluoromethyl)-5-chlorophenyl | Br × LiCl |
| A-417 | 2-chloro-3-(trifluoromethyl)phenyl | Br × LiCl |
| A-418 | 2-chloro-4-(trifluoromethyl)phenyl | Br × LiCl |
| A-419 | 2-chloro-5-(trifluoromethyl)phenyl | Br × LiCl |
| A-420 | 3-chloro-4-(trifluoromethyl)phenyl | Br × LiCl |
| A-421 | 2-(trifluoromethyl)-3-fluorophenyl | Br × LiCl |
| A-422 | 2-(trifluoromethyl)-4-fluorophenyl | Br × LiCl |
| A-423 | 2-(trifluoromethyl)-5-fluorophenyl | Br × LiCl |
| A-424 | 2-(trifluoromethyl)-6-fluorophenyl | Br × LiCl |
| A-425 | 3-(trifluoromethyl)-4-fluorophenyl | Br × LiCl |
| A-426 | 3-(trifluoromethyl)-5-fluorophenyl | Br × LiCl |
| A-427 | 2-fluoro-3-(trifluoromethyl)phenyl | Br × LiCl |
| A-428 | 2-fluoro-4-(trifluoromethyl)phenyl | Br × LiCl |
| A-429 | 2-fluoro-5-(trifluoromethyl)phenyl | Br × LiCl |
| A-430 | 3-fluoro-4-(trifluoromethyl)phenyl | Br × LiCl |
| A-431 | 2-(trifluoromethoxy)-3-chlorophenyl | Br × LiCl |
| A-432 | 2-(trifluoromethoxy)-4-chlorophenyl | Br × LiCl |
| A-433 | 2-(trifluoromethoxy)-5-chlorophenyl | Br × LiCl |
| A-434 | 2-(trifluoromethoxy)-6-chlorophenyl | Br × LiCl |
| A-435 | 3-(trifluoromethoxy)-4-chlorophenyl | Br × LiCl |
| A-436 | 3-(trifluoromethoxy)-5-chlorophenyl | Br × LiCl |
| A-437 | 2-chloro-3-(trifluoromethoxy)phenyl | Br × LiCl |
| A-438 | 2-chloro-4-(trifluoromethoxy)phenyl | Br × LiCl |
| A-439 | 2-chloro-5-(trifluoromethoxy)phenyl | Br × LiCl |
| A-440 | 3-chloro-4-(trifluoromethoxy)phenyl | Br × LiCl |
| A-441 | 2-(trifluoromethoxy)-3-fluorophenyl | Br × LiCl |
| A-442 | 2-(trifluoromethoxy)-4-fluorophenyl | Br × LiCl |
| A-443 | 2-(trifluoromethoxy)-5-fluorophenyl | Br × LiCl |
| A-444 | 2-(trifluoromethoxy)-6-fluorophenyl | Br × LiCl |
| A-445 | 3-(trifluoromethoxy)-4-fluorophenyl | Br × LiCl |
| A-446 | 3-(trifluoromethoxy)-5-fluorophenyl | Br × LiCl |
| A-447 | 2-fluoro-3-(trifluoromethoxy)phenyl | Br × LiCl |
| A-448 | 2-fluoro-4-(trifluoromethoxy)phenyl | Br × LiCl |
| A-449 | 2-fluoro-5-(trifluoromethoxy)phenyl | Br × LiCl |
| A-450 | 3-fluoro-4-(trifluoromethoxy)phenyl | Br × LiCl |
| A-451 | 2-(difluoromethoxy)-3-chlorophenyl | Br × LiCl |
| A-452 | 2-(difluoromethoxy)-4-chlorophenyl | Br × LiCl |
| A-453 | 2-(difluoromethoxy)-5-chlorophenyl | Br × LiCl |
| A-454 | 2-(difluoromethoxy)-6-chlorophenyl | Br × LiCl |
| A-455 | 3-(difluoromethoxy)-4-chlorophenyl | Br × LiCl |
| A-456 | 3-(difluoromethoxy)-5-chlorophenyl | Br × LiCl |
| A-457 | 2-chloro-3-(difluoromethoxy)phenyl | Br × LiCl |
| A-458 | 2-chloro-4-(difluoromethoxy)phenyl | Br × LiCl |
| A-459 | 2-chloro-5-(difluoromethoxy)phenyl | Br × LiCl |
| A-460 | 3-chloro-4-(difluoromethoxy)phenyl | Br × LiCl |
| A-461 | 2-(difluoromethoxy)-3-fluorophenyl | Br × LiCl |
| A-462 | 2-(difluoromethoxy)-4-fluorophenyl | Br × LiCl |
| A-463 | 2-(difluoromethoxy)-5-fluorophenyl | Br × LiCl |
| A-464 | 2-(difluoromethoxy)-6-fluorophenyl | Br × LiCl |
| A-465 | 3-(difluoromethoxy)-4-fluorophenyl | Br × LiCl |
| A-466 | 3-(difluoromethoxy)-5-fluorophenyl | Br × LiCl |
| A-467 | 2-fluoro-3-(difluoromethoxy)phenyl | Br × LiCl |
| A-468 | 2-fluoro-4-(difluoromethoxy)phenyl | Br × LiCl |
| A-469 | 2-fluoro-5-(difluoromethoxy)phenyl | Br × LiCl |
| A-470 | 3-fluoro-4-(difluoromethoxy)phenyl | Br × LiCl |
| A-471 | 2-(trifluoromethylthio)-3- | Br × LiCl |
| | chlorophenyl | |
| A-472 | 2-(trifluoromethylthio)-4- | Br × LiCl |
| | chlorophenyl | |
| A-473 | 2-(trifluoromethylthio)-5- | Br × LiCl |
| | chlorophenyl | |
| A-474 | 2-(trifluoromethylthio)-6- | Br × LiCl |
| | chlorophenyl | |
| A-475 | 3-(trifluoromethylthio)-4- | Br × LiCl |
| | chlorophenyl | |
| A-476 | 3-(trifluoromethylthio)-5- | Br × LiCl |
| | chlorophenyl | |
| A-477 | 2-chloro-3- | Br × LiCl |
| | (trifluoromethylthio)phenyl | |
| A-478 | 2-chloro-4- | Br × LiCl |
| | (trifluoromethylthio)phenyl | |
| A-479 | 2-chloro-5- | Br × LiCl |
| | (trifluoromethylthio)phenyl | |
| A-480 | 3-chloro-4- | Br × LiCl |
| | (trifluoromethylthio)phenyl | |
| A-481 | 2-(trifluoromethylthio)-3-fluorophenyl | Br × LiCl |
| A-482 | 2-(trifluoromethylthio)-4-fluorophenyl | Br × LiCl |
| A-483 | 2-(trifluoromethylthio)-5-fluorophenyl | Br × LiCl |
| A-484 | 2-(trifluoromethylthio)-6-fluorophenyl | Br × LiCl |
| A-485 | 3-(trifluoromethylthio)-4-fluorophenyl | Br × LiCl |
| A-486 | 3-(trifluoromethylthio)-5-fluorophenyl | Br × LiCl |
| A-487 | 2-fluoro-3-(trifluoromethylthio)phenyl | Br × LiCl |
| A-488 | 2-fluoro-4-(trifluoromethylthio)phenyl | Br × LiCl |
| A-489 | 2-fluoro-5-(trifluoromethylthio)phenyl | Br × LiCl |
| A-490 | 3-fluoro-4-(trifluoromethylthio)phenyl | Br × LiCl |
| A-491 | 2,3,4-trichlorophenyl | Br × LiCl |
| A-492 | 2,3,5-trichlorophenyl | Br × LiCl |
| A-493 | 2,3,6-trichlorophenyl | Br × LiCl |
| A-494 | 2,4,5-trichlorophenyl | Br × LiCl |
| A-495 | 2,4,6-trichlorophenyl | Br × LiCl |
| A-496 | 3,4,5-trichlorophenyl | Br × LiCl |
| A-497 | 2,3,4-trifluorophenyl | Br × LiCl |
| A-498 | 2,3,5-trifluorophenyl | Br × LiCl |
| A-499 | 2,3,6-trifluorophenyl | Br × LiCl |
| A-500 | 2,4,5-trifluorophenyl | Br × LiCl |
| A-501 | 2,4,6-trifluorophenyl | Br × LiCl |
| A-502 | 3,4,5-trifluorophenyl | Br × LiCl |
| A-503 | 2,3,4-trimethylphenyl | Br × LiCl |
| A-504 | 2,3,5-trimethylphenyl | Br × LiCl |
| A-505 | 2,3,6-trimethylphenyl | Br × LiCl |
| A-506 | 2,4,5-trimethylphenyl | Br × LiCl |
| A-507 | 2,4,6-trimethylphenyl | Br × LiCl |
| A-508 | 3,4,5-trimethylphenyl | Br × LiCl |
| A-509 | 2,3,4-trimethoxyphenyl | Br × LiCl |
| A-510 | 2,3,5-trimethoxyphenyl | Br × LiCl |
| A-511 | 2,3,6-trimethoxyphenyl | Br × LiCl |
| A-512 | 2,4,5-trimethoxyphenyl | Br × LiCl |
| A-513 | 2,4,6-trimethoxyphenyl | Br × LiCl |
| A-514 | 3,4,5-trimethoxyphenyl | Br × LiCl |
| A-515 | phenyl | 2-propyl |
| A-516 | 2-chlorophenyl | 2-propyl |
| A-517 | 3-chlorophenyl | 2-propyl |
| A-518 | 4-chlorophenyl | 2-propyl |
| A-519 | 2-fluorophenyl | 2-propyl |
| A-520 | 3-fluorophenyl | 2-propyl |
| A-521 | 4-fluorophenyl | 2-propyl |
| A-522 | 2-methylphenyl | 2-propyl |
| A-523 | 3-methylphenyl | 2-propyl |
| A-524 | 4-methylphenyl | 2-propyl |
| A-525 | 2-ethylphenyl | 2-propyl |
| A-526 | 3-ethylphenyl | 2-propyl |
| A-527 | 4-ethylphenyl | 2-propyl |
| A-528 | 2-methoxyphenyl | 2-propyl |
| A-529 | 3-methoxyphenyl | 2-propyl |
| A-530 | 4-methoxyphenyl | 2-propyl |
| A-531 | 2-trifluoromethylphenyl | 2-propyl |
| A-532 | 3-trifluoromethylphenyl | 2-propyl |
| A-533 | 4-trifluoromethylphenyl | 2-propyl |
| A-534 | 2-trifluoromethoxyphenyl | 2-propyl |
| A-535 | 3-trifluoromethoxyphenyl | 2-propyl |
| A-536 | 4-trifluoromethoxyphenyl | 2-propyl |
| A-537 | 2-difluoromethoxyphenyl | 2-propyl |
| A-538 | 3-difluoromethoxyphenyl | 2-propyl |
| A-539 | 4-difluoromethoxyphenyl | 2-propyl |
| A-540 | 2-trifluoromethylthiophenyl | 2-propyl |
| A-541 | 3-trifluoromethylthiophenyl | 2-propyl |
| A-542 | 4-trifluoromethylthiophenyl | 2-propyl |
| A-543 | 2,3-dichlorophenyl | 2-propyl |
| A-544 | 2,4-dichlorophenyl | 2-propyl |
| A-545 | 2,5-dichlorophenyl | 2-propyl |
| A-546 | 2,6-dichlorophenyl | 2-propyl |
| A-547 | 3,4-dichlorophenyl | 2-propyl |
| A-548 | 3,5-dichlorophenyl | 2-propyl |
| A-549 | 2,3-difluorophenyl | 2-propyl |
| A-550 | 2,4-difluorophenyl | 2-propyl |
| A-551 | 2,5-difluorophenyl | 2-propyl |
| A-552 | 2,6-difluorophenyl | 2-propyl |
| A-553 | 3,4-difluorophenyl | 2-propyl |
| A-554 | 3,5-difluorophenyl | 2-propyl |
| A-555 | 2,3-dimethylphenyl | 2-propyl |
| A-556 | 2,4-dimethylphenyl | 2-propyl |
| A-557 | 2,5-dimethylphenyl | 2-propyl |
| A-558 | 2,6-dimethylphenyl | 2-propyl |
| A-559 | 3,4-dimethylphenyl | 2-propyl |
| A-560 | 3,5-dimethylphenyl | 2-propyl |
| A-561 | 2,3-diethylphenyl | 2-propyl |
| A-562 | 2,4-diethylphenyl | 2-propyl |
| A-563 | 2,5-diethylphenyl | 2-propyl |
| A-564 | 2,6-diethylphenyl | 2-propyl |
| A-565 | 3,5-diethylphenyl | 2-propyl |
| A-566 | 3,4-diethylphenyl | 2-propyl |
| A-567 | 2,3-dimethoxyphenyl | 2-propyl |
| A-568 | 2,4-dimethoxyphenyl | 2-propyl |
| A-569 | 2,5-dimethoxyphenyl | 2-propyl |
| A-570 | 2,6-dimethoxyphenyl | 2-propyl |
| A-571 | 3,4-dimethoxyphenyl | 2-propyl |
| A-572 | 3,5-dimethoxyphenyl | 2-propyl |
| A-573 | 2,3-di(trifluoromethyl)phenyl | 2-propyl |
| A-574 | 2,4-di(trifluoromethyl)phenyl | 2-propyl |
| A-575 | 2,5-di(trifluoromethyl)phenyl | 2-propyl |
| A-576 | 2,6-di(trifluoromethyl)phenyl | 2-propyl |
| A-577 | 3,4-di(trifluoromethyl)phenyl | 2-propyl |
| A-578 | 3,5-di(trifluoromethyl)phenyl | 2-propyl |
| A-579 | 2,3-di(trifluoromethoxy)phenyl | 2-propyl |
| A-580 | 2,4-di(trifluoromethoxy)phenyl | 2-propyl |
| A-581 | 2,5-di(trifluoromethoxy)phenyl | 2-propyl |
| A-582 | 2,6-di(trifluoromethoxy)phenyl | 2-propyl |
| A-583 | 3,4-di(trifluoromethoxy)phenyl | 2-propyl |
| A-584 | 3,5-di(trifluoromethoxy)phenyl | 2-propyl |
| A-585 | 2,3-di(difluoromethoxy)phenyl | 2-propyl |
| A-586 | 2,4-di(difluoromethoxy)phenyl | 2-propyl |
| A-587 | 2,5-di(difluoromethoxy)phenyl | 2-propyl |
| A-588 | 2,6-di(difluoromethoxy)phenyl | 2-propyl |
| A-589 | 3,4-di(difluoromethoxy)phenyl | 2-propyl |
| A-590 | 3,5-di(difluoromethoxy)phenyl | 2-propyl |
| A-591 | 2,3-di(trifluoromethylthio)phenyl | 2-propyl |
| A-592 | 2,4-di(trifluoromethylthio)phenyl | 2-propyl |
| A-593 | 2,5-di(trifluoromethylthio)phenyl | 2-propyl |
| A-594 | 2,6-di(trifluoromethylthio)phenyl | 2-propyl |
| A-595 | 3,4-di(trifluoromethylthio)phenyl | 2-propyl |
| A-596 | 3,5-di(trifluoromethylthio)phenyl | 2-propyl |
| A-597 | 2-fluoro-3-chlorophenyl | 2-propyl |
| A-598 | 2-fluoro-4-chlorophenyl | 2-propyl |
| A-599 | 2-fluoro-5-chlorophenyl | 2-propyl |
| A-600 | 2-fluoro-6-chlorophenyl | 2-propyl |
| A-601 | 3-fluoro-4-chlorophenyl | 2-propyl |
| A-602 | 3-fluoro-5-chlorophenyl | 2-propyl |
| A-603 | 2-chloro-3-fluorophenyl | 2-propyl |
| A-604 | 2-chloro-4-fluorophenyl | 2-propyl |
| A-605 | 2-chloro-5-fluorophenyl | 2-propyl |
| A-606 | 3-chloro-4-fluorophenyl | 2-propyl |
| A-607 | 2-methyl-3-chlorophenyl | 2-propyl |
| A-608 | 2-methyl-4-chlorophenyl | 2-propyl |
| A-609 | 2-methyl-5-chlorophenyl | 2-propyl |
| A-610 | 2-methyl-6-chlorophenyl | 2-propyl |
| A-611 | 3-methyl-4-chlorophenyl | 2-propyl |
| A-612 | 3-methyl-5-chlorophenyl | 2-propyl |
| A-613 | 2-chloro-3-methylphenyl | 2-propyl |
| A-614 | 2-chloro-4-methylphenyl | 2-propyl |
| A-615 | 2-chloro-5-methylphenyl | 2-propyl |
| A-616 | 3-chloro-4-methylphenyl | 2-propyl |
| A-617 | 2-methyl-3-fluorophenyl | 2-propyl |
| A-618 | 2-methyl-4-fluorophenyl | 2-propyl |
| A-619 | 2-methyl-5-fluorophenyl | 2-propyl |
| A-620 | 2-methyl-6-fluorophenyl | 2-propyl |
| A-621 | 3-methyl-4-fluorophenyl | 2-propyl |
| A-622 | 3-methyl-5-fluorophenyl | 2-propyl |
| A-623 | 2-fluoro-3-methylphenyl | 2-propyl |
| A-624 | 2-fluoro-4-methylphenyl | 2-propyl |
| A-625 | 2-fluoro-5-methylphenyl | 2-propyl |
| A-626 | 3-fluoro-4-methylphenyl | 2-propyl |
| A-627 | 2-chloro-3-ethylphenyl | 2-propyl |
| A-628 | 2-chloro-4-ethylphenyl | 2-propyl |
| A-629 | 2-chloro-5-ethylphenyl | 2-propyl |
| A-630 | 3-chloro-4-ethylphenyl | 2-propyl |
| A-631 | 2-ethyl-3-chlorophenyl | 2-propyl |
| A-632 | 2-ethyl-4-chlorophenyl | 2-propyl |
| A-633 | 2-ethyl-5-chlorophenyl | 2-propyl |
| A-634 | 2-ethyl-6-chlorophenyl | 2-propyl |
| A-635 | 3-ethyl-4-chlorophenyl | 2-propyl |
| A-636 | 3-ethyl-5-chlorophenyl | 2-propyl |
| A-637 | 2-ethyl-3-fluorophenyl | 2-propyl |
| A-638 | 2-ethyl-4-fluorophenyl | 2-propyl |
| A-639 | 2-ethyl-5-fluorophenyl | 2-propyl |
| A-640 | 2-ethyl-6-fluorophenyl | 2-propyl |
| A-641 | 3-ethyl-4-fluorophenyl | 2-propyl |
| A-642 | 3-ethyl-5-fluorophenyl | 2-propyl |
| A-643 | 2-fluoro-3-ethylphenyl | 2-propyl |
| A-644 | 2-fluoro-4-ethylphenyl | 2-propyl |
| A-645 | 2-fluoro-5-ethylphenyl | 2-propyl |
| A-646 | 3-fluoro-4-ethylphenyl | 2-propyl |
| A-647 | 2-methoxy-3-chlorophenyl | 2-propyl |
| A-648 | 2-methoxy-4-chlorophenyl | 2-propyl |
| A-649 | 2-methoxy-5-chlorophenyl | 2-propyl |
| A-650 | 2-methoxy-6-chlorophenyl | 2-propyl |
| A-651 | 3-methoxy-4-chlorophenyl | 2-propyl |
| A-652 | 3-methoxy-5-chlorophenyl | 2-propyl |
| A-653 | 2-chloro-3-methoxyphenyl | 2-propyl |
| A-654 | 2-chloro-4-methoxyphenyl | 2-propyl |
| A-655 | 2-chloro-5-methoxyphenyl | 2-propyl |
| A-656 | 3-chloro-4-methoxyphenyl | 2-propyl |
| A-657 | 2-methoxy-3-fluorophenyl | 2-propyl |
| A-658 | 2-methoxy-4-fluorophenyl | 2-propyl |
| A-659 | 2-methoxy-5-fluorophenyl | 2-propyl |
| A-660 | 2-methoxy-6-fluorophenyl | 2-propyl |
| A-661 | 3-methoxy-4-fluorophenyl | 2-propyl |
| A-662 | 3-methoxy-5-fluorophenyl | 2-propyl |
| A-663 | 2-fluoro-3-methoxyphenyl | 2-propyl |
| A-664 | 2-fluoro-4-methoxyphenyl | 2-propyl |
| A-665 | 2-fluoro-5-methoxyphenyl | 2-propyl |
| A-666 | 3-fluoro-4-methoxyphenyl | 2-propyl |
| A-667 | 3-fluoro-5-methoxyphenyl | 2-propyl |
| A-668 | 2-(trifluoromethyl)-3-chlorophenyl | 2-propyl |
| A-669 | 2-(trifluoromethyl)-4-chlorophenyl | 2-propyl |
| A-670 | 2-(trifluoromethyl)-5-chlorophenyl | 2-propyl |
| A-671 | 2-(trifluoromethyl)-6-chlorophenyl | 2-propyl |
| A-672 | 3-(trifluoromethyl)-4-chlorophenyl | 2-propyl |
| A-673 | 3-(trifluoromethyl)-5-chlorophenyl | 2-propyl |
| A-674 | 2-chloro-3-(trifluoromethyl)phenyl | 2-propyl |
| A-675 | 2-chloro-4-(trifluoromethyl)phenyl | 2-propyl |
| A-676 | 2-chloro-5-(trifluoromethyl)phenyl | 2-propyl |
| A-677 | 3-chloro-4-(trifluoromethyl)phenyl | 2-propyl |
| A-678 | 2-(trifluoromethyl)-3-fluorophenyl | 2-propyl |
| A-679 | 2-(trifluoromethyl)-4-fluorophenyl | 2-propyl |
| A-680 | 2-(trifluoromethyl)-5-fluorophenyl | 2-propyl |
| A-681 | 2-(trifluoromethyl)-6-fluorophenyl | 2-propyl |
| A-682 | 3-(trifluoromethyl)-4-fluorophenyl | 2-propyl |
| A-683 | 3-(trifluoromethyl)-5-fluorophenyl | 2-propyl |
| A-684 | 2-fluoro-3-(trifluoromethyl)phenyl | 2-propyl |
| A-685 | 2-fluoro-4-(trifluoromethyl)phenyl | 2-propyl |
| A-686 | 2-fluoro-5-(trifluoromethyl)phenyl | 2-propyl |
| A-687 | 3-fluoro-4-(trifluoromethyl)phenyl | 2-propyl |
| A-688 | 2-(trifluoromethoxy)-3-chlorophenyl | 2-propyl |
| A-689 | 2-(trifluoromethoxy)-4-chlorophenyl | 2-propyl |
| A-690 | 2-(trifluoromethoxy)-5-chlorophenyl | 2-propyl |
| A-691 | 2-(trifluoromethoxy)-6-chlorophenyl | 2-propyl |
| A-692 | 3-(trifluoromethoxy)-4-chlorophenyl | 2-propyl |
| A-693 | 3-(trifluoromethoxy)-5-chlorophenyl | 2-propyl |
| A-694 | 2-chloro-3-(trifluoromethoxy)phenyl | 2-propyl |
| A-695 | 2-chloro-4-(trifluoromethoxy)phenyl | 2-propyl |
| A-696 | 2-chloro-5-(trifluoromethoxy)phenyl | 2-propyl |
| A-697 | 3-chloro-4-(trifluoromethoxy)phenyl | 2-propyl |
| A-698 | 2-(trifluoromethoxy)-3-fluorophenyl | 2-propyl |
| A-699 | 2-(trifluoromethoxy)-4-fluorophenyl | 2-propyl |
| A-700 | 2-(trifluoromethoxy)-5-fluorophenyl | 2-propyl |
| A-701 | 2-(trifluoromethoxy)-6-fluorophenyl | 2-propyl |
| A-702 | 3-(trifluoromethoxy)-4-fluorophenyl | 2-propyl |
| A-703 | 3-(trifluoromethoxy)-5-fluorophenyl | 2-propyl |
| A-704 | 2-fluoro-3-(trifluoromethoxy)phenyl | 2-propyl |
| A-705 | 2-fluoro-4-(trifluoromethoxy)phenyl | 2-propyl |
| A-706 | 2-fluoro-5-(trifluoromethoxy)phenyl | 2-propyl |
| A-707 | 3-fluoro-4-(trifluoromethoxy)phenyl | 2-propyl |
| A-708 | 2-(difluoromethoxy)-3-chlorophenyl | 2-propyl |
| A-709 | 2-(difluoromethoxy)-4-chlorophenyl | 2-propyl |
| A-710 | 2-(difluoromethoxy)-5-chlorophenyl | 2-propyl |
| A-711 | 2-(difluoromethoxy)-6-chlorophenyl | 2-propyl |
| A-712 | 3-(difluoromethoxy)-4-chlorophenyl | 2-propyl |
| A-713 | 3-(difluoromethoxy)-5-chlorophenyl | 2-propyl |
| A-714 | 2-chloro-3-(difluoromethoxy)phenyl | 2-propyl |
| A-715 | 2-chloro-4-(difluoromethoxy)phenyl | 2-propyl |
| A-716 | 2-chloro-5-(difluoromethoxy)phenyl | 2-propyl |
| A-717 | 3-chloro-4-(difluoromethoxy)phenyl | 2-propyl |
| A-718 | 2-(difluoromethoxy)-3-fluorophenyl | 2-propyl |
| A-719 | 2-(difluoromethoxy)-4-fluorophenyl | 2-propyl |
| A-720 | 2-(difluoromethoxy)-5-fluorophenyl | 2-propyl |
| A-721 | 2-(difluoromethoxy)-6-fluorophenyl | 2-propyl |
| A-722 | 3-(difluoromethoxy)-4-fluorophenyl | 2-propyl |
| A-723 | 3-(difluoromethoxy)-5-fluorophenyl | 2-propyl |
| A-724 | 2-fluoro-3-(difluoromethoxy)phenyl | 2-propyl |
| A-725 | 2-fluoro-4-(difluoromethoxy)phenyl | 2-propyl |
| A-726 | 2-fluoro-5-(difluoromethoxy)phenyl | 2-propyl |
| A-727 | 3-fluoro-4-(difluoromethoxy)phenyl | 2-propyl |
| A-728 | 2-(trifluoromethylthio)-3- | 2-propyl |
| | chlorophenyl | |
| A-729 | 2-(trifluoromethylthio)-4- | 2-propyl |
| | chlorophenyl | |
| A-730 | 2-(trifluoromethylthio)-5- | 2-propyl |
| | chlorophenyl | |
| A-731 | 2-(trifluoromethylthio)-6- | 2-propyl |
| | chlorophenyl | |
| A-732 | 3-(trifluoromethylthio)-4- | 2-propyl |
| | chlorophenyl | |
| A-733 | 3-(trifluoromethylthio)-5- | 2-propyl |
| | chlorophenyl | |
| A-734 | 2-chloro-3- | 2-propyl |
| | (trifluoromethylthio)phenyl | |
| A-735 | 2-chloro-4- | 2-propyl |
| | (trifluoromethylthio)phenyl | |
| A-736 | 2-chloro-5- | 2-propyl |
| | (trifluoromethylthio)phenyl | |
| A-737 | 3-chloro-4- | 2-propyl |
| | (trifluoromethylthio)phenyl | |
| A-738 | 2-(trifluoromethylthio)-3-fluorophenyl | 2-propyl |
| A-739 | 2-(trifluoromethylthio)-4-fluorophenyl | 2-propyl |
| A-740 | 2-(trifluoromethylthio)-5-fluorophenyl | 2-propyl |
| A-741 | 2-(trifluoromethylthio)-6-fluorophenyl | 2-propyl |
| A-742 | 3-(trifluoromethylthio)-4-fluorophenyl | 2-propyl |
| A-743 | 3-(trifluoromethylthio)-5-fluorophenyl | 2-propyl |
| A-744 | 2-fluoro-3-(trifluoromethylthio)phenyl | 2-propyl |
| A-745 | 2-fluoro-4-(trifluoromethylthio)phenyl | 2-propyl |
| A-746 | 2-fluoro-5-(trifluoromethylthio)phenyl | 2-propyl |
| A-747 | 3-fluoro-4-(trifluoromethylthio)phenyl | 2-propyl |
| A-748 | 2,3,4-trichlorophenyl | 2-propyl |
| A-749 | 2,3,5-trichlorophenyl | 2-propyl |
| A-750 | 2,3,6-trichlorophenyl | 2-propyl |
| A-751 | 2,4,5-trichlorophenyl | 2-propyl |
| A-752 | 2,4,6-trichlorophenyl | 2-propyl |
| A-753 | 3,4,5-trichlorophenyl | 2-propyl |
| A-754 | 2,3,4-trifluorophenyl | 2-propyl |
| A-755 | 2,3,5-trifluorophenyl | 2-propyl |
| A-756 | 2,3,6-trifluorophenyl | 2-propyl |
| A-757 | 2,4,5-trifluorophenyl | 2-propyl |
| A-758 | 2,4,6-trifluorophenyl | 2-propyl |
| A-759 | 3,4,5-trifluorophenyl | 2-propyl |
| A-760 | 2,3,4-trimethylphenyl | 2-propyl |
| A-761 | 2,3,5-trimethylphenyl | 2-propyl |
| A-762 | 2,3,6-trimethylphenyl | 2-propyl |
| A-763 | 2,4,5-trimethylphenyl | 2-propyl |
| A-764 | 2,4,6-trimethylphenyl | 2-propyl |
| A-765 | 3,4,5-trimethylphenyl | 2-propyl |
| A-766 | 2,3,4-trimethoxyphenyl | 2-propyl |
| A-767 | 2,3,5-trimethoxyphenyl | 2-propyl |
| A-768 | 2,3,6-trimethoxyphenyl | 2-propyl |
| A-769 | 2,4,5-trimethoxyphenyl | 2-propyl |
| A-770 | 2,4,6-trimethoxyphenyl | 2-propyl |
| A-771 | 3,4,5-trimethoxyphenyl | 2-propyl |
| A-772 | phenyl | butyl |
| A-773 | 2-chlorophenyl | butyl |
| A-774 | 3-chlorophenyl | butyl |
| A-775 | 4-chlorophenyl | butyl |
| A-776 | 2-fluorophenyl | butyl |
| A-777 | 3-fluorophenyl | butyl |
| A-778 | 4-fluorophenyl | butyl |
| A-779 | 2-methylphenyl | butyl |
| A-780 | 3-methylphenyl | butyl |
| A-781 | 4-methylphenyl | butyl |
| A-782 | 2-ethylphenyl | butyl |
| A-783 | 3-ethylphenyl | butyl |
| A-784 | 4-ethylphenyl | butyl |
| A-785 | 2-methoxyphenyl | butyl |
| A-786 | 3-methoxyphenyl | butyl |
| A-787 | 4-methoxyphenyl | butyl |
| A-788 | 2-trifluoromethylphenyl | butyl |
| A-789 | 3-trifluoromethylphenyl | butyl |
| A-790 | 4-trifluoromethylphenyl | butyl |
| A-791 | 2-trifluoromethoxyphenyl | butyl |
| A-792 | 3-trifluoromethoxyphenyl | butyl |
| A-793 | 4-trifluoromethoxyphenyl | butyl |
| A-794 | 2-difluoromethoxyphenyl | butyl |
| A-795 | 3-difluoromethoxyphenyl | butyl |
| A-796 | 4-difluoromethoxyphenyl | butyl |
| A-797 | 2-trifluoromethylthiophenyl | butyl |
| A-798 | 3-trifluoromethylthiophenyl | butyl |
| A-799 | 4-trifluoromethylthiophenyl | butyl |
| A-800 | 2,3-dichlorophenyl | butyl |
| A-801 | 2,4-dichlorophenyl | butyl |
| A-802 | 2,5-dichlorophenyl | butyl |
| A-803 | 2,6-dichlorophenyl | butyl |
| A-804 | 3,4-dichlorophenyl | butyl |
| A-805 | 3,5-dichlorophenyl | butyl |
| A-806 | 2,3-difluorophenyl | butyl |
| A-807 | 2,4-difluorophenyl | butyl |
| A-808 | 2,5-difluorophenyl | butyl |
| A-809 | 2,6-difluorophenyl | butyl |
| A-810 | 3,4-difluorophenyl | butyl |
| A-811 | 3,5-difluorophenyl | butyl |
| A-812 | 2,3-dimethylphenyl | butyl |
| A-813 | 2,4-dimethylphenyl | butyl |
| A-814 | 2,5-dimethylphenyl | butyl |
| A-815 | 2,6-dimethylphenyl | butyl |
| A-816 | 3,4-dimethylphenyl | butyl |
| A-817 | 3,5-dimethylphenyl | butyl |
| A-818 | 2,3-diethylphenyl | butyl |
| A-819 | 2,4-diethylphenyl | butyl |
| A-820 | 2,5-diethylphenyl | butyl |
| A-821 | 2,6-diethylphenyl | butyl |
| A-822 | 3,5-diethylphenyl | butyl |
| A-823 | 3,4-diethylphenyl | butyl |
| A-824 | 2,3-dimethoxyphenyl | butyl |
| A-825 | 2,4-dimethoxyphenyl | butyl |
| A-826 | 2,5-dimethoxyphenyl | butyl |
| A-827 | 2,6-dimethoxyphenyl | butyl |
| A-828 | 3,4-dimethoxyphenyl | butyl |
| A-829 | 3,5-dimethoxyphenyl | butyl |
| A-830 | 2,3-di(trifluoromethyl)phenyl | butyl |
| A-831 | 2,4-di(trifluoromethyl)phenyl | butyl |
| A-832 | 2,5-di(trifluoromethyl)phenyl | butyl |
| A-833 | 2,6-di(trifluoromethyl)phenyl | butyl |
| A-834 | 3,4-di(trifluoromethyl)phenyl | butyl |
| A-835 | 3,5-di(trifluoromethyl)phenyl | butyl |
| A-836 | 2,3-di(trifluoromethoxy)phenyl | butyl |
| A-837 | 2,4-di(trifluoromethoxy)phenyl | butyl |
| A-838 | 2,5-di(trifluoromethoxy)phenyl | butyl |
| A-839 | 2,6-di(trifluoromethoxy)phenyl | butyl |
| A-840 | 3,4-di(trifluoromethoxy)phenyl | butyl |
| A-841 | 3,5-di(trifluoromethoxy)phenyl | butyl |
| A-842 | 2,3-di(difluoromethoxy)phenyl | butyl |
| A-843 | 2,4-di(difluoromethoxy)phenyl | butyl |
| A-844 | 2,5-di(difluoromethoxy)phenyl | butyl |
| A-845 | 2,6-di(difluoromethoxy)phenyl | butyl |
| A-846 | 3,4-di(difluoromethoxy)phenyl | butyl |
| A-847 | 3,5-di(difluoromethoxy)phenyl | butyl |
| A-848 | 2,3-di(trifluoromethylthio)phenyl | butyl |
| A-849 | 2,4-di(trifluoromethylthio)phenyl | butyl |
| A-850 | 2,5-di(trifluoromethylthio)phenyl | butyl |
| A-851 | 2,6-di(trifluoromethylthio)phenyl | butyl |
| A-852 | 3,4-di(trifluoromethylthio)phenyl | butyl |
| A-853 | 3,5-di(trifluoromethylthio)phenyl | butyl |
| A-854 | 2-fluoro-3-chlorophenyl | butyl |
| A-855 | 2-fluoro-4-chlorophenyl | butyl |
| A-856 | 2-fluoro-5-chlorophenyl | butyl |
| A-857 | 2-fluoro-6-chlorophenyl | butyl |
| A-858 | 3-fluoro-4-chlorophenyl | butyl |
| A-859 | 3-fluoro-5-chlorophenyl | butyl |
| A-860 | 2-chloro-3-fluorophenyl | butyl |
| A-861 | 2-chloro-4-fluorophenyl | butyl |
| A-862 | 2-chloro-5-fluorophenyl | butyl |
| A-863 | 3-chloro-4-fluorophenyl | butyl |
| A-864 | 2-methyl-3-chlorophenyl | butyl |
| A-865 | 2-methyl-4-chlorophenyl | butyl |
| A-866 | 2-methyl-5-chlorophenyl | butyl |
| A-867 | 2-methyl-6-chlorophenyl | butyl |
| A-868 | 3-methyl-4-chlorophenyl | butyl |
| A-869 | 3-methyl-5-chlorophenyl | butyl |
| A-870 | 2-chloro-3-methylphenyl | butyl |
| A-871 | 2-chloro-4-methylphenyl | butyl |
| A-872 | 2-chloro-5-methylphenyl | butyl |
| A-873 | 3-chloro-4-methylphenyl | butyl |
| A-874 | 2-methyl-3-fluorophenyl | butyl |
| A-875 | 2-methyl-4-fluorophenyl | butyl |
| A-876 | 2-methyl-5-fluorophenyl | butyl |
| A-877 | 2-methyl-6-fluorophenyl | butyl |
| A-878 | 3-methyl-4-fluorophenyl | butyl |
| A-879 | 3-methyl-5-fluorophenyl | butyl |
| A-880 | 2-fluoro-3-methylphenyl | butyl |
| A-881 | 2-fluoro-4-methylphenyl | butyl |
| A-882 | 2-fluoro-5-methylphenyl | butyl |
| A-883 | 3-fluoro-4-methylphenyl | butyl |
| A-884 | 2-chloro-3-ethylphenyl | butyl |
| A-885 | 2-chloro-4-ethylphenyl | butyl |
| A-886 | 2-chloro-5-ethylphenyl | butyl |
| A-887 | 3-chloro-4-ethylphenyl | butyl |
| A-888 | 2-ethyl-3-chlorophenyl | butyl |
| A-889 | 2-ethyl-4-chlorophenyl | butyl |
| A-890 | 2-ethyl-5-chlorophenyl | butyl |
| A-891 | 2-ethyl-6-chlorophenyl | butyl |
| A-892 | 3-ethyl-4-chlorophenyl | butyl |
| A-893 | 3-ethyl-5-chlorophenyl | butyl |
| A-894 | 2-ethyl-3-fluorophenyl | butyl |
| A-895 | 2-ethyl-4-fluorophenyl | butyl |
| A-896 | 2-ethyl-5-fluorophenyl | butyl |
| A-897 | 2-ethyl-6-fluorophenyl | butyl |
| A-898 | 3-ethyl-4-fluorophenyl | butyl |
| A-899 | 3-ethyl-5-fluorophenyl | butyl |
| A-900 | 2-fluoro-3-ethylphenyl | butyl |
| A-901 | 2-fluoro-4-ethylphenyl | butyl |
| A-902 | 2-fluoro-5-ethylphenyl | butyl |
| A-903 | 3-fluoro-4-ethylphenyl | butyl |
| A-904 | 2-methoxy-3-chlorophenyl | butyl |
| A-905 | 2-methoxy-4-chlorophenyl | butyl |
| A-906 | 2-methoxy-5-chlorophenyl | butyl |
| A-907 | 2-methoxy-6-chlorophenyl | butyl |
| A-908 | 3-methoxy-4-chlorophenyl | butyl |
| A-909 | 3-methoxy-5-chlorophenyl | butyl |
| A-910 | 2-chloro-3-methoxyphenyl | butyl |
| A-911 | 2-chloro-4-methoxyphenyl | butyl |
| A-912 | 2-chloro-5-methoxyphenyl | butyl |
| A-913 | 3-chloro-4-methoxyphenyl | butyl |
| A-914 | 2-methoxy-3-fluorophenyl | butyl |
| A-915 | 2-methoxy-4-fluorophenyl | butyl |
| A-916 | 2-methoxy-5-fluorophenyl | butyl |
| A-917 | 2-methoxy-6-fluorophenyl | butyl |
| A-918 | 3-methoxy-4-fluorophenyl | butyl |
| A-919 | 3-methoxy-5-fluorophenyl | butyl |
| A-920 | 2-fluoro-3-methoxyphenyl | butyl |
| A-921 | 2-fluoro-4-methoxyphenyl | butyl |
| A-922 | 2-fluoro-5-methoxyphenyl | butyl |
| A-923 | 3-fluoro-4-methoxyphenyl | butyl |
| A-924 | 3-fluoro-5-methoxyphenyl | butyl |
| A-925 | 2-(trifluoromethyl)-3-chlorophenyl | butyl |
| A-926 | 2-(trifluoromethyl)-4-chlorophenyl | butyl |
| A-927 | 2-(trifluoromethyl)-5-chlorophenyl | butyl |
| A-928 | 2-(trifluoromethyl)-6-chlorophenyl | butyl |
| A-929 | 3-(trifluoromethyl)-4-chlorophenyl | butyl |
| A-930 | 3-(trifluoromethyl)-5-chlorophenyl | butyl |
| A-931 | 2-chloro-3-(trifluoromethyl)phenyl | butyl |
| A-932 | 2-chloro-4-(trifluoromethyl)phenyl | butyl |
| A-933 | 2-chloro-5-(trifluoromethyl)phenyl | butyl |
| A-934 | 3-chloro-4-(trifluoromethyl)phenyl | butyl |
| A-935 | 2-(trifluoromethyl)-3-fluorophenyl | butyl |
| A-936 | 2-(trifluoromethyl)-4-fluorophenyl | butyl |
| A-937 | 2-(trifluoromethyl)-5-fluorophenyl | butyl |
| A-938 | 2-(trifluoromethyl)-6-fluorophenyl | butyl |
| A-939 | 3-(trifluoromethyl)-4-fluorophenyl | butyl |
| A-940 | 3-(trifluoromethyl)-5-fluorophenyl | butyl |
| A-941 | 2-fluoro-3-(trifluoromethyl)phenyl | butyl |
| A-942 | 2-fluoro-4-(trifluoromethyl)phenyl | butyl |
| A-943 | 2-fluoro-5-(trifluoromethyl)phenyl | butyl |
| A-944 | 3-fluoro-4-(trifluoromethyl)phenyl | butyl |
| A-945 | 2-(trifluoromethoxy)-3-chlorophenyl | butyl |
| A-946 | 2-(trifluoromethoxy)-4-chlorophenyl | butyl |
| A-947 | 2-(trifluoromethoxy)-5-chlorophenyl | butyl |
| A-948 | 2-(trifluoromethoxy)-6-chlorophenyl | butyl |
| A-949 | 3-(trifluoromethoxy)-4-chlorophenyl | butyl |
| A-950 | 3-(trifluoromethoxy)-5-chlorophenyl | butyl |
| A-951 | 2-chloro-3-(trifluoromethoxy)phenyl | butyl |
| A-952 | 2-chloro-4-(trifluoromethoxy)phenyl | butyl |
| A-953 | 2-chloro-5-(trifluoromethoxy)phenyl | butyl |
| A-954 | 3-chloro-4-(trifluoromethoxy)phenyl | butyl |
| A-955 | 2-(trifluoromethoxy)-3-fluorophenyl | butyl |
| A-956 | 2-(trifluoromethoxy)-4-fluorophenyl | butyl |
| A-957 | 2-(trifluoromethoxy)-5-fluorophenyl | butyl |
| A-958 | 2-(trifluoromethoxy)-6-fluorophenyl | butyl |
| A-959 | 3-(trifluoromethoxy)-4-fluorophenyl | butyl |
| A-960 | 3-(trifluoromethoxy)-5-fluorophenyl | butyl |
| A-961 | 2-fluoro-3-(trifluoromethoxy)phenyl | butyl |
| A-962 | 2-fluoro-4-(trifluoromethoxy)phenyl | butyl |
| A-963 | 2-fluoro-5-(trifluoromethoxy)phenyl | butyl |
| A-964 | 3-fluoro-4-(trifluoromethoxy)phenyl | butyl |
| A-965 | 2-(difluoromethoxy)-3-chlorophenyl | butyl |
| A-966 | 2-(difluoromethoxy)-4-chlorophenyl | butyl |
| A-967 | 2-(difluoromethoxy)-5-chlorophenyl | butyl |
| A-968 | 2-(difluoromethoxy)-6-chlorophenyl | butyl |
| A-969 | 3-(difluoromethoxy)-4-chlorophenyl | butyl |
| A-970 | 3-(difluoromethoxy)-5-chlorophenyl | butyl |
| A-971 | 2-chloro-3-(difluoromethoxy)phenyl | butyl |
| A-972 | 2-chloro-4-(difluoromethoxy)phenyl | butyl |
| A-973 | 2-chloro-5-(difluoromethoxy)phenyl | butyl |
| A-974 | 3-chloro-4-(difluoromethoxy)phenyl | butyl |
| A-975 | 2-(difluoromethoxy)-3-fluorophenyl | butyl |
| A-976 | 2-(difluoromethoxy)-4-fluorophenyl | butyl |
| A-977 | 2-(difluoromethoxy)-5-fluorophenyl | butyl |
| A-978 | 2-(difluoromethoxy)-6-fluorophenyl | butyl |
| A-979 | 3-(difluoromethoxy)-4-fluorophenyl | butyl |
| A-980 | 3-(difluoromethoxy)-5-fluorophenyl | butyl |
| A-981 | 2-fluoro-3-(difluoromethoxy)phenyl | butyl |
| A-982 | 2-fluoro-4-(difluoromethoxy)phenyl | butyl |
| A-983 | 2-fluoro-5-(difluoromethoxy)phenyl | butyl |
| A-984 | 3-fluoro-4-(difluoromethoxy)phenyl | butyl |
| A-985 | 2-(trifluoromethylthio)-3- | butyl |
| | chlorophenyl | |
| A-986 | 2-(trifluoromethylthio)-4- | butyl |
| | chlorophenyl | |
| A-987 | 2-(trifluoromethylthio)-5- | butyl |
| | chlorophenyl | |
| A-988 | 2-(trifluoromethylthio)-6- | butyl |
| | chlorophenyl | |
| A-989 | 3-(trifluoromethylthio)-4- | butyl |
| | chlorophenyl | |
| A-990 | 3-(trifluoromethylthio)-5- | butyl |
| | chlorophenyl | |
| A-991 | 2-chloro-3- | butyl |
| | (trifluoromethylthio)phenyl | |
| A-992 | 2-chloro-4- | butyl |
| | (trifluoromethylthio)phenyl | |
| A-993 | 2-chloro-5- | butyl |
| | (trifluoromethylthio)phenyl | |
| A-994 | 3-chloro-4- | butyl |
| | (trifluoromethylthio)phenyl | |
| A-995 | 2-(trifluoromethylthio)-3-fluorophenyl | butyl |
| A-996 | 2-(trifluoromethylthio)-4-fluorophenyl | butyl |
| A-997 | 2-(trifluoromethylthio)-5-fluorophenyl | butyl |
| A-998 | 2-(trifluoromethylthio)-6-fluorophenyl | butyl |
| A-999 | 3-(trifluoromethylthio)-4-fluorophenyl | butyl |
| A-1000 | 3-(trifluoromethylthio)-5-fluorophenyl | butyl |
| A-1001 | 2-fluoro-3-(trifluoromethylthio)phenyl | butyl |
| A-1002 | 2-fluoro-4-(trifluoromethylthio)phenyl | butyl |
| A-1003 | 2-fluoro-5-(trifluoromethylthio)phenyl | butyl |
| A-1004 | 3-fluoro-4-(trifluoromethylthio)phenyl | butyl |
| A-1005 | 2,3,4-trichlorophenyl | butyl |
| A-1006 | 2,3,5-trichlorophenyl | butyl |
| A-1007 | 2,3,6-trichlorophenyl | butyl |
| A-1008 | 2,4,5-trichlorophenyl | butyl |
| A-1009 | 2,4,6-trichlorophenyl | butyl |
| A-1010 | 3,4,5-trichlorophenyl | butyl |
| A-1011 | 2,3,4-trifluorophenyl | butyl |
| A-1012 | 2,3,5-trifluorophenyl | butyl |
| A-1013 | 2,3,6-trifluorophenyl | butyl |
| A-1014 | 2,4,5-trifluorophenyl | butyl |
| A-1015 | 2,4,6-trifluorophenyl | butyl |
| A-1016 | 3,4,5-trifluorophenyl | butyl |
| A-1017 | 2,3,4-trimethylphenyl | butyl |
| A-1018 | 2,3,5-trimethylphenyl | butyl |
| A-1019 | 2,3,6-trimethylphenyl | butyl |
| A-1020 | 2,4,5-trimethylphenyl | butyl |
| A-1021 | 2,4,6-trimethylphenyl | butyl |
| A-1022 | 3,4,5-trimethylphenyl | butyl |
| A-1023 | 2,3,4-trimethoxyphenyl | butyl |
| A-1024 | 2,3,5-trimethoxyphenyl | butyl |
| A-1025 | 2,3,6-trimethoxyphenyl | butyl |
| A-1026 | 2,4,5-trimethoxyphenyl | butyl |
| A-1027 | 2,4,6-trimethoxyphenyl | butyl |
| A-1028 | 3,4,5-trimethoxyphenyl | butyl |
| A-1029 | phenyl | methyl |
| A-1030 | 2-chlorophenyl | methyl |
| A-1031 | 3-chlorophenyl | methyl |
| A-1032 | 4-chlorophenyl | methyl |
| A-1033 | 2-fluorophenyl | methyl |
| A-1034 | 3-fluorophenyl | methyl |
| A-1035 | 4-fluorophenyl | methyl |
| A-1036 | 2-methylphenyl | methyl |
| A-1037 | 3-methylphenyl | methyl |
| A-1038 | 4-methylphenyl | methyl |
| A-1039 | 2-ethylphenyl | methyl |
| A-1040 | 3-ethylphenyl | methyl |
| A-1041 | 4-ethylphenyl | methyl |
| A-1042 | 2-methoxyphenyl | methyl |
| A-1043 | 3-methoxyphenyl | methyl |
| A-1044 | 4-methoxyphenyl | methyl |
| A-1045 | 2-trifluoromethylphenyl | methyl |
| A-1046 | 3-trifluoromethylphenyl | methyl |
| A-1047 | 4-trifluoromethylphenyl | methyl |
| A-1048 | 2-trifluoromethoxyphenyl | methyl |
| A-1049 | 3-trifluoromethoxyphenyl | methyl |
| A-1050 | 4-trifluoromethoxyphenyl | methyl |
| A-1051 | 2-difluoromethoxyphenyl | methyl |
| A-1052 | 3-difluoromethoxyphenyl | methyl |
| A-1053 | 4-difluoromethoxyphenyl | methyl |
| A-1054 | 2-trifluoromethylthiophenyl | methyl |
| A-1055 | 3-trifluoromethylthiophenyl | methyl |
| A-1056 | 4-trifluoromethylthiophenyl | methyl |
| A-1057 | 2,3-dichlorophenyl | methyl |
| A-1058 | 2,4-dichlorophenyl | methyl |
| A-1059 | 2,5-dichlorophenyl | methyl |
| A-1060 | 2,6-dichlorophenyl | methyl |
| A-1061 | 3,4-dichlorophenyl | methyl |
| A-1062 | 3,5-dichlorophenyl | methyl |
| A-1063 | 2,3-difluorophenyl | methyl |
| A-1064 | 2,4-difluorophenyl | methyl |
| A-1065 | 2,5-difluorophenyl | methyl |
| A-1066 | 2,6-difluorophenyl | methyl |
| A-1067 | 3,4-difluorophenyl | methyl |
| A-1068 | 3,5-difluorophenyl | methyl |
| A-1069 | 2,3-dimethylphenyl | methyl |
| A-1070 | 2,4-dimethylphenyl | methyl |
| A-1071 | 2,5-dimethylphenyl | methyl |
| A-1072 | 2,6-dimethylphenyl | methyl |
| A-1073 | 3,4-dimethylphenyl | methyl |
| A-1074 | 3,5-dimethylphenyl | methyl |
| A-1075 | 2,3-diethylphenyl | methyl |
| A-1076 | 2,4-diethylphenyl | methyl |
| A-1077 | 2,5-diethylphenyl | methyl |
| A-1078 | 2,6-diethylphenyl | methyl |
| A-1079 | 3,5-diethylphenyl | methyl |
| A-1080 | 3,4-diethylphenyl | methyl |
| A-1081 | 2,3-dimethoxyphenyl | methyl |
| A-1082 | 2,4-dimethoxyphenyl | methyl |
| A-1083 | 2,5-dimethoxyphenyl | methyl |
| A-1084 | 2,6-dimethoxyphenyl | methyl |
| A-1085 | 3,4-dimethoxyphenyl | methyl |
| A-1086 | 3,5-dimethoxyphenyl | methyl |
| A-1087 | 2,3-di(trifluoromethyl)phenyl | methyl |
| A-1088 | 2,4-di(trifluoromethyl)phenyl | methyl |
| A-1089 | 2,5-di(trifluoromethyl)phenyl | methyl |
| A-1090 | 2,6-di(trifluoromethyl)phenyl | methyl |
| A-1091 | 3,4-di(trifluoromethyl)phenyl | methyl |
| A-1092 | 3,5-di(trifluoromethyl)phenyl | methyl |
| A-1093 | 2,3-di(trifluoromethoxy)phenyl | methyl |
| A-1094 | 2,4-di(trifluoromethoxy)phenyl | methyl |
| A-1095 | 2,5-di(trifluoromethoxy)phenyl | methyl |
| A-1096 | 2,6-di(trifluoromethoxy)phenyl | methyl |
| A-1097 | 3,4-di(trifluoromethoxy)phenyl | methyl |
| A-1098 | 3,5-di(trifluoromethoxy)phenyl | methyl |
| A-1099 | 2,3-di(difluoromethoxy)phenyl | methyl |
| A-1100 | 2,4-di(difluoromethoxy)phenyl | methyl |
| A-1101 | 2,5-di(difluoromethoxy)phenyl | methyl |
| A-1102 | 2,6-di(difluoromethoxy)phenyl | methyl |
| A-1103 | 3,4-di(difluoromethoxy)phenyl | methyl |
| A-1104 | 3,5-di(difluoromethoxy)phenyl | methyl |
| A-1105 | 2,3-di(trifluoromethylthio)phenyl | methyl |
| A-1106 | 2,4-di(trifluoromethylthio)phenyl | methyl |
| A-1107 | 2,5-di(trifluoromethylthio)phenyl | methyl |
| A-1108 | 2,6-di(trifluoromethylthio)phenyl | methyl |
| A-1109 | 3,4-di(trifluoromethylthio)phenyl | methyl |
| A-1110 | 3,5-di(trifluoromethylthio)phenyl | methyl |
| A-1111 | 2-fluoro-3-chlorophenyl | methyl |
| A-1112 | 2-fluoro-4-chlorophenyl | methyl |
| A-1113 | 2-fluoro-5-chlorophenyl | methyl |
| A-1114 | 2-fluoro-6-chlorophenyl | methyl |
| A-1115 | 3-fluoro-4-chlorophenyl | methyl |
| A-1116 | 3-fluoro-5-chlorophenyl | methyl |
| A-1117 | 2-chloro-3-fluorophenyl | methyl |
| A-1118 | 2-chloro-4-fluorophenyl | methyl |
| A-1119 | 2-chloro-5-fluorophenyl | methyl |
| A-1120 | 3-chloro-4-fluorophenyl | methyl |
| A-1121 | 2-methyl-3-chlorophenyl | methyl |
| A-1122 | 2-methyl-4-chlorophenyl | methyl |
| A-1123 | 2-methyl-5-chlorophenyl | methyl |
| A-1124 | 2-methyl-6-chlorophenyl | methyl |
| A-1125 | 3-methyl-4-chlorophenyl | methyl |
| A-1126 | 3-methyl-5-chlorophenyl | methyl |
| A-1127 | 2-chloro-3-methylphenyl | methyl |
| A-1128 | 2-chloro-4-methylphenyl | methyl |
| A-1129 | 2-chloro-5-methylphenyl | methyl |
| A-1130 | 3-chloro-4-methylphenyl | methyl |
| A-1131 | 2-methyl-3-fluorophenyl | methyl |
| A-1132 | 2-methyl-4-fluorophenyl | methyl |
| A-1133 | 2-methyl-5-fluorophenyl | methyl |
| A-1134 | 2-methyl-6-fluorophenyl | methyl |
| A-1135 | 3-methyl-4-fluorophenyl | methyl |
| A-1136 | 3-methyl-5-fluorophenyl | methyl |
| A-1137 | 2-fluoro-3-methylphenyl | methyl |
| A-1138 | 2-fluoro-4-methylphenyl | methyl |
| A-1139 | 2-fluoro-5-methylphenyl | methyl |
| A-1140 | 3-fluoro-4-methylphenyl | methyl |
| A-1141 | 2-chloro-3-ethylphenyl | methyl |
| A-1142 | 2-chloro-4-ethylphenyl | methyl |
| A-1143 | 2-chloro-5-ethylphenyl | methyl |
| A-1144 | 3-chloro-4-ethylphenyl | methyl |
| A-1145 | 2-ethyl-3-chlorophenyl | methyl |
| A-1146 | 2-ethyl-4-chlorophenyl | methyl |
| A-1147 | 2-ethyl-5-chlorophenyl | methyl |
| A-1148 | 2-ethyl-6-chlorophenyl | methyl |
| A-1149 | 3-ethyl-4-chlorophenyl | methyl |
| A-1150 | 3-ethyl-5-chlorophenyl | methyl |
| A-1151 | 2-ethyl-3-fluorophenyl | methyl |
| A-1152 | 2-ethyl-4-fluorophenyl | methyl |
| A-1153 | 2-ethyl-5-fluorophenyl | methyl |
| A-1154 | 2-ethyl-6-fluorophenyl | methyl |
| A-1155 | 3-ethyl-4-fluorophenyl | methyl |
| A-1156 | 3-ethyl-5-fluorophenyl | methyl |
| A-1157 | 2-fluoro-3-ethylphenyl | methyl |
| A-1158 | 2-fluoro-4-ethylphenyl | methyl |
| A-1159 | 2-fluoro-5-ethylphenyl | methyl |
| A-1160 | 3-fluoro-4-ethylphenyl | methyl |
| A-1161 | 2-methoxy-3-chlorophenyl | methyl |
| A-1162 | 2-methoxy-4-chlorophenyl | methyl |
| A-1163 | 2-methoxy-5-chlorophenyl | methyl |
| A-1164 | 2-methoxy-6-chlorophenyl | methyl |
| A-1165 | 3-methoxy-4-chlorophenyl | methyl |
| A-1166 | 3-methoxy-5-chlorophenyl | methyl |
| A-1167 | 2-chloro-3-methoxyphenyl | methyl |
| A-1168 | 2-chloro-4-methoxyphenyl | methyl |
| A-1169 | 2-chloro-5-methoxyphenyl | methyl |
| A-1170 | 3-chloro-4-methoxyphenyl | methyl |
| A-1171 | 2-methoxy-3-fluorophenyl | methyl |
| A-1172 | 2-methoxy-4-fluorophenyl | methyl |
| A-1173 | 2-methoxy-5-fluorophenyl | methyl |
| A-1174 | 2-methoxy-6-fluorophenyl | methyl |
| A-1175 | 3-methoxy-4-fluorophenyl | methyl |
| A-1176 | 3-methoxy-5-fluorophenyl | methyl |
| A-1177 | 2-fluoro-3-methoxyphenyl | methyl |
| A-1178 | 2-fluoro-4-methoxyphenyl | methyl |
| A-1179 | 2-fluoro-5-methoxyphenyl | methyl |
| A-1180 | 3-fluoro-4-methoxyphenyl | methyl |
| A-1181 | 3-fluoro-5-methoxyphenyl | methyl |
| A-1182 | 2-(trifluoromethyl)-3-chlorophenyl | methyl |
| A-1183 | 2-(trifluoromethyl)-4-chlorophenyl | methyl |
| A-1184 | 2-(trifluoromethyl)-5-chlorophenyl | methyl |
| A-1185 | 2-(trifluoromethyl)-6-chlorophenyl | methyl |
| A-1186 | 3-(trifluoromethyl)-4-chlorophenyl | methyl |
| A-1187 | 3-(trifluoromethyl)-5-chlorophenyl | methyl |
| A-1188 | 2-chloro-3-(trifluoromethyl)phenyl | methyl |
| A-1189 | 2-chloro-4-(trifluoromethyl)phenyl | methyl |
| A-1190 | 2-chloro-5-(trifluoromethyl)phenyl | methyl |
| A-1191 | 3-chloro-4-(trifluoromethyl)phenyl | methyl |
| A-1192 | 2-(trifluoromethyl)-3-fluorophenyl | methyl |
| A-1193 | 2-(trifluoromethyl)-4-fluorophenyl | methyl |
| A-1194 | 2-(trifluoromethyl)-5-fluorophenyl | methyl |
| A-1195 | 2-(trifluoromethyl)-6-fluorophenyl | methyl |
| A-1196 | 3-(trifluoromethyl)-4-fluorophenyl | methyl |
| A-1197 | 3-(trifluoromethyl)-5-fluorophenyl | methyl |
| A-1198 | 2-fluoro-3-(trifluoromethyl)phenyl | methyl |
| A-1199 | 2-fluoro-4-(trifluoromethyl)phenyl | methyl |
| A-1200 | 2-fluoro-5-(trifluoromethyl)phenyl | methyl |
| A-1201 | 3-fluoro-4-(trifluoromethyl)phenyl | methyl |
| A-1202 | 2-(trifluoromethoxy)-3-chlorophenyl | methyl |
| A-1203 | 2-(trifluoromethoxy)-4-chlorophenyl | methyl |
| A-1204 | 2-(trifluoromethoxy)-5-chlorophenyl | methyl |
| A-1205 | 2-(trifluoromethoxy)-6-chlorophenyl | methyl |
| A-1206 | 3-(trifluoromethoxy)-4-chlorophenyl | methyl |
| A-1207 | 3-(trifluoromethoxy)-5-chlorophenyl | methyl |
| A-1208 | 2-chloro-3-(trifluoromethoxy)phenyl | methyl |
| A-1209 | 2-chloro-4-(trifluoromethoxy)phenyl | methyl |
| A-1210 | 2-chloro-5-(trifluoromethoxy)phenyl | methyl |
| A-1211 | 3-chloro-4-(trifluoromethoxy)phenyl | methyl |
| A-1212 | 2-(trifluoromethoxy)-3-fluorophenyl | methyl |
| A-1213 | 2-(trifluoromethoxy)-4-fluorophenyl | methyl |
| A-1214 | 2-(trifluoromethoxy)-5-fluorophenyl | methyl |
| A-1215 | 2-(trifluoromethoxy)-6-fluorophenyl | methyl |
| A-1216 | 3-(trifluoromethoxy)-4-fluorophenyl | methyl |
| A-1217 | 3-(trifluoromethoxy)-5-fluorophenyl | methyl |
| A-1218 | 2-fluoro-3-(trifluoromethoxy)phenyl | methyl |
| A-1219 | 2-fluoro-4-(trifluoromethoxy)phenyl | methyl |
| A-1220 | 2-fluoro-5-(trifluoromethoxy)phenyl | methyl |
| A-1221 | 3-fluoro-4-(trifluoromethoxy)phenyl | methyl |
| A-1222 | 2-(difluoromethoxy)-3-chlorophenyl | methyl |
| A-1223 | 2-(difluoromethoxy)-4-chlorophenyl | methyl |
| A-1224 | 2-(difluoromethoxy)-5-chlorophenyl | methyl |
| A-1225 | 2-(difluoromethoxy)-6-chlorophenyl | methyl |
| A-1226 | 3-(difluoromethoxy)-4-chlorophenyl | methyl |
| A-1227 | 3-(difluoromethoxy)-5-chlorophenyl | methyl |
| A-1228 | 2-chloro-3-(difluoromethoxy)phenyl | methyl |
| A-1229 | 2-chloro-4-(difluoromethoxy)phenyl | methyl |
| A-1230 | 2-chloro-5-(difluoromethoxy)phenyl | methyl |
| A-1231 | 3-chloro-4-(difluoromethoxy)phenyl | methyl |
| A-1232 | 2-(difluoromethoxy)-3-fluorophenyl | methyl |
| A-1233 | 2-(difluoromethoxy)-4-fluorophenyl | methyl |
| A-1234 | 2-(difluoromethoxy)-5-fluorophenyl | methyl |
| A-1235 | 2-(difluoromethoxy)-6-fluorophenyl | methyl |
| A-1236 | 3-(difluoromethoxy)-4-fluorophenyl | methyl |
| A-1237 | 3-(difluoromethoxy)-5-fluorophenyl | methyl |
| A-1238 | 2-fluoro-3-(difluoromethoxy)phenyl | methyl |
| A-1239 | 2-fluoro-4-(difluoromethoxy)phenyl | methyl |
| A-1240 | 2-fluoro-5-(difluoromethoxy)phenyl | methyl |
| A-1241 | 3-fluoro-4-(difluoromethoxy)phenyl | methyl |
| A-1242 | 2-(trifluoromethylthio)-3- | methyl |
| | chlorophenyl | |
| A-1243 | 2-(trifluoromethylthio)-4- | methyl |
| | chlorophenyl | |
| A-1244 | 2-(trifluoromethylthio)-5- | methyl |
| | chlorophenyl | |
| A-1245 | 2-(trifluoromethylthio)-6- | methyl |
| | chlorophenyl | |
| A-1246 | 3-(trifluoromethylthio)-4- | methyl |
| | chlorophenyl | |
| A-1247 | 3-(trifluoromethylthio)-5- | methyl |
| | chlorophenyl | |
| A-1248 | 2-chloro-3- | methyl |
| | (trifluoromethylthio)phenyl | |
| A-1249 | 2-chloro-4- | methyl |
| | (trifluoromethylthio)phenyl | |
| A-1250 | 2-chloro-5- | methyl |
| | (trifluoromethylthio)phenyl | |
| A-1251 | 3-chloro-4- | methyl |
| | (trifluoromethylthio)phenyl | |
| A-1252 | 2-(trifluoromethylthio)-3-fluorophenyl | methyl |
| A-1253 | 2-(trifluoromethylthio)-4-fluorophenyl | methyl |
| A-1254 | 2-(trifluoromethylthio)-5-fluorophenyl | methyl |
| A-1255 | 2-(trifluoromethylthio)-6-fluorophenyl | methyl |
| A-1256 | 3-(trifluoromethylthio)-4-fluorophenyl | methyl |
| A-1257 | 3-(trifluoromethylthio)-5-fluorophenyl | methyl |
| A-1258 | 2-fluoro-3-(trifluoromethylthio)phenyl | methyl |
| A-1259 | 2-fluoro-4-(trifluoromethylthio)phenyl | methyl |
| A-1260 | 2-fluoro-5-(trifluoromethylthio)phenyl | methyl |
| A-1261 | 3-fluoro-4-(trifluoromethylthio)phenyl | methyl |
| A-1262 | 2,3,4-trichlorophenyl | methyl |
| A-1263 | 2,3,5-trichlorophenyl | methyl |
| A-1264 | 2,3,6-trichlorophenyl | methyl |
| A-1265 | 2,4,5-trichlorophenyl | methyl |
| A-1266 | 2,4,6-trichlorophenyl | methyl |
| A-1267 | 3,4,5-trichlorophenyl | methyl |
| A-1268 | 2,3,4-trifluorophenyl | methyl |
| A-1269 | 2,3,5-trifluorophenyl | methyl |
| A-1270 | 2,3,6-trifluorophenyl | methyl |
| A-1271 | 2,4,5-trifluorophenyl | methyl |
| A-1272 | 2,4,6-trifluorophenyl | methyl |
| A-1273 | 3,4,5-trifluorophenyl | methyl |
| A-1274 | 2,3,4-trimethylphenyl | methyl |
| A-1275 | 2,3,5-trimethylphenyl | methyl |
| A-1276 | 2,3,6-trimethylphenyl | methyl |
| A-1277 | 2,4,5-trimethylphenyl | methyl |
| A-1278 | 2,4,6-trimethylphenyl | methyl |
| A-1279 | 3,4,5-trimethylphenyl | methyl |
| A-1280 | 2,3,4-trimethoxyphenyl | methyl |
| A-1281 | 2,3,5-trimethoxyphenyl | methyl |
| A-1282 | 2,3,6-trimethoxyphenyl | methyl |
| A-1283 | 2,4,5-trimethoxyphenyl | methyl |
| A-1284 | 2,4,6-trimethoxyphenyl | methyl |
| A-1285 | 3,4,5-trimethoxyphenyl | methyl |
| A-1286 | phenyl | ethyl |
| A-1287 | 2-chlorophenyl | ethyl |
| A-1288 | 3-chlorophenyl | ethyl |
| A-1289 | 4-chlorophenyl | ethyl |
| A-1290 | 2-fluorophenyl | ethyl |
| A-1291 | 3-fluorophenyl | ethyl |
| A-1292 | 4-fluorophenyl | ethyl |
| A-1293 | 2-methylphenyl | ethyl |
| A-1294 | 3-methylphenyl | ethyl |
| A-1295 | 4-methylphenyl | ethyl |
| A-1296 | 2-ethylphenyl | ethyl |
| A-1297 | 3-ethylphenyl | ethyl |
| A-1298 | 4-ethylphenyl | ethyl |
| A-1299 | 2-methoxyphenyl | ethyl |
| A-1300 | 3-methoxyphenyl | ethyl |
| A-1301 | 4-methoxyphenyl | ethyl |
| A-1302 | 2-trifluoromethylphenyl | ethyl |
| A-1303 | 3-trifluoromethylphenyl | ethyl |
| A-1304 | 4-trifluoromethylphenyl | ethyl |
| A-1305 | 2-trifluoromethoxyphenyl | ethyl |
| A-1306 | 3-trifluoromethoxyphenyl | ethyl |
| A-1307 | 4-trifluoromethoxyphenyl | ethyl |
| A-1308 | 2-difluoromethoxyphenyl | ethyl |
| A-1309 | 3-difluoromethoxyphenyl | ethyl |
| A-1310 | 4-difluoromethoxyphenyl | ethyl |
| A-1311 | 2-trifluoromethylthiophenyl | ethyl |
| A-1312 | 3-trifluoromethylthiophenyl | ethyl |
| A-1313 | 4-trifluoromethylthiophenyl | ethyl |
| A-1314 | 2,3-dichlorophenyl | ethyl |
| A-1315 | 2,4-dichlorophenyl | ethyl |
| A-1316 | 2,5-dichlorophenyl | ethyl |
| A-1317 | 2,6-dichlorophenyl | ethyl |
| A-1318 | 3,4-dichlorophenyl | ethyl |
| A-1319 | 3,5-dichlorophenyl | ethyl |
| A-1320 | 2,3-difluorophenyl | ethyl |
| A-1321 | 2,4-difluorophenyl | ethyl |
| A-1322 | 2,5-difluorophenyl | ethyl |
| A-1323 | 2,6-difluorophenyl | ethyl |
| A-1324 | 3,4-difluorophenyl | ethyl |
| A-1325 | 3,5-difluorophenyl | ethyl |
| A-1326 | 2,3-dimethylphenyl | ethyl |
| A-1327 | 2,4-dimethylphenyl | ethyl |
| A-1328 | 2,5-dimethylphenyl | ethyl |
| A-1329 | 2,6-dimethylphenyl | ethyl |
| A-1330 | 3,4-dimethylphenyl | ethyl |
| A-1331 | 3,5-dimethylphenyl | ethyl |
| A-1332 | 2,3-diethylphenyl | ethyl |
| A-1333 | 2,4-diethylphenyl | ethyl |
| A-1334 | 2,5-diethylphenyl | ethyl |
| A-1335 | 2,6-diethylphenyl | ethyl |
| A-1336 | 3,5-diethylphenyl | ethyl |
| A-1337 | 3,4-diethylphenyl | ethyl |
| A-1338 | 2,3-dimethoxyphenyl | ethyl |
| A-1339 | 2,4-dimethoxyphenyl | ethyl |
| A-1340 | 2,5-dimethoxyphenyl | ethyl |
| A-1341 | 2,6-dimethoxyphenyl | ethyl |
| A-1342 | 3,4-dimethoxyphenyl | ethyl |
| A-1343 | 3,5-dimethoxyphenyl | ethyl |
| A-1344 | 2,3-di(trifluoromethyl)phenyl | ethyl |
| A-1345 | 2,4-di(trifluoromethyl)phenyl | ethyl |
| A-1346 | 2,5-di(trifluoromethyl)phenyl | ethyl |
| A-1347 | 2,6-di(trifluoromethyl)phenyl | ethyl |
| A-1348 | 3,4-di(trifluoromethyl)phenyl | ethyl |
| A-1349 | 3,5-di(trifluoromethyl)phenyl | ethyl |
| A-1350 | 2,3-di(trifluoromethoxy)phenyl | ethyl |
| A-1351 | 2,4-di(trifluoromethoxy)phenyl | ethyl |
| A-1352 | 2,5-di(trifluoromethoxy)phenyl | ethyl |
| A-1353 | 2,6-di(trifluoromethoxy)phenyl | ethyl |
| A-1354 | 3,4-di(trifluoromethoxy)phenyl | ethyl |
| A-1355 | 3,5-di(trifluoromethoxy)phenyl | ethyl |
| A-1356 | 2,3-di(difluoromethoxy)phenyl | ethyl |
| A-1357 | 2,4-di(difluoromethoxy)phenyl | ethyl |
| A-1358 | 2,5-di(difluoromethoxy)phenyl | ethyl |
| A-1359 | 2,6-di(difluoromethoxy)phenyl | ethyl |
| A-1360 | 3,4-di(difluoromethoxy)phenyl | ethyl |
| A-1361 | 3,5-di(difluoromethoxy)phenyl | ethyl |
| A-1362 | 2,3-di(trifluoromethylthio)phenyl | ethyl |
| A-1363 | 2,4-di(trifluoromethylthio)phenyl | ethyl |
| A-1364 | 2,5-di(trifluoromethylthio)phenyl | ethyl |
| A-1365 | 2,6-di(trifluoromethylthio)phenyl | ethyl |
| A-1366 | 3,4-di(trifluoromethylthio)phenyl | ethyl |
| A-1367 | 3,5-di(trifluoromethylthio)phenyl | ethyl |
| A-1368 | 2-fluoro-3-chlorophenyl | ethyl |
| A-1369 | 2-fluoro-4-chlorophenyl | ethyl |
| A-1370 | 2-fluoro-5-chlorophenyl | ethyl |
| A-1371 | 2-fluoro-6-chlorophenyl | ethyl |
| A-1372 | 3-fluoro-4-chlorophenyl | ethyl |
| A-1373 | 3-fluoro-5-chlorophenyl | ethyl |
| A-1374 | 2-chloro-3-fluorophenyl | ethyl |
| A-1375 | 2-chloro-4-fluorophenyl | ethyl |
| A-1376 | 2-chloro-5-fluorophenyl | ethyl |
| A-1377 | 3-chloro-4-fluorophenyl | ethyl |
| A-1378 | 2-methyl-3-chlorophenyl | ethyl |
| A-1379 | 2-methyl-4-chlorophenyl | ethyl |
| A-1380 | 2-methyl-5-chlorophenyl | ethyl |
| A-1381 | 2-methyl-6-chlorophenyl | ethyl |
| A-1382 | 3-methyl-4-chlorophenyl | ethyl |
| A-1383 | 3-methyl-5-chlorophenyl | ethyl |
| A-1384 | 2-chloro-3-methylphenyl | ethyl |
| A-1385 | 2-chloro-4-methylphenyl | ethyl |
| A-1386 | 2-chloro-5-methylphenyl | ethyl |
| A-1387 | 3-chloro-4-methylphenyl | ethyl |
| A-1388 | 2-methyl-3-fluorophenyl | ethyl |
| A-1389 | 2-methyl-4-fluorophenyl | ethyl |
| A-1390 | 2-methyl-5-fluorophenyl | ethyl |
| A-1391 | 2-methyl-6-fluorophenyl | ethyl |
| A-1392 | 3-methyl-4-fluorophenyl | ethyl |
| A-1393 | 3-methyl-5-fluorophenyl | ethyl |
| A-1394 | 2-fluoro-3-methylphenyl | ethyl |
| A-1395 | 2-fluoro-4-methylphenyl | ethyl |
| A-1396 | 2-fluoro-5-methylphenyl | ethyl |
| A-1397 | 3-fluoro-4-methylphenyl | ethyl |
| A-1398 | 2-chloro-3-ethylphenyl | ethyl |
| A-1399 | 2-chloro-4-ethylphenyl | ethyl |
| A-1400 | 2-chloro-5-ethylphenyl | ethyl |
| A-1401 | 3-chloro-4-ethylphenyl | ethyl |
| A-1402 | 2-ethyl-3-chlorophenyl | ethyl |
| A-1403 | 2-ethyl-4-chlorophenyl | ethyl |
| A-1404 | 2-ethyl-5-chlorophenyl | ethyl |
| A-1405 | 2-ethyl-6-chlorophenyl | ethyl |
| A-1406 | 3-ethyl-4-chlorophenyl | ethyl |
| A-1407 | 3-ethyl-5-chlorophenyl | ethyl |
| A-1408 | 2-ethyl-3-fluorophenyl | ethyl |
| A-1409 | 2-ethyl-4-fluorophenyl | ethyl |
| A-1410 | 2-ethyl-5-fluorophenyl | ethyl |
| A-1411 | 2-ethyl-6-fluorophenyl | ethyl |
| A-1412 | 3-ethyl-4-fluorophenyl | ethyl |
| A-1413 | 3-ethyl-5-fluorophenyl | ethyl |
| A-1414 | 2-fluoro-3-ethylphenyl | ethyl |
| A-1415 | 2-fluoro-4-ethylphenyl | ethyl |
| A-1416 | 2-fluoro-5-ethylphenyl | ethyl |
| A-1417 | 3-fluoro-4-ethylphenyl | ethyl |
| A-1418 | 2-methoxy-3-chlorophenyl | ethyl |
| A-1419 | 2-methoxy-4-chlorophenyl | ethyl |
| A-1420 | 2-methoxy-5-chlorophenyl | ethyl |
| A-1421 | 2-methoxy-6-chlorophenyl | ethyl |
| A-1422 | 3-methoxy-4-chlorophenyl | ethyl |
| A-1423 | 3-methoxy-5-chlorophenyl | ethyl |
| A-1424 | 2-chloro-3-methoxyphenyl | ethyl |
| A-1425 | 2-chloro-4-methoxyphenyl | ethyl |
| A-1426 | 2-chloro-5-methoxyphenyl | ethyl |
| A-1427 | 3-chloro-4-methoxyphenyl | ethyl |
| A-1428 | 2-methoxy-3-fluorophenyl | ethyl |
| A-1429 | 2-methoxy-4-fluorophenyl | ethyl |
| A-1430 | 2-methoxy-5-fluorophenyl | ethyl |
| A-1431 | 2-methoxy-6-fluorophenyl | ethyl |
| A-1432 | 3-methoxy-4-fluorophenyl | ethyl |
| A-1433 | 3-methoxy-5-fluorophenyl | ethyl |
| A-1434 | 2-fluoro-3-methoxyphenyl | ethyl |
| A-1435 | 2-fluoro-4-methoxyphenyl | ethyl |
| A-1436 | 2-fluoro-5-methoxyphenyl | ethyl |
| A-1437 | 3-fluoro-4-methoxyphenyl | ethyl |
| A-1438 | 3-fluoro-5-methoxyphenyl | ethyl |
| A-1439 | 2-(trifluoromethyl)-3-chlorophenyl | ethyl |
| A-1440 | 2-(trifluoromethyl)-4-chlorophenyl | ethyl |
| A-1441 | 2-(trifluoromethyl)-5-chlorophenyl | ethyl |
| A-1442 | 2-(trifluoromethyl)-6-chlorophenyl | ethyl |
| A-1443 | 3-(trifluoromethyl)-4-chlorophenyl | ethyl |
| A-1444 | 3-(trifluoromethyl)-5-chlorophenyl | ethyl |
| A-1445 | 2-chloro-3-(trifluoromethyl)phenyl | ethyl |
| A-1446 | 2-chloro-4-(trifluoromethyl)phenyl | ethyl |
| A-1447 | 2-chloro-5-(trifluoromethyl)phenyl | ethyl |
| A-1448 | 3-chloro-4-(trifluoromethyl)phenyl | ethyl |
| A-1449 | 2-(trifluoromethyl)-3-fluorophenyl | ethyl |
| A-1450 | 2-(trifluoromethyl)-4-fluorophenyl | ethyl |
| A-1451 | 2-(trifluoromethyl)-5-fluorophenyl | ethyl |
| A-1452 | 2-(trifluoromethyl)-6-fluorophenyl | ethyl |
| A-1453 | 3-(trifluoromethyl)-4-fluorophenyl | ethyl |
| A-1454 | 3-(trifluoromethyl)-5-fluorophenyl | ethyl |
| A-1455 | 2-fluoro-3-(trifluoromethyl)phenyl | ethyl |
| A-1456 | 2-fluoro-4-(trifluoromethyl)phenyl | ethyl |
| A-1457 | 2-fluoro-5-(trifluoromethyl)phenyl | ethyl |
| A-1458 | 3-fluoro-4-(trifluoromethyl)phenyl | ethyl |
| A-1459 | 2-(trifluoromethoxy)-3-chlorophenyl | ethyl |
| A-1460 | 2-(trifluoromethoxy)-4-chlorophenyl | ethyl |
| A-1461 | 2-(trifluoromethoxy)-5-chlorophenyl | ethyl |
| A-1462 | 2-(trifluoromethoxy)-6-chlorophenyl | ethyl |
| A-1463 | 3-(trifluoromethoxy)-4-chlorophenyl | ethyl |
| A-1464 | 3-(trifluoromethoxy)-5-chlorophenyl | ethyl |
| A-1465 | 2-chloro-3-(trifluoromethoxy)phenyl | ethyl |
| A-1466 | 2-chloro-4-(trifluoromethoxy)phenyl | ethyl |
| A-1467 | 2-chloro-5-(trifluoromethoxy)phenyl | ethyl |
| A-1468 | 3-chloro-4-(trifluoromethoxy)phenyl | ethyl |
| A-1469 | 2-(trifluoromethoxy)-3-fluorophenyl | ethyl |
| A-1470 | 2-(trifluoromethoxy)-4-fluorophenyl | ethyl |
| A-1471 | 2-(trifluoromethoxy)-5-fluorophenyl | ethyl |
| A-1472 | 2-(trifluoromethoxy)-6-fluorophenyl | ethyl |
| A-1473 | 3-(trifluoromethoxy)-4-fluorophenyl | ethyl |
| A-1474 | 3-(trifluoromethoxy)-5-fluorophenyl | ethyl |
| A-1475 | 2-fluoro-3-(trifluoromethoxy)phenyl | ethyl |
| A-1476 | 2-fluoro-4-(trifluoromethoxy)phenyl | ethyl |
| A-1477 | 2-fluoro-5-(trifluoromethoxy)phenyl | ethyl |
| A-1478 | 3-fluoro-4-(trifluoromethoxy)phenyl | ethyl |
| A-1479 | 2-(difluoromethoxy)-3-chlorophenyl | ethyl |
| A-1480 | 2-(difluoromethoxy)-4-chlorophenyl | ethyl |
| A-1481 | 2-(difluoromethoxy)-5-chlorophenyl | ethyl |
| A-1482 | 2-(difluoromethoxy)-6-chlorophenyl | ethyl |
| A-1483 | 3-(difluoromethoxy)-4-chlorophenyl | ethyl |
| A-1484 | 3-(difluoromethoxy)-5-chlorophenyl | ethyl |
| A-1485 | 2-chloro-3-(difluoromethoxy)phenyl | ethyl |
| A-1486 | 2-chloro-4-(difluoromethoxy)phenyl | ethyl |
| A-1487 | 2-chloro-5-(difluoromethoxy)phenyl | ethyl |
| A-1488 | 3-chloro-4-(difluoromethoxy)phenyl | ethyl |
| A-1489 | 2-(difluoromethoxy)-3-fluorophenyl | ethyl |
| A-1490 | 2-(difluoromethoxy)-4-fluorophenyl | ethyl |
| A-1491 | 2-(difluoromethoxy)-5-fluorophenyl | ethyl |
| A-1492 | 2-(difluoromethoxy)-6-fluorophenyl | ethyl |
| A-1493 | 3-(difluoromethoxy)-4-fluorophenyl | ethyl |
| A-1494 | 3-(difluoromethoxy)-5-fluorophenyl | ethyl |
| A-1495 | 2-fluoro-3-(difluoromethoxy)phenyl | ethyl |
| A-1496 | 2-fluoro-4-(difluoromethoxy)phenyl | ethyl |
| A-1497 | 2-fluoro-5-(difluoromethoxy)phenyl | ethyl |
| A-1498 | 3-fluoro-4-(difluoromethoxy)phenyl | ethyl |
| A-1499 | 2-(trifluoromethylthio)-3- | ethyl |
| | chlorophenyl | |
| A-1500 | 2-(trifluoromethylthio)-4- | ethyl |
| | chlorophenyl | |
| A-1501 | 2-(trifluoromethylthio)-5- | ethyl |
| | chlorophenyl | |
| A-1502 | 2-(trifluoromethylthio)-6- | ethyl |
| | chlorophenyl | |
| A-1503 | 3-(trifluoromethylthio)-4- | ethyl |
| | chlorophenyl | |
| A-1504 | 3-(trifluoromethylthio)-5- | ethyl |
| | chlorophenyl | |
| A-1505 | 2-chloro-3- | ethyl |
| | (trifluoromethylthio)phenyl | |
| A-1506 | 2-chloro-4- | ethyl |
| | (trifluoromethylthio)phenyl | |
| A-1507 | 2-chloro-5- | ethyl |
| | (trifluoromethylthio)phenyl | |
| A-1508 | 3-chloro-4- | ethyl |
| | (trifluoromethylthio)phenyl | |
| A-1509 | 2-(trifluoromethylthio)-3-fluorophenyl | ethyl |
| A-1510 | 2-(trifluoromethylthio)-4-fluorophenyl | ethyl |
| A-1511 | 2-(trifluoromethylthio)-5-fluorophenyl | ethyl |
| A-1512 | 2-(trifluoromethylthio)-6-fluorophenyl | ethyl |
| A-1513 | 3-(trifluoromethylthio)-4-fluorophenyl | ethyl |
| A-1514 | 3-(trifluoromethylthio)-5-fluorophenyl | ethyl |
| A-1515 | 2-fluoro-3-(trifluoromethylthio)phenyl | ethyl |
| A-1516 | 2-fluoro-4-(trifluoromethylthio)phenyl | ethyl |
| A-1517 | 2-fluoro-5-(trifluoromethylthio)phenyl | ethyl |
| A-1518 | 3-fluoro-4-(trifluoromethylthio)phenyl | ethyl |
| A-1519 | 2,3,4-trichlorophenyl | ethyl |
| A-1520 | 2,3,5-trichlorophenyl | ethyl |
| A-1521 | 2,3,6-trichlorophenyl | ethyl |
| A-1522 | 2,4,5-trichlorophenyl | ethyl |
| A-1523 | 2,4,6-trichlorophenyl | ethyl |
| A-1524 | 3,4,5-trichlorophenyl | ethyl |
| A-1525 | 2,3,4-trifluorophenyl | ethyl |
| A-1526 | 2,3,5-trifluorophenyl | ethyl |
| A-1527 | 2,3,6-trifluorophenyl | ethyl |
| A-1528 | 2,4,5-trifluorophenyl | ethyl |
| A-1529 | 2,4,6-trifluorophenyl | ethyl |
| A-1530 | 3,4,5-trifluorophenyl | ethyl |
| A-1531 | 2,3,4-trimethylphenyl | ethyl |
| A-1532 | 2,3,5-trimethylphenyl | ethyl |
| A-1533 | 2,3,6-trimethylphenyl | ethyl |
| A-1534 | 2,4,5-trimethylphenyl | ethyl |
| A-1535 | 2,4,6-trimethylphenyl | ethyl |
| A-1536 | 3,4,5-trimethylphenyl | ethyl |
| A-1537 | 2,3,4-trimethoxyphenyl | ethyl |
| A-1538 | 2,3,5-trimethoxyphenyl | ethyl |
| A-1539 | 2,3,6-trimethoxyphenyl | ethyl |
| A-1540 | 2,4,5-trimethoxyphenyl | ethyl |
| A-1541 | 2,4,6-trimethoxyphenyl | ethyl |
| A-1542 | 3,4,5-trimethoxyphenyl | ethyl |
| A-1543 | phenyl | TMP |
| A-1544 | 2-chlorophenyl | TMP |
| A-1545 | 3-chlorophenyl | TMP |
| A-1546 | 4-chlorophenyl | TMP |
| A-1547 | 2-fluorophenyl | TMP |
| A-1548 | 3-fluorophenyl | TMP |
| A-1549 | 4-fluorophenyl | TMP |
| A-1550 | 2-methylphenyl | TMP |
| A-1551 | 3-methylphenyl | TMP |
| A-1552 | 4-methylphenyl | TMP |
| A-1553 | 2-ethylphenyl | TMP |
| A-1554 | 3-ethylphenyl | TMP |
| A-1555 | 4-ethylphenyl | TMP |
| A-1556 | 2-methoxyphenyl | TMP |
| A-1557 | 3-methoxyphenyl | TMP |
| A-1558 | 4-methoxyphenyl | TMP |
| A-1559 | 2-trifluoromethylphenyl | TMP |
| A-1560 | 3-trifluoromethylphenyl | TMP |
| A-1561 | 4-trifluoromethylphenyl | TMP |
| A-1562 | 2-trifluoromethoxyphenyl | TMP |
| A-1563 | 3-trifluoromethoxyphenyl | TMP |
| A-1564 | 4-trifluoromethoxyphenyl | TMP |
| A-1565 | 2-difluoromethoxyphenyl | TMP |
| A-1566 | 3-difluoromethoxyphenyl | TMP |
| A-1567 | 4-difluoromethoxyphenyl | TMP |
| A-1568 | 2-trifluoromethylthiophenyl | TMP |
| A-1569 | 3-trifluoromethylthiophenyl | TMP |
| A-1570 | 4-trifluoromethylthiophenyl | TMP |
| A-1571 | 2,3-dichlorophenyl | TMP |
| A-1572 | 2,4-dichlorophenyl | TMP |
| A-1573 | 2,5-dichlorophenyl | TMP |
| A-1574 | 2,6-dichlorophenyl | TMP |
| A-1575 | 3,4-dichlorophenyl | TMP |
| A-1576 | 3,5-dichlorophenyl | TMP |
| A-1577 | 2,3-difluorophenyl | TMP |
| A-1578 | 2,4-difluorophenyl | TMP |
| A-1579 | 2,5-difluorophenyl | TMP |
| A-1580 | 2,6-difluorophenyl | TMP |
| A-1581 | 3,4-difluorophenyl | TMP |
| A-1582 | 3,5-difluorophenyl | TMP |
| A-1583 | 2,3-dimethylphenyl | TMP |
| A-1584 | 2,4-dimethylphenyl | TMP |
| A-1585 | 2,5-dimethylphenyl | TMP |
| A-1586 | 2,6-dimethylphenyl | TMP |
| A-1587 | 3,4-dimethylphenyl | TMP |
| A-1588 | 3,5-dimethylphenyl | TMP |
| A-1589 | 2,3-diethylphenyl | TMP |
| A-1590 | 2,4-diethylphenyl | TMP |
| A-1591 | 2,5-diethylphenyl | TMP |
| A-1592 | 2,6-diethylphenyl | TMP |
| A-1593 | 3,5-diethylphenyl | TMP |
| A-1594 | 3,4-diethylphenyl | TMP |
| A-1595 | 2,3-dimethoxyphenyl | TMP |
| A-1596 | 2,4-dimethoxyphenyl | TMP |
| A-1597 | 2,5-dimethoxyphenyl | TMP |
| A-1598 | 2,6-dimethoxyphenyl | TMP |
| A-1599 | 3,4-dimethoxyphenyl | TMP |
| A-1600 | 3,5-dimethoxyphenyl | TMP |
| A-1601 | 2,3-di(trifluoromethyl)phenyl | TMP |
| A-1602 | 2,4-di(trifluoromethyl)phenyl | TMP |
| A-1603 | 2,5-di(trifluoromethyl)phenyl | TMP |
| A-1604 | 2,6-di(trifluoromethyl)phenyl | TMP |
| A-1605 | 3,4-di(trifluoromethyl)phenyl | TMP |
| A-1606 | 3,5-di(trifluoromethyl)phenyl | TMP |
| A-1607 | 2,3-di(trifluoromethoxy)phenyl | TMP |
| A-1608 | 2,4-di(trifluoromethoxy)phenyl | TMP |
| A-1609 | 2,5-di(trifluoromethoxy)phenyl | TMP |
| A-1610 | 2,6-di(trifluoromethoxy)phenyl | TMP |
| A-1611 | 3,4-di(trifluoromethoxy)phenyl | TMP |
| A-1612 | 3,5-di(trifluoromethoxy)phenyl | TMP |
| A-1613 | 2,3-di(difluoromethoxy)phenyl | TMP |
| A-1614 | 2,4-di(difluoromethoxy)phenyl | TMP |
| A-1615 | 2,5-di(difluoromethoxy)phenyl | TMP |
| A-1616 | 2,6-di(difluoromethoxy)phenyl | TMP |
| A-1617 | 3,4-di(difluoromethoxy)phenyl | TMP |
| A-1618 | 3,5-di(difluoromethoxy)phenyl | TMP |
| A-1619 | 2,3-di(trifluoromethylthio)phenyl | TMP |
| A-1620 | 2,4-di(trifluoromethylthio)phenyl | TMP |
| A-1621 | 2,5-di(trifluoromethylthio)phenyl | TMP |
| A-1622 | 2,6-di(trifluoromethylthio)phenyl | TMP |
| A-1623 | 3,4-di(trifluoromethylthio)phenyl | TMP |
| A-1624 | 3,5-di(trifluoromethylthio)phenyl | TMP |
| A-1625 | 2-fluoro-3-chlorophenyl | TMP |
| A-1626 | 2-fluoro-4-chlorophenyl | TMP |
| A-1627 | 2-fluoro-5-chlorophenyl | TMP |
| A-1628 | 2-fluoro-6-chlorophenyl | TMP |
| A-1629 | 3-fluoro-4-chlorophenyl | TMP |
| A-1630 | 3-fluoro-5-chlorophenyl | TMP |
| A-1631 | 2-chloro-3-fluorophenyl | TMP |
| A-1632 | 2-chloro-4-fluorophenyl | TMP |
| A-1633 | 2-chloro-5-fluorophenyl | TMP |
| A-1634 | 3-chloro-4-fluorophenyl | TMP |
| A-1635 | 2-methyl-3-chlorophenyl | TMP |
| A-1636 | 2-methyl-4-chlorophenyl | TMP |
| A-1637 | 2-methyl-5-chlorophenyl | TMP |
| A-1638 | 2-methyl-6-chlorophenyl | TMP |
| A-1639 | 3-methyl-4-chlorophenyl | TMP |
| A-1640 | 3-methyl-5-chlorophenyl | TMP |
| A-1641 | 2-chloro-3-methylphenyl | TMP |
| A-1642 | 2-chloro-4-methylphenyl | TMP |
| A-1643 | 2-chloro-5-methylphenyl | TMP |
| A-1644 | 3-chloro-4-methylphenyl | TMP |
| A-1645 | 2-methyl-3-fluorophenyl | TMP |
| A-1646 | 2-methyl-4-fluorophenyl | TMP |
| A-1647 | 2-methyl-5-fluorophenyl | TMP |
| A-1648 | 2-methyl-6-fluorophenyl | TMP |
| A-1649 | 3-methyl-4-fluorophenyl | TMP |
| A-1650 | 3-methyl-5-fluorophenyl | TMP |
| A-1651 | 2-fluoro-3-methylphenyl | TMP |
| A-1652 | 2-fluoro-4-methylphenyl | TMP |
| A-1653 | 2-fluoro-5-methylphenyl | TMP |
| A-1654 | 3-fluoro-4-methylphenyl | TMP |
| A-1655 | 2-chloro-3-ethylphenyl | TMP |
| A-1656 | 2-chloro-4-ethylphenyl | TMP |
| A-1657 | 2-chloro-5-ethylphenyl | TMP |
| A-1658 | 3-chloro-4-ethylphenyl | TMP |
| A-1659 | 2-ethyl-3-chlorophenyl | TMP |
| A-1660 | 2-ethyl-4-chlorophenyl | TMP |
| A-1661 | 2-ethyl-5-chlorophenyl | TMP |
| A-1662 | 2-ethyl-6-chlorophenyl | TMP |
| A-1663 | 3-ethyl-4-chlorophenyl | TMP |
| A-1664 | 3-ethyl-5-chlorophenyl | TMP |
| A-1665 | 2-ethyl-3-fluorophenyl | TMP |
| A-1666 | 2-ethyl-4-fluorophenyl | TMP |
| A-1667 | 2-ethyl-5-fluorophenyl | TMP |
| A-1668 | 2-ethyl-6-fluorophenyl | TMP |
| A-1669 | 3-ethyl-4-fluorophenyl | TMP |
| A-1670 | 3-ethyl-5-fluorophenyl | TMP |
| A-1671 | 2-fluoro-3-ethylphenyl | TMP |
| A-1672 | 2-fluoro-4-ethylphenyl | TMP |
| A-1673 | 2-fluoro-5-ethylphenyl | TMP |
| A-1674 | 3-fluoro-4-ethylphenyl | TMP |
| A-1675 | 2-methoxy-3-chlorophenyl | TMP |
| A-1676 | 2-methoxy-4-chlorophenyl | TMP |
| A-1677 | 2-methoxy-5-chlorophenyl | TMP |
| A-1678 | 2-methoxy-6-chlorophenyl | TMP |
| A-1679 | 3-methoxy-4-chlorophenyl | TMP |
| A-1680 | 3-methoxy-5-chlorophenyl | TMP |
| A-1681 | 2-chloro-3-methoxyphenyl | TMP |
| A-1682 | 2-chloro-4-methoxyphenyl | TMP |
| A-1683 | 2-chloro-5-methoxyphenyl | TMP |
| A-1684 | 3-chloro-4-methoxyphenyl | TMP |
| A-1685 | 2-methoxy-3-fluorophenyl | TMP |
| A-1686 | 2-methoxy-4-fluorophenyl | TMP |
| A-1687 | 2-methoxy-5-fluorophenyl | TMP |
| A-1688 | 2-methoxy-6-fluorophenyl | TMP |
| A-1689 | 3-methoxy-4-fluorophenyl | TMP |
| A-1690 | 3-methoxy-5-fluorophenyl | TMP |
| A-1691 | 2-fluoro-3-methoxyphenyl | TMP |
| A-1692 | 2-fluoro-4-methoxyphenyl | TMP |
| A-1693 | 2-fluoro-5-methoxyphenyl | TMP |
| A-1694 | 3-fluoro-4-methoxyphenyl | TMP |
| A-1695 | 3-fluoro-5-methoxyphenyl | TMP |
| A-1696 | 2-(trifluoromethyl)-3-chlorophenyl | TMP |
| A-1697 | 2-(trifluoromethyl)-4-chlorophenyl | TMP |
| A-1698 | 2-(trifluoromethyl)-5-chlorophenyl | TMP |
| A-1699 | 2-(trifluoromethyl)-6-chlorophenyl | TMP |
| A-1700 | 3-(trifluoromethyl)-4-chlorophenyl | TMP |
| A-1701 | 3-(trifluoromethyl)-5-chlorophenyl | TMP |
| A-1702 | 2-chloro-3-(trifluoromethyl)phenyl | TMP |
| A-1703 | 2-chloro-4-(trifluoromethyl)phenyl | TMP |
| A-1704 | 2-chloro-5-(trifluoromethyl)phenyl | TMP |
| A-1705 | 3-chloro-4-(trifluoromethyl)phenyl | TMP |
| A-1706 | 2-(trifluoromethyl)-3-fluorophenyl | TMP |
| A-1707 | 2-(trifluoromethyl)-4-fluorophenyl | TMP |
| A-1708 | 2-(trifluoromethyl)-5-fluorophenyl | TMP |
| A-1709 | 2-(trifluoromethyl)-6-fluorophenyl | TMP |
| A-1710 | 3-(trifluoromethyl)-4-fluorophenyl | TMP |
| A-1711 | 3-(trifluoromethyl)-5-fluorophenyl | TMP |
| A-1712 | 2-fluoro-3-(trifluoromethyl)phenyl | TMP |
| A-1713 | 2-fluoro-4-(trifluoromethyl)phenyl | TMP |
| A-1714 | 2-fluoro-5-(trifluoromethyl)phenyl | TMP |
| A-1715 | 3-fluoro-4-(trifluoromethyl)phenyl | TMP |
| A-1716 | 2-(trifluoromethoxy)-3-chlorophenyl | TMP |
| A-1717 | 2-(trifluoromethoxy)-4-chlorophenyl | TMP |
| A-1718 | 2-(trifluoromethoxy)-5-chlorophenyl | TMP |
| A-1719 | 2-(trifluoromethoxy)-6-chlorophenyl | TMP |
| A-1720 | 3-(trifluoromethoxy)-4-chlorophenyl | TMP |
| A-1721 | 3-(trifluoromethoxy)-5-chlorophenyl | TMP |
| A-1722 | 2-chloro-3-(trifluoromethoxy)phenyl | TMP |
| A-1723 | 2-chloro-4-(trifluoromethoxy)phenyl | TMP |
| A-1724 | 2-chloro-5-(trifluoromethoxy)phenyl | TMP |
| A-1725 | 3-chloro-4-(trifluoromethoxy)phenyl | TMP |
| A-1726 | 2-(trifluoromethoxy)-3-fluorophenyl | TMP |
| A-1727 | 2-(trifluoromethoxy)-4-fluorophenyl | TMP |
| A-1728 | 2-(trifluoromethoxy)-5-fluorophenyl | TMP |
| A-1729 | 2-(trifluoromethoxy)-6-fluorophenyl | TMP |
| A-1730 | 3-(trifluoromethoxy)-4-fluorophenyl | TMP |
| A-1731 | 3-(trifluoromethoxy)-5-fluorophenyl | TMP |
| A-1732 | 2-fluoro-3-(trifluoromethoxy)phenyl | TMP |
| A-1733 | 2-fluoro-4-(trifluoromethoxy)phenyl | TMP |
| A-1734 | 2-fluoro-5-(trifluoromethoxy)phenyl | TMP |
| A-1735 | 3-fluoro-4-(trifluoromethoxy)phenyl | TMP |
| A-1736 | 2-(difluoromethoxy)-3-chlorophenyl | TMP |
| A-1737 | 2-(difluoromethoxy)-4-chlorophenyl | TMP |
| A-1738 | 2-(difluoromethoxy)-5-chlorophenyl | TMP |
| A-1739 | 2-(difluoromethoxy)-6-chlorophenyl | TMP |
| A-1740 | 3-(difluoromethoxy)-4-chlorophenyl | TMP |
| A-1741 | 3-(difluoromethoxy)-5-chlorophenyl | TMP |
| A-1742 | 2-chloro-3-(difluoromethoxy)phenyl | TMP |
| A-1743 | 2-chloro-4-(difluoromethoxy)phenyl | TMP |
| A-1744 | 2-chloro-5-(difluoromethoxy)phenyl | TMP |
| A-1745 | 3-chloro-4-(difluoromethoxy)phenyl | TMP |
| A-1746 | 2-(difluoromethoxy)-3-fluorophenyl | TMP |
| A-1747 | 2-(difluoromethoxy)-4-fluorophenyl | TMP |
| A-1748 | 2-(difluoromethoxy)-5-fluorophenyl | TMP |
| A-1749 | 2-(difluoromethoxy)-6-fluorophenyl | TMP |
| A-1750 | 3-(difluoromethoxy)-4-fluorophenyl | TMP |
| A-1751 | 3-(difluoromethoxy)-5-fluorophenyl | TMP |
| A-1752 | 2-fluoro-3-(difluoromethoxy)phenyl | TMP |
| A-1753 | 2-fluoro-4-(difluoromethoxy)phenyl | TMP |
| A-1754 | 2-fluoro-5-(difluoromethoxy)phenyl | TMP |
| A-1755 | 3-fluoro-4-(difluoromethoxy)phenyl | TMP |
| A-1756 | 2-(trifluoromethylthio)-3- | TMP |
| | chlorophenyl | |
| A-1757 | 2-(trifluoromethylthio)-4- | TMP |
| | chlorophenyl | |
| A-1758 | 2-(trifluoromethylthio)-5- | TMP |
| | chlorophenyl | |
| A-1759 | 2-(trifluoromethylthio)-6- | TMP |
| | chlorophenyl | |
| A-1760 | 3-(trifluoromethylthio)-4- | TMP |
| | chlorophenyl | |
| A-1761 | 3-(trifluoromethylthio)-5- | TMP |
| | chlorophenyl | |
| A-1762 | 2-chloro-3- | TMP |
| | (trifluoromethylthio)phenyl | |
| A-1763 | 2-chloro-4- | TMP |
| | (trifluoromethylthio)phenyl | |
| A-1764 | 2-chloro-5- | TMP |
| | (trifluoromethylthio)phenyl | |
| A-1765 | 3-chloro-4- | TMP |
| | (trifluoromethylthio)phenyl | |
| A-1766 | 2-(trifluoromethylthio)-3-fluorophenyl | TMP |
| A-1767 | 2-(trifluoromethylthio)-4-fluorophenyl | TMP |
| A-1768 | 2-(trifluoromethylthio)-5-fluorophenyl | TMP |
| A-1769 | 2-(trifluoromethylthio)-6-fluorophenyl | TMP |
| A-1770 | 3-(trifluoromethylthio)-4-fluorophenyl | TMP |
| A-1771 | 3-(trifluoromethylthio)-5-fluorophenyl | TMP |
| A-1772 | 2-fluoro-3-(trifluoromethylthio)phenyl | TMP |
| A-1773 | 2-fluoro-4-(trifluoromethylthio)phenyl | TMP |
| A-1774 | 2-fluoro-5-(trifluoromethylthio)phenyl | TMP |
| A-1775 | 3-fluoro-4-(trifluoromethylthio)phenyl | TMP |
| A-1776 | 2,3,4-trichlorophenyl | TMP |
| A-1777 | 2,3,5-trichlorophenyl | TMP |
| A-1778 | 2,3,6-trichlorophenyl | TMP |
| A-1779 | 2,4,5-trichlorophenyl | TMP |
| A-1780 | 2,4,6-trichlorophenyl | TMP |
| A-1781 | 3,4,5-trichlorophenyl | TMP |
| A-1782 | 2,3,4-trifluorophenyl | TMP |
| A-1783 | 2,3,5-trifluorophenyl | TMP |
| A-1784 | 2,3,6-trifluorophenyl | TMP |
| A-1785 | 2,4,5-trifluorophenyl | TMP |
| A-1786 | 2,4,6-trifluorophenyl | TMP |
| A-1787 | 3,4,5-trifluorophenyl | TMP |
| A-1788 | 2,3,4-trimethylphenyl | TMP |
| A-1789 | 2,3,5-trimethylphenyl | TMP |
| A-1790 | 2,3,6-trimethylphenyl | TMP |
| A-1791 | 2,4,5-trimethylphenyl | TMP |
| A-1792 | 2,4,6-trimethylphenyl | TMP |
| A-1793 | 3,4,5-trimethylphenyl | TMP |
| A-1794 | 2,3,4-trimethoxyphenyl | TMP |
| A-1795 | 2,3,5-trimethoxyphenyl | TMP |
| A-1796 | 2,3,6-trimethoxyphenyl | TMP |
| A-1797 | 2,4,5-trimethoxyphenyl | TMP |
| A-1798 | 2,4,6-trimethoxyphenyl | TMP |
| A-1799 | 3,4,5-trimethoxyphenyl | TMP |
| A-1800 | phenyl | N(2-propyl)₂ |
| A-1801 | 2-chlorophenyl | N(2-propyl)₂ |
| A-1802 | 3-chlorophenyl | N(2-propyl)₂ |
| A-1803 | 4-chlorophenyl | N(2-propyl)₂ |
| A-1804 | 2-fluorophenyl | N(2-propyl)₂ |
| A-1805 | 3-fluorophenyl | N(2-propyl)₂ |
| A-1806 | 4-fluorophenyl | N(2-propyl)₂ |
| A-1807 | 2-methylphenyl | N(2-propyl)₂ |
| A-1808 | 3-methylphenyl | N(2-propyl)₂ |
| A-1809 | 4-methylphenyl | N(2-propyl)₂ |
| A-1810 | 2-ethylphenyl | N(2-propyl)₂ |
| A-1811 | 3-ethylphenyl | N(2-propyl)₂ |
| A-1812 | 4-ethylphenyl | N(2-propyl)₂ |
| A-1813 | 2-methoxyphenyl | N(2-propyl)₂ |
| A-1814 | 3-methoxyphenyl | N(2-propyl)₂ |
| A-1815 | 4-methoxyphenyl | N(2-propyl)₂ |
| A-1816 | 2-trifluoromethylphenyl | N(2-propyl)₂ |
| A-1817 | 3-trifluoromethylphenyl | N(2-propyl)₂ |
| A-1818 | 4-trifluoromethylphenyl | N(2-propyl)₂ |
| A-1819 | 2-trifluoromethoxyphenyl | N(2-propyl)₂ |
| A-1820 | 3-trifluoromethoxyphenyl | N(2-propyl)₂ |
| A-1821 | 4-trifluoromethoxyphenyl | N(2-propyl)₂ |
| A-1822 | 2-difluoromethoxyphenyl | N(2-propyl)₂ |
| A-1823 | 3-difluoromethoxyphenyl | N(2-propyl)₂ |
| A-1824 | 4-difluoromethoxyphenyl | N(2-propyl)₂ |
| A-1825 | 2-trifluoromethylthiophenyl | N(2-propyl)₂ |
| A-1826 | 3-trifluoromethylthiophenyl | N(2-propyl)₂ |
| A-1827 | 4-trifluoromethylthiophenyl | N(2-propyl)₂ |
| A-1828 | 2,3-dichlorophenyl | N(2-propyl)₂ |
| A-1829 | 2,4-dichlorophenyl | N(2-propyl)₂ |
| A-1830 | 2,5-dichlorophenyl | N(2-propyl)₂ |
| A-1831 | 2,6-dichlorophenyl | N(2-propyl)₂ |
| A-1832 | 3,4-dichlorophenyl | N(2-propyl)₂ |
| A-1833 | 3,5-dichlorophenyl | N(2-propyl)₂ |
| A-1834 | 2,3-difluorophenyl | N(2-propyl)₂ |
| A-1835 | 2,4-difluorophenyl | N(2-propyl)₂ |
| A-1836 | 2,5-difluorophenyl | N(2-propyl)₂ |
| A-1837 | 2,6-difluorophenyl | N(2-propyl)₂ |
| A-1838 | 3,4-difluorophenyl | N(2-propyl)₂ |
| A-1839 | 3,5-difluorophenyl | N(2-propyl)₂ |
| A-1840 | 2,3-dimethylphenyl | N(2-propyl)₂ |
| A-1841 | 2,4-dimethylphenyl | N(2-propyl)₂ |
| A-1842 | 2,5-dimethylphenyl | N(2-propyl)₂ |
| A-1843 | 2,6-dimethylphenyl | N(2-propyl)₂ |
| A-1844 | 3,4-dimethylphenyl | N(2-propyl)₂ |
| A-1845 | 3,5-dimethylphenyl | N(2-propyl)₂ |
| A-1846 | 2,3-diethylphenyl | N(2-propyl)₂ |
| A-1847 | 2,4-diethylphenyl | N(2-propyl)₂ |
| A-1848 | 2,5-diethylphenyl | N(2-propyl)₂ |
| A-1849 | 2,6-diethylphenyl | N(2-propyl)₂ |
| A-1850 | 3,5-diethylphenyl | N(2-propyl)₂ |
| A-1851 | 3,4-diethylphenyl | N(2-propyl)₂ |
| A-1852 | 2,3-dimethoxyphenyl | N(2-propyl)₂ |
| A-1853 | 2,4-dimethoxyphenyl | N(2-propyl)₂ |
| A-1854 | 2,5-dimethoxyphenyl | N(2-propyl)₂ |
| A-1855 | 2,6-dimethoxyphenyl | N(2-propyl)₂ |
| A-1856 | 3,4-dimethoxyphenyl | N(2-propyl)₂ |
| A-1857 | 3,5-dimethoxyphenyl | N(2-propyl)₂ |
| A-1858 | 2,3-di(trifluoromethyl)phenyl | N(2-propyl)₂ |
| A-1859 | 2,4-di(trifluoromethyl)phenyl | N(2-propyl)₂ |
| A-1860 | 2,5-di(trifluoromethyl)phenyl | N(2-propyl)₂ |
| A-1861 | 2,6-di(trifluoromethyl)phenyl | N(2-propyl)₂ |
| A-1862 | 3,4-di(trifluoromethyl)phenyl | N(2-propyl)₂ |
| A-1863 | 3,5-di(trifluoromethyl)phenyl | N(2-propyl)₂ |
| A-1864 | 2,3-di(trifluoromethoxy)phenyl | N(2-propyl)₂ |
| A-1865 | 2,4-di(trifluoromethoxy)phenyl | N(2-propyl)₂ |
| A-1866 | 2,5-di(trifluoromethoxy)phenyl | N(2-propyl)₂ |
| A-1867 | 2,6-di(trifluoromethoxy)phenyl | N(2-propyl)₂ |
| A-1868 | 3,4-di(trifluoromethoxy)phenyl | N(2-propyl)₂ |
| A-1869 | 3,5-di(trifluoromethoxy)phenyl | N(2-propyl)₂ |
| A-1870 | 2,3-di(difluoromethoxy)phenyl | N(2-propyl)₂ |
| A-1871 | 2,4-di(difluoromethoxy)phenyl | N(2-propyl)₂ |
| A-1872 | 2,5-di(difluoromethoxy)phenyl | N(2-propyl)₂ |
| A-1873 | 2,6-di(difluoromethoxy)phenyl | N(2-propyl)₂ |
| A-1874 | 3,4-di(difluoromethoxy)phenyl | N(2-propyl)₂ |
| A-1875 | 3,5-di(difluoromethoxy)phenyl | N(2-propyl)₂ |
| A-1876 | 2,3-di(trifluoromethylthio)phenyl | N(2-propyl)₂ |
| A-1877 | 2,4-di(trifluoromethylthio)phenyl | N(2-propyl)₂ |
| A-1878 | 2,5-di(trifluoromethylthio)phenyl | N(2-propyl)₂ |
| A-1879 | 2,6-di(trifluoromethylthio)phenyl | N(2-propyl)₂ |
| A-1880 | 3,4-di(trifluoromethylthio)phenyl | N(2-propyl)₂ |
| A-1881 | 3,5-di(trifluoromethylthio)phenyl | N(2-propyl)₂ |
| A-1882 | 2-fluoro-3-chlorophenyl | N(2-propyl)₂ |
| A-1883 | 2-fluoro-4-chlorophenyl | N(2-propyl)₂ |
| A-1884 | 2-fluoro-5-chlorophenyl | N(2-propyl)₂ |
| A-1885 | 2-fluoro-6-chlorophenyl | N(2-propyl)₂ |
| A-1886 | 3-fluoro-4-chlorophenyl | N(2-propyl)₂ |
| A-1887 | 3-fluoro-5-chlorophenyl | N(2-propyl)₂ |
| A-1888 | 2-chloro-3-fluorophenyl | N(2-propyl)₂ |
| A-1889 | 2-chloro-4-fluorophenyl | N(2-propyl)₂ |
| A-1890 | 2-chloro-5-fluorophenyl | N(2-propyl)₂ |
| A-1891 | 3-chloro-4-fluorophenyl | N(2-propyl)₂ |
| A-1892 | 2-methyl-3-chlorophenyl | N(2-propyl)₂ |
| A-1893 | 2-methyl-4-chlorophenyl | N(2-propyl)₂ |
| A-1894 | 2-methyl-5-chlorophenyl | N(2-propyl)₂ |
| A-1895 | 2-methyl-6-chlorophenyl | N(2-propyl)₂ |
| A-1896 | 3-methyl-4-chlorophenyl | N(2-propyl)₂ |
| A-1897 | 3-methyl-5-chlorophenyl | N(2-propyl)₂ |
| A-1898 | 2-chloro-3-methylphenyl | N(2-propyl)₂ |
| A-1899 | 2-chloro-4-methylphenyl | N(2-propyl)₂ |
| A-1900 | 2-chloro-5-methylphenyl | N(2-propyl)₂ |
| A-1901 | 3-chloro-4-methylphenyl | N(2-propyl)₂ |
| A-1902 | 2-methyl-3-fluorophenyl | N(2-propyl)₂ |
| A-1903 | 2-methyl-4-fluorophenyl | N(2-propyl)₂ |
| A-1904 | 2-methyl-5-fluorophenyl | N(2-propyl)₂ |
| A-1905 | 2-methyl-6-fluorophenyl | N(2-propyl)₂ |
| A-1906 | 3-methyl-4-fluorophenyl | N(2-propyl)₂ |
| A-1907 | 3-methyl-5-fluorophenyl | N(2-propyl)₂ |
| A-1908 | 2-fluoro-3-methylphenyl | N(2-propyl)₂ |
| A-1909 | 2-fluoro-4-methylphenyl | N(2-propyl)₂ |
| A-1910 | 2-fluoro-5-methylphenyl | N(2-propyl)₂ |
| A-1911 | 3-fluoro-4-methylphenyl | N(2-propyl)₂ |
| A-1912 | 2-chloro-3-ethylphenyl | N(2-propyl)₂ |
| A-1913 | 2-chloro-4-ethylphenyl | N(2-propyl)₂ |
| A-1914 | 2-chloro-5-ethylphenyl | N(2-propyl)₂ |
| A-1915 | 3-chloro-4-ethylphenyl | N(2-propyl)₂ |
| A-1916 | 2-ethyl-3-chlorophenyl | N(2-propyl)₂ |
| A-1917 | 2-ethyl-4-chlorophenyl | N(2-propyl)₂ |
| A-1918 | 2-ethyl-5-chlorophenyl | N(2-propyl)₂ |
| A-1919 | 2-ethyl-6-chlorophenyl | N(2-propyl)₂ |
| A-1920 | 3-ethyl-4-chlorophenyl | N(2-propyl)₂ |
| A-1921 | 3-ethyl-5-chlorophenyl | N(2-propyl)₂ |
| A-1922 | 2-ethyl-3-fluorophenyl | N(2-propyl)₂ |
| A-1923 | 2-ethyl-4-fluorophenyl | N(2-propyl)₂ |
| A-1924 | 2-ethyl-5-fluorophenyl | N(2-propyl)₂ |
| A-1925 | 2-ethyl-6-fluorophenyl | N(2-propyl)₂ |
| A-1926 | 3-ethyl-4-fluorophenyl | N(2-propyl)₂ |
| A-1927 | 3-ethyl-5-fluorophenyl | N(2-propyl)₂ |
| A-1928 | 2-fluoro-3-ethylphenyl | N(2-propyl)₂ |
| A-1929 | 2-fluoro-4-ethylphenyl | N(2-propyl)₂ |
| A-1930 | 2-fluoro-5-ethylphenyl | N(2-propyl)₂ |
| A-1931 | 3-fluoro-4-ethylphenyl | N(2-propyl)₂ |
| A-1932 | 2-methoxy-3-chlorophenyl | N(2-propyl)₂ |
| A-1933 | 2-methoxy-4-chlorophenyl | N(2-propyl)₂ |
| A-1934 | 2-methoxy-5-chlorophenyl | N(2-propyl)₂ |
| A-1935 | 2-methoxy-6-chlorophenyl | N(2-propyl)₂ |
| A-1936 | 3-methoxy-4-chlorophenyl | N(2-propyl)₂ |
| A-1937 | 3-methoxy-5-chlorophenyl | N(2-propyl)₂ |
| A-1938 | 2-chloro-3-methoxyphenyl | N(2-propyl)₂ |
| A-1939 | 2-chloro-4-methoxyphenyl | N(2-propyl)₂ |
| A-1940 | 2-chloro-5-methoxyphenyl | N(2-propyl)₂ |
| A-1941 | 3-chloro-4-methoxyphenyl | N(2-propyl)₂ |
| A-1942 | 2-methoxy-3-fluorophenyl | N(2-propyl)₂ |
| A-1943 | 2-methoxy-4-fluorophenyl | N(2-propyl)₂ |
| A-1944 | 2-methoxy-5-fluorophenyl | N(2-propyl)₂ |
| A-1945 | 2-methoxy-6-fluorophenyl | N(2-propyl)₂ |
| A-1946 | 3-methoxy-4-fluorophenyl | N(2-propyl)₂ |
| A-1947 | 3-methoxy-5-fluorophenyl | N(2-propyl)₂ |
| A-1948 | 2-fluoro-3-methoxyphenyl | N(2-propyl)₂ |
| A-1949 | 2-fluoro-4-methoxyphenyl | N(2-propyl)₂ |
| A-1950 | 2-fluoro-5-methoxyphenyl | N(2-propyl)₂ |
| A-1951 | 3-fluoro-4-methoxyphenyl | N(2-propyl)₂ |
| A-1952 | 3-fluoro-5-methoxyphenyl | N(2-propyl)₂ |
| A-1953 | 2-(trifluoromethyl)-3-chlorophenyl | N(2-propyl)₂ |
| A-1954 | 2-(trifluoromethyl)-4-chlorophenyl | N(2-propyl)₂ |
| A-1955 | 2-(trifluoromethyl)-5-chlorophenyl | N(2-propyl)₂ |
| A-1956 | 2-(trifluoromethyl)-6-chlorophenyl | N(2-propyl)₂ |
| A-1957 | 3-(trifluoromethyl)-4-chlorophenyl | N(2-propyl)₂ |
| A-1958 | 3-(trifluoromethyl)-5-chlorophenyl | N(2-propyl)₂ |
| A-1959 | 2-chloro-3-(trifluoromethyl)phenyl | N(2-propyl)₂ |
| A-1960 | 2-chloro-4-(trifluoromethyl)phenyl | N(2-propyl)₂ |
| A-1961 | 2-chloro-5-(trifluoromethyl)phenyl | N(2-propyl)₂ |
| A-1962 | 3-chloro-4-(trifluoromethyl)phenyl | N(2-propyl)₂ |
| A-1963 | 2-(trifluoromethyl)-3-fluorophenyl | N(2-propyl)₂ |
| A-1964 | 2-(trifluoromethyl)-4-fluorophenyl | N(2-propyl)₂ |
| A-1965 | 2-(trifluoromethyl)-5-fluorophenyl | N(2-propyl)₂ |
| A-1966 | 2-(trifluoromethyl)-6-fluorophenyl | N(2-propyl)₂ |
| A-1967 | 3-(trifluoromethyl)-4-fluorophenyl | N(2-propyl)₂ |
| A-1968 | 3-(trifluoromethyl)-5-fluorophenyl | N(2-propyl)₂ |
| A-1969 | 2-fluoro-3-(trifluoromethyl)phenyl | N(2-propyl)₂ |
| A-1970 | 2-fluoro-4-(trifluoromethyl)phenyl | N(2-propyl)₂ |
| A-1971 | 2-fluoro-5-(trifluoromethyl)phenyl | N(2-propyl)₂ |
| A-1972 | 3-fluoro-4-(trifluoromethyl)phenyl | N(2-propyl)₂ |
| A-1973 | 2-(trifluoromethoxy)-3-chlorophenyl | N(2-propyl)₂ |
| A-1974 | 2-(trifluoromethoxy)-4-chlorophenyl | N(2-propyl)₂ |
| A-1975 | 2-(trifluoromethoxy)-5-chlorophenyl | N(2-propyl)₂ |
| A-1976 | 2-(trifluoromethoxy)-6-chlorophenyl | N(2-propyl)₂ |
| A-1977 | 3-(trifluoromethoxy)-4-chlorophenyl | N(2-propyl)₂ |
| A-1978 | 3-(trifluoromethoxy)-5-chlorophenyl | N(2-propyl)₂ |
| A-1979 | 2-chloro-3-(trifluoromethoxy)phenyl | N(2-propyl)₂ |
| A-1980 | 2-chloro-4-(trifluoromethoxy)phenyl | N(2-propyl)₂ |
| A-1981 | 2-chloro-5-(trifluoromethoxy)phenyl | N(2-propyl)₂ |
| A-1982 | 3-chloro-4-(trifluoromethoxy)phenyl | N(2-propyl)₂ |
| A-1983 | 2-(trifluoromethoxy)-3-fluorophenyl | N(2-propyl)₂ |
| A-1984 | 2-(trifluoromethoxy)-4-fluorophenyl | N(2-propyl)₂ |
| A-1985 | 2-(trifluoromethoxy)-5-fluorophenyl | N(2-propyl)₂ |
| A-1986 | 2-(trifluoromethoxy)-6-fluorophenyl | N(2-propyl)₂ |
| A-1987 | 3-(trifluoromethoxy)-4-fluorophenyl | N(2-propyl)₂ |
| A-1988 | 3-(trifluoromethoxy)-5-fluorophenyl | N(2-propyl)₂ |
| A-1989 | 2-fluoro-3-(trifluoromethoxy)phenyl | N(2-propyl)₂ |
| A-1990 | 2-fluoro-4-(trifluoromethoxy)phenyl | N(2-propyl)₂ |
| A-1991 | 2-fluoro-5-(trifluoromethoxy)phenyl | N(2-propyl)₂ |
| A-1992 | 3-fluoro-4-(trifluoromethoxy)phenyl | N(2-propyl)₂ |
| A-1993 | 2-(difluoromethoxy)-3-chlorophenyl | N(2-propyl)₂ |
| A-1994 | 2-(difluoromethoxy)-4-chlorophenyl | N(2-propyl)₂ |
| A-1995 | 2-(difluoromethoxy)-5-chlorophenyl | N(2-propyl)₂ |
| A-1996 | 2-(difluoromethoxy)-6-chlorophenyl | N(2-propyl)₂ |
| A-1997 | 3-(difluoromethoxy)-4-chlorophenyl | N(2-propyl)₂ |
| A-1998 | 3-(difluoromethoxy)-5-chlorophenyl | N(2-propyl)₂ |
| A-1999 | 2-chloro-3-(difluoromethoxy)phenyl | N(2-propyl)₂ |
| A-2000 | 2-chloro-4-(difluoromethoxy)phenyl | N(2-propyl)₂ |
| A-2001 | 2-chloro-5-(difluoromethoxy)phenyl | N(2-propyl)₂ |
| A-2002 | 3-chloro-4-(difluoromethoxy)phenyl | N(2-propyl)₂ |
| A-2003 | 2-(difluoromethoxy)-3-fluorophenyl | N(2-propyl)₂ |
| A-2004 | 2-(difluoromethoxy)-4-fluorophenyl | N(2-propyl)₂ |
| A-2005 | 2-(difluoromethoxy)-5-fluorophenyl | N(2-propyl)₂ |
| A-2006 | 2-(difluoromethoxy)-6-fluorophenyl | N(2-propyl)₂ |
| A-2007 | 3-(difluoromethoxy)-4-fluorophenyl | N(2-propyl)₂ |
| A-2008 | 3-(difluoromethoxy)-5-fluorophenyl | N(2-propyl)₂ |
| A-2009 | 2-fluoro-3-(difluoromethoxy)phenyl | N(2-propyl)₂ |
| A-2010 | 2-fluoro-4-(difluoromethoxy)phenyl | N(2-propyl)₂ |
| A-2011 | 2-fluoro-5-(difluoromethoxy)phenyl | N(2-propyl)₂ |
| A-2012 | 3-fluoro-4-(difluoromethoxy)phenyl | N(2-propyl)₂ |
| A-2013 | 2-(trifluoromethylthio)-3- | N(2-propyl)₂ |
| | chlorophenyl | |
| A-2014 | 2-(trifluoromethylthio)-4- | N(2-propyl)₂ |
| | chlorophenyl | |
| A-2015 | 2-(trifluoromethylthio)-5- | N(2-propyl)₂ |
| | chlorophenyl | |
| A-2016 | 2-(trifluoromethylthio)-6- | N(2-propyl)₂ |
| | chlorophenyl | |
| A-2017 | 3-(trifluoromethylthio)-4- | N(2-propyl)₂ |
| | chlorophenyl | |
| A-2018 | 3-(trifluoromethylthio)-5- | N(2-propyl)₂ |
| | chlorophenyl | |
| A-2019 | 2-chloro-3- | N(2-propyl)₂ |
| | (trifluoromethylthio)phenyl | |
| A-2020 | 2-chloro-4- | N(2-propyl)₂ |
| | (trifluoromethylthio)phenyl | |
| A-2021 | 2-chloro-5- | N(2-propyl)₂ |
| | (trifluoromethylthio)phenyl | |
| A-2022 | 3-chloro-4- | N(2-propyl)₂ |
| | (trifluoromethylthio)phenyl | |
| A-2023 | 2-(trifluoromethylthio)-3-fluorophenyl | N(2-propyl)₂ |
| A-2024 | 2-(trifluoromethylthio)-4-fluorophenyl | N(2-propyl)₂ |
| A-2025 | 2-(trifluoromethylthio)-5-fluorophenyl | N(2-propyl)₂ |
| A-2026 | 2-(trifluoromethylthio)-6-fluorophenyl | N(2-propyl)₂ |
| A-2027 | 3-(trifluoromethylthio)-4-fluorophenyl | N(2-propyl)₂ |
| A-2028 | 3-(trifluoromethylthio)-5-fluorophenyl | N(2-propyl)₂ |
| A-2029 | 2-fluoro-3-(trifluoromethylthio)phenyl | N(2-propyl)₂ |
| A-2030 | 2-fluoro-4-(trifluoromethylthio)phenyl | N(2-propyl)₂ |
| A-2031 | 2-fluoro-5-(trifluoromethylthio)phenyl | N(2-propyl)₂ |
| A-2032 | 3-fluoro-4-(trifluoromethylthio)phenyl | N(2-propyl)₂ |
| A-2033 | 2,3,4-trichlorophenyl | N(2-propyl)₂ |
| A-2034 | 2,3,5-trichlorophenyl | N(2-propyl)₂ |
| A-2035 | 2,3,6-trichlorophenyl | N(2-propyl)₂ |
| A-2036 | 2,4,5-trichlorophenyl | N(2-propyl)₂ |
| A-2037 | 2,4,6-trichlorophenyl | N(2-propyl)₂ |
| A-2038 | 3,4,5-trichlorophenyl | N(2-propyl)₂ |
| A-2039 | 2,3,4-trifluorophenyl | N(2-propyl)₂ |
| A-2040 | 2,3,5-trifluorophenyl | N(2-propyl)₂ |
| A-2041 | 2,3,6-trifluorophenyl | N(2-propyl)₂ |
| A-2042 | 2,4,5-trifluorophenyl | N(2-propyl)₂ |
| A-2043 | 2,4,6-trifluorophenyl | N(2-propyl)₂ |
| A-2044 | 3,4,5-trifluorophenyl | N(2-propyl)₂ |
| A-2045 | 2,3,4-trimethylphenyl | N(2-propyl)₂ |
| A-2046 | 2,3,5-trimethylphenyl | N(2-propyl)₂ |
| A-2047 | 2,3,6-trimethylphenyl | N(2-propyl)₂ |
| A-2048 | 2,4,5-trimethylphenyl | N(2-propyl)₂ |
| A-2049 | 2,4,6-trimethylphenyl | N(2-propyl)₂ |
| A-2050 | 3,4,5-trimethylphenyl | N(2-propyl)₂ |
| A-2051 | 2,3,4-trimethoxyphenyl | N(2-propyl)₂ |
| A-2052 | 2,3,5-trimethoxyphenyl | N(2-propyl)₂ |
| A-2053 | 2,3,6-trimethoxyphenyl | N(2-propyl)₂ |
| A-2054 | 2,4,5-trimethoxyphenyl | N(2-propyl)₂ |
| A-2055 | 2,4,6-trimethoxyphenyl | N(2-propyl)₂ |
| A-2056 | 3,4,5-trimethoxyphenyl | N(2-propyl)₂ |
| A-2057 | phenyl | N(ethyl)₂ |
| A-2058 | 2-chlorophenyl | N(ethyl)₂ |
| A-2059 | 3-chlorophenyl | N(ethyl)₂ |
| A-2060 | 4-chlorophenyl | N(ethyl)₂ |
| A-2061 | 2-fluorophenyl | N(ethyl)₂ |
| A-2062 | 3-fluorophenyl | N(ethyl)₂ |
| A-2063 | 4-fluorophenyl | N(ethyl)₂ |
| A-2064 | 2-methylphenyl | N(ethyl)₂ |
| A-2065 | 3-methylphenyl | N(ethyl)₂ |
| A-2066 | 4-methylphenyl | N(ethyl)₂ |
| A-2067 | 2-ethylphenyl | N(ethyl)₂ |
| A-2068 | 3-ethylphenyl | N(ethyl)₂ |
| A-2069 | 4-ethylphenyl | N(ethyl)₂ |
| A-2070 | 2-methoxyphenyl | N(ethyl)₂ |
| A-2071 | 3-methoxyphenyl | N(ethyl)₂ |
| A-2072 | 4-methoxyphenyl | N(ethyl)₂ |
| A-2073 | 2-trifluoromethylphenyl | N(ethyl)₂ |
| A-2074 | 3-trifluoromethylphenyl | N(ethyl)₂ |
| A-2075 | 4-trifluoromethylphenyl | N(ethyl)₂ |
| A-2076 | 2-trifluoromethoxyphenyl | N(ethyl)₂ |
| A-2077 | 3-trifluoromethoxyphenyl | N(ethyl)₂ |
| A-2078 | 4-trifluoromethoxyphenyl | N(ethyl)₂ |
| A-2079 | 2-difluoromethoxyphenyl | N(ethyl)₂ |
| A-2080 | 3-difluoromethoxyphenyl | N(ethyl)₂ |
| A-2081 | 4-difluoromethoxyphenyl | N(ethyl)₂ |
| A-2082 | 2-trifluoromethylthiophenyl | N(ethyl)₂ |
| A-2083 | 3-trifluoromethylthiophenyl | N(ethyl)₂ |
| A-2084 | 4-trifluoromethylthiophenyl | N(ethyl)₂ |
| A-2085 | 2,3-dichlorophenyl | N(ethyl)₂ |
| A-2086 | 2,4-dichlorophenyl | N(ethyl)₂ |
| A-2087 | 2,5-dichlorophenyl | N(ethyl)₂ |
| A-2088 | 2,6-dichlorophenyl | N(ethyl)₂ |
| A-2089 | 3,4-dichlorophenyl | N(ethyl)₂ |
| A-2090 | 3,5-dichlorophenyl | N(ethyl)₂ |
| A-2091 | 2,3-difluorophenyl | N(ethyl)₂ |
| A-2092 | 2,4-difluorophenyl | N(ethyl)₂ |
| A-2093 | 2,5-difluorophenyl | N(ethyl)₂ |
| A-2094 | 2,6-difluorophenyl | N(ethyl)₂ |
| A-2095 | 3,4-difluorophenyl | N(ethyl)₂ |
| A-2096 | 3,5-difluorophenyl | N(ethyl)₂ |
| A-2097 | 2,3-dimethylphenyl | N(ethyl)₂ |
| A-2098 | 2,4-dimethylphenyl | N(ethyl)₂ |
| A-2099 | 2,5-dimethylphenyl | N(ethyl)₂ |
| A-2100 | 2,6-dimethylphenyl | N(ethyl)₂ |
| A-2101 | 3,4-dimethylphenyl | N(ethyl)₂ |
| A-2102 | 3,5-dimethylphenyl | N(ethyl)₂ |
| A-2103 | 2,3-diethylphenyl | N(ethyl)₂ |
| A-2104 | 2,4-diethylphenyl | N(ethyl)₂ |
| A-2105 | 2,5-diethylphenyl | N(ethyl)₂ |
| A-2106 | 2,6-diethylphenyl | N(ethyl)₂ |
| A-2107 | 3,5-diethylphenyl | N(ethyl)₂ |
| A-2108 | 3,4-diethylphenyl | N(ethyl)₂ |
| A-2109 | 2,3-dimethoxyphenyl | N(ethyl)₂ |
| A-2110 | 2,4-dimethoxyphenyl | N(ethyl)₂ |
| A-2111 | 2,5-dimethoxyphenyl | N(ethyl)₂ |
| A-2112 | 2,6-dimethoxyphenyl | N(ethyl)₂ |
| A-2113 | 3,4-dimethoxyphenyl | N(ethyl)₂ |
| A-2114 | 3,5-dimethoxyphenyl | N(ethyl)₂ |
| A-2115 | 2,3-di(trifluoromethyl)phenyl | N(ethyl)₂ |
| A-2116 | 2,4-di(trifluoromethyl)phenyl | N(ethyl)₂ |
| A-2117 | 2,5-di(trifluoromethyl)phenyl | N(ethyl)₂ |
| A-2118 | 2,6-di(trifluoromethyl)phenyl | N(ethyl)₂ |
| A-2119 | 3,4-di(trifluoromethyl)phenyl | N(ethyl)₂ |
| A-2120 | 3,5-di(trifluoromethyl)phenyl | N(ethyl)₂ |
| A-2121 | 2,3-di(trifluoromethoxy)phenyl | N(ethyl)₂ |
| A-2122 | 2,4-di(trifluoromethoxy)phenyl | N(ethyl)₂ |
| A-2123 | 2,5-di(trifluoromethoxy)phenyl | N(ethyl)₂ |
| A-2124 | 2,6-di(trifluoromethoxy)phenyl | N(ethyl)₂ |
| A-2125 | 3,4-di(trifluoromethoxy)phenyl | N(ethyl)₂ |
| A-2126 | 3,5-di(trifluoromethoxy)phenyl | N(ethyl)₂ |
| A-2127 | 2,3-di(difluoromethoxy)phenyl | N(ethyl)₂ |
| A-2128 | 2,4-di(difluoromethoxy)phenyl | N(ethyl)₂ |
| A-2129 | 2,5-di(difluoromethoxy)phenyl | N(ethyl)₂ |
| A-2130 | 2,6-di(difluoromethoxy)phenyl | N(ethyl)₂ |
| A-2131 | 3,4-di(difluoromethoxy)phenyl | N(ethyl)₂ |
| A-2132 | 3,5-di(difluoromethoxy)phenyl | N(ethyl)₂ |
| A-2133 | 2,3-di(trifluoromethylthio)phenyl | N(ethyl)₂ |
| A-2134 | 2,4-di(trifluoromethylthio)phenyl | N(ethyl)₂ |
| A-2135 | 2,5-di(trifluoromethylthio)phenyl | N(ethyl)₂ |
| A-2136 | 2,6-di(trifluoromethylthio)phenyl | N(ethyl)₂ |
| A-2137 | 3,4-di(trifluoromethylthio)phenyl | N(ethyl)₂ |
| A-2138 | 3,5-di(trifluoromethylthio)phenyl | N(ethyl)₂ |
| A-2139 | 2-fluoro-3-chlorophenyl | N(ethyl)₂ |
| A-2140 | 2-fluoro-4-chlorophenyl | N(ethyl)₂ |
| A-2141 | 2-fluoro-5-chlorophenyl | N(ethyl)₂ |
| A-2142 | 2-fluoro-6-chlorophenyl | N(ethyl)₂ |
| A-2143 | 3-fluoro-4-chlorophenyl | N(ethyl)₂ |
| A-2144 | 3-fluoro-5-chlorophenyl | N(ethyl)₂ |
| A-2145 | 2-chloro-3-fluorophenyl | N(ethyl)₂ |
| A-2146 | 2-chloro-4-fluorophenyl | N(ethyl)₂ |
| A-2147 | 2-chloro-5-fluorophenyl | N(ethyl)₂ |
| A-2148 | 3-chloro-4-fluorophenyl | N(ethyl)₂ |
| A-2149 | 2-methyl-3-chlorophenyl | N(ethyl)₂ |
| A-2150 | 2-methyl-4-chlorophenyl | N(ethyl)₂ |
| A-2151 | 2-methyl-5-chlorophenyl | N(ethyl)₂ |
| A-2152 | 2-methyl-6-chlorophenyl | N(ethyl)₂ |
| A-2153 | 3-methyl-4-chlorophenyl | N(ethyl)₂ |
| A-2154 | 3-methyl-5-chlorophenyl | N(ethyl)₂ |
| A-2155 | 2-chloro-3-methylphenyl | N(ethyl)₂ |
| A-2156 | 2-chloro-4-methylphenyl | N(ethyl)₂ |
| A-2157 | 2-chloro-5-methylphenyl | N(ethyl)₂ |
| A-2158 | 3-chloro-4-methylphenyl | N(ethyl)₂ |
| A-2159 | 2-methyl-3-fluorophenyl | N(ethyl)₂ |
| A-2160 | 2-methyl-4-fluorophenyl | N(ethyl)₂ |
| A-2161 | 2-methyl-5-fluorophenyl | N(ethyl)₂ |
| A-2162 | 2-methyl-6-fluorophenyl | N(ethyl)₂ |
| A-2163 | 3-methyl-4-fluorophenyl | N(ethyl)₂ |
| A-2164 | 3-methyl-5-fluorophenyl | N(ethyl)₂ |
| A-2165 | 2-fluoro-3-methylphenyl | N(ethyl)₂ |
| A-2166 | 2-fluoro-4-methylphenyl | N(ethyl)₂ |
| A-2167 | 2-fluoro-5-methylphenyl | N(ethyl)₂ |
| A-2168 | 3-fluoro-4-methylphenyl | N(ethyl)₂ |
| A-2169 | 2-chloro-3-ethylphenyl | N(ethyl)₂ |
| A-2170 | 2-chloro-4-ethylphenyl | N(ethyl)₂ |
| A-2171 | 2-chloro-5-ethylphenyl | N(ethyl)₂ |
| A-2172 | 3-chloro-4-ethylphenyl | N(ethyl)₂ |
| A-2173 | 2-ethyl-3-chlorophenyl | N(ethyl)₂ |
| A-2174 | 2-ethyl-4-chlorophenyl | N(ethyl)₂ |
| A-2175 | 2-ethyl-5-chlorophenyl | N(ethyl)₂ |
| A-2176 | 2-ethyl-6-chlorophenyl | N(ethyl)₂ |
| A-2177 | 3-ethyl-4-chlorophenyl | N(ethyl)₂ |
| A-2178 | 3-ethyl-5-chlorophenyl | N(ethyl)₂ |
| A-2179 | 2-ethyl-3-fluorophenyl | N(ethyl)₂ |
| A-2180 | 2-ethyl-4-fluorophenyl | N(ethyl)₂ |
| A-2181 | 2-ethyl-5-fluorophenyl | N(ethyl)₂ |
| A-2182 | 2-ethyl-6-fluorophenyl | N(ethyl)₂ |
| A-2183 | 3-ethyl-4-fluorophenyl | N(ethyl)₂ |
| A-2184 | 3-ethyl-5-fluorophenyl | N(ethyl)₂ |
| A-2185 | 2-fluoro-3-ethylphenyl | N(ethyl)₂ |
| A-2186 | 2-fluoro-4-ethylphenyl | N(ethyl)₂ |
| A-2187 | 2-fluoro-5-ethylphenyl | N(ethyl)₂ |
| A-2188 | 3-fluoro-4-ethylphenyl | N(ethyl)₂ |
| A-2189 | 2-methoxy-3-chlorophenyl | N(ethyl)₂ |
| A-2190 | 2-methoxy-4-chlorophenyl | N(ethyl)₂ |
| A-2191 | 2-methoxy-5-chlorophenyl | N(ethyl)₂ |
| A-2192 | 2-methoxy-6-chlorophenyl | N(ethyl)₂ |
| A-2193 | 3-methoxy-4-chlorophenyl | N(ethyl)₂ |
| A-2194 | 3-methoxy-5-chlorophenyl | N(ethyl)₂ |
| A-2195 | 2-chloro-3-methoxyphenyl | N(ethyl)₂ |
| A-2196 | 2-chloro-4-methoxyphenyl | N(ethyl)₂ |
| A-2197 | 2-chloro-5-methoxyphenyl | N(ethyl)₂ |
| A-2198 | 3-chloro-4-methoxyphenyl | N(ethyl)₂ |
| A-2199 | 2-methoxy-3-fluorophenyl | N(ethyl)₂ |
| A-2200 | 2-methoxy-4-fluorophenyl | N(ethyl)₂ |
| A-2201 | 2-methoxy-5-fluorophenyl | N(ethyl)₂ |
| A-2202 | 2-methoxy-6-fluorophenyl | N(ethyl)₂ |
| A-2203 | 3-methoxy-4-fluorophenyl | N(ethyl)₂ |
| A-2204 | 3-methoxy-5-fluorophenyl | N(ethyl)₂ |
| A-2205 | 2-fluoro-3-methoxyphenyl | N(ethyl)₂ |
| A-2206 | 2-fluoro-4-methoxyphenyl | N(ethyl)₂ |
| A-2207 | 2-fluoro-5-methoxyphenyl | N(ethyl)₂ |
| A-2208 | 3-fluoro-4-methoxyphenyl | N(ethyl)₂ |
| A-2209 | 3-fluoro-5-methoxyphenyl | N(ethyl)₂ |
| A-2210 | 2-(trifluoromethyl)-3-chlorophenyl | N(ethyl)₂ |
| A-2211 | 2-(trifluoromethyl)-4-chlorophenyl | N(ethyl)₂ |
| A-2212 | 2-(trifluoromethyl)-5-chlorophenyl | N(ethyl)₂ |
| A-2213 | 2-(trifluoromethyl)-6-chlorophenyl | N(ethyl)₂ |
| A-2214 | 3-(trifluoromethyl)-4-chlorophenyl | N(ethyl)₂ |
| A-2215 | 3-(trifluoromethyl)-5-chlorophenyl | N(ethyl)₂ |
| A-2216 | 2-chloro-3-(trifluoromethyl)phenyl | N(ethyl)₂ |
| A-2217 | 2-chloro-4-(trifluoromethyl)phenyl | N(ethyl)₂ |
| A-2218 | 2-chloro-5-(trifluoromethyl)phenyl | N(ethyl)₂ |
| A-2219 | 3-chloro-4-(trifluoromethyl)phenyl | N(ethyl)₂ |
| A-2220 | 2-(trifluoromethyl)-3-fluorophenyl | N(ethyl)₂ |
| A-2221 | 2-(trifluoromethyl)-4-fluorophenyl | N(ethyl)₂ |
| A-2222 | 2-(trifluoromethyl)-5-fluorophenyl | N(ethyl)₂ |
| A-2223 | 2-(trifluoromethyl)-6-fluorophenyl | N(ethyl)₂ |
| A-2224 | 3-(trifluoromethyl)-4-fluorophenyl | N(ethyl)₂ |
| A-2225 | 3-(trifluoromethyl)-5-fluorophenyl | N(ethyl)₂ |
| A-2226 | 2-fluoro-3-(trifluoromethyl)phenyl | N(ethyl)₂ |
| A-2227 | 2-fluoro-4-(trifluoromethyl)phenyl | N(ethyl)₂ |
| A-2228 | 2-fluoro-5-(trifluoromethyl)phenyl | N(ethyl)₂ |
| A-2229 | 3-fluoro-4-(trifluoromethyl)phenyl | N(ethyl)₂ |
| A-2230 | 2-(trifluoromethoxy)-3-chlorophenyl | N(ethyl)₂ |
| A-2231 | 2-(trifluoromethoxy)-4-chlorophenyl | N(ethyl)₂ |
| A-2232 | 2-(trifluoromethoxy)-5-chlorophenyl | N(ethyl)₂ |
| A-2233 | 2-(trifluoromethoxy)-6-chlorophenyl | N(ethyl)₂ |
| A-2234 | 3-(trifluoromethoxy)-4-chlorophenyl | N(ethyl)₂ |
| A-2235 | 3-(trifluoromethoxy)-5-chlorophenyl | N(ethyl)₂ |
| A-2236 | 2-chloro-3-(trifluoromethoxy)phenyl | N(ethyl)₂ |
| A-2237 | 2-chloro-4-(trifluoromethoxy)phenyl | N(ethyl)₂ |
| A-2238 | 2-chloro-5-(trifluoromethoxy)phenyl | N(ethyl)₂ |
| A-2239 | 3-chloro-4-(trifluoromethoxy)phenyl | N(ethyl)₂ |
| A-2240 | 2-(trifluoromethoxy)-3-fluorophenyl | N(ethyl)₂ |
| A-2241 | 2-(trifluoromethoxy)-4-fluorophenyl | N(ethyl)₂ |
| A-2242 | 2-(trifluoromethoxy)-5-fluorophenyl | N(ethyl)₂ |
| A-2243 | 2-(trifluoromethoxy)-6-fluorophenyl | N(ethyl)₂ |
| A-2244 | 3-(trifluoromethoxy)-4-fluorophenyl | N(ethyl)₂ |
| A-2245 | 3-(trifluoromethoxy)-5-fluorophenyl | N(ethyl)₂ |
| A-2246 | 2-fluoro-3-(trifluoromethoxy)phenyl | N(ethyl)₂ |
| A-2247 | 2-fluoro-4-(trifluoromethoxy)phenyl | N(ethyl)₂ |
| A-2248 | 2-fluoro-5-(trifluoromethoxy)phenyl | N(ethyl)₂ |
| A-2249 | 3-fluoro-4-(trifluoromethoxy)phenyl | N(ethyl)₂ |
| A-2250 | 2-(difluoromethoxy)-3-chlorophenyl | N(ethyl)₂ |
| A-2251 | 2-(difluoromethoxy)-4-chlorophenyl | N(ethyl)₂ |
| A-2252 | 2-(difluoromethoxy)-5-chlorophenyl | N(ethyl)₂ |
| A-2253 | 2-(difluoromethoxy)-6-chlorophenyl | N(ethyl)₂ |
| A-2254 | 3-(difluoromethoxy)-4-chlorophenyl | N(ethyl)₂ |
| A-2255 | 3-(difluoromethoxy)-5-chlorophenyl | N(ethyl)₂ |
| A-2256 | 2-chloro-3-(difluoromethoxy)phenyl | N(ethyl)₂ |
| A-2257 | 2-chloro-4-(difluoromethoxy)phenyl | N(ethyl)₂ |
| A-2258 | 2-chloro-5-(difluoromethoxy)phenyl | N(ethyl)₂ |
| A-2259 | 3-chloro-4-(difluoromethoxy)phenyl | N(ethyl)₂ |
| A-2260 | 2-(difluoromethoxy)-3-fluorophenyl | N(ethyl)₂ |
| A-2261 | 2-(difluoromethoxy)-4-fluorophenyl | N(ethyl)₂ |
| A-2262 | 2-(difluoromethoxy)-5-fluorophenyl | N(ethyl)₂ |
| A-2263 | 2-(difluoromethoxy)-6-fluorophenyl | N(ethyl)₂ |
| A-2264 | 3-(difluoromethoxy)-4-fluorophenyl | N(ethyl)₂ |
| A-2265 | 3-(difluoromethoxy)-5-fluorophenyl | N(ethyl)₂ |
| A-2266 | 2-fluoro-3-(difluoromethoxy)phenyl | N(ethyl)₂ |
| A-2267 | 2-fluoro-4-(difluoromethoxy)phenyl | N(ethyl)₂ |
| A-2268 | 2-fluoro-5-(difluoromethoxy)phenyl | N(ethyl)₂ |
| A-2269 | 3-fluoro-4-(difluoromethoxy)phenyl | N(ethyl)₂ |
| A-2270 | 2-(trifluoromethylthio)-3- | N(ethyl)₂ |
| | chlorophenyl | |
| A-2271 | 2-(trifluoromethylthio)-4- | N(ethyl)₂ |
| | chlorophenyl | |
| A-2272 | 2-(trifluoromethylthio)-5- | N(ethyl)₂ |
| | chlorophenyl | |
| A-2273 | 2-(trifluoromethylthio)-6- | N(ethyl)₂ |
| | chlorophenyl | |
| A-2274 | 3-(trifluoromethylthio)-4- | N(ethyl)₂ |
| | chlorophenyl | |
| A-2275 | 3-(trifluoromethylthio)-5- | N(ethyl)₂ |
| | chlorophenyl | |
| A-2276 | 2-chloro-3- | N(ethyl)₂ |
| | (trifluoromethylthio)phenyl | |
| A-2277 | 2-chloro-4- | N(ethyl)₂ |
| | (trifluoromethylthio)phenyl | |
| A-2278 | 2-chloro-5- | N(ethyl)₂ |
| | (trifluoromethylthio)phenyl | |
| A-2279 | 3-chloro-4- | N(ethyl)₂ |
| | (trifluoromethylthio)phenyl | |
| A-2280 | 2-(trifluoromethylthio)-3-fluorophenyl | N(ethyl)₂ |
| A-2281 | 2-(trifluoromethylthio)-4-fluorophenyl | N(ethyl)₂ |
| A-2282 | 2-(trifluoromethylthio)-5-fluorophenyl | N(ethyl)₂ |
| A-2283 | 2-(trifluoromethylthio)-6-fluorophenyl | N(ethyl)₂ |
| A-2284 | 3-(trifluoromethylthio)-4-fluorophenyl | N(ethyl)₂ |
| A-2285 | 3-(trifluoromethylthio)-5-fluorophenyl | N(ethyl)₂ |
| A-2286 | 2-fluoro-3-(trifluoromethylthio)phenyl | N(ethyl)₂ |
| A-2287 | 2-fluoro-4-(trifluoromethylthio)phenyl | N(ethyl)₂ |
| A-2288 | 2-fluoro-5-(trifluoromethylthio)phenyl | N(ethyl)₂ |
| A-2289 | 3-fluoro-4-(trifluoromethylthio)phenyl | N(ethyl)₂ |
| A-2290 | 2,3,4-trichlorophenyl | N(ethyl)₂ |
| A-2291 | 2,3,5-trichlorophenyl | N(ethyl)₂ |
| A-2292 | 2,3,6-trichlorophenyl | N(ethyl)₂ |
| A-2293 | 2,4,5-trichlorophenyl | N(ethyl)₂ |
| A-2294 | 2,4,6-trichlorophenyl | N(ethyl)₂ |
| A-2295 | 3,4,5-trichlorophenyl | N(ethyl)₂ |
| A-2296 | 2,3,4-trifluorophenyl | N(ethyl)₂ |
| A-2297 | 2,3,5-trifluorophenyl | N(ethyl)₂ |
| A-2298 | 2,3,6-trifluorophenyl | N(ethyl)₂ |
| A-2299 | 2,4,5-trifluorophenyl | N(ethyl)₂ |
| A-2300 | 2,4,6-trifluorophenyl | N(ethyl)₂ |
| A-2301 | 3,4,5-trifluorophenyl | N(ethyl)₂ |
| A-2302 | 2,3,4-trimethylphenyl | N(ethyl)₂ |
| A-2303 | 2,3,5-trimethylphenyl | N(ethyl)₂ |
| A-2304 | 2,3,6-trimethylphenyl | N(ethyl)₂ |
| A-2305 | 2,4,5-trimethylphenyl | N(ethyl)₂ |
| A-2306 | 2,4,6-trimethylphenyl | N(ethyl)₂ |
| A-2307 | 3,4,5-trimethylphenyl | N(ethyl)₂ |
| A-2308 | 2,3,4-trimethoxyphenyl | N(ethyl)₂ |
| A-2309 | 2,3,5-trimethoxyphenyl | N(ethyl)₂ |
| A-2310 | 2,3,6-trimethoxyphenyl | N(ethyl)₂ |
| A-2311 | 2,4,5-trimethoxyphenyl | N(ethyl)₂ |
| A-2312 | 2,4,6-trimethoxyphenyl | N(ethyl)₂ |
| A-2313 | 3,4,5-trimethoxyphenyl | N(ethyl)₂ |

One undesired side product in the synthesis of compounds (I)-(1) that may occur in undesired amounts with prior art processes and that can be reduced or even avoided using the new inventive process is the cyclizised hydroxy compound IA:

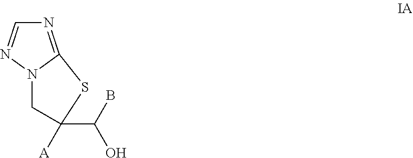

wherein A and B are as defined and preferably defined as for compounds (I)-(1). In conventional processes, for example using high temperature or n-butyllithium as a base, product IA may occur to up to 100%, leading, consequently, to very low yields of the desired product of formula (I). According to the inventive process, in particular when carrying out the process steps (ii) and (iii-1), more particular steps (i), (ii) and (iii-1), in case R has the meaning (1), side product IA is formed preferably to equal or less than 10%, more preferably equal or less than 8%, even more preferably equal or less than 5%, even more preferably equal or less than 3%.

Another undesired side product in the synthesis of compounds (I)-(1) that may occur in undesired amounts with prior art processes and that can be reduced or even avoided using the new inventive process is the cyclizised hydroxy compound IB:

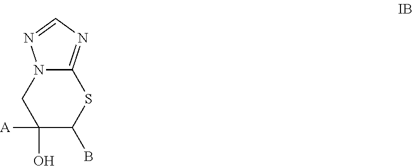

wherein A and B are as defined and preferably defined as for compounds (I)-(1). In conventional processes, for example using high temperature or n-butyllithium as a base, product IB may occur to up to 100%, leading, consequently, to very low yields of the desired product of formula (I). According to the inventive process, in particular when carrying out the process steps (ii) and (iii-1), more particular steps (i), (ii) and (iii-1), in case R has the meaning (1), side product IA is formed preferably to equal or less than 10%, more preferably equal or less than 8%, even more preferably equal or less than 5%, even more preferably equal or less than 3%.

According to another embodiment of the present invention, the organic group R in the compounds (I) and the precursors thereof carries a free hydroxy group and compounds (1) are from the triazole class of fungicides. In a particular embodiment thereof, R stands for a group of formula (2):

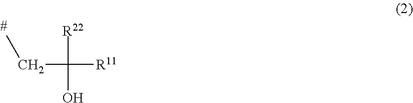

wherein R¹¹ and R²² have the following meanings:
R¹¹, R²² independently of one another C₁-C₆-alkyl, C₁-C₆-haloalkyl, C₃-C₆-cycloalkyl, C₃-C₆-halocycloalkyl or phenyl, wherein the alkyl, cycloalkyl and phenyl moieties may be unsubstituted or substituted by one, two, three or four substituents L as defined or preferably defined above for compounds, wherein R is a group (1); or
R¹¹ and R²², together with the carbon atom to which they are attached, form a five- or six-membered saturated or partially unsaturated ring, that can be unsubstituted or substituted by one, two, three, four or five substituents L′, wherein L′ stands for L as defi ned above or stands for a group

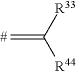

wherein R³³ and R⁴⁴ independently are selected from the group of hydrogen and the meaning for L as defined above.

According to one embodiment, R¹¹ and R¹² are preferably independently selected from C₁-C₄-alkyl and phenyl, wherein the alkyl and phenyl group independently may contain one, two, three or four substitutents, independently selected from F, Cl, Br, methoxy, ethoxy, propoxy, isopropoxy, C₁-C₂-alkoximino, cyclopropyl, cyclobutyl, cyclopentyl and/or cyclohexyl. Specifically, R¹¹ stands for C₁-C₄-alkyl that is substituted by one or two substituents independently selected from F, Cl, methoxy, cyclopropyl, cyclopentyl and/or cyclohexyl and R¹² stands for phenyl, that is substituted by one, two, three or four substituents independently selected from F, Cl, Br and methoxy. In one specific embodiment, R¹¹ is 1-ethyl that is 1-substituted by cyclopropyl and R¹² is 4-chlorophenyl. According to another specific embodiment, R¹¹ is n-butyl and R¹² is 2,4-dichlorophenyl.

According to another embodiment, R¹¹ and R¹² are preferably independently selected from C₁-C₄-alkyl, phenyl-C₁-C₄-alkyl and C₃-C₆-cycloalkyl, preferably phenyl-C₁-C₄-alkyl and C₃-C₆-cycloalkyl, wherein the alkyl, phenyl and cycloalkyl groups independently may contain one, two, three or four substitutents, independently selected from F, Cl, Br, CN, methyl, ethyl, propyl, isopropyl and/or tert-butyl. Specifically, R¹¹ stands for phenyl-C₁-C₄-alkyl that is substituted in the phenyl moiety by one, two, three or four substituents independently selected from F, Cl and methoxy and R¹² stands for C₃-C₆-cycloalkyl, that is substituted by one, two, three or four substituents independently selected from F, Cl, Br and methoxy. In one specific embodiment, R¹¹ is 2-chlorophenylmethyl and R¹² is 1-chlorocyclopropyl.

According to still another embodiment, R¹¹ and R¹² are preferably independently selected from C₁-C₄-alkyl and phenyl-C₁-C₄-alkyl, wherein the alkyl and phenyl groups may contain one, two, three or four substitutents, independently selected from F, Cl, Br, CN, methyl, ethyl, propyl, isopropyl, tert-butyl, methoxy, ethoxy, methylthio, trifluoromethyl, trifluoromethoxy, trifluoromethylthio, chlorodifluoromethoxy, difluoromethoxy, chlorodifluoromethylthio, methoxycarbonyl, ethoxyvarbonyl, methoxyiminomethyl, 1-methoximinoethyl and nitro. Specifically, R¹¹ stands for C₁-C₄-alkyl that may be substituted by one or two substituents, independently selected from methyl, ethyl, propyl, isopropyl and tert-butyl and R¹² stands for phenyl-C₁-C₄-alkyl, that is substituted in the phenyl moiety by one, two, three or four substituents independently selected from F, Cl, Br, CN, methyl, trifluoromethyl and methoxy. In one specific embodiment R¹¹ is tert-butyl and R¹² is 2-(4-chlorophenyl)-1-ethyl.

According to still another embodiment, R¹¹ and R¹² are preferably independently selected from phenyl, wherein the phenyl moieties may contain one, two, three or four substitutents, independently selected from F, Cl, Br, CN, methyl, ethyl, propyl, isopropyl, tert-butyl, methoxy, ethoxy, methylthio, trifluoromethyl, trifluoromethoxy, trifluoromethylthio, chlorodifluoromethoxy, difluoromethoxy, chlorodifluoromethylthio, methoxycarbonyl, ethoxyvarbonyl, methoxyiminomethyl, 1-methoximinoethyl and nitro. Specifically, R¹¹ and R¹² independently stand for phenyl, that may contain one, two or three substitutents, independently selected from F, Cl and Br. In one specific embodiment R¹¹ is 2-fluorophenyl and R¹² is 4-fluorophenyl.

According to still another embodiment, preferably R¹¹ and R²², together with the carbon atom to which they are attached, form a five- or six-membered saturated ring, that can be unsubstituted or substituted by one, two or three substituents L′, wherein L′ stands for L as defined above or stands for a group

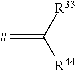

wherein R³³ and R⁴⁴ independently are selected from the group of hydrogen, C₁-C₄-alkyl and phenyl, wherein the alkyl and phenyl groups may contain one, two, three or four substitutents, independently selected from F, Cl, Br, CN, methyl, ethyl, propyl, isopropyl, tert-butyl, methoxy, ethoxy, methylthio, trifluoromethyl, trifluoromethoxy, trifluoromethylthio, chlorodifluoromethoxy, difluoromethoxy and nitro. Specifically, R¹¹ and R²², together with the carbon atom to which they are attached, form a five-membered saturated ring, that is substituted by one, two or three substituents L′, wherein L′ stands for C₁-C₄-alkyl or for a group

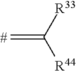

wherein R³³ and R⁴⁴ independently are selected from the group of hydrogen, C₁-C₄-alkyl and phenyl, wherein the alkyl and phenyl groups may contain one, two, three or four substitutents, independently selected from F, Cl, CN, methyl, isopropyl, tert-butyl and methoxy. In one specific embodiment R¹¹ and R²², together with the carbon atom to which they are attached, form a five-membered saturated ring, that is substituted in 5-position by two methyl groups and contains a group

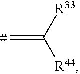

wherein R³³ is hydrogen and R⁴⁴ is 4-chlorophenyl in 2-position.

According to still another embodiment, R¹¹ and R²², together with the carbon atom to which they are attached, form a five- or six-membered saturated ring, that can be un-substituted or substituted by one, two or three substituents, independently selected from F, Cl, Br, CN, methyl, ethyl, propyl, isopropyl, tert-butyl, methoxy, ethoxy, methylthio, trifluoromethyl, trifluoromethoxy, trifluoromethylthio, chlorodifluoromethoxy, difluoromethoxy, nitro, benzyl, wherein the phenyl moiety itself may contain on, two, three or four substituents, independently selected from F, Cl, CN, methyl, isopropyl, tert-butyl and methoxy. In one specific embodiment R¹¹ and R²², together with the carbon atom to which they are attached, form a five-membered saturated ring, that is substituted in 5-position by two methyl groups and contains a 4-chlorobenzyl group in 2-position. Regarding compounds (I)-(2) and the synthesis of precursors thereof see also WO 96/16048, WO 96/38423, EP378953, EP655443, DE 4030039, DE 3337937, DE3315681, U.S. Pat. No. 4,414,210.

According to another embodiment of the present invention, R stands for a group of formula (3):

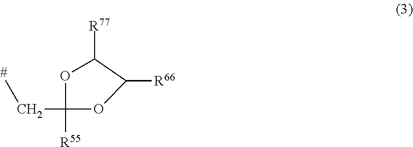

wherein R⁵⁵, R⁶⁶ and R⁷⁷ have the following meanings:

R⁵⁵ phenyl-C₁-C₈-alkyl, phenyl or a five- or six-membered saturated, partially unsaturated or aromatic heterocycle which contains one, two, three or four heteroatoms from the group consisting of O, N and S; where the aliphatic and/or aromatic and/or heterocyclic groups for their part may carry one, two, three or four identical or different groups selected from halogen, cyano, nitro, C₁-C₈-alkyl, C₁-C₈-haloalkyl, C₁-C₈-alkoxy, C₁-C₈-haloalkoxy, C₃-C₈-cycloalkyl, C₃-C₈-halocycloalkyl, C₃-C₈-cycloalkenyl, C₃-C₈-cycloalkoxy, C₃-C₈-halocycloalkoxy, C₁-C₈-alkylcarbonyl, C₁-C₈-alkylcarbonyloxy, C₁-C₈-alkoxycarbonyl, amino, C₁-C₈-alkylamino, di-C₁-C₈-alkylamino, phenyl, halophenyl, phenyloxy, halophenyloxy;
R⁶⁶, R⁷⁷ independently of one another hydrogen, C₁-C₆-alkyl, C₁-C₆-haloalkyl, C₃-C₆-cycloalkyl, C₃-C₆-halocycloalkyl or phenyl, wherein the alkyl, cycloalkyl or phenyl moieties may be unsubstituted or substituted by one, two or three substituents selected from halogen, cyano, nitro, C₁-C₈-alkyl, C₁-C₈-haloalkyl, C₁-C₈-alkoxy, C₁-C₈-haloalkoxy

According to one embodiment, R⁵⁵ is phenyl, that is unsubstituted or substituted by one, two, three or four substituents independently selected from halogen, C₁-C₆-alkyl, C₁-C₆-haloalkyl, phenoxy-C₁-C₆-alkyl and halophenyloxy, and R⁶⁶ and R⁷⁷ are independently selected from hydrogen, methyl, ethyl, n-propyl and n-butyl. Specifically, R⁵⁵ is phenyl, that contains one, two or three substituents independently selected from F, Cl and halophenoxy, wherein the phenoxy moiety contains one or two halogen atoms selected from Cl and F; and R⁶⁶ is hydrogen and R⁷⁷ is C₁-C₄-alkyl. In one specific embodiment R⁵⁵ is 4-(4-chlorophenoxy)-2-chlorophenyl, R⁶⁶ is hydrogen and R⁷⁷ is methyl. In another specific embodiment R⁵⁵ is 2,4-dichlorophenyl, R⁶⁶ is hydrogen and R⁷⁷ is n-propyl.

Regarding compounds (I)-(3) and the synthesis of precursors thereof see also WO 96/41804 and Pestic. Sci, 1980, 11, 95 and Research Disclosure 1989, 297,13.

According to another embodiment of the present invention, R stands for a group of formula (4):

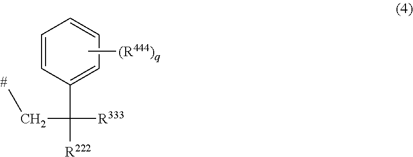

wherein R²²², R³³³ and R⁴⁴⁴ have the following meanings:

R²²² and R³³³ are independently selected from hydrogen, cyano, C₁-C₆-alkyl and C₁-C₆-haloalkyl, wherein the alkyl moieties may be unsubstituted or substituted by one, two, three or four substituents L as defined or preferably defined above for compounds, wherein R is a group (1). In particular, R²²² and R³³³ are independently selected from hydrogen, cyano and C₁-C₄-alkyl, wherein the alkyl moiety may contain one, two, three or four substituents independently selected from F, Cl, CN, C₁-C₄-alkoxy and C₁-C₄-haloalkoxy. q is one, two three or five, preferably one or two, and R⁴⁴⁴ are independently selected from L as defined or preferably defined above for compounds, wherein R is a group (1), in particular independently selected from F, Cl, CN, methyl, isopropyl, tert-butyl and methoxy, more specifically independently selected from Cl and F. According to one specific embodiment, R²²², is hydrogen, R³³³ is methyl, substituted by 1,1,2,2-tetrafluoroethoxy, and R⁴⁴⁴ is 2,4-dichlorophenyl. According to another specific embodiment, R²²² is cyano, R³³³ is n-butyl and R⁴⁴⁴ is 4-chlorophenyl. According to still another specific embodiment, R²²² is hydrogen, R³³³ is n-propyl and R⁴⁴⁴ is 2,4-dichlorophenyl. Regarding compounds (I)-(4) and the synthesis of precursors thereof see also DE19528300, DE19529089.

According to another embodiment of the present invention, R stands for a group of formula (5):

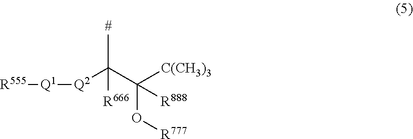

wherein # shall mean the point of attachment to the triazolo group and Q¹, Q², R⁵⁵⁵, R⁶⁶⁶, R⁷⁷⁷ and R⁸⁸⁸ are as defined as follows:
Q¹ O or a single bond to R⁵⁵⁵;
Q² saturated hydrocarbon chain containing two to five carbon atoms, which may contain one, two or three substituents R^{z}, wherein R^{z} has the meaning:
   R^{z} halogen, C₁-C₈-alkyl, C₁-C₈-haloalkyl, C₂-C₈-alkenyl, C₂-C₈-haloalkenyl, C₁-C₈-alkoxy, C₁-C₈-haloalkoxy, C₁-C₈-alkylcarbonyloxy, C₂-C₈-alkenyloxy, C₂-C₈-haloalkenyloxy, C₃-C₈-cycloalkyl, C₃-C₈-halo cycloalkyl, C₃-C₈-cycloalkenyl, C₃-C₈-halocycloalkenyl, C₃-C₈-cycloalkoxy, C₃-C₆-cycloalkenyloxy, C₁-C₆-alkylen, oxy-C₂-C₄-alkylen, phenoxy, phenyl; wherein R^{z} in each case is unsubstituted or contains one, two or three substituents, independently selected from L¹;
R⁵⁵⁵ phenyl, which is unsubstituted or contains one, two, three, four or five independently selected substituents L¹, wherein L¹ has the meanings:
   L¹ halogen, C₁-C₈-alkyl, C₁-C₈-haloalkyl, C₂-C₈-alkenyl, C₂-C₈-haloalkenyl, C₄-C₁₀-alkadienyl, C₄-C₁₀-haloalkadienyl, C₁-C₈-alkoxy, C₁-C₈-haloalkoxy, C₁-C₈-alkylcarbonyloxy, C₂-C₈-alkenyloxy, C₂-C₈-haloalkenyloxy, C₃-C₈-cycloalkyl, C₃-C₈-halocycloalkyl, C₃-C₈-cycloalkenyl, C₃-C₈-halocycloalkenyl, C₃-C₈-cycloalkoxy, C₃-C₆-cycloalkenyloxy, C₁-C₆-alkylen,
      the aliphatic and/or alicyclic and/or aromatic groups of the definitions of L¹ may contain one, two, three or four groups R^{L1} that are the same or different from each other:
      R^{L1} halogen, hydroxy, cyano, nitro, C₁-C₄-alkyl, C₁-C₄-haloalkyl, C₁-C₄-alkoxy, C₁-C₄-haloalkoxy, C₃-C₆-cycloalkyl, C₃-C₆-halocycloalkyl, C₃-C₈-cycloalkenyl, C₃-C₈-cycloalkoxy, C₃-C₈-halocycloalkoxy, C₁-C₆-alkylen, oxy-C₂-C₄-alkylen, oxy-C₁-C₃-alkylenoxy, C₁-C₈-alkylcarbonyl, C₁-C₈-alkylcarbonyloxy, C₁-C₈-alkoxycarbonyl, amino, C₁-C₈-alkylamino, di-C₁-C₈-alkylamino;
R⁶⁶⁶ hydrogen, halogen, C₁-C₁₀-alkyl, C₁-C₁₀-haloalkyl, C₂-C₁₀-alkenyl, C₂-C₁₀-haloalkenyl, C₃-C₁₀-cycloalkyl, C₃-C₁₀-halocycloalkyl;
R⁷⁷⁷ hydrogen, C₁-C₁₀-alkyl, C₁-C₁₀-haloalkyl, C₂-C₁₀-alkenyl, C₂-C₁₀-haloalkenyl, C₂-C₁₀-alkinyl, C₃-C₁₀-cycloalkyl, C₃-C₁₀-halocycloalkyl, C₃-C₁₀-cycloalkenyl, C₃-C₁₀-halocycloalkenyl, tri-C₁-C₁₀-alkylsilyl;
R⁸⁸⁸ hydrogen, C₁-C₁₀-alkyl, C₁-C₁₀-haloalkyl, C₂-C₁₀-alkenyl, C₂-C₁₀-halogenalkenyl, C₃-C₁₀-cycloalkyl;
   R⁶⁶⁶, R⁷⁷⁷ and R⁸⁸⁸ are, if it is not indicated otherwise, independently from each other unsubstituted or substituted with one, two, three, four or five L¹, as defined above.

For compounds (I)-(5) and their precursors (in particular wherein the triazole group does not contain SH or a derivatized sulfur group) and the preparation of the same, see WO2010/029001, WO2010/029002, WO 2010/029000, WO 2010/029003, WO2010/031721, WO 2010/031847, WO 2010/031848, WO 2010/031842 (PCT/EP 2009/062122) and/or WO 2010/040718 (PCT/EP2009/062909).

Y in the compounds (I) is hydrogen, halogen, (C₁-C₈-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₆-C₁₀)-aryl, a five-, six-, seven-, eight-, nine- or ten-membered, in particular five- or six-membered, aromatic heterocycle that contains one, two, three or four heteroatoms from the group consisting of O, N and S, C(═S)R⁹, SO₂R¹⁰ or CN; wherein
R⁹ is NA⁴A⁵; wherein A⁴, A⁵ independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, C₃-C₈-cycloalkenyl, C₃-C₈-halocycloalkenyl, (C₆-C₁₀)-aryl, a five-, six-, seven-, eight-, nine- or ten-membered, in particular five- or six-membered, aromatic heterocycle that contains one, two, three or four heteroatoms from the group consisting of O, N and S;
R¹⁰ is (C₁-C₈-alkyl, phenyl-(C₁-C₈)-alkyl or phenyl, where the phenyl groups are in each case unsubstituted or substituted by one, two or three groups independently selected from the group consisting of halogen and (C₁-C₄)-alkyl.

According to one embodiment, Y in compounds (I) is hydrogen.

According to a further embodiment of the invention, Y in compounds (I) is (C₁-C₈-alkyl, (C₂-C₈)-alkenyl or CN.

According to a further embodiment of the invention, Y in compounds (I) is C₁-C₈-alkyl, preferably C₁-C₅-alkyl or C₁-C₄-alkyl. According to one specific embodiment, Y in compounds (I) is C₃-alkyl, according to another specific embodiment, Y in compounds (I) is C₅-alkyl. Particular examples of preferred Y are methyl, ethyl, iso-propyl, n-butyl or n-pentyl.

According to still a further embodiment of the invention Y in compounds (I) is (C₂-C₈)-alkenyl, in particular (C₃-C₆)-alkenyl such as Y=allyl.

According to still a further embodiment of the invention Y in compounds (I) is CN.

One key step of the present invention is providing a triazole magnesium compound of formula (IIIa)

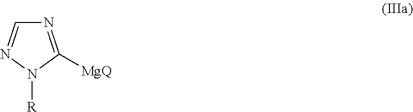

by means of a process comprising the step
(i) reacting a triazolo compound of formula (IV)

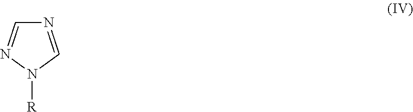

with a magnesium amide reagent (R¹R²N)MgQ (Va), wherein the variables are defined above.

The present invention, thus, provides a use of a reagent (R¹R²N)MgQ (Va), wherein the variables are defined or preferably defined herein, for the synthesis of thio-triazolo group-containing compounds of the formula (I) as defined or preferably defined herein.

According to one aspect of the invention, the amide reagent (R¹R²N)MgQ (Va) is used, wherein Q is (C₁-C₁₀)-alkyl, (C₂-C₁₀)-alkenyl, (C₂-C₁₀)-alkynyl, (C₃-C₈)-cycloalkyl, (C₆-C₁₀)-aryl, wherein the aryl is unsubstituted or substituted by one, two or three groups independently selected from the group consisting of halogen and (C₁-C₄)-alkyl, NR¹R² or X¹, wherein X¹ is halogen.

According to another aspect of the invention, the amide reagent (R¹R²N)MgQ (Va) is used, wherein Q is X³.zLiX², wherein X³, X² are independently halogen (=amide reagent (Vb)).

Also both, amide reagents (Va) and (Vb), in any suitable weight ratio can be used according to the present invention.

In the R¹R²N group, R¹ and R² are, according to one embodiment, in particular independently selected from (C₁-C₆)-alkyl, Si(A¹A²A³), (C₃-C₆)-cycloalkyl and (C₆-C₁₀)-aryl, wherein A¹, A², A³ are preferably independently selected from C₁-C₄-alkyl, trimethylsilyl and phenyl. The groups in R¹ and R² may independently from each other bear one, two or three identical or different R^{a} groups, wherein R^{a} is in each case preferably independently selected from halogen, C₁-C₄-alkyl, C₁-C₄-alkoxy and C₁-C₄-haloalkoxy.

Particularly suitable are R¹R²N groups, wherein R¹ and R² are independently selected from methyl, ethyl, isopropyl, n-butyl, sec-butyl, tert-butyl, trimethylsilyl, triethylsilyl, triisopropylsilyl, tert-butyldimethylsilyl, tert-butyldiphenylsilyl, tris(trimethylsilyl)silyl, more particularly selected from trimethylsilyl, isopropyl and tert-butyl.

According to another embodiment of the invention in the R¹R²N group, R¹ and R², together with the nitrogen atom to which they are bonded, form a five- or six-membered saturated or partially unsaturated, in particular saturated, heterocyclyl, which is bonded via N and, if it is a six-membered heterocyclyl, which may contain one or two additional heteroatoms selected from O, N and S. According to one embodiment, R¹ and R² form a five-membered ring. According to another embodiment, R¹ and R² form a six-membered ring. According to one embodiment, the heterocyclyl is unsubstituted. According to another embodiment, the heterocyclyl carries one, two, three or four substituents, preferably selected from the group of halogen, C₁-C₄-alkyl, C₁-C₄-haloalkyl, C₁-C₄-alkoxy, C₁-C₄-haloalkoxy and C₆-C₁₀-aryl.

Particularly suitable are R¹R²N groups, wherein R¹ and R² together with the nitrogen atom to which they are bonded, form six-membered saturated heterocyclyl, which is bonded via N and which may contain one or two additional heteroatoms selected from O, N and S, and which carries one, two, three or four substituents, selected from the group of halogen, C₁-C₄-alkyl, C₁-C₄-haloalkyl and C₆-C₁₀-aryl, in particular halogen, C₁-C₄-alkyl, C₁-C₄-haloalkyl and phenyl. More particularly, R¹ and R² and the nitrogen form TMP (2,2,6,6-tetramethylpiperidine), piperidine, pyrrolidine, morpholine, thiomorpholine and N-alkyl- or N-aryl-piperazine, in particular TMP (2,2,6,6-tetramethylpiperidine), piperidine, pyrrolidine, morpholine, thiomorpholine, N-alkyl- or N-phenyl-piperazine.

According to one embodiment of the invention Q is (C₁-C₁₀)-alkyl, (C₂-C₁₀)-alkenyl, (C₂-C₁₀)-alkynyl, (C₃-C₈)-cycloalkyl or (C₆-C₁₀)-aryl, wherein the aryl is unsubstituted or substituted by one, two or three groups independently selected from the group consisting of halogen and (C₁-C₄)-alkyl. In particular, Q is (C₁-C₆)-alkyl, (C₂-C₆)-alkenyl, (C₃-C₆)-cycloalkyl or phenyl, optionally containing one, two or three substituents selected from Cl, F, methyl and ethyl. According to one embodiment, Q is (C₁-C₆)-alkyl, in particular (C₂-C₄)-alkyl. Specific examples for Q are methyl, ethyl, n-propyl, iso-propyl, n-butyl, iso-butyl and tert-butyl. It may be preferred according to the invention to use reagents, wherein Q is iso-propyl, n-butyl or cyclopentyl. According to another embodiment, Q is (C₂-C₆)-alkenyl, in particular vinyl. According to still another embodiment, Q is unsubstituted phenyl.

According to another embodiment of the invention, Q is X², wherein X² is halogen, in particular Cl or Br.

According to another embodiment, Q is NR¹R², wherein R¹ and R² are preferably defined as given above.

According to another embodiment, Q is X³.zLiX², wherein X³, X² are independently halogen (=amide reagent (Vb)), in particular Cl.

The magnesium amide reagents used according to the present invention can generally be prepared by reacting a organomagnesium halide QMgX¹ or a diorganomagnesium compound Q₂Mg, wherein Q is (C₁-C₁₀)-alkyl, (C₂-C₁₀)-alkenyl, (C₂-C₁₀)-alkynyl, (C₃-C₈)-cycloalkyl, (C₆-C₁₀)-aryl, wherein the aryl is unsubstituted or substituted by one, two or three groups independently selected from the group consisting of halogen and (C₁-C₄)-alkyl, with the respective amine.

In particular, the synthesis of the reagent (Va) wherein Q is (C₁-C₁₀)-alkyl, (C₂-C₁₀)-alkenyl, (C₂-C₁₀)-alkynyl, (C₃-C₈)-cycloalkyl, (C₆-C₁₀)-aryl, wherein the aryl is unsubstituted or substituted by one, two or three groups independently selected from the group consisting of halogen and (C₁-C₄)-alkyl, can be carried out starting from (n-butyl)₂Mg or any similar dialkyl magnesium compound, that is commercially available with 1.0 equivalent of the respective amine, see for example M.-X. Zhang, P. E. Eaton, Angew. Chem. Int. Ed. 2002, 41, 2169.

The synthesis of the reagent (Va) wherein Q is NR¹R² can be carried out starting from (n-butyl)₂Mg or any similar dialkyl magnesium compound, that is commercially available with 0.5 equivalents of the respective amine, see for example P. E. Eaton, C.-H. Lee, Y. Xiong, J. Am. Chem. Soc. 1989, 111, 8016.

The synthesis of the reagent (Va) wherein Q is halogen can for example be carried out starting from EtMgBr or any similar organomagnesium halide, that is commercially available with 1.0 equivalent of the respective amine, see for example F. C. Frostick, C. R. Hauser, J. Am. Chem. Soc. 1949, 71, 1350.

The synthesis of the reagent (Vb) wherein Q is halide can for example be carried out starting from iPrMgCl.LiCl or any similar organomagnesium halide, that is commercially available with 1.0 equivalents of the respective amine, see for example A. Krasovskiy, V. Krasovskaya, P. Knochel, Angew. Chem. Int. Ed. 2006, 45, 2958.

The synthesis of the reagent (Vb) wherein Q is NR¹R² can for example be carried out starting from iPrMgCl.LiCl or any similar organomagnesium halide, that is commercially available with 0.5 equivalents of the respective amine, see for example G. C. Clososki, C. J. Rohbogner, P. Knochel, Angew. Chem. Int. Ed. 2007, 46, 7681.

If, according to the above mentioned aspect of the invention, the amide reagent (R¹R²N)MgX³.zLiX² (Vb) is used, z is >0, preferably in the range from 0.001 to 5, more particularly in the range from 0.5 to 2, even more particularly in the range from 0.9 to 1.2 and it may be preferred if z is about 1.

According to one embodiment of the inventive process, zLiX² is added to the reaction mixture of step (i). According to an alternative, before contacting the magnesium amide reagent (Va) with a compound of formula (I), it is brought together with the respective amount of LiX², thereby forming an addition product (R¹R²N)MgX³.zLiX² (Vb). According to this alternative, (R¹R²N)MgX³.zLiX² (Vb) is then used in step (i). The use of LiX² together with magnesium amide reagents is generally known in the art, see for example Angew. Chem. Int. Ed. 2006, 45, 159 and WO 2007/082911 and the literature cited therein.

According to still another aspect of the invention, the magnesium amide reagent (Va) or (Vb), respectively, is used in catalytic amounts, and the reagent is recycled in situ.

The process step (i) according to the invention can be carried out in any organic solvent that is suitable for magnesium amide reagents. In general, the use of ethers is advantageous. Possible solvents are for example tetrahydrofuran (THF), 2-methyl-tetrahydrofuran (2-Me-THF), diethyl ether, TBME (tert-butyl methyl ether), CPME (cyclopentyl methyl ether), DME (1,2-dimethoxyethane) and 1,4-dioxane. Further solvents that may be suitable are, for example, diisopropyl ether, di-n-butyl ether and/or diglyme. Often, the use of THF or 2-methyl-THF is particularly suitable. Furthermore, it may also be suitable to use combinations of two or more different solvents, such as for example any combination of the solvents listed above or any one of the listed ethers with aliphatic hydrocarbons like n-hexane, heptane or aromatic hydrocarbons like toluene or xylenes.

As mentioned above, one advantage of the inventive process is, that it can be carried out in a large temperature range. This especially applies to step (i). In particular, there is no need for strongly cooling the reaction mixture, although it is sometimes beneficial to run the reaction under slight cooling. On the other hand, it can also be advantageous to work at elevated temperatures. This can be favourable in order to achieve higher conversion of the reagents to the products. Suitable temperature ranges are −40° C. to 80° C., in particular −30° C. to 60° C., more particularly −20° C. to 20° C. It may be preferred to carry out the reaction at temperatures of −20° C. to 0° C. It may be also preferred to work at temperatures of 0° C. to 20° C.

The reaction components in step (i) are usually employed in amounts such that 1 to 10 moles, in particular 1,1 to 5, more particularly 1,2 to 3 moles of magnesium amide reagent are used per mole of the compound (IV). It may be preferred if 1 to 2,5 moles of the magnesium amide reagent are used per mole of the compound (IV).

Compounds of formula (IIIa), wherein Q is NR¹R² or X³.zLiX² are novel. Accordingly, a further aspect of the present invention is a compound of formula (IIIa)

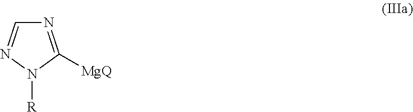

wherein Q is NR¹R² or X³.zLiX², as defined and preferably defined above and R is as defined or preferably defined above, wherein it is preferred if R is one of sub-groups (1), (2), (3), (4) or (5) as defined and preferably defined above. According to one specific embodiment, R in compounds (IIIa) is a group (1) as defined above, including the specific embodiments thereof.

A further aspect of the present invention is a use of a compound of formula (IIIa) as defined and preferably defined herein, for the synthesis of a thio-triazolo group-containing compound of the formula (I) as defined herein.

The process of the present invention may be described as the synthesis of thio-triazolo group containing compounds (I), particularly pesticidal compounds of the triazole class having phytopathogenic activity,

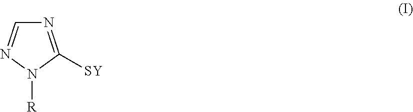

by a process comprising either step (ii) together with step (iii-1) or (iii-2); or comprising step (iv):
(ii) reacting a compound of formula (IIIa)

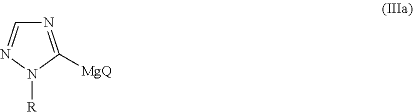

with sulfur, wherein R and Q are as defined above, in order to obtain a compound of formula (IIa)
and

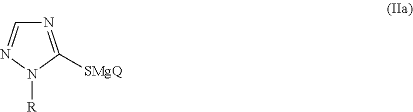

(iii-1) protonating said compound of formula (IIa), in order to obtain compounds of formula (I), wherein Y is hydrogen; or
(iii-2) reacting said compound of formula (IIa) with an electrophilic compound Y¹-LG in order to obtain compounds of formula (I), wherein Y is Y¹, wherein
   Y¹ is (C₁-C₈)-alkyl, (C₁-C₈)-haloalkyl, (C₂-C₈-alkenyl, (C₂-C₈-haloalkenyl, (C₂-C₈-alkynyl, (C₂-C₈-haloalkynyl, C(═S)R⁹, SO₂R¹⁰ or CN; wherein R⁹ and R¹⁰ are as defined below; and
   LG is a leaving group;
      or
(iv) reacting a compound of formula (IIIa)

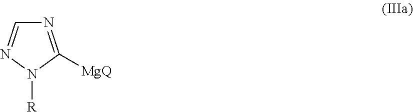

with an electrophile selected from
(VI) a disulfide R³—S—S—R³, in order to obtain a compound of formula (I), wherein Y is R³;
(VII) R⁴—S—SO₂—R⁴, in order to obtain a compound of formula (I), wherein Y is R⁴;
(VIII) R⁵—S—Cl, in order to obtain a compound of formula (I), wherein Y is R⁵;
(IX) BrSCN in order to obtain a compound (I), wherein Y is CN;
   wherein R, Q, and Y are as defined and preferably defined above. The remaining variables have the following meanings:
R³, R⁴ are independently from another (C₁-C₈-alkyl, (C₁-C₈-haloalkyl, (C₂-C₈-alkenyl, (C₂-C₈-haloalkenyl, (C₂-C₈-alkynyl, (C₂-C₈-haloalkynyl, (C₆-C₁₀)-aryl, a five-, six-, seven-, eight-, nine- or ten-membered, in particular five- or six-membered, aromatic heterocycle that contains one, two, three or four heteroatoms from the group consisting of O, N and S, C(═S)R⁹ or CN; and
R⁵ is halogen, (C₁-C₈)-alkyl, (C₁-C₈)-haloalkyl, (C₂-C₈-alkenyl, (C₂-C₈-haloalkenyl, (C₂-C₈-alkynyl, (C₂-C₈-haloalkynyl, (C₆-C₁₀)-aryl and a five-, six-, seven-, eight-, nine- or ten-membered, in particular five- or six-membered, aromatic heterocycle that contains one, two, three or four heteroatoms from the group consisting of O, N and S.

According to step (ii), a compound (IIIa) is reacted with sulfur, thereby forming magnesium thiolates of formula (IIa). Sulfur (Ss) is preferably used as a powder. The reaction components are usually employed in amounts such that 1 to 20 moles, in particular 1.2 to 10, more particularly 1.3 to 5 moles of sulfur are used per mole of the compound (IIIa). It may be preferred if 1 to 4 moles of sulfur are used per mole of the compound (IIIa).

Suitable solvents for step (ii) are all inert organic solvents, where preferably ethers such as tetrahydrofuran, 1,4-dioxane, diethyl ether and 1,2-dimethoxyethane can be used. Furthermore, it may also be suitable to use combinations of two or more different solvents, such as for example any combination of the solvents listed above or any one of the listed ethers with aliphatic hydrocarbons like n-hexane, heptane or aromatic hydrocarbons like toluene or xylenes.

The reaction temperature is preferably between −40° C. and 80° C., in particular between −30° C. and 60° C. It may be preferred to work at temperatures of −20° C. to 20° C.

The reaction is generally carried out under atmospheric pressure.

Usually, the reaction mixture resulting from step (ii) is directly used for subsequent steps (iii-1) or (iii-2). However, in case a work-up is suitable, it can be carried out according to procedures generally known to the person skilled in the art.

According to step (iii-1), the respective compound (IIa) is protonated in order to obtain compounds of formula (I), wherein Y is hydrogen (in the following also called compounds (I.1):

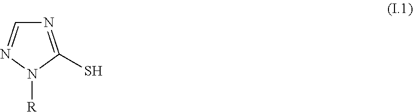

Suitable reagents for the protonation are for example hydrohalic acids, such as hydrogen fluoride, hydrogen chloride, hydrogen bromide and hydrogen iodide, carbonic acid, sulfuric acid, phosphoric acid and nitric acid. The latter acids are generally used in an aqueous medium. Also organic acids can be used for step (iii-1), for example formic acid and alkanoic acids, such as acetic acid, trifluoroacetic acid, trichloroacetic acid and propionic acid, and also glycolic acid, lactic acid, succinic acid, citric acid, benzoic acid and other arylcarboxylic acids, cinnamic acid, oxalic acid, alkylsulfonic acids (sulfonic acids having straight-chain or branched alkyl radicals of 1 to 20 carbon atoms), arylsulfonic acids or aryldisulfonic acids (aromatic radicals, such as phenyl and naphthyl, which carry one or two sulfonic acid groups), alkylphosphonic acids (phosphonic acids having straight-chain or branched alkyl radicals of 1 to 20 carbon atoms), arylphosphonic acids or aryldiphosphonic acids (aromatic radicals, such as phenyl and naphthyl, which carry one or two phosphoric acid radicals), where the alkyl or aryl radicals may carry further substituents, for example p-toluenesulfonic acid, salicylic acid, p-aminosalicylic acid, 2-phenoxybenzoic acid, 2-acetoxybenzoic acid etc.

Furthermore, the protonation step (iii-1) of the inventive process may be carried out using other protonating agents, such as alcohols, for example (C₁-C₆)-alcohols, in particular methanol, ethanol, isopropanol or isobutanol. Also water as such may be used. It may be preferred to use water, if appropriate in the presence of an organic or inorganic acid such as, for example, acetic acid, dilute sulfuric acid or dilute hydrochloric acid.

According to step (iii-2), the respective compound (IIa) is reacted with the respective electophilic reagent Y¹-LG in order to obtain compounds of formula (I), wherein Y is Y¹, which is (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, C(═S)R⁹, SO₂R¹⁰ or CN; wherein R⁹ and R¹⁰ are as defined and preferably defined above.

LG stands for a leaving group, such as, for example, halogen, such as Cl, Br or I, or alkyl or arylsulfonates like methanesulfonate, benzenesulfonate, 4-toluenesulfonate, 2-nitrobenzenesulfonate, 4-nitrobenzenesulfonate and 4-bromobenzenesulfonate, or perfluorinated alkylsulfonates like trifluoromethanesulfonate or nonafluorobutanesulfonate. Cl, Br and I are mostly preferably used.

In order to obtain target compounds, wherein Y is C₁-C₈-alkyl, preferably C₁-C₅-alkyl or C₁-C₄-alkyl, in particular C₃-alkyl or C₅-alkyl, specifically methyl, ethyl, iso-propyl, n-butyl or n-pentyl, a compound (IIa) is preferably reacted with the corresponding alkyl halide.

Target compounds (I) with Y being (C₂-C₈)-alkenyl, in particular (C₃-C₆)-alkenyl such as Y=allyl, are similarly accessible by reacting compounds (IIa) with the respective (C₂-C₈)-alkenyl-LG, LG preferably being Br, Cl or I, wherein one particularly suitable reagent is prop-2-enyl bromide.

For target compounds, wherein Y═CN the reagent BrCN is suitable for the inventive process.

In general, from 1 to 3 equivalents, preferably from 1 to 2.5 equivalents, of reagent Y¹-LG are employed per mole of the compound of the formula II.

Suitable solvents for steps (iii-1) and (iii-2) are all inert organic solvents, where preferably ethers such as tetrahydrofuran, dioxane, diethyl ether and 1,2-dimethoxyethane can be used. Further solvents that may be suitable are, for example, diisopropyl ether, di-n-butyl ether and/or diglyme. Often, the use of THF or 2-methyl-THF is particularly suitable. Furthermore, it may also be suitable to use combinations of two or more different solvents, such as for example any combination of the solvents listed above or any one of the listed ethers with aliphatic hydrocarbons like n-hexane, heptane or aromatic hydrocarbons like toluene or xylenes.

The reaction of step (iii-1) or (iii-2) is generally carried out under atmospheric pressure. The protonation step (iii-1) or the trapping reaction using an electrophile Y¹-LG (iii-2), respectively, may be carried out at temperatures of −30° C. to 80° C., preferably −10° C. to 60° C., more preferably 0° C. to 40° C. In some cases it may be preferred, if temperatures of −30° C. to 40° C., preferably −10° C. to 20° C., more preferably 0° C. to 40° C. are used.

Work-up of the reaction mixture resulting from reaction step (iii-1) or (iii-2), respectively, is carried out by procedures known in a general manner to the person skilled in the art. Usually, the reaction mixture is extracted with a suitable organic solvent (for example aromatic hydrocarbons such as toluene and xylenes) and the residue is, if appropriate, purified by recrystallization and/or chromatography.

According to one embodiment of step (iv), an inventive magnesium compound (IIIa) is reacted with a disulfide R³—S—S—R³, in order to obtain a compound of formula (I), wherein Y is R³ and R³ is (C₁-C₈-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₆-C₁₀)-aryl, a five-, six-, seven-, eight-, nine- or ten-membered, in particular five- or six-membered, aromatic heterocycle that contains one, two, three or four heteroatoms from the group consisting of O, N and S, C(═S)R⁹ or CN, in particular (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, C(═S)R⁹ or CN. Preferably, R³ is (C₁-C₅)-alkyl, in particular methyl, ethyl, iso-propyl, n-propyl, n-butyl or n-pentyl, (C₃-C₆)-alkenyl, in particular allyl, or CN. According to a specific embodiment thereof, dirhodane NC—S—S—CN is used in order to result in compounds (I) with Y═CN.

According to a further embodiment of step (iv), an inventive magnesium compound (IIIa) is reacted with a reagent (VII) R⁴—S—SO₂—R⁴, in order to obtain a compound of formula (I), wherein Y is R⁴ and R⁴ is (C₁-C₈-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₆-C₁₀)-aryl, a five-, six-, seven-, eight-, nine- or ten-membered, in particular five- or six-membered, aromatic heterocycle that contains one, two, three or four heteroatoms from the group consisting of O, N and S, C(═S)R⁹ or CN, in particular (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, C(═S)R⁹ or CN. Preferably, R⁴ is (C₁-C₅)-alkyl, in particular methyl, ethyl, iso-propyl, n-propyl, n-butyl or n-pentyl, (C₃-C₆)-alkenyl, in particular allyl or CN.

According to still a further embodiment of step (iv), an inventive magnesium compound (IIIa) is reacted with a reagent (VIII) R⁵—S-Hal, wherein Hal is halogen, in particular Cl or Br, in order to obtain a compound of formula (I), wherein Y is R⁵, wherein R⁵ is halogen, (C₁-C₈-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₆-C₁₀)-aryl or a five-, six-, seven-, eight-, nine- or ten-membered, in particular five- or six-membered, aromatic heterocycle that contains one, two, three or four heteroatoms from the group consisting of O, N and S, or CN. Specific examples are Y═R⁵═CN or CCl₃. One further particular reagent is SHal₂ (R⁵=Hal), in order to obtain a compound of formula (I), wherein Y is Halogen, in particular Cl. According to still a further embodiment a reagent BrSCN is used in order to obtain a compound (I), wherein Y═R⁵═CN.

Accordingly, a further aspect of the present invention is a use of a compound of formula (IIa) as defined and preferably defined herein, for the synthesis of a thio-triazolo group-containing compound of the formula (I) as defined herein.

Suitable solvents for step (iv) and are all inert organic solvents, where preferably ethers such as tetrahydrofuran, 1,4-dioxane, diethyl ether and 1,2-dimethoxyethane can be used. Furthermore, it may also be suitable to use combinations of two or more different solvents, such as for example any combination of the solvents listed above or any one of the listed ethers with aliphatic hydrocarbons like n-hexane, heptane or aromatic hydrocarbons like toluene or xylenes. The reaction temperature is preferably between −30° C. and 80° C., in particular between −10° C. and 60° C. It may be preferred to work at temperatures of −5° C. to 20° C. or 0° C. to 40° C.

The reaction is generally carried out under atmospheric pressure.

The electrophile, in particular the disulfide or BrSCN, is usually employed in equivalent amounts compared to of the compound (IIIa) and/or (IIIb) or in excess, such that usually 1 to 8 moles, in particular 2 to 6 or 3 to 5 moles are used per mole of the compound (IIIa) and/or (IIIb).

In case a work-up is suitable, it can be carried out according to procedures generally known to the person skilled in the art. Usually, the reaction mixture is extracted with a suitable organic solvent, and the residue is, if appropriate, purified by recrystallization and/or chromatography.

Furthermore, it is possible by means of the inventive process as described above to insert a group S-M¹, (Y=M¹ in compounds (I)) wherein M¹ is as defined below.

Compounds of formula (IIa), wherein Q is NR¹R² or Xe.zLiX² are novel. Accordingly, a further aspect of the present invention is compound of formula (IIa)

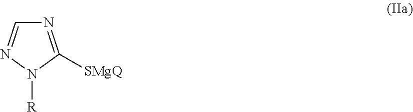

wherein Q is NR¹R² or Xe.zLiX² wherein R¹ and R², Xe, X² and z are in particular as defined and preferably defined above (Xe, X² are preferably Cl), and R is as defined or preferably defined above, wherein R=group (1), (2), (3), (4) or (5) is preferred.

According to another aspect of the present invention, thio-triazolo-group containing compounds (I), particularly pesticidal compounds of the triazole class having phytopathogenic activity of formula (I)

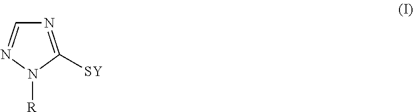

are synthesized from a compound of formula (IIa)

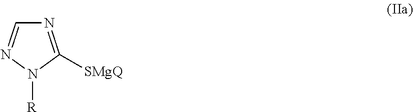

by means of the inventive process comprising the step (iii-1) or (iii-2) as defined above.

According to another aspect of the present invention, compounds of formula (IIa) can be obtained by a process comprising the step of reacting a compound (IIIa) with sulfur according to step (ii) as defined above.

According to one embodiment of the inventive process, step (i), then step (ii) and then (iii-1) or (iii-2) are carried out. Thus, according to this embodiment, the inventive process comprises the steps (i), (ii) and, subsequently, (iii-1) or (iii-2).

According to another embodiment of the inventive process, step (i), then step (iv) is carried out. Thus, according to this embodiment, the inventive process comprises the steps (i) and (iv).

A further advantage of the inventive process is that thio-triazolo compounds (I) are accessible in a one-pot reaction. Furthermore, if desired, the reaction can be carried out without cooling or at slightly elevated temperatures and that the conversion to the desired products is high. Thereby, only few side-products or even no significant side-products are formed. The process is thus very economic.

Still a further advantage of the inventive process is that the magnesium amide reagent (Va) or (Vb), respectively, can, according to another aspect of the invention, be used in catalytic amounts, and the reagent can be recycled in situ through reaction with an organomagnesium compound.

The novel compounds according to the invention contain chiral centers and are generally obtained in the form of racemates or as diastereomeric mixtures of erythro and threo forms. The erythro and threo diastereomers of the compounds according to the invention can be separated and isolated in pure form, for example, on the basis of their different solubilities or by column chromatography. Using known methods, such uniform pairs of diastereomers can be used to obtain uniform enantiomers.

Accordingly, the invention provides both the pure enantiomers or diastereomers and mixtures thereof. This applies to the compounds according to the invention. The scope of the present invention includes in particular the (R) and (S) isomers and the racemates of the compounds according to the invention, which have centers of chirality. Suitable compounds according to the invention also include all possible stereoisomers (cis/trans isomers) and mixtures thereof.

The compounds according to the invention may be present in various crystal modifications. They are likewise provided by the present invention.

Furthermore, in the inventive process, the reactants used, contain chiral centers and are generally used in the form of racemates or as diastereomeric mixtures of erythro and threo forms. The erythro and threo diastereomers of these compounds can be separated and isolated in pure form, for example, on the basis of their different solubilities or by column chromatography. Using known methods, such uniform pairs of diastereomers can be used to obtain uniform enantiomers.

Accordingly, the invention provides both the use of pure enantiomers or diastereomers and mixtures thereof. The scope of the present invention includes in particular the use of the (R) and (S) isomers and the racemates of the respective reactants, which have centers of chirality. Suitable compounds used according to the invention also include all possible stereoisomers (cis/trans isomers) and mixtures thereof.

The compounds used according to the invention may be present in various crystal modifications. They are likewise possible to be used in the inventive process.

In order to obtain compounds of formula (I) that contain a derivatized sulfur group (Y other that hydrogen), the compounds of formula (I), wherein Y=hydrogen (compounds (1.1)) can be further reacted according to processes known in the art.

For example, by further reaction of compounds (I.1) with R^{8A}-LG, where R^{8A} is as defined below and LG is a leaving group such as, for example, halogen, such as Cl, Br or I, or perfluoroalkylsulfonate, e.g. trifluoromethylsulfonate or nonafluorobutanesulfonate, it is possible to prepare various compounds of the formula (I) carrying a S—R^{8A} group instead of “S—H”. To prepare compounds containing a group SR^{8A} where R^{8A} is C₁-C₈-alkyl, preferably C₁-C₅-alkyl or C₁-C₄-alkyl, in particular C₃-alkyl or C₅-alkyl, specifically methyl, ethyl, iso-propyl, n-butyl or n-pentyl, a compound (1.1) is reacted with the corresponding alkyl halide (see also WO 96/38440). Further, the following S-residues may be formed from the respective SH-derivative of formula (I):

S—R^{8A}, where
R^{8A} is C₁-C₈-alkyl, C₁-C₈-haloalkyl, C₂-C₈-alkenyl, C₂-C₈-haloalkenyl, C₂-C₈-alkynyl, C₂-C₈-haloalkynyl, C(═O)R^{5A}, C(═S)R^{5A}, SO₂R^{6A} or CN; where
R^{5A} is C₁-C₈-alkyl, C₁-C₈-haloalkyl, C₁-C₈-alkoxy, C₁-C₈-haloalkoxy or NA^{3A}A^{4A}; and
R^{6A} is C₁-C₈-alkyl, phenyl-C₁-C₈-alkyl or phenyl, where the phenyl groups are in each case unsubstituted or substituted by one, two or three groups independently selected from the group consisting of halogen and C₁-C₄-alkyl;
A^{3A}, A^{4A} independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, C₃-C₈-cycloalkenyl or C₃-C₈-halocycloalkenyl
   S-DII, wherein DII is

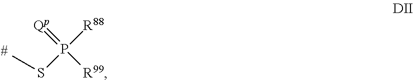

where # is the point of attachment to the triazolyl ring and Q, R⁸⁸ and R⁹⁹ are as defined below:
Q^{P} is O or S;
R⁸⁸, R⁹⁹ independently of one another are C₁-C₈-alkyl, C₁-C₈-haloalkyl, C₁-C₈-alkoxy, C₁-C₈-alkoxy-C₁-C₈-alkoxy, C₁-C₈-haloalkoxy, C₁-C₈-alkoxy-C₁-C₈-alkyl, C₁-C₈-alkylthio, C₂-C₈-alkenylthio, C₂-C₈-alkynylthio, C₃-C₈-cycloalkyl, C₃-C₈-cycloalkylthio, phenyl, phenyl-C₁-C₄-alkyl, phenoxy, phenylthio, phenyl-C₁-C₄-alkoxy or NR¹¹¹R²²², where R¹¹¹ is H or C₁-C₈-alkyl and R²²² is C₁-C₈-alkyl, phenyl-C₁-C₄-alkyl or phenyl or R¹¹¹ and R²²² together are an alkylene chain having four or five carbon atoms or form a radical of the formula —CH₂—CH₂—O—CH₂—CH₂— or —CH₂—CH₂—NR³³³—CH₂—CH₂— in which R³³³ is hydrogen or C₁-C₄-alkyl; where the aromatic groups in the radicals mentioned above are in each case independently of one another unsubstituted or substituted by one, two or three groups selected from the group consisting of halogen and C₁-C₄-alkyl;
   SM¹, wherein
M¹ is an alkali metal cation, an equivalent of an alkaline earth metal cation, an equivalent of a copper, zinc, iron or nickel cation or an ammonium cation of the formula (E)

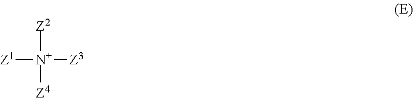

in which
Z¹ and Z² independently are hydrogen or C₁-C₈-alkyl;
Z³ and Z⁴ independently are hydrogen, C₁-C₈-alkyl, benzyl or phenyl; where the phenyl groups are in each case unsubstituted or substituted by one, two or three groups independently selected from the group consisting of halogen and C₁-C₄-alkyl.

According to an embodiment of the invention Y in compounds (I) is derivatized into Na, ½ Cu or an ammonium cation of the formula (E), wherein Z¹ and Z² preferably are independently selected from hydrogen and C₁-C₄-alkyl and Z³ and Z⁴ are preferably independently selected from hydrogen, C₁-C₄-alkyl, benzyl and phenyl; where the phenyl groups are in each case unsubstituted or substituted by one, two or three groups independently selected from the group consisting of halogen and C₁-C₄-alkyl. It may be preferred, if in group (E), Z¹, Z², Z³ and Z⁴ are independently selected from hydrogen and C₁-C₄-alkyl, in particular hydrogen, methyl and ethyl. One particular suitable group (E) is HN(Et)₃.

Compounds of the formula I which contain a group S—C(═O)NA^{3A}A^{4A} can be synthesized analogously to the process described in WO 99/21853.

Compounds of the formula I which contain a group DII can be synthesized analogously to the process described in WO 99/05149.

Compounds of the formula I which contain a group S—SO₂R^{6A} can be synthesized analogously to the process described in WO 97/44332.

Compounds of the formula I which contain a group S—CN can be synthesized analogously to the process described in WO 99/44331.

Compounds of the formula I which contain a group DI can be synthesized analogously to the process described in WO 97/43269.

Compounds of the formula I which contain a group S—C(═O)R^{5A} where R^{5A}=C₁-C₈-alkyl, C₁-C₈-haloalkyl, C₁-C₈-alkoxy or C₁-C₈-haloalkoxy can be synthesized analogously to the process described in WO 97/42178.

Compounds of the formula I which contain a group SM¹ can be synthesized analogously to the process described in WO 97/41107.

According to one aspect of the present invention, one of the steps for derivatizing the sulfur in the triazole ring as detailed above is carried out following the process of the present invention, wherein Y═H. According to one specific aspect, following the synthesis of compounds (I)-(1) with Y═H according to the process of the present invention, one of the steps for derivatizing the sulfur in the triazole ring is carried out. This represents a very useful approach for the synthesis of further fungicidal compounds, in particular where SH is derivatized into SR^{8A}, R^{8A} being C₁-C₈-alkyl, in particular C₁-C₅-alkyl, C₂-C₈-alkenyl or CN (see specific examples above). According to one further specific aspect, following the synthesis of compounds (I)-(1) with Y═H according to the process of the present invention, the step of derivatizing the sulfur in the triazole ring is derivatized into SMS, wherein M¹ is as defined and preferably defined above. See WO 97/41107.

### EXAMPLES

The following examples further illustrate the present invention and do not restrict the invention in any manner.

### Example 1

### Comparison of Different Mg Amides for the Deprotonation Reaction

A solution of (2RS, 3SR)-2-[3-(2-chloro-phenyl)-2-(2,4-difluoro-phenyl)-oxiranylmethyl]-[1,2,4]triazole (0.5 g, 1.44 mmol) in 4 mL of THF was cooled to 0° C. A solution of the respective Mg amide base in THF (1.73 mol, 1.2 eq.) was injected while cooling with ice. The solution was then agitated at that temperature for 60 minutes. Then, sulfur (92 mg, 2.88 mmol) was added at once. Stirring was continued for another 60 min at 0° C. Then, the reaction mixture was quenched through the addition of 5% HCl and extracted with TBME. The crude reaction mixtures were analyzed by HPLC,
i) Et₂NM Cl: starting material: 9.7 area-%
triazolthione: 31.4 area-%
ii) iPr₂NMgCl: starting material: 78.2 area-%
triazoithione: 14.2 area-%
iii) Et₂NMgCl.LiCl: starting material: 17.7 area-%
triazoithione: 30.5 area-%
iv) iPr₂NMgCl.LiCl: starting material: 3.8 area-%
triazolthione: 78.9 area-%
v) starting material: 1.9 area-%
triazolthione: 87.3 area-%

### Example 2

### Synthesis of (2RS, 3SR)-2-[3-(2-chloro-phenyl)-2-(2,4-difluoro-phenyl)-oxiranylmethyl]-2,4-dihydro-[1,2,4]triazole-3-thione on a Preparative Scale

A solution of (2RS, 3SR)-2-[3-(2-chloro-phenyl)-2-(2,4-difluoro-phenyl)-oxiranylmethyl]-[1,2,4]triazole (4.0 g, 11.5 mmol) in 16 mL of THF was cooled to 0° C. A solution of TMPMgCl.LiCl in THF (1.1 aloft in THF, 12.5 mL, 13.8 mmol) was injected within 5 minutes while cooling with ice. The solution was then agitated at that temperature for 60 minutes. Then, sulfur (0.75 g, 23.4 mmol) was added portionwise over 5 minutes. Stirring was continued for another 60 min at 0° C. Then, the reaction mixture was poured onto ice cold 4% HCl (20 ml) and 20 ml TBME were added. The phases were separated and the aqueous phase was extracted with TBME (20 mL). The combined organic phases were washed with water and brine and dried over Na₂SO₄. All volatiles were removed under reduced pressure and the raw residue was recrystallized from xylene (isomer mixture). The crystals were filtered off, rinsed with xylenes and n-hexane and dried at a pressure of <20 mbar overnight to give the product as a powder (3.68 g, purity 93.4% by HPLC, 78.7% yield).

¹H NMR (CDCl₃, 500 MHz): δ(ppm)=13.31 (bs, 1H); 8.24 (s, 1H); 7.55-7.61 (m, 2H); 7.45-7.50 (m, 2H); 7.34 (q, J=9.5 Hz, 1H); 7.27 (dt, 3.0 Hz, a 12.5 Hz, 1H); 7.05 (ddd, a 3.0 Hz, 9.5 Hz, J=10.5 Hz, 1H); 4.46 (d, 18.0 Hz, 1H); 4.39 (s, 1H); 4.12 (d, 18.0 Hz, 1H).

Melting point: 180° C.

1-14. (canceled)

15. A process for the preparation of a thio-triazolo group-containing compound of the formula (I) 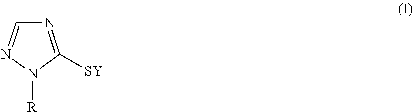
wherein:
R is an organic group;
Y is hydrogen, halogen, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₆-C₁₀)-aryl, C(═S)R⁹, SO₂R¹⁰, CN or a five-, six-, seven-, eight-, nine- or ten-membered aromatic heterocycle that contains one, two, three or four heteroatoms selected from the group consisting of O, N and S; wherein
R⁹ is NA⁴A⁵; wherein A⁴ and A⁵ independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, C₃-C₈-cycloalkenyl, C₃-C₈-halocycloalkenyl, (C₆-C₁₀)-aryl, or a five-, six-, seven-, eight-, nine- or ten-membered aromatic heterocycle that contains one, two, three or four heteroatoms selected from the group consisting of O, N and S;
R¹⁰ is (C₁-C₈)-alkyl, phenyl-(C₁-C₈)-alkyl or phenyl, where the phenyl groups are in each case unsubstituted or substituted by one, two or three groups independently selected from the group consisting of halogen and (C₁-C₄)-alkyl; comprising either step (i) together with (ii) and (iii-1) or step (i) together with (ii) and (iii-2); or comprising step (i) together with (iv):
(i) reacting a compound of formula (IV) 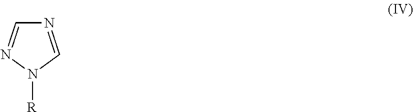
with a reagent
(R¹R²N)MgQ (Va),
wherein:
Q is (C₁-C₁₀-alkyl, (C₂-C₁₀)-alkenyl, (C₂-C₁₀)-alkynyl, (C₃-C₈)-cycloalkyl, NR¹R², X¹, X³.zLiX², or (C₆-C₁₀)-aryl, wherein the aryl is unsubstituted or substituted by one, two or three groups independently selected from the group consisting of halogen and (C₁-C₄)-alkyl,
R¹ and R² are independently selected from the group consisting of (C₁-C₁₀)-alkyl, Si(A¹A²A³), (C₃-C₈)-cycloalkyl and (C₆-C₁₀)-aryl, wherein said groups may bear one, two or three identical or different substituents R^{a}; or
R¹ and R², together with the nitrogen atom to which they are bonded form a five- or six-membered saturated or partially unsaturated heterocyclyl, which is bonded via N and, if it is a six-membered heterocyclyl, which may contain one or two additional heteroatoms selected from the group consisting of O, N and S, and wherein in each case, the heterocyclyl is unsubstituted or carries one, two, three or four substituents selected from the group of halogen, C₁-C₄-alkyl, C₁-C₄-haloalkyl, C₂-C₆-alkenyl, C₂-C₆-haloalkenyl, C₁-C₄-alkoxy, C₁-C₄-alkoxycarbonyl, C₁-C₄-haloalkoxy, C₃-C₆-alkenyloxy, C₃-C₆-haloalkenyloxy and C₆-C₁₀-aryl; wherein
A¹, A², A³ are independently selected from the group consisting of C₁-C₆-alkyl, trimethylsilyl and phenyl;
R^{a} is in each case independently selected from the group consisting of halogen, C₁-C₄-alkyl, C₁-C₄-haloalkyl, C₁-C₄-alkoxy, C₁-C₄-haloalkoxy, C₁-C₄-alkylthio, C₁-C₄-alkylamino and di-C₁-C₄-alkylamino;
z is >0;
X¹ halogen; and
X³, X² are independently halogen;
(ii) reacting the reaction mixture resulting from step (i) with sulfur; and
(iii-1) reacting the product of step (ii) with a protonating agent, in order to obtain compounds of formula (I), wherein Y is hydrogen; or
(iii-2) reacting the product of step (ii) with an electrophilic compound Y¹-LG in order to obtain compounds of formula (I), wherein Y is Y¹, wherein
Y¹ is (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, C(═S)R⁹, SO₂R¹⁰ or CN; wherein R⁹ and R¹⁰ are as defined above; and
LG is a leaving group;
or
(iv) reacting the reaction mixture resulting from step (i) with an electrophile selected from
(VI) a disulfide R³—S—S—R³, in order to obtain a compound of formula (I), wherein Y is R³;
(VII) R⁴—S—SO₂—R⁴, in order to obtain a compound of formula (I), wherein Y is R⁴; or
(VIII) R⁵—S-Hal, wherein Hal is halogen, in order to obtain a compound of formula (I), wherein Y is R⁵;
wherein
R³, R⁴ are independently from another (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₆-C₁₀)-aryl, C(═S)R⁹ or CN, or a five-, six-, seven-, eight-, nine- or ten-membered aromatic heterocycle that contains one, two, three or four heteroatoms from the group consisting of O, N and S; and
R⁵ is halogen, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₆-C₁₀)-aryl, CN, or a five-, six-, seven-, eight-, nine- or ten-membered aromatic heterocycle that contains one, two, three or four heteroatoms from the group consisting of O, N and S.

16. A process for the preparation of a compound (IIIa) 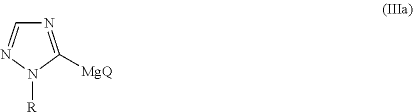
comprising
(i) reacting a triazolo compound of formula (IV) 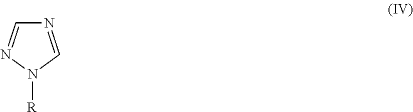
with a reagent
(R¹R²N)MgQ (Va),
wherein
R is an organic group;
Q is (C₁-C₁₀)-alkyl, (C₂-C₁₀)-alkenyl, (C₂-C₁₀)-alkynyl, (C₃-C₈)-cycloalkyl, NR¹R², X¹, X³.zLiX², or (C₆-C₁₀)-aryl, wherein the aryl is unsubstituted or substituted by one, two or three groups independently selected from the group consisting of halogen and (C₁-C₄)-alkyl, and
R¹ and R² are independently selected from the group consisting of (C₁-C₁₀)-alkyl, Si(A¹A²A³), (C₃-C₈)-cycloalkyl and (C₆-C₁₀)-aryl, wherein said groups may bear one, two or three identical or different substituents R^{a}; or
R¹ and R², together with the nitrogen atom to which they are bonded form a five- or six-membered saturated or partially unsaturated heterocyclyl, which is bonded via N and, if it is a six-membered heterocyclyl, which may contain one or two additional heteroatoms selected from the group consisting of O, N and S, and wherein in each case, the heterocyclyl is unsubstituted or carries one, two, three or four substituents selected from the group of halogen, C₁-C₄-alkyl, C₁-C₄-haloalkyl, C₂-C₆-alkenyl, C₂-C₆-haloalkenyl, C₁-C₄-alkoxy, C₁-C₄-alkoxycarbonyl, C₁-C₄-haloalkoxy, C₃-C₆-alkenyloxy, C₃-C₆-haloalkenyloxy and C₆-C₁₀-aryl; wherein
A¹, A², A³ are independently selected from the group consisting of C₁-C₆-alkyl, trimethylsilyl and phenyl;
R^{a} is in each case independently selected from the group consisting of halogen, C₁-C₄-alkyl, C₁-C₄-haloalkyl, C₁-C₄-alkoxy, C₁-C₄-haloalkoxy, C₁-C₄-alkylthio, C₁-C₄-alkylamino and di-C₁-C₄-alkylamino.

17. A compound of formula (IIIa) as defined in claim 15, wherein Q is NR¹R² or X³.zLiX².

18. A compound of formula (IIa) 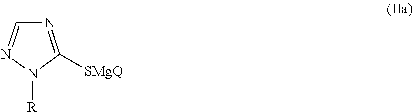
wherein
Q is NR¹R² or X³.zLiX²;
R is an organic group;
R¹ and R² are independently selected from the group consisting of (C₁-C₁₀)-alkyl, Si(A¹A²A³), (C₃-C₈)-cycloalkyl and (C₆-C₁₀)-aryl, wherein said groups may bear one, two or three identical or different substituents R^{a}; or
R¹ and R², together with the nitrogen atom to which they are bonded form a five- or six-membered saturated or partially unsaturated heterocyclyl, which is bonded via N and, if it is a six-membered heterocyclyl, which may contain one or two additional heteroatoms selected from the group consisting of O, N and S, and wherein in each case, the heterocyclyl is unsubstituted or carries one, two, three or four substituents selected from the group of halogen, C₁-C₄-alkyl, C₂-C₆-alkenyl, C₂-C₆-haloalkenyl, C₁-C₄-alkoxy, C₁-C₄-alkoxycarbonyl, C₁-C₄-haloalkoxy, C₃-C₆-alkenyloxy, C₃-C₆-haloalkenyloxy and C₆-C₁₀-aryl; wherein
A¹, A², A³ are independently selected from the group consisting of C₁-C₆-alkyl, trimethylsilyl and phenyl;
R^{a} is in each case independently selected from the group consisting of halogen, C₁-C₄-alkyl, C₁-C₄-haloalkyl, C₁-C₄-alkoxy, C₁-C₄-haloalkoxy, C₁-C₄-alkylthio, C₁-C₄-alkylamino and di-C₁-C₄-alkylamino;
z is >0;
X³, X² are independently halogen.

19. A process for the preparation of a compound of formula (IIa) as defined in claim 18, comprising
(ii) reacting a compound of formula (Ma) 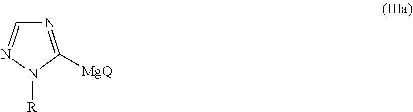
wherein
R is an organic group;
Q is NR¹R² or X³.zLiX²,
R¹ and R² are independently selected from the group consisting of (C₁-C₁₀)-alkyl, Si(A¹A²A³), (C₃-C₈)-cycloalkyl and (C₆-C₁₀)-aryl, wherein said groups may bear one, two or three identical or different substituents R^{a}; or
R¹ and R², together with the nitrogen atom to which they are bonded form a five- or six-membered saturated or partially unsaturated heterocyclyl, which is bonded via N and, if it is a six-membered heterocyclyl, which may contain one or two additional heteroatoms selected from the group consisting of O, N and S, and wherein in each case, the heterocyclyl is unsubstituted or carries one, two, three or four substituents selected from the group of halogen, C₁-C₄-alkyl, C₁-C₄-haloalkyl, C₂-C₆-alkenyl, C₂-C₆-haloalkenyl, C₁-C₄-alkoxy, C₁-C₄-alkoxycarbonyl, C₁-C₄-haloalkoxy, C₃-C₆-alkenyloxy, C₃-C₆-haloalkenyloxy and C₆-C₁₀-aryl; wherein
A¹, A², A³ are independently selected from the group consisting of C₁-C₆-alkyl, trimethylsilyl and phenyl;
R^{a} is in each case independently selected from the group consisting of halogen, C₁-C₄-alkyl, C₁-C₄-haloalkyl, C₁-C₄-alkoxy, C₁-C₄-haloalkoxy, C₁-C₄-alkylthio, C₁-C₄-alkylamino and di-C₁-C₄-alkyl-amino;
z is >0;
X³, X² are independently halogen.
with sulfur to obtain a compound of formula (IIa).

20. The process of claim 15, wherein the reaction is carried out as a one-pot reaction.

21. The process of claim 15, wherein R is a group (1): 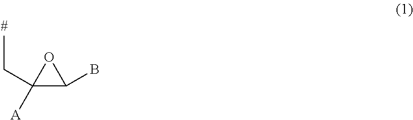
wherein # shall mean the point of attachment to the triazolo group, and;
A or B is a three-, four-, five-, six-, seven-, eight-, nine- or ten-membered saturated or partially unsaturated heterocycle or five-, six-, seven-, eight-, nine- or ten-membered aromatic heterocycle, where the heterocycle contains in each case one, two, three or four heteroatoms from the group consisting of O, N and S; is naphthyl or phenyl;
and the respective other variable B or A has one of the meanings mentioned above for A or B or is C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, naphthyl or benzodioxolyl;
where A and/or B independently of one another are unsubstituted or substituted by one, two, three or four independently selected substituents L; wherein
L is halogen, cyano, nitro, cyanato (OCN), C₁-C₈-alkyl, C₁-C₈-haloalkyl, phenyl-C₁-C₆-alkyloxy, C₂-C₈-alkenyl, C₂-C₈-haloalkenyl, C₂-C₈-alkynyl, C₂-C₈-haloalkynyl, C₄-C₁₀-alkadienyl, C₄-C₁₀-haloalkadienyl, C₁-C₈-alkoxy, C₁-C₈-haloalkoxy, C₁-C₈-alkylcarbonyloxy, C₁-C₈alkylsulfonyloxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkenyl, C₃-C₈-halocycloalkenyl, C₃-C₈-cycloalkoxy, C₃-C₆-cycloalkenyloxy, hydroxyimino-C₁-C₈-alkyl, C₁-C₆-alkylene, oxy-C₂-C₄-alkylene, oxy-C₁-C₃-alkyleneoxy, C₁-C₈-alkoximino-C₁-C₈-alkyl, C₂-C₈-alkenyloximino-C₁-C₈-alkyl, C₂-C₈-alkynyloximino-C₁-C₈-alkyl, S(═O)ₙA⁶, C(═O)A⁷, C(═S)A⁷, NA⁸A⁹, phenyl-C₁-C₈-alkyl, phenyl, phenyloxy or a five- or six-membered saturated, partially unsaturated or aromatic heterocycle which contains one, two, three or four heteroatoms from the group consisting of O, N and S; wherein
n is 0, 1 or 2;
A⁶ is hydrogen, hydroxyl, C₁-C₈-alkyl, C₁-C₈-haloalkyl, amino, C₁-C₈-alkylamino or di-C₁-C₈-alkylamino,
A⁷ is one of the groups mentioned for A¹ or C₂-C₈-alkenyl, C₂-C₈-haloalkenyl, C₂-C₈-alkynyl, C₂-C₈-haloalkynyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkoxy or C₃-C₈-halocycloalkoxy;
A⁸, A⁹ independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, C₃-C₈-cycloalkenyl or C₃-C₈-halocycloalkenyl;
where the aliphatic and/or alicyclic and/or aromatic groups of the radical definitions of L for their part may carry one, two, three or four identical or different groups R^{L}:
R^{L} is halogen, cyano, nitro, C₁-C₈-alkyl, C₁-C₈-haloalkyl, C₁-C₈-alkoxy, C₁-C₈-haloalkoxy, C₃-C₈-cycloalkyl, C₃-C₈-halocycloalkyl, C₃-C₈-cycloalkenyl, C₃-C₈-cycloalkoxy, C₃-C₈-halocycloalkoxy, C₁-C₈-alkylcarbonyl, C₁-C₈-alkylcarbonyloxy, C₁-C₈-alkoxycarbonyl, amino, C₁-C₈-alkylamino, or di-C₁-C₈-alkylamino.

22. The process of claim 21, wherein A is 2,4-difluorophenyl and B is 2-chlorophenyl or A is 4-fluorophenyl and B is 2-chlorophenyl.

23. The process of claim 15, wherein R is a group (2): 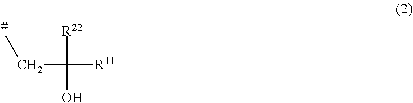
wherein # shall mean the point of attachment to the triazolo group and
R¹¹ and R²² independently of one another are C₁-C₆-alkyl, C₁-C₆-haloalkyl, C₃-C₆-cycloalkyl, C₃-C₆-halocycloalkyl or phenyl, wherein the alkyl, cycloalkyl and phenyl moieties may be unsubstituted or substituted by one, two, three or four substituents L wherein
L is halogen, cyano, nitro, cyanato (OCN), C₁-C₈-alkyl, C₁-C₈-haloalkyl, phenyl-C₁-C₆-alkyloxy, C₂-C₈-alkenyl, C₂-C₈-haloalkenyl, C₂-C₈-alkynyl, C₂-C₈-haloalkynyl, C₄-C₁₀-alkadienyl, C₄-C₁₀-haloalkadienyl, C₁-C₈-alkoxy, C₁-C₈-haloalkoxy, C₁-C₈-alkylcarbonyloxy, C₁-C₈-alkylsulfonyloxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkenyl, C₃-C₈-halocycloalkenyl, C₃-C₈-cycloalkoxy, C₃-C₆-cycloalkenyloxy, hydroxyimino-C₁-C₈-alkyl, C₁-C₆-alkylene, oxy-C₂-C₄-alkylene, oxy-C₁-C₃-alkyleneoxy, C₁-C₈-alkoximino-C₁-C₈-alkyl, C₂-C₈-alkenyloximino-C₁-C₈-alkyl, C₂-C₈-alkynyloximino-C₁-C₈-alkyl, S(═O)ₙA⁶, C(═O)A⁷, C(═S)A⁷, NA⁸A⁹, phenyl-C₁-C₈-alkyl, phenyl, phenyloxy or a five- or six-membered saturated, partially unsaturated or aromatic heterocycle which contains one, two, three or four heteroatoms from the group consisting of O, N and S; wherein
n is 0, 1 or 2;
A⁶ is hydrogen, hydroxyl, C₁-C₈-alkyl, C₁-C₈-haloalkyl, amino, C₁-C₈-alkylamino or di-C₁-C₈-alkylamino,
A⁷ is one of the groups mentioned for A¹ or C₂-C₈-alkenyl, C₂-C₈-haloalkenyl, C₂-C₈-alkynyl, C₂-C₈-haloalkynyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkoxy or C₃-C₈-halocycloalkoxy;
A⁸, A⁹ independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, C₃-C₈-cycloalkenyl or C₃-C₈-halocycloalkenyl; or
R¹¹ and R²², together with the carbon atom to which they are attached, form a five- or six-membered saturated or partially unsaturated ring, that can be unsubstituted or substituted by one, two, three, four or five substituents L′, wherein L′ stands for L as defined above or stands for a group 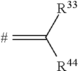
wherein R³³ and R⁴⁴ independently are selected from the group of hydrogen and the meaning for L as defined above.

24. The process of claim 15, wherein R is a group (3): 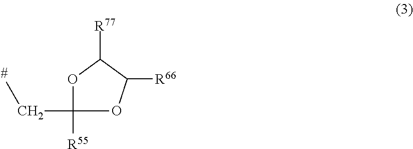
wherein # shall mean the point of attachment to the triazolo group;
R⁵⁵ phenyl-C₁-C₈-alkyl, phenyl or a five- or six-membered saturated, partially unsaturated or aromatic heterocycle which contains one, two, three or four heteroatoms from the group consisting of O, N and S; where the aliphatic and/or aromatic and/or heterocyclic groups for their part may carry one, two, three or four identical or different groups selected from the group consisting of halogen, cyano, nitro, C₁-C₈-alkyl, C₁-C₈-haloalkyl, C₁-C₈alkoxy, C₁-C₈-haloalkoxy, C₃-C₈-cycloalkyl, C₃-C₈-halocycloalkyl, C₃-C₈-cycloalkenyl, C₃-C₈-cycloalkoxy, C₃-C₈-halocycloalkoxy, C₁-C₈-alkylcarbonyl, C₁-C₈-alkylcarbonyloxy, C₁-C₈-alkoxycarbonyl, amino, C₁-C₈-alkylamino, di-C₁-C₈-alkylamino, phenyl, halophenyl, phenyloxy, and halophenyloxy;
R⁶⁶ and R⁷⁷ independently of one another are hydrogen, C₁-C₆-alkyl, C₁-C₆-haloalkyl, C₃-C₆-cycloalkyl, C₃-C₆-halocycloalkyl or phenyl, wherein the alkyl, cycloalkyl or phenyl moieties may be unsubstituted or substituted by one, two or three substituents selected from halogen, cyano, nitro, C₁-C₈-alkyl, C₁-C₈-haloalkyl, C₁-C₈-alkoxy, or C₁-C₈-haloalkoxy.

25. The process of claim 15, wherein R is a group (4): 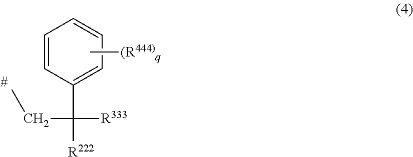
wherein # shall mean the point of attachment to the triazolo group;
R²²² and R³³³ are independently selected from hydrogen, cyano, C₁-C₆-alkyl or C₁-C₆-haloalkyl, wherein the alkyl moieties may be unsubstituted or substituted by one, two, three or four substituents L;
wherein
L is halogen, cyano, nitro, cyanato (OCN), C₁-C₈-alkyl, C₁-C₈-haloalkyl, phenyl-C₁-C₆-alkyloxy, C₂-C₈-alkenyl, C₂-C₈-haloalkenyl, C₂-C₈-alkynyl, C₂-C₈-haloalkynyl, C₄-C₁₀-alkadienyl, C₄-C₁₀-haloalkadienyl, C₁-C₈-alkoxy, C₁-C₈-haloalkoxy, C₁-C₈-alkylcarbonyloxy, C₁-C₈-alkylsulfonyloxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkenyl, C₃-C₈-halocycloalkenyl, C₃-C₈-cycloalkoxy, C₃-C₆-cycloalkenyloxy, hydroxyimino-C₁-C₈-alkyl, C₁-C₆-alkylene, oxy-C₂-C₄-alkylene, oxy-C₁-C₃-alkyleneoxy, C₁-C₈-alkoximino-C₁-C₈-alkyl, C₂-C₈-alkenyloximino-C₁-C₈-alkyl, C₂-C₈-alkynyloximino-C₁-C₈-alkyl, S(═O)ₙA⁶, C(═O)A⁷, C(═S)A⁷, NA⁸A⁹, phenyl-C₁-C₈-alkyl, phenyl, phenyloxy or a five- or six-membered saturated, partially unsaturated or aromatic heterocycle which contains one, two, three or four heteroatoms from the group consisting of O, N and S; wherein
n is 0, 1 or 2;
A⁶ is hydrogen, hydroxyl, C₁-C₈-alkyl, C₁-C₈-haloalkyl, amino, C₁-C₈-alkylamino or di-C₁-C₈-alkylamino,
A⁷ is one of the groups mentioned for A¹ or C₂-C₈-alkenyl, C₂-C₈-haloalkenyl, C₂-C₈-alkynyl, C₂-C₈-haloalkynyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkoxy or C₃-C₈-halocycloalkoxy;
A⁸, A⁹ independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, C₃-C₈-cycloalkenyl or C₃-C₈-halocycloalkenyl;
q is one, two three or five; and
R⁴⁴⁴ are independently selected from L as defined above.
